(19) United States
(12) Patent Application Publication
(10) Pub. No.: US 2025/0043306 A1
(43) Pub. Date: Feb. 6, 2025

(54) GENE-EDITING METHODS FOR GENETIC IMPROVEMENT IN LIVESTOCK

(71) Applicant: ABS Global, Inc., DeForest, WI (US)

(72) Inventors: Andrew Mark Cigan, Madison, WI (US); Brian Timothy Burger, Madison, WI (US)

(73) Assignee: ABS Global, Inc., DeForest, WI (US)

(21) Appl. No.: 18/364,935

(22) Filed: Aug. 3, 2023

(51) Int. Cl. C12N 15/85; A01K 67/027; C12N 9/22; C12N 15/11; C12N 15/90

(52) U.S. Cl. CPC C12N 15/8509; A01K 67/0275; C12N 9/22; C12N 15/11; C12N 15/907; A01K 2217/07; A01K 2227/101; A01K 2227/108; C12N 2310/20; C12N 2800/80

(57) ABSTRACT

Disclosed are gene edited ungulate animals and methods of editing genes that control various ungulate traits. Such methods include CRISPR, zinc fingers, or TALENS. Exemplary traits for editing include polled, sterility or fertility, milk production, growth (which increases meat production), fat production, conception rates, stillborn rates, calving ease, or content of produced milk such as the amount of protein or the amount of fat. Other traits include backfat thickness, intramuscular fat, ultrasound loin muscle area, loin muscle area and intramuscular fat content, chest circumference, withers height, body length, hip height, rump length, and heart girth. Other exemplary native traits include, but are not limited to, high altitude adaptation and response to hypoxia, cold acclimation, body size and stature, resistance to disease and bacterial infection, reproduction, milk yield and components, and feed efficiency.

### INCORPORATION OF THE SEQUENCE LISTING

The Sequence Listing, including the file named TD-10-2023-US1-SEQLST.xml, is hereby incorporated by reference in its entirety.

### TECHNICAL FIELD

The present technology relates generally to methods for improving genetic traits or fitness in livestock animals, particularly in ungulates such as pigs and cattle via gene editing and edited animals thereof. In particular, the methods comprise using various systems to target genes of interest in animal cells for the purpose of promoting pathogen resistance, fertility, lactation, and native traits that support more rapid growth or feed efficiency.

### BACKGROUND

Gene editing is a group of molecular biology techniques that allow scientists to make precise changes in the genome of an organism. Prior to the introduction of these techniques, genomic changes could only be made in organisms by replacing sections of DNA using homologous recombination. This technique, while reasonably efficient for unicellular organisms and mice, was not feasible for other organisms such as Drosophila. The need for complete genomic replacement of a region also allowed for higher error rates, and was more efficient at knocking out gene function than making precise changes, such as preventing an endogenous protein from interacting with a virus while preserving the endogenous function of the gene or enhancing an endogenous trait.

### SUMMARY OF THE PRESENT TECHNOLOGY

In general, the present teachings provide methods of editing target genes in ungulate livestock. In some embodiments, the present teachings can include an ungulate comprising one or more edits in one or more genes that affects a livestock trait. In some configurations, the ungulate can be a porcine animal. In various configurations, the ungulate can be a bovine animal. In various configurations, the ungulate can be a pig. In various configurations, the ungulate can be a Bos taurus animal. In various configurations, the one or more genes can be a PRLR, NANOS2, Deadend (Dnd), APAF1, SMC2, GART, TFBIM, SIRT1, SIRT2, LPL, CRTC2, SIX4, UCP2, UCP3, URB1, EVAIC, TMEM68, TGS1, LYN, XKR4, FOXA2, GBP2, GBP5, FGD6, NPC1L1, NUDCD3, ACSS1, FCHSD2, PPPIR12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, TIGAR, CD18, ANP32, ANPEP, TMPRSS1, TMPRSS2, NANOS2, CD163, Melanocortin-4 receptor (MC4R), HMGA, IGF2, HAL, RN, Mx1, BAT2, EHMT2, PRDM1, PRDM14, or ESR. In various configurations, the livestock trait can be a high altitude adaptation and response to hypoxia, cold acclimation, body size and stature, resistance to disease and bacterial infection, reproduction, milk yield and components, growth and feed efficiency, or polled phenotype.

In various configurations, the present teachings can include a method of editing a gene affecting a livestock trait of an ungulate comprising: contacting an editing reagent with an isolated cell of the ungulate. In some configurations, the editing reagent can comprise a DNA editing system selected from the group consisting of a meganuclease, a zinc finger nucleases (ZFN), a transcription-activator like effector nuclease (TALEN) and CRISPR. In various configurations, the editing reagent can comprise one or more gRNAs and a CAS9 protein or a polynucleotide encoding a CAS9 protein. In various configurations, the editing reagent can comprise a ribonucleoprotein complex comprising a gRNA and a CAS9 protein. In various configurations, the ungulate can be a bovine animal. In various configurations, the ungulate can be a porcine animal. In various configurations, the ungulate can be a swine. In various configurations, the ungulate can be a Bos taurus, Bos indicus, or Bubalus bubalis animal. In various configurations, the ungulate can be a pig.

In various embodiments, the present disclosure provides a gene edited ungulate produced by any and all embodiments of the methods disclosed herein.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Recent advances in genome editing techniques have made it possible to alter DNA sequences in living cells by editing a few nucleotides in cells of organisms such as by creating a site-specific DNA double-strand break (DSB) in the genome and then allowing the cell's endogenous DSB repair machinery to fix the break. This repair can comprise non-homologous end-joining (NHEJ) or homologous recombination (HR) (Porteus, M., Annu Rev Pharmacol Toxicol., 2016, 56, 163-190). NHEJ directly joins the DNA ends in a double-stranded break (DSB) with or without minimal ends trimming, while HR utilizes a homologous donor sequence as a template for copying the missing DNA sequence at the break site. Three primary approaches use genome editing (NHEJ) of cells as potential therapeutics: (a) knocking out functional genetic elements by creating spatially precise insertions or deletions, (b) creating insertions or deletions that compensate for underlying frameshift mutations; hence reactivating partly- or non-functional genes, and (c) creating defined genetic deletions. Alternatively or in addition, the ends of a defined deletion can be designed to form a particular sequence such as a stop codon. Currently the four major types of therapeutic applications to HR-mediated genome editing are: (a) gene correction (i.e. correction of diseases that are caused by point mutations in single genes), (b) functional gene correction (i.e. correction of diseases that are caused by mutations scattered throughout the gene), (c) safe harbor gene addition (i.e. when precise regulation is not required or when supra physiologic levels of a therapeutic transgene are desired), and (d) targeted transgene addition (i.e. when precise regulation is required) (Porteus, M., Annu Rev Pharmacol Toxicol., 2016, 56, 163-190).

### Definitions

Throughout this application, various embodiments of the present technology may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the term “effective amount” refers to an amount of a biologically active agent that is sufficient to elicit a desired biological response.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the terms “individual” or “subject” can be an individual organism, a vertebrate, or a non-human mammal. In some embodiments, the individual, patient or subject is a non-human animal.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons. Nucleic acids may be naturally occurring, for example, in the context of a genome, a transcript, an mRNA, RNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “ungulate” refers to a diverse group of large mammals that includes but is not limited to equines, bovines/cattle, porcines/swine, goats, buffalo, sheep, giraffes, camels, deer, and hippopotamuses. Most terrestrial ungulates use the tips of their toes, usually hoofed, to sustain their whole body weight while moving. In some embodiments, the term means, roughly, “being hoofed” or “hoofed animal.”

### Gene Editing Methods of the Present Technology

The present disclosure provides methods for improving a genetic trait or improve fitness in a non-human animal subject comprising contacting a eukaryotic cell of the non-human animal subject with an effective amount of a DNA editing agent.

The non-human animal subject may be an ungulate. The methods disclosed herein are useful for generating gene edited ungulates, such as pigs and cattle, that show an improvement in one or more genetic traits or fitness without negatively affecting survival of the ungulate animals.

Additionally or alternatively, in some embodiments, the eukaryotic cell may be a primary cell, a cell line, a somatic cell, a germ cell, a stem cell, an embryonic stem cell, an adult stem cell, a hematopoietic stem cell, a mesenchymal stem cell, an induced pluripotent stem cell (iPSC), a gamete cell, a zygote, a blastocyst cell, an embryonic cell, a fetal cell, or a donor cell. In some embodiments, the eukaryotic cell is isolated from its natural environment. Additionally or alternatively, in some embodiments, the eukaryotic cell is a healthy cell, an immune cell (e.g. T cell, B cell, macrophage, NK cell, etc.) or a cell infected by a pathogen (e.g. by a bacterial, viral or fungal pathogen).

Following is a description of various non-limiting examples of methods and DNA editing agents used to introduce nucleic acid alterations to a gene encoding a non-coding RNA molecule (e.g. RNA silencing molecule) and agents for implementing same that can be used according to specific embodiments of the present disclosure.

### Targeted Integration of a Nucleic Acid at a Desired Locus

Site-specific integration of an exogenous nucleic acid at a particular locus may be accomplished by any technique known to those of skill in the art. In some embodiments, integration of an exogenous nucleic acid at a locus comprises contacting a cell with a nucleic acid molecule comprising the exogenous nucleic acid. In various embodiments, such a nucleic acid molecule may comprise nucleotide sequences flanking the exogenous nucleic acid that facilitate homologous recombination between the nucleic acid molecule and the desired locus. In particular embodiments, the nucleotide sequences flanking the exogenous nucleic acid that facilitate homologous recombination may be complementary to endogenous nucleotides of the targeted locus. In particular examples, the nucleotide sequences flanking the exogenous nucleic acid that facilitate homologous recombination may be complementary to previously integrated exogenous nucleotides.

Integration of a nucleic acid at a targeted locus may be facilitated in some embodiments by endogenous cellular machinery of a host cell, such as, for example and without limitation, endogenous DNA and endogenous recombinase enzymes. In some embodiments, integration of a nucleic acid at a targeted locus may be facilitated by one or more factors that are provided to a host cell. For example, nuclease(s), recombinase(s), and/or ligase polypeptides may be provided (either independently or as part of a chimeric polypeptide) by contacting the polypeptides with the host cell, or by expressing the polypeptides within the host cell. Accordingly, in some examples, a nucleic acid comprising a nucleotide sequence encoding at least one nuclease, recombinase, and/or ligase polypeptide may be introduced into the host cell, either concurrently or sequentially with a nucleic acid to be integrated site-specifically at a targeted locus, wherein the at least one nuclease, recombinase, and/or ligase polypeptide is expressed from the nucleotide sequence in the host cell.

Genome Editing using engineered endonucleases—this approach refers to a reverse genetics method using artificially engineered nucleases to typically cut and create specific double-stranded breaks (DSBs) at a desired location(s) in the genome, which are then repaired by cellular endogenous processes such as, homologous recombination (HR) or non-homologous end-joining (NHEJ). In order to introduce specific nucleotide modifications to the genomic DNA, a donor DNA repair template containing the desired sequence must be present during HR. In general, donor DNA repair templates are exogenously provided single stranded or double stranded DNA.

Gene editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single or double stranded breaks (DSBs), several distinct classes of nucleases have been discovered and bioengineered to date. These include the meganucleases, zinc finger nucleases (ZFNs), transcription-activator like effector nucleases (TALENs), and CRISPR/Cas9 system and variants thereof.

Meganucleases-Meganucleases are commonly grouped into four families: the LAGLIDADG family, the GIY-YIG family, the His-Cys box family, and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks (DSBs) in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see, e.g., U.S. Pat. No. 8,021,867). Meganucleases can be designed using the methods described in e.g. Certo, M. T., et al., Nature Methods, 2012, 9, 973-975; U.S. Pat. Nos. 8,304,222; 8,021,867; 8,119,381; 8,124,369; 8,129,134; 8,133,697; 8,143,015; 8,143,016; 8,148,098; or 8,163,514; the contents of each are incorporated herein by reference in their entirety. Alternatively, meganucleases with site specific cutting characteristics can be obtained using commercially available technologies such as Precision Biosciences' DIRECTED NUCLEASE EDITOR™ genome editing technology.

ZFNs and TALENs-Two distinct classes of engineered nucleases, zinc-finger nucleases (ZFNs) and transcription activator-like effector nucleases (TALENs), have both proven to be effective at producing targeted double-stranded breaks (DSBs) (Christian, M., et al., Genetics, 2010, 186, 757-761; Kim, Y. G., et al., Proc. Natl. Acad. Sci. USA, 1996, 93, 1156-1160; Li, T., et al., Nucleic Acids Res., 2011, 39, 359-372; Mahfouz, M. M., et al., Proc. Natl. Acad. Sci. USA, 2011, 108, 2623-2628; Miller, J. C., et al., Nat. Biotechnol., 2011, 29, 143-148).

ZFNs and TALENs restriction endonuclease technology utilizes a non-specific DNA cutting enzyme which is linked to a specific DNA binding domain (either a series of zinc finger domains or TALE repeats, respectively). Typically a restriction enzyme that has a separate DNA recognition site is selected. The cleaving portion is separated and then linked to a DNA binding domain, thereby yielding an endonuclease with very high specificity for a desired sequence. An exemplary restriction enzyme with such properties is Fok I. Additionally, Fok I has the advantage of requiring dimerization to have nuclease activity and this means the specificity increases dramatically as each nuclease partner can be engineered to recognize a unique DNA sequence. To enhance this effect, Fok I nucleases have been engineered that can only function as heterodimers and have increased catalytic activity. The heterodimer functioning nucleases avoid the possibility of unwanted homodimer activity and thus increase specificity of the double-stranded break (DSB).

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fok I domains heterodimerize to create a double-stranded break (DSB). Repair of these double-stranded breaks (DSBs) through the non-homologous end-joining (NHEJ) pathway often results in small deletions or small sequence insertions (indels). Since each repair made by NHEJ is unique, the use of a single nuclease pair can produce an allelic series with a range of different insertions or deletions at the target site.

In general NHEJ is relatively accurate—about 85% of DSBs in human cells are repaired by NHEJ within about 30 min of detection. In gene editing, erroneous NHEJ can be relied upon as when the repair is accurate the nuclease will keep cutting until the recognition, cut site, or PAM motif or the transiently introduced nuclease is no longer present.

The deletions typically range anywhere from a few base pairs to a few hundred base pairs in length, but larger deletions have been successfully generated in cell culture by using two pairs of nucleases simultaneously (Carlson, D. F., et al., Proc. Natl. Acad. Sci. USA, 2012, 109:17382-173827; Lee, H. J., et al., Genome Res., 2010, 20, 81-89). In addition, when a fragment of DNA with homology to the targeted region is introduced in conjunction with the nuclease pair, the double-stranded break (DSB) can be repaired via homologous recombination (HR) to generate specific modifications (Li, T., et al., Nucleic Acids Res., 2011, 39, 359-372; Miller, J. C., et al., Nat. Biotechnol., 2011, 29, 143-148; Urnov, F. D., et al., Nature, 2005, 435, 646-651).

Although the nuclease portions of both ZFNs and TALENs have similar properties, the difference between these engineered nucleases is in their DNA recognition peptide. ZFNs rely on Cys₂-His₂ zinc fingers and TALENs on TALEs. Both of these DNA recognizing peptide domains have the characteristic that they are naturally found in combinations in their proteins. Cys₂-His₂ zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include modular assembly (where zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from vendors such as SANGAMO BIOSCIENCES™ (Richmond, CA).

Methods for designing and obtaining TALENs are described in the literature, for example in Reyon, D., et al., Nature Biotechnology, 2012, 30, 460-465; Miller, J. C., et al., Nat. Biotechnol., 2011, 29, 143-148; Cermak, T., et al., Nucleic Acids Research, 2011, 39, e82; and Zhang, F., et al., Nature Biotechnology, 2011, 29, 149-153. A recently developed web-based program named Mojo Hand was introduced by Mayo Clinic for designing TAL and TALEN constructs for genome editing applications (can be accessed through www.talendesign.org). TALEN can also be designed and obtained commercially from vendors such as SANGAMO BIOSCIENCES™ (Richmond, CA).

T-GEE system (TargetGene's Genome Editing Engine)—A programmable nucleoprotein molecular complex containing a polypeptide moiety and a specificity conferring nucleic acid (SCNA) which assembles in vivo, in a target cell, and is capable of interacting with the predetermined target nucleic acid sequence is provided. The programmable nucleoprotein molecular complex is capable of specifically editing a target site within the target nucleic acid sequence or modifying the function of the target nucleic acid sequence. The nucleoprotein composition can comprise (a) a polynucleotide molecule encoding a chimeric polypeptide that can comprise (i) a functional domain capable of modifying the target site and (ii) a linking domain that is capable of interacting with a specificity conferring nucleic acid; and (b) a specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base pairing of specificity conferring nucleic acid and a target nucleic acid. The composition is less genotoxic, modular in their assembly, utilize single platform without customization, practical for independent use outside of specialized core-facilities, and has shorter development time frame and reduced costs.

Triplex-forming Oligonucleotides (TFOs) Triplex-forming oligonucleotides (TFOs) are defined as oligonucleotides which bind as third strands to duplex DNA in a sequence specific manner. The oligonucleotides can be synthetic or isolated nucleic acid molecules which selectively bind to or hybridize with a predetermined target sequence, target region, or target site within or adjacent to a gene so as to form a triple-stranded structure.

The triplex-forming molecules can be used to induce site-specific homologous recombination in mammalian cells when combined with donor oligonucleotide (DNA) molecules. The donor DNA molecules can contain edited nucleic acids relative to the target DNA sequence. This is useful to activate, inactivate, or otherwise alter the function of a polypeptide or protein encoded by the targeted duplex DNA.

The triplex-forming molecules bind to a predetermined target region referred to herein as the “target sequence,” “target region,” or “target site.” The target sequence for the triplex-forming molecules can be within or adjacent to a target gene for editing as described herein. The target sequence can be within the coding DNA sequence of the gene or within an intron. The target sequence can also be within DNA sequences which regulate expression of the target gene, including promoter or enhancer sequences or sites that regulate RNA splicing.

Triplex forming molecules are described in more detail below and in U.S. Pat. Nos. 5,962,426, 6,303,376, 7,078,389, 7,279,463, 8,658,608, U.S. Published Application Numbers 2003/0148352, 2010/0172882, 2011/0268810, 2011/0262406, 2011/0293585, and published PCT application numbers WO 1995/001364, WO 1996/040898, WO 1996/039195, WO 2003/052071, WO 2008/086529, WO 2010/123983, WO 2011/053989, WO 2011/133802, WO 2011/13380, WO 2017/143042, WO 2017/143061, WO 2018/187493, WO 2020/257776, Rogers, F. A., et al., Proc. Natl. Acad. Sci USA, 2002, 99, 16695-16700, Majumdar, A., et al., Nature Genetics, 1998, 20, 212-214, Chin, J. Y., et al., Proc. Natl. Acad. Sci. USA, 2008, 105, 13514-13519, and Schleifman, E. B., et al., Chem Biol., 2011, 18, 1189-1198.

The triplex forming molecules are typically single stranded and bind to a double stranded nucleic acid molecule, for example duplex DNA, in a sequence-specific manner to form a triple-stranded structure. The single-stranded oligonucleotide/oligomer typically includes a sequence substantially complementary to the polypurine strand of the polypyrimidine:polypurine target motif.

The nucleobase sequence of the oligonucleotides/oligomer can be selected based on the sequence of the target sequence, the physical constraints imposed by the need to achieve binding of the oligonucleotide/oligomer within the major groove of the target region, and the need to have a low dissociation constant (K_{d}) for the oligo/target sequence complex. The oligonucleotides/oligomers have a nucleobase composition which can be conducive to triple-helix formation and can be generated based on one of the known structural motifs for third strand binding such as Hoogsteen binding. Stable complexes can be formed on polypurine:polypyrimidine elements, which are relatively abundant in mammalian genomes. Triplex formation by TFOs can occur with the third strand oriented either parallel or anti-parallel to the purine strand of the nucleic acid duplex. In the anti-parallel, purine motif, the triplets are G.G:C and A.A:T, whereas in the parallel pyrimidine motif, the canonical triplets are C+.G:C and T.A:T. The triplex structures can be stabilized by one, two or three Hoogsteen hydrogen bonds (depending on the nucleobase) between the bases in the TFO strand and the purine strand in the duplex. A review of base compositions and binding properties for third strand binding oligonucleotides is provided in, for example, U.S. Pat. No. 5,422,251. Preferably, the oligonucleotide/oligomer binds to or hybridizes to the target sequence under conditions of high stringency and specificity. In some embodiments, the oligonucleotides/oligomers bind in a sequence specific manner within the major groove of duplex DNA. Reaction conditions for in vitro triple helix formation of an oligonucleotide/oligomer to a double stranded nucleic acid sequence vary from oligo to oligo, depending on factors such as polymer length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, a triplex forming molecule is said to be substantially complementary to a target region when the oligonucleotide has a nucleobase composition which allows for the formation of a triple-helix with the target region. As such, an oligonucleotide/oligomer can be substantially complementary to a target region even when there are non-complementary bases present in the oligonucleotide/oligomer. As stated above, there are a variety of structural motifs available which can be used to determine the nucleobase sequence of a substantially complementary oligonucleotide/oligomer.

Protein Nucleic Acid (PNA) gene editing Exemplary triplex forming molecules include peptide nucleic acids. Peptide nucleic acids are polymeric molecules in which the sugar phosphate backbone of an oligonucleotide has been replaced in its entirety by repeating substituted or unsubstituted N-(2-aminoethyl)-glycine residues that are linked by amide bonds-similar to those found in proteins or other polypeptides. The various nucleobases can be linked to the backbone by methylene carbonyl linkages. Peptide nucleic acids can be prepared using known methodologies, generally as adapted from peptide synthesis processes.

PNAs maintain spacing of the nucleobases in a manner that is similar to that of an oligonucleotide (DNA or RNA), but because the sugar phosphate backbone has been replaced, classic (unsubstituted) PNAs can be achiral and neutrally charged molecules. Peptide nucleic acid oligomers can be composed of peptide nucleic acid residues. The nucleobases within each PNA residue can include any of the standard bases (uracil, thymine, cytosine, adenine and guanine) or any modified heterocyclic nucleobase (see WO 2020/257776 for detailed descriptions of modified heterocyclic nucleobases). PNA oligomers can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand. Common modifications to PNA oligomers are discussed in Sugiyama, T. and Kittaka, A., 2013, Molecules, 18, 287-310 and Sahu, B., et al., J. Org. Chem., 2011, 76, 5614-5627, each of which are specifically incorporated by reference in their entireties. These modifications include, but are not limited to, incorporation of charged amino acid residues such as lysine at the termini or in the interior part of the oligomer, inclusion of polar groups in the backbone, a carboxymethylene bridge in the nucleobases, chiral PNA oligomers bearing substituents on the original N-(2-aminoethyl)glycine backbone, replacement of the original aminoethylglycyl backbone skeleton with a negatively-charged scaffold, conjugation of high molecular weight polyethylene glycol (PEG) to one of the termini, fusion of a PNA oligomer to DNA to generate a chimeric oligomer, redesign of the backbone architecture, or conjugation of PNA to DNA or RNA. These modifications improve solubility but often result in reduced binding affinity and/or sequence specificity.

Triplex-forming peptide nucleic acids (PNAs) loaded into biodegradable nanoparticles (NPs) have been explored as a tool for editing of the CFTR gene (McNeer, N. A., et al., Nat Commun., 2015, 6, 6952; Fields, R. J., et al., Adv. Healthc. Mater., 2015, 4, 361-366). PNAs can bind to duplex DNA via both Hoogsteen hydrogen bonding and Watson-Crick bonding to form PNA/DNA/PNA triple helices (with a displaced DNA strand) in a sequence-specific manner (Nielsen, P. E., Current opinion in molecular therapeutics, 2010, 12, 184-191). The formation of a site-directed triple helix by a PNA creates a helical alteration that provokes DNA repair and stimulates DNA recombination in the region of the triplex (Rogers, F. A., et al., Proc. Natl. Acad. Sci. USA, 2002, 99, 16695-16700). Improved gene editing results have been achieved by using tail clamp PNA (tcPNA) designs which incorporate an extended Watson-Crick binding domain (McNeer, N. A., et al., Nat Commun., 2015, 6, 6952; Fields, R. J., et al., Adv. Healthc. Mater., 2015, 4, 361-366; McNeer, N. A., et al., Mol. Ther., 2011, 19, 172-180; McNeer, N. A., et al., Gene Ther., 2013, 20, 658-669; Schleifman, E. B., et al., Mol. Ther. Nucleic Acids, 2013, 2, e135; Bahal, R., et al., Nat. Commun., 7, 2016, 13304; Quijano, E., et al., Yale J. Biol. Med., 2017, 90, 583-598; Ricciardi, A. S., et al., Nat. Commun., 2018, 9, 2481; Ricciardi, A. S., et al., Molecules, 2018, 23, 632). Triplex-forming molecules including peptide nucleic acid (PNA) oligomers with or without a substitution at the gamma position of one or more of PNA residues of the Hoogsteen binding segment, and optionally the Watson-Crick binding segment, of a PNA oligomer are contemplated.

PNAs can bind to DNA via Watson-Crick hydrogen bonds with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules (see Bentin, T., et al., Nucl. Acids Res., 2006, 34, 5790-5799 and Hansen, M. E., et al., Nucl. Acids Res., 2009, 37, 4498-4507), or two PNA molecules linked together by a linker of sufficient flexibility to form a single bis-PNA molecule (See: U.S. Pat. No. 6,441,130). In both cases, the PNA molecule(s) forms a triplex “clamp” with one of the strands of the target duplex while displacing the other strand of the duplex target. In this structure, one strand forms Watson-Crick base pairs with the DNA strand in the antiparallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

In some embodiments, the oligonucleotide can be a single-stranded peptide nucleic acid molecule between 7 and 40 nucleotides in length; frequently the peptide nucleic acid molecule can be 10 to 20 nucleotides in length for in vitro editing and 20 to 30 nucleotides in length for in vivo editing. The nucleobase (sometimes referred to herein simply as “base”) composition in the oligonucleotide may be homopurine or homopyrimidine. Alternatively, the nucleobase composition in the oligonucleotide may be polypurine or polypyrimidine. However, other compositions are also useful.

Suitable molecules for use in linkers of bis-PNA molecules include, but are not limited to, 8-amino-3,6-dioxaoctanoic acid, referred to as an O-linker, and 6-aminohexanoic acid. Poly(ethylene)glycol monomers can also be used in bis-PNA linkers. A bis-PNA linker can contain multiple linker residues in any combination of two or more of the foregoing.

PNAs can also include other positively charged moieties to increase the solubility of the PNA and increase the affinity of the PNA for duplex DNA. Commonly used positively charged moieties include the amino acids lysine and arginine as additional substituents attached to the C- or N-terminus of the PNA oligomer (or a segment thereof) or as a side-chain modification of the backbone (see Huang, H., et al., Arch. Pharm. Res., 2012, 35, 517-522 and Jain, D., et al., 2014, J. Org. Chem., 79, 9567-9577), although other positively charged moieties may also be useful (See U.S. Pat. No. 6,326,479). In some embodiments, the PNA oligomer can have one or more ‘miniPEG’ side chain modifications of the backbone (see, U.S. Pat. No. 9,193,758 and Sahu, B., et al., J. Org. Chem., 2011, 76, 5614-5627).

Alternatively or in addition, any triplex-forming sequence can be modified to include guanidine-G-clamp (“G-clamp”) PNA residues(s) to enhance PNA oligomer binding to a target site, wherein the G-clamp is linked to the backbone as any other nucleobase would be. gPNAs with substitution of cytosine by G-clamp (9-(2-guanidinoethoxy) phenoxazine), a cytosine analog that can form five H-bonds with guanine, and can also provide extra base stacking due to the expanded phenoxazine ring system and substantially increased binding affinity. In vitro studies indicate that a single G-clamp substitution for C can substantially enhance the binding of a PNA-DNA duplex by 23 C (Kuhn, H., et al., Artificial DNA, PNA & XNA, 2010, 1, 45-53). As a result, gPNAs containing G-clamp substitutions can have further increased activity.

CRISPR-Cas system (also referred to herein as “CRISPR”)—Many bacteria and archaea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form a RNA/protein complex and together are sufficient for sequence-specific nuclease activity: the Cas9 nuclease, a crRNA containing 20 base pairs of homology to the target sequence, and a trans-activating crRNA (tracrRNA) (Jinek, M., et al., Science, 2012, 337, 816-821).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded breaks (DSBs) in a variety of different species (Cho, S. W., et al., Genetics, 2013, 195, 1177-1180; Cong, L., et al., Science, 2013, 339, 819-823; DiCarlo, J. E., et al., Nucleic Acids Res., 2013, 41, 4336-43; Hwang, W. Y., et al., Nat. Biotechnol., 2013, 31, 227-229.; Hwang, W. Y., et al., PLOS One, 2013, 8, e68708; Jinek, M., et al., eLife. 2013; 2: e00471; Mali, P., et al., Science, 2013, 339, 823-826).

The CRISPR/Cas system for genome editing contains two distinct components: a gRNA and a CRISPR endonuclease. A variety of CRISPR endonucleases are available for use in conjunction with the gRNA. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof.

The gRNA (also referred to herein as single guide RNA (sgRNA)) is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks (DSBs) in the genomic DNA.

A significant advantage of CRISPR/Cas is that the high efficiency of this system is coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain—either inactive RuvC or inactive HNH—are called ‘nickases’. With only one active nuclease domain, the Cas9 nickase cuts only one strand of the target DNA, creating a single-strand break or ‘nick’. A single-strand break, or nick, is mostly repaired by the single strand break repair mechanism-including proteins such as PARP and the XRCC1/LIG III complex. If a single strand break (SSB) is generated by topoisomerase I, poisons, or by drugs that trap PARP1 on naturally occurring SSBs, then these SSBs could persist. When the cell enters into S-phase and the replication fork encounters such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick, which is basically a non-parallel DSB, can be repaired like other DSBs by HR or NHEJ depending on the presence of a donor sequence and the cell cycle stage-HR occurs at a much lower frequency and only during the S and G2 stages of the cell cycle. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that are not likely to change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Base editing-Base editing applications, such as those disclosed in US Patent number U.S. Pat. No. 11,053,481B2, use fusion proteins that comprise a) a Cas9 domain that binds to a guide RNA, which, in turn, binds a target nucleic acid sequence via strand hybridization and b) a DNA-editing domain which chemically changes the nucleotide base. For example, the DNA-editing domain can be a deaminase domain that deaminates a nucleobase, such as cytidine. The deamination of a nucleobase by a deaminase can lead to a point mutation at the targeted residue. The deamination of cytosine (C) is catalyzed by cytidine deaminases and results in uracil (U), which has the base-pairing properties of thymine (T). Thus, deaminating a cytosine results in a uracil base in RNA and a thymidine base in DNA. Alternatively, guanine (G) deaminases remove an amine group from guanine (G) to create adenine (A). Fusion proteins comprising a Cas9 variant or domain and a DNA editing domain can thus be used for the targeted editing of nucleic acid sequences. Such fusion proteins are useful for targeted editing of DNA in vitro for the generation of cells or animals with desirable traits. Typically, the Cas9 domain of the fusion proteins described herein does not have any nuclease activity but instead is a Cas9 fragment or a dCas9 protein or domain. Base editors may also include proteins or protein domains that alter cellular DNA repair processes to increase the efficiency and stability of the resulting single-nucleotide change, for example a Uracil DNA glycosylase inhibitor (UGI) domain. Two classes of base editors have been generally described to date: cytidine base editors convert target C·G base pairs to T·A base pairs, and adenine base editors convert A·T base pairs to G·C base pairs. Collectively, these two classes of base editors enable the targeted installation of all four transition mutations (C-to-T, G-to-A, A-to-G, and T-to-C) by targeting either DNA strand.

Additionally, more sophisticated paths may be taken to make other mutations, such as those discussed in published application US2021/0230577A1: a C to G base editor can include a fusion protein containing a nucleic acid programmable DNA binding protein domain such as a Cas9 domain, a uracil DNA glycosylase (UDG) domain, and a cytidine deaminase domain. Without wishing to be bound by any particular theory, such a base editing fusion protein is capable of binding to a specific nucleic acid sequence (e.g., via the Cas9 domain), deaminating a cytosine within the nucleic acid sequence to a uridine, which can then be excised from the nucleic acid molecule by UDG. The nucleobase opposite the abasic site can then be replaced with another base (e.g., cytosine), for example by an endogenous translesion polymerase. Typically, cellular base repair machinery replaces a nucleobase opposite an abasic site with a cytosine, although other bases may replace a nucleobase opposite an abasic site. Furthermore, it was found that incorporating a translesion polymerase into the base editor can increase the cytosine incorporation opposite an abasic site. Accordingly, base editors were engineered to incorporate various translesion polymerases to improve base editing efficiency. Translesion polymerases that increase the preference for C integration opposite an abasic site can improve C to G nucleobase editing. It should be appreciated that other translesion polymerases that preferentially integrate non-C nucleobases, may be used to generate alternative base substitutions.

Alternatively, or in addition, base editing fusion proteins may include a nucleic acid programmable DNA binding protein such as a Cas9 domain, and a base excision enzyme that removes a nucleobase. For example, rather than deaminating a cytosine to uridine and excising the uridine using a UDG, as described above, a base editor may include a base excision enzyme that recognizes and removes a nucleobase such as a cytosine or a thymine without first deaminating it. Accordingly, base editors have been engineered by fusing a nucleic acid programmable DNA binding protein to a base excision enzyme that removes cytosine or thymine from a nucleic acid molecule. Furthermore, as with the base editor described above, translesion polymerases were incorporated into this base editor to increase the cytosine incorporation opposite an abasic site generated by the base excision enzyme of the base editor.

Exemplary base editing fusion proteins include the following: (i) a nucleic acid programmable DNA binding protein (napDNAbp), (ii) a cytidine deaminase domain, and (iii) a uracil binding protein (UBP). In some embodiments, the fusion protein further comprises (iv) a nucleic acid polymerase domain (NAP). As another example, a fusion protein may comprise (i) a nucleic acid programmable DNA binding protein (napDNAbp), (ii) a cytidine deaminase domain, and (iii) a nucleic acid polymerase (NAP) domain. As another example, a fusion protein may comprise (i) a nucleic acid programmable DNA binding protein (napDNAbp), and (ii) a base excision enzyme (BEE). In some embodiments, the fusion protein further includes (iii) a nucleic acid polymerase (NAP) domain.

Prime Editing-Prime editing can be used to make nucleotide base pair substitutions, small insertions, and/or small deletions. Prime editing (PE) is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand. Cas protein-reverse transcriptase fusions or related systems can be used to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptases as the DNA polymerase component, the prime editors described herein are not limited to reverse transcriptases but may include the use of virtually any DNA polymerase. The prime editors can also comprise Cas9 (or an equivalent napDNAbp) which is programmed to target a DNA sequence by associating it with a PEgRNA containing a spacer sequence that anneals to a complementary protospacer in the target DNA. The PEgRNA can also contain new genetic information in the form of an extension that encodes a replacement strand of DNA containing a desired genetic alteration which can be used to replace a corresponding endogenous DNA strand at the target site. To transfer information from the PEgRNA to the target DNA, the mechanism of prime editing can involve nicking the target site in one strand of the DNA to expose a 3′-hydroxyl group. The exposed 3′-hydroxyl group can then be used to prime the DNA polymerization of the edit-encoding extension on PERNA directly into the target site. The napDNAbp can be Cas9, Casl2e, Casl2d, Casl2a, Casl2bl, Casl3a, Casl2c, or Argonaute and can have a nickase activity.

Prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). The prime editing guide RNA (PEgRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule and the extended gRNA guides the napDNAbp to bind to a target locus. In step (b), a nick in one of the strands of DNA of the target locus is introduced by a nuclease, thereby creating an available 3′ end in one of the strands of the target locus. In certain embodiments, the nick is created in the strand of DNA that corresponds to the R-loop strand, which is the strand that is not hybridized to the guide RNA sequence, termed the “non-target strand.” The nick, however, could be introduced in either of the strands. That is, the nick could be introduced into the R-loop “target strand” (the strand hybridized to the protospacer of the extended gRNA) or the “non-target strand.” In step (c), the 3′ end of the DNA strand (formed by the nick) interacts with the extended portion of the guide RNA in order to prime reverse transcription—“target-primed RT.” In certain embodiments, the 3′ end DNA strand hybridizes to a specific RT priming sequence on the extended portion of the guide RNA, termed the “reverse transcriptase priming sequence” or “primer binding site” on the PEgRNA. In step (d), a reverse transcriptase (or other suitable DNA polymerase) is introduced which synthesizes a single strand of DNA from the 3′ end of the primed site towards the 5′ end of the prime editing guide RNA. The DNA polymerase can be fused to the napDNAbp or alternatively can be provided in trans to the napDNAbp. This forms a single strand DNA flap comprising the desired nucleotide change which is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolves the mismatched DNA to incorporate the nucleotide change(s) to form the desired altered product. The process can also be driven towards product formation with “second strand nicking.”

The prime editors of the present disclosure may also comprise Cas9 variants with modified PAM specificities. Some aspects of this disclosure provide Cas9 proteins that exhibit activity on a target sequence that does not comprise the canonical PAM (5′-NGG-3′, where Nis A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

It should be appreciated that any of the amino acid substitutions described herein, from a first amino acid residue (e.g., A) to a second amino acid residue (e.g., T) may also include substitutions from the first amino acid residue to an amino acid residue that is similar to the second amino acid residue, which can be referred to as a conservative substitution.

The desired nucleotide change can be installed in an editing window that is between about −5 to +5 residues of the nick site, or between about −10 to +10 residues of the nick site, or between about −20 to +20 residues of the nick site, or between about −30 to +30 residues of the nick site, or between about −40 to +40 residues of the nick site, or between about −50 to +50 residues of the nick site, or between about −60 to +60 residues of the nick site, or between about −70 to +70 residues of the nick site, or between about −80 to +80 residues of the nick site, or between about −90 to +90 residues of the nick site, or between about −100 to +100 residues of the nick site, or between about −200 to +200 residues of the nick site.

The method can also comprise introducing one or more changes in the nucleotide sequence of a DNA molecule at a target locus, comprising contacting the DNA molecule with a nucleic acid programmable DNA binding protein (napDNAbp) and a guide RNA which targets the napDNAbp to the target locus, wherein the guide RNA comprises a reverse transcriptase (RT) template sequence comprising at least one desired nucleotide change. Next, the method involves forming an exposed 3′ end in a DNA strand at the target locus and then hybridizing the exposed 3′ end to the RT template sequence to prime reverse transcription. Next, a single strand DNA flap comprising the at least one desired nucleotide change based on the RT template sequence can be synthesized or polymerized by reverse transcriptase. Lastly, the at least one desired nucleotide change can be incorporated into the corresponding endogenous DNA, thereby introducing one or more changes in the nucleotide sequence of the DNA molecule at the target locus.

Several suitable prime editing nucleases are described in the art. PEI is a PE complex comprising a fusion protein comprising Cas9 (H840A) and a wild type Moloney Murine Leukemia Virus (MMLV) RT having the following structure: {NLS}−{Cas9 (H840A)}−{linker}−{MMLV_RT (wt)}+a desired PEgRNA. PE2 is a PE complex comprising a fusion protein comprising Cas9 (H840A) and a variant MMLV RT having the following structure: {NLS}-{Cas9 (H840A)}−{linker}−{MMLV_RT (D200N) (T330P) (L603W) (T306K) (W313F)}+a desired PERNA. PE3 is a complex comprising the elements of PE2 plus a second-strand nicking guide RNA that complexes with the PE2 and introduces a nick in the non-edited DNA strand in order to induce preferential replacement of the edited strand. PE-short refers to a PE construct that is fused to a C-terminally truncated reverse transcriptase. The amino acid sequences of these proteins are published in WO2020191245A1, which is hereby incorporated by reference in its entirety.

There are a number of publicly available tools available to help choose and/or design target sequences as well as lists of bioinformatically determined unique gRNAs for different genes in different species, such as but not limited to, Feng Zhang lab's Target Finder, Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and a Cas endonuclease such as, but without limitation Cas9, should be expressed or present—for example as a ribonucleoprotein complex—in a target cell. The insertion vector can contain both cassettes on a single plasmid or the cassettes are expressed from two separate plasmids. CRISPR plasmids are commercially available such as the px330 plasmid from Addgene (75 Sidney St, Suite 550A·Cambridge, MA 02139). Use of clustered regularly interspaced short palindromic repeats (CRISPR)-associated (Cas)-guide RNA technology and a Cas endonuclease for modifying mammalian genomes are also at least disclosed by Bauer, D. E., et al., (Vis Exp., 2015, 95, e52118), which is specifically incorporated herein by reference in its entirety. Cas endonucleases that can be used to effect DNA editing with gRNA include, but are not limited to, Cas9, Cpf1 (Zetsche, B., et al., Cell, 2015, 163, 759-771), C2c1, C2c2, and C2c3 (Shmakov, S., et al., Mol. Cell., 2015, 60, 385-397).

### Vector and Oligo Insertional Applications

The “Hit and run” or “in-out” process involves a two-step recombination procedure. In the first step, an insertion-type vector containing a dual positive/negative selectable marker cassette can be used to introduce the desired sequence alteration. The insertion vector can contain a single continuous region of homology to the targeted locus and can be modified to carry the nucleotide change of interest. This targeting construct can be linearized with a restriction enzyme site within the region of homology, introduced into the cells, and positive selection can be performed to isolate homologous recombination mediated events. The DNA carrying the homologous sequence can be provided as a plasmid or a single or double stranded oligo. These homologous recombinants contain a local duplication that can be separated by intervening vector sequence, including the selection cassette. In the second step, targeted clones can be subjected to negative selection to identify cells that have lost the selection cassette via intra-chromosomal recombination between the duplicated sequences. The local recombination event can remove the duplication and, depending on the site of recombination, the allele either retains the introduced nucleotide change or reverts to wild type. The end result can be the introduction of the desired modification without the retention of any exogenous sequences.

The “double-replacement” or “tag and exchange” strategy involves a two-step selection procedure similar to the hit and run approach, but requires the use of two different targeting constructs. In the first step, a standard targeting vector with 3′ and 5′ homology arms can be used to insert a dual positive/negative selectable cassette near the location where the nucleotide change is to be introduced. After the system components have been introduced to the cell and positive selection applied, HR mediated events can be identified. Next, a second targeting vector that contains a region of homology with the desired mutation can be introduced into targeted clones, and negative selection can be applied to remove the selection cassette and introduce the mutation. The final allele contains the desired mutation while eliminating unwanted exogenous sequences.

According to a specific embodiment, the DNA editing agent can comprise a DNA targeting module, for example, but without limitation a gRNA. The DNA editing agent may or may not further comprise an endonuclease. In some embodiments of the method, the DNA editing agent can comprise a nuclease such as an endonuclease and a DNA targeting module such as gRNA. In certain embodiments, the DNA editing agent can be CRISPR/Cas, e.g. gRNA and Cas9.

According to a specific embodiment, the DNA editing agent can be a TALEN. According to a specific embodiment, the DNA editing agent can be a ZFN. According to a specific embodiment, the DNA editing agent can be a meganuclease.

Additionally or alternatively, in some embodiments, the edit can comprise an edit of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides.

According to one embodiment, the editing agent can comprise an edit of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides.

According to one embodiment, the edit can be in a consecutive nucleic acid sequence (e.g. at least 5, 10, 20, 30, 40, 50, 100, 150, 200 bases). According to one embodiment, the edit can be in a non-consecutive manner, e.g. throughout a 20, 50, 100, 150, 200, 500, 1000 nucleic acid sequence.

In some embodiments, the edit comprises an edit of at most 200 nucleotides, at most 150 nucleotides, at most 100 nucleotides, at most 50 nucleotides, at most 25 nucleotides, at most 20 nucleotides, at most 15 nucleotides, at most 10 nucleotides, or at most 5 nucleotides.

Additionally or alternatively, in some embodiments, the edit can comprise an insertion. According to a specific embodiment, the insertion can comprise an insertion of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native DNA). In certain embodiments, the insertion comprises an insertion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190 or 200 or more nucleotides (as compared to the native DNA).

In some embodiments, the insertion comprises an insertion of at most 200 nucleotides, at most 150 nucleotides, at most 100 nucleotides, at most 50 nucleotides, at most 25 nucleotides, at most 20 nucleotides, at most 15 nucleotides, at most 12 nucleotides, at most 10 nucleotides, at most 9 nucleotides, at most 6 nucleotides, at most 5 nucleotides, or at most 3 nucleotides.

Additionally or alternatively, in some embodiments, the modification can comprise a deletion. According to a specific embodiment, the deletion can comprise a deletion of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native DNA). In some embodiments, the deletion comprises a deletion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, or 10000 nucleotides (as compared to the native non-coding RNA molecule, e.g. RNA silencing molecule).

In some embodiments, the deletion comprises a deletion of at most 200 nucleotides, at most 150 nucleotides, at most 100 nucleotides, at most 50 nucleotides, at most 25 nucleotides, at most 20 nucleotides, at most 15 nucleotides, at most 10 nucleotides, or at most 5 nucleotides.

Additionally or alternatively, in some embodiments, the modification comprises a point mutation.

Additionally or alternatively, in some embodiments, the modification comprises a combination of any of a deletion, an insertion and/or a point mutation.

Additionally or alternatively, in some embodiments, the modification can comprise nucleotide substitutions. According to a specific embodiment, the substitution can comprise substitution of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native DNA). In some embodiments, the nucleotide substitution can comprise a nucleotide replacement in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native DNA).

In some embodiments, the nucleotide substitution can comprise a nucleotide replacement in at most 200 nucleotides, at most 150 nucleotides, at most 100 nucleotides, at most 50 nucleotides, at most 25 nucleotides, at most 20 nucleotides, at most 15 nucleotides, at most 10 nucleotides, or at most 5 nucleotides.

Delivery of DNA editing agents. Additionally or alternatively, in some embodiments of the methods disclosed herein, the DNA editing agent may be introduced into eukaryotic cells using DNA delivery methods like expression vectors or using DNA-free methods. According to one embodiment, the DNA recognition molecule, such as a gRNA, can be provided as RNA to the cell.

Thus, it will be appreciated that the present techniques relate to introducing the DNA editing agent using DNA-free methods such as RNA transfection, or ribonucleoprotein (RNP) transfection. For example, Cas9 can be introduced as a DNA expression plasmid, in vitro RNA transcript, or as a recombinant protein bound to the gRNA in a ribonucleoprotein particle (RNP). gRNA can be delivered either as a DNA plasmid or as an in vitro RNA transcript.

Any method known in the art for RNA or RNP transfection can be used in accordance with the present teachings, such as, but not limited to microinjection (an exemplary procedure may be found in Cho, S. W., et al., Genetics, 2013, 195, 1177-1180), electroporation (an exemplary procedure may be found in Kim, S., et al., Genome Res., 2014, 24, 1012-1019), or lipid-mediated transfection (an exemplary procedure using liposomes may be found in Zuris, J. A., et al., Nat. Biotechnol., 2015, 33, 73-80). Additional methods of RNA transfection are described in U.S. Publication Number 20160289675.

One advantage of RNA transfection methods of the present technology is that RNA transfection is essentially transient and vector-free. An RNA transgene can be delivered to a cell and expressed therein, as a minimal expressing cassette without the need for any additional sequences.

According to one embodiment, for expression of exogenous DNA editing agents of the present technology in mammalian cells, a polynucleotide sequence encoding the DNA editing agent can be ligated into a nucleic acid construct suitable for mammalian cell expression. Such a nucleic acid construct can include a promoter sequence for directing transcription of the polynucleotide sequence in the cell in a constitutive or inducible manner.

The nucleic acid construct (also referred to herein as an “expression vector”) of some embodiments of the present technology includes additional sequences which render this vector suitable for replication and integration in eukaryotes, for example, shuttle vectors. In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the present technology is active in the specific cell population transformed. Examples of cell type-specific and/or tissue-specific promoters include promoters such as albumin that is liver specific (Pinkert, C. A., et al., Genes Dev., 1987, 1, 268-277), lymphoid specific promoters (Calame, K and Eaton, S., Adv. Immunol., 1988, 43, 235-275); in particular promoters of T-cell receptors (Winoto, A. and Baltimore, D., EMBO J., 1989, 8, 729-733) and immunoglobulins; (Banerji, J., et al., 1983, Cell, 33729-33740), neuron-specific promoters such as the neurofilament promoter (Byrne, G. H. and Ruddle, F. H., Proc. Natl. Acad. Sci. USA, 1989, 86, 5473-5477), pancreas-specific promoters (Edlund, T., et al., Science, 1985, 230, 912-916) or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the present technology include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N. Y. 1983.

In the construction of the expression vector, the promoter may be positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the present technology include those derived from SV40.

In addition to the elements already described, the expression vector of some embodiments of the present technology may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the present technology can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding a DNA editing agent can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1 (+/−), pGL3, pZeoSV2 (+/−), pSecTag2, pDisplay, pEF/myc/cyto, pCMV/myc/cyto, pCR3.1, pSinRep5, DH26S, DHBB, pNMTI, pNMT41, pNMT81, which are available from Invitrogen, pCI which is available from Promega, pMbac, pPbac, pBK-RSV, and pBK-CMV, which are available from Stratagene, and pTRES which is available from Clontech. Derivatives of these plasmids or any other mammalian expression vector known in the art are also acceptable for the present teachings.

Expression vectors containing regulatory elements from eukaryotic viruses such as retroviruses can be also used. SV40 vectors include pSVT7 and pMT2. Vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the present technology will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) (Liang, C-Y., et al., Arch Virol., 2004, 149, 51-60).

Recombinant viral vectors are useful for in vivo expression of DNA editing agents since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This contrasts with vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

According to one embodiment, in order to express a functional DNA editing agent, in cases where the cleaving module (nuclease) is not an integral part of the DNA recognition unit, the expression vector may encode the cleaving module as well as the DNA recognition unit (e.g. gRNA in the case of CRISPR/Cas). Alternatively, the cleaving module and the DNA recognition unit may be cloned into separate expression vectors. In such a case, if expression vectors are to be used, at least two different expression vectors must be transformed into the same eukaryotic cell.

Alternatively, when a nuclease is not utilized (i.e. not administered from an exogenous source to the cell), the DNA recognition unit (e.g. gRNA) may be cloned and expressed using a single expression vector.

According to one embodiment, the DNA editing agent comprises a nucleic acid agent encoding at least one DNA recognition unit operatively linked to a cis-acting regulatory element active in eukaryotic cells.

According to one embodiment, the nuclease and the DNA recognition unit (e.g. gRNA) are encoded from the same expression vector. Such a vector may comprise a single cis-acting regulatory element active in eukaryotic cells for expression of both the nuclease and the DNA recognition unit. Alternatively, the nuclease and the DNA recognition unit may each be operably linked to a cis-acting regulatory element active in eukaryotic cells.

According to one embodiment, the nuclease and the DNA recognition unit are encoded from different expression vectors whereby each is operably linked to a cis-acting regulatory element active in eukaryotic cells.

According to one embodiment, the method of some embodiments of the present technology further comprises introducing into the eukaryotic cell donor oligonucleotides. According to one embodiment, when the modification is an insertion and/or a deletion, the method further comprises introducing into the eukaryotic cell donor oligonucleotides. According to certain embodiments, when the modification is a point mutation, the method further comprises introducing into the eukaryotic cell donor oligonucleotides.

As used herein, the term “donor oligonucleotides” or “donor oligos” refers to exogenous nucleotides, most often in the form of externally introduced into the eukaryotic cell which matches an endogenous genome sequence aside from the small change comprising the desired edit. The donor oligonucleotides or donor oligos are then used as templates for HR in the cell to generate a precise change in the genome. According to one embodiment, the donor oligonucleotides are synthetic.

The donor oligos may be RNA oligos, DNA oligos, or synthetic oligos. In some embodiments, the donor oligonucleotides comprise single-stranded donor oligonucleotides (ssODN), double-stranded donor oligonucleotides (dsODN), double-stranded DNA (dsDNA), double-stranded DNA-RNA duplex (DNA-RNA duplex), double-stranded DNA-RNA hybrid, single-stranded DNA-RNA hybrid, single-stranded DNA (ssDNA), double-stranded RNA (dsRNA), or single-stranded RNA (ssRNA).

Additionally or alternatively, in some embodiments, the donor oligonucleotides comprise the DNA or RNA sequence for swapping (as discussed above).

The donor oligonucleotides may be provided in a non-expressed vector format (e.g., via DNA donor plasmid) or oligo. In some embodiments, the donor oligonucleotides comprise about 50-5000, about 100-5000, about 250-5000, about 500-5000, about 750-5000, about 1000-5000, about 1500-5000, about 2000-5000, about 2500-5000, about 3000-5000, about 4000-5000, about 50-4000, about 100-4000, about 250-4000, about 500-4000, about 750-4000, about 1000-4000, about 1500-4000, about 2000-4000, about 2500-4000, about 3000-4000, about 50-3000, about 100-3000, about 250-3000, about 500-3000, about 750-3000, about 1000-3000, about 1500-3000, about 2000-3000, about 50-2000, about 100-2000, about 250-2000, about 500-2000, about 750-2000, about 1000-2000, about 1500-2000, about 50-1000, about 100-1000, about 250-1000, about 500-1000, about 750-1000, about 50-750, about 150-750, about 250-750, about 500-750, about 50-500, about 150-500, about 200-500, about 250-500, about 350-500, about 50-250, about 150-250, or about 200-250 nucleotides.

According to a specific embodiment, the donor oligonucleotides comprising the ssODN comprise about 200-500 nucleotides. According to a specific embodiment, the donor oligonucleotides comprising the dsODN comprise about 250-5000 nucleotides.

According to one embodiment, for gene swapping of an endogenous RNA silencing molecule (e.g. miRNA) with a RNA silencing sequence of choice (e.g. siRNA), the expression vector, ssODN or dsODN need not to be expressed in a eukaryotic cell and only serves as a non-expressing template. According to a specific embodiment, in such a case only the DNA editing agent need to be expressed if provided in a DNA form.

According to one embodiment, introducing into the eukaryotic cell donor oligonucleotides is executed using any of the methods described above.

According to one embodiment, the gRNA and the DNA donor oligonucleotides are co-introduced into the eukaryotic cell. It will be appreciated that any additional factors may be co-introduced therewith. According to one embodiment, the gRNA can be introduced into the eukaryotic cell a few minutes or a few hours prior to the DNA donor oligonucleotides. It will be appreciated that any additional factors may be introduced prior to, concomitantly with, or following the gRNA or the DNA donor oligonucleotides.

According to one embodiment, the gRNA is introduced into the eukaryotic cell subsequent to the DNA donor oligonucleotides. It will be appreciated that any additional factors may be introduced prior to, concomitantly with, or following the gRNA or the DNA donor oligonucleotides.

In some embodiments, the methods of the present technology comprise providing a composition comprising at least one gRNA and DNA donor oligonucleotide for genome editing. In some embodiments, the methods of the present technology comprise providing a composition comprising at least one gRNA, a nuclease and DNA donor oligonucleotides for genome editing. In alternate embodiments, multiple gRNAs may be introduced with a nuclease in the absence of a donor molecule in order to introduce small deletions by creating multiple double stranded breaks on the same molecule.

Various methods can be used to introduce the expression vector or donor oligos of some embodiments of the present technology into eukaryotic cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa, E., et al., Biotechniques, 4 504-512, 1986 and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Choi (Tonkinson, J. L. and Stein, C. A., Cancer Investigation, 1996, 14, 54-65). For gene therapy, the preferred constructs are viruses, most preferably adenoviruses, AAV, lentiviruses, or retroviruses. A viral construct such as a retroviral construct includes at least one transcriptional promoter/enhancer or locus-defining element(s), or other elements that controls gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the present technology. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of some embodiments of the present technology can also include sequences engineered to enhance stability, production, purification, yield or toxicity of the expressed peptide.

According to a specific embodiment, a bombardment method is used to introduce foreign genes into eukaryotic cells. According to one embodiment, the method is transient. An exemplary bombardment method which can be used in accordance with some embodiments of the present technology is discussed in the examples section which follows. Bombardment of mammalian cells is also taught by Uchida, M., et al., Biochim. Biophys. Acta., 2009, 1790, 754-764.

Regardless of the transformation/infection method employed, the present teachings further select transformed cells comprising a genome editing event. According to a specific embodiment, selection can be carried out such that only cells comprising a successful accurate edit in the specific locus are selected. Accordingly, cells comprising any event that includes an edit in an unintended locus are not selected.

According to one embodiment, selection of edited cells can be performed at the phenotypic level or by detection of a molecular event.

According to one embodiment, selection of edited cells is performed by analyzing eukaryotic cells or clones comprising the DNA editing event also referred to herein as an “edit.” The type of analysis is dependent on the type of editing sought e.g., insertion, deletion, insertion-deletion (Indel), inversion, substitution and combinations thereof.

Methods for detecting sequence alteration are well known in the art and include, but are not limited to, DNA and RNA sequencing, electrophoresis, an enzyme-based mismatch detection assay, and a hybridization assay such as PCR, RT-PCR, RNase protection, in situ hybridization, primer extension, Southern blot, northern blot, and dot blot analysis. Various methods used for detection of single nucleotide polymorphisms (SNPs) can also be used, such as PCR based T7 endonuclease, heteroduplex, and Sanger sequencing, or PCR followed by restriction digest to detect appearance or disappearance of one or more unique restriction sites.

Another method of validating the presence of a DNA editing event comprises a mismatch cleavage assay that makes use of a structure selective enzyme that recognizes and cleaves mismatched DNA.

Alternatively, eukaryotic cells may be further cultured and maintained, for example, in an undifferentiated state for extended periods of time or may be induced to differentiate into other cell types, tissues, organs or organisms as required.

The DNA editing agents and optionally the donor oligos of some embodiments of the present technology can be administered to a single cell, to a group of cells (e.g. primary cells or cell lines as discussed above) or to an organism.

### Uses of the Gene Editing Agents of the Present Technology

In one aspect, the present disclosure provides a method for improving fitness in a non-human animal subject comprising contacting a eukaryotic cell of the non-human animal subject with an effective amount of a DNA editing agent, wherein the DNA editing agent is configured to modify a gene into an allele that confers resistance to a pathogen (e.g., bacteria, fungus, virus, protozoa). The pathogen may swine or cattle. In some embodiments, the pathogen is a virus such as PRRS virus, African Swine Fever (ASF) virus, H1N1 virus, coronavirus, Bovine viral diarrhea virus, Bovine leukemia virus, Bovine herpesvirus, Lumpky skin disease virus, Caprine arthritis and encephalitis virus, Peste-des-petits-ruminants virus, Sheeppox and goatpox viruses, Classical swine fever virus, Swine vesicular disease virus, Transmissible gastroenteritis virus of swine, West Nile fever virus, Vesicular stomatitis virus, Japanese encephalitis virus, Bluetongue virus, Rinderpest virus, Rift Valley fever virus, Rabies virus, Foot-and-mouth disease virus, and Pseudorabies virus. In certain embodiments, the pathogen is a bacterium, such as Mannheimia haemolytica, Escherichia coli, Salmonella, Listeria monocytogenes, Clostridium spp., Campylobacter, Yersinia enterocolitica, Moraxella bovis, Brucella abortus, Streptococcus agalactiae, Leptospira spp., Pasteurella multocida, Mycoplasma mycoides, Trueperella pyogenes, Mycoplasma bovis, Mycobacterium bovis, Chlamydophila abortus, or Coxiella burnetii. In other embodiments, the pathogen is a protozoa, such as Neospora caninum, Sarcocystis spp., Tritrichomonas foetus, Neoparamoeba perurans, Cyptosporidium parvum, and Giardia lamblia. Additionally or alternatively, in some embodiments, the pathogen causes a disease selected from among Porcine Reproductive and Respiratory Syndrome (PRRS), African Swine Fever, influenza, coronavirus, Cardiomyopathy syndrome (CMS), hemorrhagic disease, hepatitis, pancreatitis, respiratory disease, Streptococcosis, furunculosis, Leptospirosis, Brucellosis, pink eye, Johne's disease, Salmonellosis, Campylobacteriosis, reactive arthritis, postinfectious irritable bowel syndrome, hemorrhagic colitis, Listeriosis, meningitis, septicemia, Yersiniosis, paratuberculosis, anthrax, Hemorrhagic septicemia, Mycoplasma pneumonia, Contagious bovine pleuropneumonia, blackleg disease, malignant edema, black disease, enterotoxemia, redwater disease, Giardiasis, Gastroenteritis, cryptosporidiosis, Bovine genital campylobacteriosis, Bovine viral diarrhea, Enzootic bovine leucosis, Infectious bovine rhinotracheitis/infectious pustular vulvovaginitis, Lumpky skin disease, Classical swine fever, Nipah virus encephalitis, Swine vesicular disease, Transmissible gastroenteritis, West Nile fever, Vesicular stomatitis, Japanese encephalitis, Bluetongue, Rabies, Rift Valley fever, Rinderpest, Foot-and-mouth disease, Aujeszky's disease, Q fever, neosporosis, sarcocystosis, and trichomoniasis.

According to one aspect of the present technology, provided is a method of improving one or more genetic traits in a non-human animal subject in need thereof, the method comprising modifying a gene associated with pathogen resistance, fertility, lactation, or native traits that support more rapid growth or feed efficiency.

According to one aspect of the present technology, provided is a DNA editing agent conferring pathogen resistance, fertility, lactation, or native traits that support more rapid growth or feed efficiency, for use in improving one or more genetic traits in a non-human animal subject in need thereof.

Exemplary bovine traits include polled (lack of horns), sterility or fertility, milk production, growth (which increases meat production), fat production, conception rates, stillborn rates, calving ease, or content of produced milk such as the amount of protein or the amount of fat. Further bovine traits can include backfat thickness, intramuscular fat, ultrasound loin muscle area, loin muscle area and intramuscular fat content, chest circumference, withers height, body length, hip height, rump length, and heart girth. Other exemplary native traits include, but are not limited to, high altitude adaptation and response to hypoxia (DCAF8, PPP1R12A, SLC16A3, UCP2, UCP3, TIGAR), cold acclimation (AQP3, AQP7, HSPB8), body size and stature (PLAG1, KCNA6, NDUFA9, AKAP3, C5H12orf4, RAD51AP1, FGF6, TIGAR, CCND2, CSMD3), resistance to disease and bacterial infection (CHI3L2, GBP6, PPFIBP1, REP15, CYP4F2, TIGD2, PYURF, SLC10A2, FCHSD2, ARHGEF17, RELT, PRDM2, KDM5B), reproduction (PPP1R12A, ZFP36L2, CSPP1), milk yield and components (NPC1L1, NUDCD3, ACSS1, FCHSD2), growth and feed efficiency (TMEM68, TGS1, LYN, XKR4, FOXA2, GBP2, GBP5, FGD6), and polled phenotype (URB1, EVAIC).

Other exemplary target genes can include PRLR, NANOS2, Deadend (Dnd), APAF1, SMC2, GART, TFBIM, SIRT1, SIRT2, LPL, CRTC2, SIX4, UCP2, UCP3, URB1, EVAIC, TMEM68, TGS1, LYN, XKR4, FOXA2, GBP2, GBP5, FGD6, NPCIL1, NUDCD3, ACSS1, FCHSD2, PPPIR12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, TIGAR and ZBTB.

Exemplary porcine traits include meat production traits such as growth rate, backfat depth, muscle pH, purge loss, muscle color, firmness, marbling scores, intramuscular fat percentage, tenderness, average daily gain, average daily feed intake, feed efficiency, back fat thickness, loin muscle area, and lean percentage. Exemplary health traits include the absence of undesirable physical abnormalities or defects (like scrotal ruptures), improvement of feet and leg soundness, resistance to specific diseases or disease organisms, or general resistance to pathogens. Further health traits can include melanotic skin tumors, dermatosis vegetans, abnormal mamae, shortened vertebral column, kinky tail, rudimentary tail, hairlessness, woolly hair, hydrocephalus, tassels, legless, three-legged, syndactyly, polydactyly, pulawska factor, heterochromia iridis, congenital tremor a iii, congenital tremor a iv, congenital ataxia, hind leg paralysis, bentleg, thickleg, malignant hyperthermia, hemophilia (von Willebrand's disease), leukemia, hemolytic disease, edema, acute respiratory distress (“barker”), rickets, renal hypoplasia, renal cysts, uterus aplasia, porcine stress syndrome (pss), halothane (hal), dipped shoulder (humpy back, kinky back, kyphosis), hyperostosis, mammary hypoplasia, undeveloped udder, and epitheliogenesis imperfecta. Exemplary target genes can include, but are not limited to, CD18, ANP32, ANPEP, TMPRSS1, TMPRSS2, NANOS2, CD163, Melanocortin-4 receptor (MC4R), HMGA, IGF2, E. coli F4ab/ac, HAL, Mx1, BAT2, EHMT2, PRDM1, PRDM14, and ESR.

Exemplary nucleic acid sequences associated with pathogen resistance, fertility, lactation, or native traits that support more rapid growth or feed efficiency in swine or cattle include, but are not limited to:

| Gene | GenBank Acc. No. |
|---|---|
| CD18 | M81233 (SEQ ID NO: 1) |
| | AY452481 (SEQ ID NO: 2) |
| | DQ470837 (SEQ ID NO: 3) |
| | DQ104444 (SEQ ID NO: 4) |
| CD163 | NM_213976.1 (SEQ ID NO: 5) |
| | XM_021091121.1 (SEQ ID NO: 6) |
| | GQ184570.1 (SEQ ID NO: 7) |
| | GAAI01005873.1 (SEQ ID NO: 8) |
| ANP32 | XM_003121759 (SEQ ID NO: 9) |
| (ANP32A and | XM_021066477.1 (SEQ ID NO: 10) |
| ANP32B) | NM_001195019.1 (SEQ ID NO: 11) |
| | NM_001035074.1 (SEQ ID NO: 12) |
| | XM_004010266.4 (SEQ ID NO: 13) |
| | XM_015093130.3 (SEQ ID NO: 14) |
| ANPEP | XM_005653524.3 (SEQ ID NO: 15) |
| | XM_021098304.1 (SEQ ID NO: 16) |
| | NM_001075144.1 (SEQ ID NO: 17) |
| TMPRSS2 | NM_001386131.1 (SEQ ID NO: 18) |
| | NM_001081585.1 (SEQ ID NO: 19) |
| NANOS | XM_001928298 (SEQ ID NO: 20) |
| | XM_003127232.2 (SEQ ID NO: 21) |
| | XM_005225796 (SEQ ID NO: 22) |
| Melanocortin-4 | NM_214173.1 (SEQ ID NO: 23) |
| receptor | NM_174110.1 (SEQ ID NO: 24) |
| (MC4R) | NM_001126370.3 (SEQ ID NO: 25) |
| PRLR | NM_001039726.2 (SEQ ID NO: 26) |
| | NM_174155.3 (SEQ ID NO: 27) |
| | NM_001285669.1 (SEQ ID NO: 28) |
| | NM_001001868.1 (SEQ ID NO: 29) |
| HMGA | XM_005663957.3 (SEQ ID NO: 30) |
| | XM_021091958.1 (SEQ ID NO: 31) |
| | XM_002687648.5 (SEQ ID NO: 32) |
| IGF2 | NM_213883.2 (SEQ ID NO: 33) |
| | NM_001367627.1 (SEQ ID NO: 34) |
| HAL | XM_001925061.4 (SEQ ID NO: 35) |
| Mx1 | NM_214061.2 (SEQ ID NO: 36) |
| BAT2 | NM_001114675.1 (SEQ ID NO: 37) |
| EHMT2 | NM_001101823.1 (SEQ ID NO: 38) |
| | NM_001206263.2 (SEQ ID NO: 39) |
| PRDM1 | NM_001192936.1 (SEQ ID NO: 40) |
| | XM__005659341.3 (SEQ ID NO: 41) |
| PRDM14 | NM_001191264.1 (SEQ ID NO: 42) |
| | XM_021090784.1 (SEQ ID NO: 43) |
| ESR | NM_214220.1 (SEQ ID NO: 44) |
| APAF1 | NM_001191507.1 (SEQ ID NO: 45) |
| | XM_021093024.1 (SEQ ID NO: 46) |
| SMC2 | XM_021066949.1 (SEQ ID NO: 47) |
| | XM__024996295.1 (SEQ ID NO: 48) |
| GART | NM_001040473.2 (SEQ ID NO: 49) |
| | XM_021070945.1 (SEQ ID NO: 50) |
| TFB1M | NM_001076896.2 (SEQ ID NO: 51) |
| | NM_001128475.1 (SEQ ID NO: 52) |
| SIRT1 | NM_001145750.2 (SEQ ID NO: 53) |
| | NM_001192980.3 (SEQ ID NO: 54) |
| SIRT2 | XM_021093185.1 (SEQ ID NO: 55) |
| | NM_001113531.1 (SEQ ID NO: 56) |
| LPL | NM_001075120.1 (SEQ ID NO: 57) |
| | NM_214286.1 (SEQ ID NO: 58) |
| CRTC2 | XM_005663405.3 (SEQ ID NO: 59) |
| | NM_001076250.1 (SEQ ID NO: 60) |
| SIX4 | XM_024998078.1 (SEQ ID NO: 61) |
| | NM_001244614.1 (SEQ ID NO: 62) |
| UCP2 | NM_001033611.2 (SEQ ID NO: 63) |
| | NM_214289.1 (SEQ ID NO: 64) |
| UCP3 | NM_174210.1 (SEQ ID NO: 65) |
| | NM_214049.1 (SEQ ID NO: 66) |
| URB1 | NM_001205980.1 (SEQ ID NO: 67) |
| | XM_018047870.1 (SEQ ID NO: 68) |
| | XM_027978333.1 (SEQ ID NO: 69) |
| EVA1C | XM_024988259.1 (SEQ ID NO: 70) |
| | XM_018047722.1 (SEQ ID NO: 71) |
| TMEM68 | NM_001076009.1 (SEQ ID NO: 72) |
| TGS1 | XM_015474433.2 (SEQ ID NO: 73) |
| LYN | XM_025001638.1 (SEQ ID NO: 74) |
| XKR4 | XM_002692650.4 (SEQ ID NO: 75) |
| FOXA2 | XM_025001047.1 (SEQ ID NO: 76) |
| FGD6 | XM__005206128.4 (SEQ ID NO: 77) |
| NPC1L1 | XM_002686890.5 (SEQ ID NO: 78) |
| | XM__018047026.1 (SEQ ID NO: 79) |
| | XM_004008262.5 (SEQ ID NO: 80) |
| NUDCD3 | XM_005205595.3 (SEQ ID NO: 81) |
| | XM__018047025.1 (SEQ ID NO: 82) |
| | XM_004007984.5 (SEQ ID NO: 83) |
| ACSS1 | XM_025000325.1 (SEQ ID NO: 84) |
| | XM_018057282.1 (SEQ ID NO: 85) |
| | XM_015099680.3 (SEQ ID NO: 86) |
| FCHSD2 | XM_024975871.1 (SEQ ID NO: 87) |
| | XM_018059383.1 (SEQ ID NO: 88) |
| | XM_004016298.5 (SEQ ID NO: 89) |
| PPP1R12A | XM_024991981.1 (SEQ ID NO: 90) |
| | XM_021092811.1 (SEQ ID NO: 91) |
| ZFP36L2 | NM_001191191.1 (SEQ ID NO: 92) |
| CSPP1 | XM_025001590.1 (SEQ ID NO: 93) |
| CHI3L2 | XM_024990025.1 (SEQ ID NO: 94) |
| GBP6 | NM_001075995.1 (SEQ ID NO: 95) |
| GBP5 | HF564747.1 (SEQ ID NO: 96) |
| PPFIBP1 | XM_010805392.3 (SEQ ID NO: 97) |
| REP15 | XM_005206922.4 (SEQ ID NO: 98) |
| CYP4F2 | XM_010806565.3 (SEQ ID NO: 99) |
| TIGD2 | XM_005207816.4 (SEQ ID NO: 100) |
| PYURF | NM_001038102.2 (SEQ ID NO: 101) |
| SLC10A2 | XM_025000165.1 (SEQ ID NO: 102) |
| ARHGEF17 | NM_001282586.1 (SEQ ID NO: 103) |
| RELT | XM_002693451.5 (SEQ ID NO: 104) |
| PRDM2 | XM_015475202.2 (SEQ ID NO: 105) |
| KDM5B | XM_024976577.1 (SEQ ID NO: 106) |
| PLAG1 | XM_005215434.3 (SEQ ID NO: 107) |
| KCNA6 | NM_001206413.3 (SEQ ID NO: 108) |
| NDUFA9 | NM_205817.1 (SEQ ID NO: 109) |
| AKAP3 | XM_015471316.2 (SEQ ID NO: 110) |
| C5H12orf4 | XM_005207206.4 (SEQ ID NO: 111) |
| RAD51AP1 | NM_001076403.1 (SEQ ID NO: 112) |
| FGF6 | NM_001192400.1 (SEQ ID NO: 113) |
| | XM_001928806.4 (SEQ ID NO: 114) |
| CCND2 | XM_024992177.1 (SEQ ID NO: 115) |
| CSMD3 | NM_001105499.2 (SEQ ID NO: 116) |
| AQP3 | NM_001079794.1 (SEQ ID NO: 117) |
| AQP7 | XM_024995941.1 (SEQ ID NO: 118) |
| HSPB8 | NM_001014955.1 (SEQ ID NO: 119) |
| DCAF8 | NM_001206419.2 (SEQ ID NO: 120) |
| SLC16A3 | NM_001109980.3 (SEQ ID NO: 121) |
| TIGAR | NM_001076370.1 (SEQ ID NO: 122) |
| Deadend (Dnd) | NM_001193145.1 (SEQ ID NO: 123) |

### EXAMPLES

All patents, patent applications, provisional applications, and publications referred to or cited herein are incorporated by reference in their entirety, including all FIGURES and tables, to the extent they are not inconsistent with the explicit teachings of this specification.

### Example 1

This example illustrates making a CRISPR-CAS9 edit in URB1.

This example demonstrates the use of gRNAs, in combination with Cas protein activity, to generate double-stranded breaks in DNA, which upon non-homologous end joining, results in the introduction of an exogenous stop codon into URB1 which would create a bovine without horns.

Guides within URB1 will be tested computationally for their ability to generate double stranded breaks. Computational predictions will be subsequently tested in Bovine Embryonic Fibroblast (BEF) cells, as described below. The gRNAs will be generated by in vitro transcription and complexed with SpyCas9 in water, using 3.2 μg of Cas9 protein and 2.2 μg of gRNA in a total volume of 2.23 μl. The resulting ribonucleoprotein (RNP) complexes will then be nucleofected into cells using a Lonza electroporator. In preparation for nucleofection, cells will be harvested using TRYPLE EXPRESS™ (ThermoFisher, recombinant Trypsin): the culture medium will be removed from cells, the cells will be washed once with Hank's Balanced Salt Solution (HBSS) or Dulbecco's Phosphate-Buffered Saline (DPBS), and incubated for 3-5 minutes at 38.5° C. in the presence of TrypLE. Cells will then be harvested with complete medium. Cells will be pelleted via centrifugation (300×g for 5 minutes at room temperature), supernatant will be discarded, and then the cells were resuspended in 10 mL PBS to obtain single cell suspensions to allow cell counting using trypan blue staining. After counting, cells will be pelleted via centrifugation, the supernatant will be discarded, and the cells will be resuspended in nucleofection buffer P3 at a final concentration of 7.5×10⁶ cells/ml. 20 μl of the cell suspension will be added to each well of a nucleofection cuvette containing the RNP mixture and then mixed gently to resuspend the cells. The RNP/cell mixture will then be nucleofected with program CM138 (provided by the manufacturer). Following nucleofection, 80 μl of warm Embryonic Fibroblast Medium (EFM) (Dulbecco's Modified Eagle's Medium (DMEM) containing 2.77 mM glucose, 1.99 mM L-glutamine, and 0.5 mM sodium pyruvate, supplemented with 100 μM 2-Mercaptoethanol, 1× Eagle's minimum essential medium non-essential amino acids (MEM NEAA), 100 μg/mL Penicillin-Streptomycin, and 12% Fetal Bovine Serum) will be added to each well. The suspensions will be mixed gently by pipetting, and then 100 μl will be transferred to a 12-well plate containing 900 μl of EFM pre-incubated at 38.5° C. The plate will then be incubated at 38.5° C., 5% CO₂, 5% O₂, 90% N₂ for 48 hours. Forty-eight hours after nucleofection, genomic DNA will be prepared from transfected and control BEF: 15 μl of QUICKEXTRACT™ DNA Extraction Solution (Lucigen, Middleton, WI) will be added to pelleted cells, and the cells will then be lysed by incubating for 10 minutes at 37° C., for 8 minutes at 65° C., and for 5 minutes at 95° C. Lysate will be held at 4° C. until used for DNA sequencing.

To evaluate NHEJ repair outcomes at URB1 target sites mediated by the guide RNA/Cas endonuclease system, a region of approximately 250 bp of genomic DNA surrounding the target site will be amplified by PCR and then the PCR product will be examined by amplicon deep sequencing for the presence and nature of repairs. After transfection in triplicate, BEF genomic DNA will be extracted and the region surrounding the intended target site will be PCR amplified with Q5 Polymerase (NEB) adding sequences necessary for amplicon-specific barcodes and ILLUMINA® sequencing using tailed primers through two rounds of PCR, respectively. The resulting PCR amplification products will be deep sequenced on an ILLUMINA® MISEQ Personal Sequencer (ILLUMINA®, Inc., San Diego, CA). The resulting reads will be examined for the presence and nature of repairs at the expected sites of cleavage by comparison to control experiments where the Cas9 protein and guide RNA will be omitted from the transfection or by comparison to the reference genome. To calculate the frequency of NHEJ mutations for a target site/Cas9 protein/guide RNA combination, the total number of mutant reads (amplicon sequences containing insertions or deletions when compared to the DNA sequences from control treatments or reference genome) will be divided by total read number (wild-type plus mutant reads) of an appropriate length containing a perfect match to the barcode and forward primer. This example illustrates editing URB1 in bovine cells.

### Example 2

This example illustrates testing of gRNA efficiency in blastocyst cells.

A subset of gRNAs screened in cell lines will be additionally tested for their ability to induce an edit in the URB1 coding region in bovine embryos. Specifically, gRNAs that generated the desired edits in cells will also be tested for their ability to do the same in embryos. Edited embryos will be generated as described below. Briefly, oocytes recovered from slaughterhouse ovaries will be in vitro fertilized. The RNP solution as described in Example 1 will be injected into the cytoplasm of presumptive zygotes at 16-17 hours post-fertilization by using a single pulse from a FEMTOJET® 4i microinjector (Eppendorf SE; Hamburg, Germany). Microinjection will be performed in TL-Hepes (ABT360, LLC) supplemented with 3 mg/ml BSA (Proliant) on the heated stage of an inverted microscope equipped with Narishige micromanipulators (Narishige International USA, Amityville, NY). Following injections, presumptive zygotes will be cultured for 7 days in PZM5 (Cosmo Bio, Co LTD, Tokyo, Japan) in an incubator environment of 5% CO₂, 5% O₂, 90% N₂. Mutation frequency of blastocysts will be determined by ILLUMINA® sequencing as described for cell lines above. The frequencies of the desired edits in URB1 will be measured.

### Example 3

This example illustrates editing bovine cells.

Exemplary bovine editing will be by established methods (Ikeda, M., et al., Scientific Reports, 2017, 7, 1-9; Gao, Y., et al., Genome Biology, 2017, 18, 1-15). Briefly, a CRISPR/Cas9 sgRNA will be designed to introduce a double-strand DNA break near the components can be delivered to cells or fertilized embryos as follows: sgRNPs will be assembled and mixed with a ssODN repair template to achieve upon injection 1.56 amol of Cas9, 5.13 amol of sgRNA and 10.07 amol of ssODN. The sgRNP solution will be injected into the cytoplasm of presumptive zygotes at 16-17 hours post-fertilization by using a single pulse from a FemtoJet 4i microinjector (Eppendorf; Hamburg, Germany) with settings at pi=200 hPa, ti=0.25 s, pc=15 hPa. Glass capillary pipettes with an outer diameter of 1.2 mm and an inner diameter of 0.94 mm will be pulled to a very fine point of <0.5 μm (Sutter Instrument, Navato, CA, USA). Microinjection will be performed in TL-Hepes (ABT360, LLC) supplemented with 3 mg/ml BSA (Proliant) on the heated stage of an inverted microscope equipped with Narishige (Narishige International USA, Amityville, NY) micromanipulators. Following injections, presumptive zygotes will be cultured for 7 days in BO-IVC (IVF Bioscience, Falmouth, Cornwall, UK) in an incubator environment of 5% CO2, 5% O2, 90% N2. The fertilized embryos can then be transferred to recipient females and gestated until birth.

### Example 4

This example illustrates molecular characterization of edited animals by sequencing.

A tissue sample will be taken from an animal with a genome edited according to the examples herein. Tail, ear notch, or blood samples are suitable tissue types. The tissue sample will be frozen at −20° C. within 1 hour of sampling to preserve integrity of the DNA in the tissue sample.

DNA will be extracted from tissue samples after proteinase K digestion in lysis buffer. Characterization will be performed on two different sequence platforms, short sequence reads using the ILLUMINA® platform (ILLUMINA®, Inc., San Diego, CA) and long sequence reads on an Oxford NANOPORE™ platform (Oxford NANOPORE™ Technologies, Oxford, UK).

For short sequence reads, two-step PCR will be used to amplify and sequence the region of interest. The first step is a locus-specific PCR which amplifies the locus of interest from the DNA sample using a combined locus-specific primer with a vendor-specific primer. The second step attaches the sequencing index and adaptor sequences to the amplicon from the first step so that sequencing can occur.

The locus-specific primers for the first step PCR are chosen so that they amplify a region <300 bp such that ILLUMINA® paired-end sequencing reads could span the amplified fragment. Multiple amplicons are preferred to provide redundancy should deletions or naturally occurring point mutations prevent primers from correctly binding. Sequence data for the amplicon will be generated using an ILLUMINA® sequencing platform (MISEQ®, ILLUMINA®, Inc., San Diego, CA). Sequence reads are analyzed to characterize the outcome of the editing process.

For long sequence reads, two-step PCR will be used to amplify and sequence the region of interest. The first step is a locus-specific PCR which amplifies the locus of interest from the DNA sample using a combined locus-specific primer with a vendor-specific adapter. The second step PCR attaches the sequencing index to the amplicon from the first-step PCR so that the DNA is ready for preparing a sequencing library. The step 2 PCR products undergo a set of chemical reactions from a vendor kit to polish the ends of the DNA and ligate on the adapter containing the motor protein to allow access to the pores for DNA strand-based sequencing.

The locus specific primers for the first step PCR range will be designed to amplify different regions of the URB1 gene and will amplify regions different in length. Normalized DNA is then mixed with vendor supplied loading buffer and is loaded onto the NANOPORE™ flowcell (Oxford Nanopore Technologies, Oxford, UK).

Long sequence reads, while having lower per base accuracy than short reads, are very useful for observing the long-range context of the sequence around the target site.

### Example 5

This example illustrates the use of base editing or prime editing to generate a PRRSv-resistant pig by changing a splice site acceptor in CD163.

PRRSv is a positive-strand RNA virus known to cause PRRS. CD163 is a membrane-bound protein considered to be a fusion receptor for PRRSv. Exon 7 of CD163 encodes the SRCR domain 5 (SRCR5) that serves as an interaction site for PRRSv in vitro. Burkard et al. (Burkard, C., PLOS Pathog. 2017, 23, 13, e1006206) demonstrated that removal of CD163 exon 7 from genomic DNA via CRISPR/Cas-based gene editing confers PRRSv resistance to porcine macrophages. It would be advantageous to identify an approach that prevents inclusion of CD163 exon 7 from mature mRNA transcripts without the use of DSBs, as is the case with Cas9 and other nucleases.

The introns flanking exon 7 of CD163 contain canonical GT-AG splice sites. Therefore, alteration of the guanosine at the 3′ end of the intron preceding exon 7 is expected to result in exon skipping, preventing incorporation of exon 7 into the mature mRNA transcript. One approach to altering this guanosine is to convert the complementary cytidine to thymidine using a cytosine base editor, resulting in substitution of the target guanosine to adenosine. Several applications can be used to design gRNAs for use in base editing experiments. Using one such application, PnB Designer (Siegner, S. M., et al., BMC Bioinformatics, 2021, 22, 101), a gRNA was designed to alter the complementary cytidine to thymidine. The gRNA sequence is 5′-TGGG-C-TGAAAAATAGCATTT-3′ (SEQ ID NO: 124), with the targeted cytidine set aside by hyphens and in italics. This gRNA could be used with base editors that include, but are not limited to, BE3 (R33A), BE3 (R33A/K34A), BE3-hA3A (R128A), and Target-AID.

Another approach that could be used to facilitate CD163 exon 7 skipping is to use prime editing to alter the guanosine at the 3′ end of the intron preceding exon 7. Here, PnB Designer can be used to design components of the pegRNA needed to facilitate the guanosine to adenosine substitution. Table I shows the protospacer and the coding strand 3′ extension (primer binding site, or PBS; and reverse transcriptase template, or RTT) for two pegRNAs. Table II shows optional nicking guide protospacer sequences to be used in PE3 or PE3b prime editing systems.

TABLE I
pegRNAs for G to A substitution in CD163

| peg- | | Extension |
|---|---|---|
| RNA | Protospacer (sense) | (coding strand) |
| 1 | AACCAGCCTGGGTTTCCTGT | TTTTCAACCCACAGGAAACCCA |
| | (SEQ ID NO: 125) | GGCT (SEQ ID NO: 126) |
| 2 | CAACCAGCCTGGGTTTCCTG | TTTCAACCCACAGGAAACCCAG |
| | (SEQ ID NO: 127) | GCTG (SEQ ID NO: 128) |

TABLE II
Nicking guides for pegRNAs in CD163

| Protospacer | System |
|---|---|
| AATGCTATTTTTCAACCCAC | PE3b |
| (SEQ ID NO: 129) | |
| TTTCAACCCACAGGAAACCC | PE3b |
| (SEQ ID NO: 130) | |
| CTGTGATTCTGACTTCTCTC | PE3 |
| (SEQ ID NO: 131) | |
| AAGTACAACATGGAGACACG | PE3 |
| (SEQ ID NO: 132) | |
| GGTCGTGTTGAAGTACAACA | PE3 |
| (SEQ ID NO: 133) | |
| AGTACAACATGGAGACACGT | PE3 |
| (SEQ ID NO: 134) | |
| GTACAACATGGAGACACGTG | PE3 |
| (SEQ ID NO: 135) | |
| TGATTCTGACTTCTCTCTGG | PE3 |
| (SEQ ID NO: 136) | |
| CTTTCTCACTTTTCACTCTC | PE3 |
| (SEQ ID NO: 137) | |
| CTGGCTTACTCCTATCATGA | PE3 |
| (SEQ ID NO: 138) | |
| CCTATCATGAAGGAAAATAT | PE3 |
| (SEQ ID NO: 139) | |

These edits can be introduced into porcine embryos using similar methods to those published in U.S. Pat. No. 11,535,850 to Genus PLC. Edited embryos can be implanted into a surrogate mother for gestation until birth. Edited animals can then be identified using the methods of Example 4. Animals can then be challenged with PRRSV as described in U.S. Pat. No. 11,535,850 to Genus PLC.

It is expected that pigs having a genome comprising a CD163 gene that is edited to skip exon 7 during mRNA maturation will be resistant to the PRRSv.

### Example 6

This example illustrates the use of base or prime editing to introduce a codon change for an amino acid substitution.

Another desirable edit is in the Bos taurus CD18 gene: changing the codon for glutamine at amino acid position 18 to glycine, for example, may allow for cleavage of the CD18 signal peptide. This cleavage may confer resilience to Mannheimia haemolytica leukotoxin (Shanthalingam, S. and Srikumaran, S., Proc. Natl. Acad. Sci. USA, 2009, 106, 15448-15453). A gRNA was designed using PnB Designer to alter the codon encoding glutamine at amino acid position 18 to glycine (CAG>CGG) using base editing. The gRNA sequence is 5′-AGCAT-A-GGTGGGTCCCGCTG-3′ (SEQ ID NO: 140), with the targeted alanine italicized and surrounded by dashes. This gRNA is suitable for use with a base editor such as, but without limitation, the SpRY-ABEmax base editor.

The desired base editor or prime editing components can be delivered to cells or fertilized embryos to introduce the edit in methods similar to those published in U.S. Pat. No. 11,535,850 to Genus PLC. Edited cells or edited animals can then be evaluated for disease resistance in live pathogen animal challenges or cell-based assays using, but not limited to, whole blood, serum, monocyte-derived macrophages or pulmonary alveolar macrophages.

All publications cited herein are hereby incorporated by reference, each in their entirety.

What is claimed is:

1. An ungulate comprising one or more edits in one or more genes that affects a livestock trait.

2. The ungulate of claim 1, wherein the ungulate is a porcine animal.

3. The ungulate of claim 1, wherein the ungulate is a bovine animal.

4. The ungulate of claim 1, wherein the ungulate is a pig.

5. The ungulate of claim 1, wherein the ungulate is a Bos taurus animal.

6. The ungulate of claim 1, wherein the one or more genes is a PRLR, NANOS2, Deadend (Dnd), APAF1, SMC2, GART, TFBIM, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPP1R12A, ZFP36L2, CSPP1, CHI3L2, GBP6, PPFIBP1, REP15, CYP4F2, TIGD2, PYURF, SLC10A2, ARHGEF17, RELT, PRDM2, KDM5B, PLAG1, KCNA6, NDUFA9, AKAP3, C5H12orf4, RAD51AP1, FGF6, CCND2, CSMD3, AQP3, AQP7, HSPB8, DCAF8, SLC16A3, TIGAR, CD18, ANP32, ANPEP, TMPRSS1, TMPRSS2, NANOS2, CD163, Melanocortin-4 receptor (MC4R), HMGA, IGF2, HAL, RN, Mx1, BAT2, EHMT2, PRDM1, PRDM14, or ESR gene.

7. The ungulate of claim 1, wherein the livestock trait is high altitude adaptation and response to hypoxia, cold acclimation, body size and stature, resistance to disease and bacterial infection, reproduction, milk yield and components, growth and feed efficiency, or polled phenotype.

8. A method of editing a gene affecting a livestock trait of an ungulate comprising:
contacting an editing reagent with an isolated cell of the ungulate.

9. The method of claim 8, wherein the editing reagent comprises a DNA editing system selected from the group consisting of a meganuclease, a zinc finger nucleases (ZFN), a transcription-activator like effector nuclease (TALEN) and CRISPR.

10. The method of claim 8, wherein the editing reagent comprises one or more gRNAs and a CAS9 protein or a polynucleotide encoding a CAS9 protein.

11. The method of claim 8, wherein the editing reagent comprises a ribonucleoprotein complex comprising a gRNA and a CAS9 protein.

12. The method of claim 8, wherein the ungulate is a bovine animal.

13. The method of claim 8, wherein the ungulate is a porcine animal.

14. The method of claim 8, wherein the ungulate is a swine.

15. The method of claim 8, wherein the ungulate is a Bos taurus, Bos indicus, or Bubalus bubalis animal.

16. The method of claim 8, wherein the ungulate is a pig.


SEQUENCE LISTING (140 sequences)

SEQ ID NO: 1 - DNA, 2817 bp, Bos taurus
  source 1..2817: mol_type = other DNA
gtcttcccagatccgcgaagcaccagcctggtgaagagcagagccgaagcccctgccagtccagctgggacacccctgccgaggtctccagggcatccaggggacatgctgcgccagcgcccccagctgctgctcctagcgggcctgcttgccctccagtccgtcctgtcccaggagtgcaccaactacaaggtcagcacctgccgggactgcatcgagtcgggccccggctgcgcctggtgccagaaactgaacttcacagggcaaggggagcccgactccattcgctgtgacacacgagcggagctgctgtcaaagggctgcccagctgatgacatcatggaacccaagagcctcgctgagacccgggacagccaggcgggcagtcggaagcagctgtccccacaggaagtgacgctctacctgagaccaggtcaggcagttgcgttcaacgtgaccttccggagggccaagggctaccccatcgacctgtactacctgatggacctctcctactccatggtggatgacctcgtcaacgtcaagaagctggggggtgacctgctccgggccctcaatggcatcaccgagtcgggccgcattggtttcgggtccttcgtggacaagacggtgctccccttcgtcaacacgcaccccgagaagctgcggaacccctgccccaacaaggagaaggagtgccagcccccgttcgccttcaggcacgtgttgaagctcactgacaactccaaacagttcgagacagaagtcgggaagcagctgatctcggggaacctggacgcccctgagggtgggctggacgccatgatgcaggtggccgcgtgcccggaggaaatcggctggcgcaatgtcaccaggctgctggtgttcgccacggacgatgggttccactttgcgggcgatggaaagctgggtgccatcctcacccccaatgacggccgctgccacctggaagacaacctgtacaaaagcagcaacgaatttgactacccatcggtgggccagctggcacacaaactggcagaaagcaacatccagcccatctttgcagtaaccaagaagatggtgaaaacgtacgagaagctgacagagatcatccccaagtctgcagtcggggagctgtctgaagattccaggaacgtggtggagcttatcaagaatgcctacaataaactgtcctccagagtcttcctggatcacagcaccctccctgacaccctgaaagtcacctacgactccttctgcagtaacgggaaatcgcaggtggaccagcccagaggggactgcgacggcgtccagatcaacgtcccgatcaccttccaggtgaaggtcacagccaccgagtgcatccagcagcagtccttcaccatccgggcgctgggcttcacggacacggtgaccgtgcgggtcctcccccagtgcgagtgccaatgccgggacgccagcagggacggcagcatctgcggcggcagaggctcgatggagtgcggcgtctgcaggtgtgacgccggctacatcgggaagaactgcgagtgccagacgcagggccggagcagccaggagctggagggcagctgccgcaaggacaacagctccatcatctgctcggggctgggggactgcatctgcgggcagtgcgtgtgccacacgagcgacgtgcccaacaagaagatctacggccagttctgcgagtgcgacaacgtcaactgcgaacgctacgacggccaagtctgcgggggcgaaaagagggggctctgcttctgcggcacctgcaggtgcgacgagcagtatgagggttcggcatgccagtgcctcaagtccactcagggctgcctcaacttggacggcgtcgagtgcagcggccgcggccgatgccgctgcaatgtgtgccagtgcgaccccggctaccagccgcccctgtgcagcgagtgcccgggctgccccgtgccctgtgcgggcttcgccccctgcacagagtgcctgaagttcgacaagggccccttcgccaagaactgcagcgcagcgtgcgggcagacgaagctgctgtccagcccggtgcccggccgcaagtgcaaggagcgcgactccgagggctgctggatgacctacaccctggtgcagcgcgacgggcgggacagatacgacgtgcacgtggacgacatgctcgagtgtgtgaagggccccaacatcgctgccatcgtggggggcaccgtggggggcgtcgtgctcgtcggcatcctcctgctggtcatctggaaggccctgacacacctgagcgacctcagggagtaccatcgctttgagaaggagaagctcaagtcccagtggaacaacgataaccctcttttcaagagtgccaccacgacagtcatgaaccctaagtttgccgagagttaggggtgcccggtgaagacaaggccttctgcaccacccagatgggaacaccccctctccacgtcccctccagcaggctgaccgtgaccccgctgcctcgtggacgtggctgacaacttcaccgttaaccaaaaatgcactgctttttctgccccagaatgatgggcgtggccaggttattctatgggctcatggtaagggccagcctaccccttctgatatgaatgacttttgatagcaagtcagagaaggaattgcctacattttgtatggttacacacaggtcctttgtaaaaattagtacagcagtctgatgaagaattatttatgtgtgaacttctcagggtatgaagttacatccccttggttatgctgcccccaatcaataaaaaaaaaaaaatcaat

SEQ ID NO: 2 - DNA, 2507 bp, Capra hircus
  source 1..2507: mol_type = other DNA
cagcctggtgaagagcagagccgaagcccctgccagtccagctgggacacccctgccgtggtctccagggcatccaggggacatgctgccccagcgcccccagctgctgctcctagcaggcctgctcgccctccagtctgtcctgtcccaggagtgcaccaagtacaaagtcagcacctgccgggactgcatcgagtcgggccccggctgtgcctggtgccagaaactgaacttcacagggcaaggggagcccgactccactcgctgtgatacacgggcgcagctgctgtcaaagggctgcccagctgatgacatcatggaacccaagagcctcgctgagacccggcagagccaggcgggcaaacagaagcagctgtccccagaggaagtgactctctaccttagaccaggtcaggcagctgcgttcaatgtgaccttccagagggccaagggctaccccatcgacctgtactatctgatggatctctcctactccatggtggacgacctcgccaacgtcaagaagctggggggtgacctgctccgggccctcaatgacatcaccgagtcgggccgcatcgggttcgggtccttcgtggacaagacagtgctccccttcgtcaacacgcaccctgagaagctgaggaacccctgccccaacaaggagaagcagtgccagcccccgttcgccttcaggcacgtgttgaagctcaccgacaactccaaacagttcgagacagaagtcgggaagcagctgatctcggggaacttggacgcccctgagggtggactggacgccatgatgcaagtggccgcgtgcccggaggaaatcggctggcgcaatgtcaccaggctgctggtgttcgccacagatgatgggttccactttgcgggcgatggaaagctgggtgccatcctcacccccaacgacggccgctgccacctggaagacaacctgtacaaaagcagcaacgaatttgactacccatcggtgggccagctggcacacaaactggcagaaagcaacatccagcccatcttcgcggtaaccaagaagatggtgaaaacgtacgagaagctgacagaaatcatccccaagtctgcagtcggggagctgtctgaagattccaagaacgtggtggagcttatcaagagtgcctacaataaactgtcctccagagtattcctggatcacaacaccctccctgacaccctgaaagtcgcctacgactccttctgcagtaacggggtgtcacaggtggaccagcccagaggggactgtgacggcgtccagatcaacgtcccgatcaccttccaggtgaaggtcacagccaccgagtgcatccaggagcagtccttcaccatccgggcgctgggcttcacggacacggtgaccgtgcgggtccttccccagtgcgagtgccaatgccgggacgccagcagggaccgcagcgtctgcggtggcagaggctcgatggagtgcggcgtctgcaggtgcgacgccggctacatcgggaagaactgcgagtgccagacgcacggccggagcagccaggagctggagggcagctgccgcaaggacaacagctccatcatctgctcggggctaggggactgcatctgcgggcagtgcgtgtgccacacgagcgacgtgcccaacaagaagatctacggccagttctgcgagtgcgacaacgtcaactgcgagcgctacgacggccaagtctgcgggggcgagaagagggggctctgcttctgcggcacctgcaggtgcaacgagcagcatgagggctcggcgtgccagtgcctcaagtccactcagggctgcctcaacctggacggcgtcgagtgcagcggccggggccgatgccgctgcaacgtgtgccagtgcgaccccggctaccagccgcccctgtgcatcgactgcccgggctgccccgtgccctgcgctggcttcgccccctgcaccgagtgcctgaagttcgacaagggccccttcgccaagaactgcagcgcagcgtgcgggcagacgaagctgctgtccagcccggtgcccggcggccgcaagtgcaaggagcgcgactccgagggctgctggatgacctacaccctggtgcagcgcgacgggcggaacagatacgacgtgcacgtggacgacatgctcgagtgtgtgaagggccccaacatcgctgccatcgtggggggcaccgtggggggagttgtgctcgtcggcatcctcctgctggtcatctggaaggccctgacacacctgagcgacctcagggagtaccatcgcttcgagaaggagaagctcaagtcccagtggaacaacgataaccctcttttcaagagtgccaccacgacagtcatgaaccctaagtttgccgagagttaggggtgcctggtgaagacaaggccttctgcaccacccagacgggagcacgccctctcctcatcccctccagcaggctgaccgtgaccttgctgcttagtggacgcagctgatg

SEQ ID NO: 3 - DNA, 2313 bp, Ovis aries
  source 1..2313: mol_type = other DNA
atgctgccccagcgcccccagctgctgctcctagcgggcctgctctccctccagtccgtcctgtcccaggagtgcaccaagtacaaagtcagcacctgccgggactgcatcgagtcgggccccggctgtgcctggtgtcagaaactgaacttcacagggcaaggggagcccgactccactcgctgtgatacacgggcgcagctgctgtcgaagggctgcccagctgatgacatcatggaacccaagagcctcgctgagactcggcagagccaggcgggcaaacagaagcagctgtccccagaggaagtgaccctctacctgagaccaggtcaggcagcagcgttcaacgtgaccttccagagggccaagggctaccccatcgacctgtactatctgatggatctctcctactccatggtggacgacctcgccaacgtcaagaagctggggggtgacctgctccgggccctcaatgacatcaccgagtcaggccgcattggtttcgggtccttcgtggacaagacggtgctccccttcgtcaacacgcaccccgagaagctgaggaacccctgccccaacaaggagaaggagtgccagccgccgttcgccttcaggcacgtgctgaagctcaccgacaactccaaacagttcgagacagaagtcgggaagcagctgatctcggggaacttggacgcccctgagggtgggctggacgccatgatgcaagtggccgcgtgcccggaggaaattggctggcgcaatgtcaccaggctgctggtgttcgccacagacgatgggttccactttgcgggcgatggaaagctgggtgccatcctcacccccaacgacggccgctgccacctggaagacaacctgtacaaaagcagcaacgaatttgactacccatcggtgggccagctggcacacaaactggcagaaagcaacatccagcccatcttcgcggtaaccaagaagatggtgaaaacgtacgagaagctgacagaaatcatccccaagtctgcagtcggggagctgtctgaagattccaagaacgtggtggagcttatcaagagtgcctacaataaactgtcctccagagtattcctggatcacaacaccctccctgacaccctgaaagtcgcctacgactccttctgcagtaacggggtgtcgcaggtggaccagcccagaggggactgcgacggcgtccagatcaacgtcccgatcaccttccaggtgaaggtcacagccaccgagtgcatccaggagcagtccttcaccatccgggcgctgggcttcacggacacggtgaccgtgcgggtccttccccagtgcgagtgccaatgccgggaagccagcagggaccgcagcgtctgcggtggcagaggctcgatggagtgcggcgtctgcaggtgcgacgccggctacatcgggaagaactgcgagtgccagacgcacggccggagcagccaggagctggagggcagctgccgcaaggacaacagctccatcatctgctcggggctgggggactgcatctgcgggcagtgcgtgtgccacacgagcgacgtgcccaacaagaagatctacggccagttctgcgagtgcgacaacgtcaactgcgagcgctacgacggccaagtctgcgggggcgacaagagggggctctgcttctgcggcacctgcaggtgcaacgaccagcatgagggctcggcgtgccagtgcctcaagtccactcagggctgcctcaacctggacggcgtcgagtgcagcggccgcggccgatgccgctgcaacgtgtgccagtgcgaccccggctaccagccgcccctgtgcatcgactgcccgggctgccccgtgccctgcgctggcttcgccccctgcaccgagtgcctgaagttcgacaagggtcccttcgccaagaactgcagcgcagcgtgcgggcagacgaagctgctgtccagcccggtgcccggcggccgcaagtgcaaggagcgtgactccgagggctgctggatgacctacaccctggtgcagcgcgacgggcggaacagatacgacgtgcacgtggacgacatgctcgagtgtgtgaagggccccaacatcgctgccatcgtggggggcaccgtggggggagttgtgctcgtcggcatcctcctgctggccatctggaaggccctgacacacctgagcgacctcagggagtaccatcgcttcgagaaggagaagctcaagtcccagtggaacaacgataaccctcttttcaagagtgccaccacgacagtcatgaaccctaagtttgccgagagttag

SEQ ID NO: 4 - DNA, 2313 bp, Ovis canadensis
  source 1..2313: mol_type = other DNA
atgctgccccagcgcccccagctgctgctcctagcgggcctgctctccctccagtccgtcctgtcccaggagtgcaccaagtacaaggtcagcacctgccgggactgcatcgagtcgggccccagctgtgcctggtgccagaaactgaacttcacagggcaaggggagcccgactccactcgctgtgatacaagggcgcagctgctgtcgaagggctgcccagctgatgacatcatggaacccaagagcctcgctgagacccggcagagccaggcgggcagacagaagcagctgtccccagaggaagtgaccctctacctgagaccaggtcaggcagctgcgttcaacgtgaccttccagagggccaagggctaccccatcgacctgtactatctgatggatctctcctactccatggtggacgacctcgccaacgtcaagaagctggggggtgacctgctccgggccctcaatgacatcaccgagtcgggccgcattggtttcgggtccttcgtggacaagacggtgctccccttcgtcaacacgcaccccgagaagctgaggaacccctgccccaacaaggagaaggagtgccagccgccgttcgccttcaggcacgtgctgaagctcaccgacaactccaaacagttcgagacagaagtcgggaagcagctgatctcggggaacttggacgcccctgagggtgggctggacgccatgatgcaagtggccgcgtgcccggaggaaattggctggcgcaatgtcaccaggctgctggtgttcgccacagacgatgggttccactttgcgggcgatggaaagctgggtgccatcctcacccccaacgacggccgctgccacctggaagacaacctgtacaaaagcagcaacgaatttgactacccatcggtgggccagctggcacacaaactggcagaaagcaacatccagcccatcttcgcggtaaccaagaagatggtgaaaacgtacgagaagctgacagaaatcatccccaagtctgcagtcggggagctgtctgaagattccaagaacgtggtggagcttatcaagagtgcctacaataaactgtcctccagagtattcctggatcacaacaccctccctgacaccctgaaagtcgcctacgactccttctgcagtaacagggtgtcacaggtggaccagcccagaggggactgtgacggcgtccagatcaacgtcccgatcaccttccaggtgaaggtcacagccaccgagtgcatccaggagcagtccttcaccatccgggcgctgggcttcacggacacggtgaccgtgcgggtccttccccagtgcgagtgccaatgccgggaagccagcagggaccgcggcgtctgcggtggcagaggctcgatggagtgcggcgtctgcaggtgcgacgccggctacatcgggaagaactgcgagtgccagacgcacggccggagcagccaggagctggagggcagctgccgcaaggacaacagctccatcatctgctcggggctgggggactgcatctgcgggcagtgcgtgtgccacacgagcgacgtgcccaacaagaagatctacggccagttctgcgagtgcgacaacgtcaactgcgagcgctacgacggccaagtctgcgggggcgacaagagggggctctgcttctgcggcgcctgcaggtgcaacgaccagtatgagggctcggcgtgccagtgcctcaagtccactcagggctgcctcaacctgaacggcgtcgagtgcagcggccgcggccgatgccgctgcaacgtgtgccagtgcgaccccggctaccagccgcccctgtgcatcgactgcccgggctgccccgtgccctgcgctggcttcgccccctgcaccgagtgcctgaagttcgacaagggtcccttcgccaagaactgcagcgcagcgtgcgggcagacgaagctgctgtccagcccggtgcccggcggccgcaagtgcaaggagcgtgactccgagggctgctggatgacctacaccctggtgcagcgcgacgggcggaacagatacgacgtgcacgtggacgacatgctcgagtgtgtgaagggccccaacatcgctgccatcgtggggggcaccgtggggggagttgtgctcgtcggcatcctcctgctggccatctggaaggccctgacacacctgagcgacctcagggagtaccatcgcttcgagaaggagaagctcaagtcccagtggaacaacgataaccctcttttcaagagtgccaccacgacagtcatgaaccctaagtttgccgagagttag

SEQ ID NO: 5 - DNA, 3400 bp, Sus scrofa
  source 1..3400: mol_type = other DNA
atggtgctacttgaagactctggatctgcagactttagaagatgttctgcccatttaagttccttcacttttgctgtagtcgctgttctcagtgcctgcttggtcactagttctcttggaggaaaagacaaggagctgaggctaacgggtggtgaaaacaagtgctctggaagagtggaggtgaaagtgcaggaggagtggggaactgtgtgtaataatggctgggacatggatgtggtctctgttgtttgtaggcagctgggatgtccaactgctatcaaagccactggatgggctaattttagtgcaggttctggacgcatttggatggatcatgtttcttgtcgagggaatgagtcagctctctgggactgcaaacatgatggatggggaaagcataactgtactcaccaacaggatgctggagtaacctgctcagatggatctgatttagagatgaggctggtgaatggaggaaaccggtgcttaggaagaatagaagtcaaatttcaagagcggtggggaacagtgtgtgatgataacttcaacataaatcatgcttctgtggtttgtaaacaacttgaatgtggaagtgctgtcagtttctctggttcagctaattttggagaaggttctggaccaatctggtttgatgatcttgtatgcaatggaaatgagtcagctctctggaactgcaaacatgaaggatggggaaagcacaattgcgatcatgctgaggatgctggagtgatttgcttaaatggagcagacctgaaactgagagtggtagatggactcactgaatgttcaggaagattggaagtgaaattccaaggagaatggggaacaatctgtgatgatggctgggatagtgatgatgccgctgtggcatgtaagcaactgggatgtccaactgctgtcactgccattggtcgagttaacgccagtgagggaactggacacatttggcttgacagtgtttcttgccatggacacgagtctgctctctggcagtgtagacaccatgaatggggaaagcattattgcaatcataatgaagatgctggtgtgacatgttctgatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaagccaaaattacctgctcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcagggaaccagagtcagacactatccccgtgcaattcatcatcctcggacccatcaagctctattatttcagaagaaagtggtgttgcctgcatagggagtggtcaacttcgcctggtcgatggaggtggtcgttgtgctgggagagtagaggtctatcctggggcatcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcagagttcatgtctctgagactgatcagtgaaaacagcagagagacctgtgcagggcgcctggaagttttttacaacggagcttggggcagcgttggcaggaatagcatgtctccagccacagtgggggtggtatgcaggcagctgggctgtgcagacagaggggacatcagccctgcatcttcagacaagacagtgtccaggcacatgtgggtggacaatgttcagtgtcctaaaggacctgacacactatggcagtgcccctcatctccatggaagaagagactggccagcccctcagaggagacatggatcacatgtgccaacaaaataagacttcaagaaggaaacactaattgttctggacgtgtggagatctggtacggaggttcctggggcactgtgtgtgacgactcctgggaccttgaagatgctcaggtggtgtgccgacagctgggctgtggctcagctttggaggcaggaaaagagcccgcatttggccaggggactgggcccatatggctcaatgaagtgaagtgcaaggggaatgaaccctccttgtgggattgtcctgccagatcctggggccacagtgactgtggacacaaggaggatgctgctgtgacgtgctcagaaattgcaaagagccgagaatccctacatgccacaggtcgctcatcttttgttgcacttgcaatctttggggtcattctgttggcctgtctcatcgcattcctcatttggactcagaagcgaagacagaggcagcggctctcagttttctcaggaggagagaattctgtccatcaaattcaataccgggagatgaattcttgcctgaaagcagatgaaacggatatgctaaatccctcaggagaccactctgaagtacaatgaaaaggaaaatgggaattataacctggtgagttcagcctttaagataccttgatgaagacctggacta

SEQ ID NO: 6 - DNA, 3884 bp, Sus scrofa
  source 1..3884: mol_type = other DNA
tctgtatttttgtggaagtgaaatgatttttagaattcttagtggttctcttcttcaggagaacatttctagggaataatacaagaagatttaaatggcataaaaccttggaatggacaaactcagaatggtgctacatgaaaactctggatctgcagactttagaagatgttctgcccatttaagttccttcacttttgctgtagtcgctgttctcagtgcctgcttggtcactagttctcttggaggaaaagacaaggagctgaggctaacgggtggtgaaaacaagtgctctggaagagtggaggtgaaagtgcaggaggagtggggaactgtgtgtaataatggctgggacatggatgtggtctctgttgtttgtaggcagctgggatgtccaactgctatcaaagccactggatgggctaattttagtgcaggttctggacgcatttggatggatcatgtttcttgtcgagggaatgagtcagctctctgggactgcaaacatgatggatggggaaagcataactgtactcaccaacaggatgctggagtaacctgctcagatggatctgatttagagatgaggctggtgaatggaggaaaccggtgcttaggaagaatagaagtcaaatttcaaggacggtggggaacagtgtgtgatgataacttcaacataaatcatgcttctgtggtttgtaaacaacttgaatgtggaagtgctgtcagtttctctggttcagctaattttggagaaggttctggaccaatctggtttgatgatcttgtatgcaatggaaatgagtcagctctctggaactgcaaacatgaaggatggggaaagcacaattgtgatcatgctgaggatgctggagtgatttgcttaaatggagcagacctgaaactgagagtggtagatggagtcactgaatgttcaggaagattggaagtgaaattccaaggagaatggggaacaatctgtgatgatggctgggatagtgatgatgccgctgtggcatgtaagcaactgggatgtccaactgctgtcactgccattggtcgagttaacgccagtgagggaactggacacatttggcttgacagtgtttcttgccatggacacgagtctgctctctggcagtgtagacaccatgaatggggaaagcattattgcaatcataatgaagatgctggtgtgacatgttctgatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaagccaaaattacctgctcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcagggaaccagagtcagacactatccccgtgcaattcatcatcctcggacccatcaagctctattatttcagaagaaaatggtgttgcctgcatagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctaagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcggagttcatgtctctgagactgatcagtgaaaacagcagagagacctgtgcagggcgcctggaagttttttacaacggagcttggggcagcgttggcaagaatagcatgtctccagccacagtgggggtggtatgcaggcagctaggctgtgcagacagaggggacatcagccctgcatcttcagacaagacagtgtccaggcacatgtgggtggacaatgttcagtgtcctaaaggacctgacacactatggcagtgcccatcatctccatggaagaagagactggccagcccctcagaggagacatggatcacatgtgccaacaaaataagacttcaagaaggaaacactaattgttctggacgtgtggagatctggtacggaggttcctggggcactgtgtgtgacgactcctgggaccttgaagatgctcaggtggtgtgccgacagctgggctgtggctcagctttggaggcaggaaaagaggccgcatttggccaggggactgggcccatatggctcaatgaagtgaagtgcaaggggaatgaaacctccttgtgggattgtcctgccagatcctggggccatagtgactgtggacacaaggaggatgctgctgtgacgtgttcagaaattgcaaagagccgagaatccctacatgccacaggtcgctcatcttttgttgcacttgcaatctttggggtcattctgttggcctgtctcatcgcattcctcatttggactcagaagcgaagacagaggcagcggctctcagaagaatgctacctgtctttggggaagaaacagggttatccgatggcatcctgttgcgtttgagttcgtgacatcatgagggaaaggcttggcagcgtttactcgttttctcaggaggagagaattctgtccatcaaattcaataccgggagatgaattcttgcctgaaagcagatgaaacggatatgctaaatccctcagaaaattccaatgaataagatggttttaatgatgtgggactaatttttgtgtctaaatctcttcctatttctggatgaaaaaaaggagaccactctgaagtacaatgaaaaggaaaatgggaattataacctggtgagttcagcctttaagataccttgatgaagacctggactattgaatgagcaagaatctgcctcttacactgaagattacaatacagtcctctgtctcctggtattccaaagactgctgctgaatttctaaaaaatagattggtgaatgtgactactcaaagttgtatgtaagactttcaagggcattaaataaaaaagaatattgctga

SEQ ID NO: 7 - DNA, 3390 bp, Bos taurus
  source 1..3390: mol_type = other DNA
atggtgctacttgaagactctggatctgctggttttaaaaggtgttctgtccacttcggtcccttcactcttgctgtggtctcagttctctatgcctgtttggtcaccagtgctcttgggggaacagacaaggaactgaggctggtagctggtcaaaccaagtgttctgggagagtggaggtgaaagtccaggaagagtggggaactgtgtgcaacaccggctgggacttggctgccgtctctgtcgtttgtaagcagctgggatgtccgtctgtcatcaaagccacaggatggaccaattccagtgcaggcactgggcgcatttggatggatcatgtgtcttgtcgaggcaatgaatcagctctctgggactgcaaacatgaggggtggggaaagcataactgtactcaccaacaggatgtaggagtcacctgctcagatggatctgatttggagatgaggctgatgaatggaggaaaccggtgctcaggaagaatagagatcaaattccagggccagtggggaacagtgtgtgacgataacttcaacttggatcacgcttctgtggtttgtaaacagcttggatgtggaagtgctgtcagtttctctggctcagctaattttggagaagggtctgggccaatctggtttgatgatctggtatgccatggaaatgagtcagctctctggaactgcagacacgaaggatggggaaagcacaactgtgatcacgctgaagatgccggagtgatttgcttagagggagctgacctgagcctgagactggtagatggagtcaccaagtgttcaggaagattagaagtgagattccaaggagaatgggggacagtgtgtgatgatggctgggacagtgatgatgctgctgtagcctgtcagcaattgggatgtccgactgccatcactgccgtcggtagagtcaatgccagtgagggaactggacacatttggcttgacagtgtttcctgccaaggacatgaatctgctgtctggcagtgcagacatcacgaatggggaaagcattactgcaatcataacgaagatgctggtgtgacctgttctgatggatcagatctcgaactgagactcaaaggtggaggtagccgctgtgctgggacagtggaggtggaaattcagaaactgatagggaaagtgtgtgacagaagctggggactgaaagaagctgacgtggtttgcaagcagctgggatgtggatctgcgctcagaacatcctatcaagtttattctaaaattcaggcaacgaacacatggctgtttttaaacaactgcaatggaaatgagacttctatctgggactgcaagaactggcaatggggtgggcttagttgtgaacactatcatgaagccaaagttacttgctcagcccacagggaacccagactagttggaggggacattccttgctccggtcgtgttgaagtgaaacatggcgacacttggggcaccatctgtgattctgacttctctctggaatcggccagcgtgctatgcagggaattagagtgcggcagcgttgtctccatcctggggggagctcactttggagaaggaaatggacagatctgggctgaagaattccagtgtgaaggcaacgagtcccatctttcactctgccccgttgcaccccgcctggatgggacgtgtagccacagcaaggacattggagtagtctgctcaagatacacagaagttcgcttggtgggtggcaataccccatgtgaaggaagagtggaggtcaagattcttgggacctggggacctctctgcaactctcactgggacatggaagatgctcatgttttgtgccagcagcttaaatgtggagttgctgcttctatcccaggaagagcaccttttgggaaaggaagtgggcagccctggagacacatgttccactgcactgggacagaacagcacatgggtgattgtcctgtaactgctctgggtgcatcgctgtgtcctgaagggcaagtggcctctgtaatctgctcaggaaatcggagtcagacactatatccatgcaattcatcatcttcagatccggaaagctccgttgttttggaagagaatggtgttccttgcatagggagtggacagctccgcctggtaaatggaggtgggcgttgtgctgggagaatagaggtctatcatgagggctcctggggcaccatctgtgatgacagctgggacctggatgatgcccacgtggtgtgcaggcagctgggctgcggtgtggccattaatgccacaggttctgctcattttggggaaggatcagggcccatctggctggatgaggtgaactgtaatggaaaagaacctcgcatttctcagtgccgttcccatggctgggggcggcagaactgcaggcataaggaggatgcaggagttatctgctcagagttcatgtctctcagactcatcagtgacagcagcagcgagacctgtgcagggcgcctggaagttttttacaacggagcttggggcagcgtgggcaagagcgacatgtctgcaaccacggtgggggtggtatgcaggcagctcggctgtactgacaaagggagtatcagaccagcaccttcagacaaggtggagaacaggtacatgtgggtggacaatgtccggtgtcctaagggacctgaaaccttatggcagtgcccatcatctccgtggaagaggagactggccagcccctcagaggagacctggatcacatgtgctgataaaataaggcttcaggaaggaaccactaattgttctggacgcgtggaggtctggcacggaggttcctggggcacagtgtgtgatgactcctgggaccttaacgatacccaggtggtgtgtcgccagctgggctgtggcttagctctggaggcaggaaaagaggcagcgttcggccagggaactgggcccatatggctcaatgaagtgaagtgcaagggcaatgagtcctccttgtgggattgccctgccagatcctggggacacagtgactgcgggcacaaggaggatgcctctgtgaagtgctcagaaattgcagagagcaaaggatcagtaaaggccgcaggtcactcatccactgttgcacttggaatccttggggtcattctgttggcctttctcatcgcaaccctcttgtggattcagaggcgaagacagagacagcggcttgcagtttcctcaagaggagagaactctgtccacgaaattcaataccgggagatgaattcttgcctaaaagcagatgatctggacctatataattcctcaggtctgtgggttcttagagggtccatagccctggggttcagactggtaactgcagctgaggctgagaggcattctacttga

SEQ ID NO: 8 - DNA, 3333 bp, Ovis aries
  source 1..3333: mol_type = other DNA
anggtgctacnngacagctctggatctgctggctttaaaaggtgttctgtccacttcggtcccttcactcttgctgtggtctcagttctctgtgccngtttgatcaccagtgctcttgggggaacagacanncaaagtgttctgggagagtggaggtgaaagtccaggaagagtggggaactgtgtgcaacaanggctgggacttggcggccntctccntcntttgtaagcagctggggtgtccgactgtcntcacagccacagganggaccaattccagtgcaggcactgggcgcntttgganggatcntgtttcttgtagaggcaangaatcagctctctgggactgcaaacnngaagggtggggaaagcataactgtactcaccaacaggangtaggagtcacctgctcaganggatctgatttggagangaggctgangaatggtggaaaccggtgctcaggaagaatcgagatcaaattccagggccagtggggaacagtgtgtgangataacttcaacttgaatcncgcttctgtggtttgtaaacagcttggangtggaagtgctntcagtttctctggctcagctaattttggagaagggtctgggccaatctggtttgangatctggtttgccntggaaangagtcagctctctggaactgcagacangaagganggggaaagcacaactgtgatcangctgaagangctggagtgatttgcttagagggagctgatctgagcctgagagtggtaganggagtcaccaagtgttcaggaagattagaagtgagattccaaggagaangggggacagtgtgtgangatggctgggacagtgangangctgctgtagcangtcagcaattgggangtccgactgccntcactgccntcggtagagtcaatgccagtgagggaactggacacatttggcttgacagtgtttcctgccaaggacatgaatctgctgtctggcagtgcagacatcangaanggggaaagcnttactgcaatcntaangaagangctggtgtgacctgttctgatggatcagatctcgagctgagactcaaaggtggaggtagccgctgtgctgggacagtggaggtggaaattcagaaattgacagggaaagtgtgtgacagaggctggggactgaaagaagctgangtggtttgcaagcaactgggatgtggatctgcactcaaaannncctnncnagtttantccnaagttnnnncaacaaacacnnggctgtttttaagcaactgcaaaggaaangaaacttctatctgggactgcaagaactggcaanggggtgggcttagttgtgaacactatgangaagccaaagttacttgctcagcccacagggaacccagactagtcggaggggacnttccctgctccggtcntgttgaagtgaagcnnggcgacacttggggcaccntctgtgattctgacttctctctggaatcggccaanntgctgtgcagggaattagagtgtggcagcnttgtttccntcctggggggagctcactttggagaaggaaanggacagatctgggctgaagaattccagtgtgagggcaacgagtcccntctttcactctgccctgttgcaccccgcccggangggangtgtagccacagcaaggannttggagtagtctgctcaagatacacagaagttcgcttggtgggtggaaataccccntgtgaaggaagagtggaggtcaagattcttgggacctgggganntctttgcaactctcactgggacanggaagangctcannttttgtgccagcagcttaaangtggagttgctgcttctatcccaggaagagcaccttttgggaaaggcagtgggcagctctggagacacangttccactgcactgggacagaacagcacnngggtgattgtcctgtaactgctctgggtgcatcgctgtgttctgaagggcaagtggcctctgtaatctgctcaggaaatcggagtcagacactanntccnngcaattcntcttcttcagatctgaaaagctccnttgttttggaagaaaatggtgttccttgcatagggagtggacagctccgcttggtaaanggaggtgggcnttgtgccgggagaatagaggtctatcnngagggctcctggggcaccatctgtgatgacagctgggacctggangangcccanntggtgtgcaggcagctgggctgcggtgtggccnttaangccacgggttctgctcattttggggaaggatcagggcccntctggctggangaggtgaactgtaanggaaaagaatctcgcntttctcagtgccgttcccnnggctgggggcggcagaactgcaggcntaaggaggangcaggagttntctgctcagagttcnngtctctcagactcntcagtgacaccagcagcgagacctgtgcagggcgcctggaagtcttttacagcggagctnggggcagcntgngcaagagtgacnngtctgccaccacngtgggggtggnnngcaggcagcttggctgtgcggacaaagggagtntcagaccagcaccttcagacaaggtggagcacaggtacnnangggtggacaangtccagtgtcctaagggaccggaaaccttnnggcagtgcccntcatctccgtggaagaggagactggccagcccctcagaggagacctggatcacangtgctgataaaanaaggcttcaggaaggaaccactaattgttctggangcgtggaggtctggcanggaggctcctggggcacagtgtgtgangactcctgggaccttaaagangcccaggtgctgtgtcgccagctgggctgtggcttagctctggaggcaggaaaagaggcagcnttcggccagggaactgggcccnnanggctcaangaagtgaagtgcaagggcaangagtcttccctgtgggattgtcctgccagatactggggacacagtgactgcgggcacaaggaggangcctctgtgaagtgctcagaaattgcagaaagcaaaggatcactaaaggctgcaggtcactcntccactgttgtactcggagtccttggggtcnttctgttggcctttctcntcgcagccctcttgtggattcagaagcgaagacagagacaaagacttgcagtttcctcaagaggagagaactctgtccangaaattcaataccgggagangaattcttgcctgaaagcagangatctggacctacnnaattcctcaggtctgtgggttcttagagnntccnnngncnnnanngcagcngangcng

SEQ ID NO: 9 - DNA, 4655 bp, Sus scrofa
  source 1..4655: mol_type = other DNA
gggctgcacccatggcatatgaaacttcttgggctaggggtctaattggagataaagctgccagccacagccacagcaaaaatccgagccacatctgccacctacaccatagctcatggcaatgctggatccttaacccactgagcaaggccagggatcaaacccacaacgtcatggtttctagtgggattcgtttctgctgtgccatgacaggaactcctaaggaagcctctttcagttttccaaaaccagtacaaaagctgttatatctaaaacaactgtttgactagtaatcaagtcatgacttttttgattctaacttgttcctggacactctttttaaccaagatttgggctttattctctccagtctttgttccataaagtgagctatttgcttccgaatggcagagctggatgggggacatcttagcatttgattggttggcttttaagggattacttctctggaaaggcactgtcccatattatcacattgctagcagtgtggatttagttgtgatcaaatttttaaattgaagatgaacatttaccaccaaagaggacataaacttcatgactcctgtgttatatagattcctatacatcatattggtttaatgtgtttgtgatgaagagctctgttttaaaacaaccttaaataccacatccagaaataggaactgactctggcaaagaggttattcggggtcccaatatgtttaataacgaagtcaatacaattttaaaaagaataaattccaactacacaaatggactttaataagattaacaaaactgaggaagactgatacgtcaataagtcaacaagtatatataacatacccagatgaaataaaacaatgacttcccatttttatcttgcatttcccagctacttaagagtaaacatgatcacttttcaaatcccacttaatattttatctgaaagaagaatttactgagtaaaatgcttagcttcaggatctcacatttgcaatccatcaggttacaatattagttaccactgtttcttccctcggtgtaagcattctatatgtgacttttacttacattctgccactttttatttcttcctaacttttatatgctcaagaatgaaactgtattggtgttacgtaacatctcaaatttgcaacatacatattggtgtatctcacttgaacctacatccctgaaaaataaacacaatgtatccaaaagaactacctcggagtgtatgaggaggaagagcgtagctgtgtagaaataaaagaatggaaaaacagttttgtggagatgtatctgtccttgttgacaacaatccccatgatttattaacagacacaggcagccaacttagtacacaacaaggtcctagcacggaagttggtaatcaagtgggaattaaggctttaaggtctcttacaaccaatgtgattcaaaaaaaatccaaagcaagcaaaccgtacaacttggagaaagtgctccaaacagagcttctttggcaacccaaattgaaccaaatgactcaggtggacgattaaaataaactgtgcgccgttttatttcaccaggtcaataaagcccataaattatttttggtccagaaaaccaggagttacaagcaccttccttttcatttatttaaaatattcaatatattcagtatcaatttttagtccctgctcttcattcacttattgactaagatcctgtgtgccttttggtgatcattccttccaagctagtaaaacagcaaactgaaatgcccctccatgcagagggtcgagaccgagaggttaagtcacccaaaggtggacgcaccttgctttttatttactgaagtatgcgagggactacgatggccatggctggactacgacgaggcgggaggcgaaggggcgcgcgggctgtgggctgtagctcaggggttggaacccgcaaaagagatgggggcagcagccaccctccactagctcggcccaaaggacccagaaaaagaccacccctccccccactgcacccttgacccctccccctctttccaggcctttcatccgcccgcccgcggggcagtccgctgcccccaggcctcgcactgcctgcctctttccacaacccagtggaggggcgagggtaggcccagggcgaccccgcgaagccctccgcccggcgagaaccccaacccccggcccctccggccgcgcccctccctttcatataacgcacctgcggccccgcccccggccaaacgctgaggcggctgcggggctggtgaggccgggcggcttggccctccaggcacagtcaccttcccttcccccgccattccgcccacccccctagctcagcaaccgagctgccgccgcgctcctcagccgccccgaaatctaaaggggtccgtctccggtaccacaaccaaatggctgagcctccctggccggctgcgcagcccgcccattggtcccttcccccccgcctccgccaccattcgttgttgcccggagaccctcggcggcgattggctaaggccgctgccactcgtccgcggagccgccgatcccgcctccacggtgatcaggttagtgtgcgccgcgggtgctgggggctcgagaaccgagcggagctggttgagccttcaaagtcctaaaacgcgcggccgtgggttcggggtttattgattgaattccgccggcgcgggagcctctgcagagagagagcgcgagagatggagatggacaaacggattcatttagagctgcggaacaggacgccctctgatgtgaaagagctcgtcctggacaattgtcggtcaaatgaaggcaagattgaaggcctcacagatgaatttgaagaactggagttcttgagtacaatcaatgtaggcctcacctcagtcgcaaacttaccaaagttaaacaaacttaagaagcttgaactaagtgataacagaatctcagggggcctggaagtattggcagaaaagtgtccgaacctcacacatctaaatttaagtggcaacaaaattaaagacctcagcacagtagagccactgaaaaagttagaaaacctcaagagcttagaccttttcaattgcgaggtgaccaacctgaatgactaccgagaaaacgtgttcaagctccttccacagctcacgtatctcgatggctacgaccgggacgacaaggaggcctctgactcagacgctgagggctacgtggagggcctggacgacgacgaggaggatgaggatgaggaagagtatgatgaagatgctcaggtagtggaagacgaggaggatgaggaggaggaggaggaaggtgaagaggaggatgtgagtggagaggaagaggaggatgaggaaggttataacgatggggaagtagacgatgaagaagatgaagaggagcctggtgaagaagaaaggggtcagaagcgaaaacgagaacctgaagacgagggagaagatgatgactaagtggaataacctattttgaaaaattcctattgtgatttgactgtttttacccttacccccccaaatcctgccccctgaaacttatttttttctgattgtaacgttgctgtgggaacgagaggggaaacgtgtactgggggttgtggggggagggagggtgggagggggtggaataaaatactatttttactgccactctttattttttttcccctactttttctttgtgtctggttttgtccctgtaaatgcgatagctgagtacccacaagtgagcgtttgttctgtgccctcagagaggatggttggagatctcttacctttcttggtatttctgagtctcttgagttggaaggccatggctggcctcagtttggtttctcaaagcccaggaggggctgagcaccagccgcatcttacctcttgttctggttcccatgatttctgaatttcttggacttgctagaagcagtcagcgccctggtttgtaaatagcaacctgaaggcgtattttggcaccggtcgggggacccttcccgtctcccatcccctttccctctttgcagatggtggtgggcttcgctctccgaagaagaccctgatgtcactcttgagttgatgagccagagagctgaggacagcaggcttctgagttaggagtaaaatggatttggtaattagaatcaaatgagaagaagcaaaccctcaaacttcaacctctgatattttttaaaaagctgtcctatatcttgttctgtaaaatagtagaacgctttgttttacaaaatgacaagagaattcttgtacgtgtcctcctgtgcttagaccatttttacaaacctaagcacaggagacgctgctgcatgtcagctgggtttcgtttttgtttttttcttttttttttcatacacctgagcacggaaaaactaacgcaaaattgtattttcttacctagtggaaacctgaaatgactgcaaattcctagtgaatgtacaggtttgctttcctgtccctcttctggattgcttttgaagtgacgtgcgatttccctagcccgtgtttcatctgtataaaagacatctgcaccagttttcctccccctcagaagagccaaactttgagttttatgtctgtttgtcattgataaatttcaataaatctttttatacaattt

SEQ ID NO: 10 - DNA, 1676 bp, Sus scrofa
  source 1..1676: mol_type = other DNA
caccgcggctgccgagccgagcggcgccgcgcggggccgagtcgagccgagcagccacccgcgccgcgtcgccggtttaagcgcagtgcggagccgcggccgggggcggcggtagtgagaccagcgctgcgctccagcccccttttccctccatggtttctctccgctcccgtgagtaacttggctccgggggctccgctctcctgcccgcacgccgccagccgccccggaccgcgccgccggcctctgcctcgagcagaccccttccgacggccctcgctgcgcaggctgggagcctctcccccctccgcccccgttgcggaaagttaagtttgaagaggggggaagagggaaacatggacatgaagaagagaatccacctggagctgaggaaccggaccccggcagctgttcgagaacttgtgctggataattgcaaatcaagtgatgggaaaattgagggcttaacagctgaatttgtgaacttagagttcctcagtttaataaatgtagggttgatttcagtttcaaatctccccaaactacctaaattgaaaaagcttgagctcagtgacaatagaatctatggaggtctggatatgttagcagaaaaacttccaaatctcacacatctaaacttaagtggaaataaactgaaagatatcagcaccctggaacctttgaaaaagttggagtgtctgaaaagcctggatctctttaactgtgaggttactaacctgaatgactatcgagagagtgtcttcaagctcctgccccagctgagctacctggatggctatgacagagacgatggtgaagcccctgactcagatgccgaggtggatggtgtggatgaagaagaggatgatgaagaaggagaagatgaagacaaggaggaggaagaggacggcgaggaagaagagtttgacgatgaagaggatgaagatgaagatgaagatgtagaaggggaggaggatgaagatgaagtcagtggggaggaagaagaatttggacatgatggagaagtcgatgaagatgaagacgatgaggatgaagatgaggatgaggatgaagaggaggaagaaaacgagaaaggtgaaaagaggaagagagaaacagatgatgaaggagaagatgattaagatcccaaataaccaacaaaaaaaagaactgttcagtattggttggactgctcatatggatttggtagctgtttaaaaaaaaaaagtagctgtgatacaaaccccaggacacccacccacccaaagagccaaagaatagttcctgtgacattccgccttcctccattttagtccctcttggaaatccaccaccaagcttggggacttaccccaacagaattgtaagcgatgttttaggttctcagtgtaagactcttgctgtagcgtggatagctgtgattggtgagtcaaccacctgtggctaccagttacaccaagattgtaacagcatttttactttctgtacaacaaagaagctttgtaaataaaatcttaacattttgggtctgtttttttttttttgggggttttttttttttttcatgcctttcttttcttcttaatgaaaattttttacattaggacattttatgtgacaactgccaaaaaagtatttttaagaatttaagtgaaataaacacgttcctctttggt

SEQ ID NO: 11 - DNA, 2061 bp, Bos taurus
  source 1..2061: mol_type = other DNA
agtgtgcgccgcgggtgctgggggctcgagaaccgagcggagctggttgagccttcaaagtcctaaaacgcgcggccgtgggttcggggtttattgattgaattccgccggcgcgggagcctctgcagagagagagcgcgagagatggacatggacaaacggattcatttagagctgcggaacaggacgccctctgatgtgaaagaactcgtcctggacaactgtcggtcgaatgaaggcaaaatcgaaggcctcacagatgaatttgaagaactggagttcttgagtacaatcaacgtcggcctcacctcagtcgcaaacttaccaaagttaaacaaacttaagaagcttgaactaagtgataacagaatctcagggggcctggaagtattggcagaaaagtgtccgaacctcacgcatctaaatttaagtggcaacaaaattaaagacctcagcacaatagagccactgaaaaagttagaaaacctcaagagcttagaccttttcaattgtgaggtgaccaacctaaacgactaccgagaaaacgtgttcaagctccttccgcagctcacgtatcttgatggctacgaccgggacgacaaggaggcccccgactcagatgccgagggctacgtggagggcctggacgatgacgaagaggacgaggatgaggaagagtatgatgaagatgctcaggtagtggaagacgaggaggacgaggaggaggaggaggaaggggaagaggaggacgtgagtggagaggaagaggaggatgaggaaggttataacgacggggaagtagacgatgaggaagatgaagaggagcttggtgaagaagaaaggggtcagaagcgaaaacgagaacctgaagatgagggagaagacgatgactaagtggaataacctattttgaaaaattcctattgtgatttgactgtttttacccgtacccccccaaatcccgccccctgaaacttatttttttctgattgtaacgttgctgtgggaacgagaggggaaaagtgtactgggggttgtggggggagggagggtgggagggggtggaataaaatactatttttactgccactctttatttttttcccctaatttttctttgtgtctggttctgtccctgtaaatgcaatagctgagtacacacacgtgaacgtttgttccttaccctcagagaggacaatttggagatctctgacatttcctgatacttcccaagtctcttgagttgattggaaggccggggctggcctcagtttggtttctcaaagcccaggagggctgagcaccagccgcgtcttacctcttgttccggatcccatgattttcacgtttcttgggcctgctagaggcagcaagcgccttggcttgtaaatagcaacctaaaggcgaatcttggccctggtctggggaccttccctgtcttccatcccctttctctcttcccagacggtggtgggctttgctctcctgagaagactcttatgtcactcttgagtgatgagccagagagctggaaacagcaggcttcagagtttaggagtaaaatggattcagtaattctaattaaaagagaagaagcaaaccctcaaacttcaacctctttcaaagatataaaaaaacaaaactctccctatcttgttgtgtaaaatattagaacactttgttttacaaaatgacaggagaactcttctgtgcgtcctgctcggcctagaccatttttacagacctaggcacgggacacactgctgcatgtcggctgggtttttttttcatacacccgagcacggaaaaacttaacgcaaaattgtattttcttacctagtggagatctgaagtgactgcaaattcctagtgaatgtacaggctcgctttcctgtccctcttctggattgctttccaagtgacgcgtgacttcctagcccatgtttcatctgtataaaagaacatctgcaccggttttcctccccctcagaagagccaaacttttgagttttatgtctgtttgtcattgataaatttcaataaatctttttatacaattgt

SEQ ID NO: 12 - DNA, 1058 bp, Bos taurus
  source 1..1058: mol_type = other DNA
tgagctgcgctccagcccccttttccctccatggtttctctccgctcccgcgagtaacttggctccgggggctccgctcgcctgcccgtacgccgcccgccactcaggaccgcgccgtcggcctctgcctcgagcagaccccttccgacggccctcgctgcgcaggctgggacgcctctcccccctccgcccccgccgcggaaagttaagtttgaagaggggggaagagagagaaacatggacatgaagaggaggatccacctggagctgaggaaccggaccccggcagctgttcgagaacttgttctggacaactgcaaatcaaatgatggaaaaattgagggcttaacagctgaatttgtgaacttagagttcctcagtttaataaatgtaggcttgatttcagtttcaaatcttcccaaactacctaaattgaaaaagcttgagctcagtgataatagaatctgtggaggtctggatatgttagcagaaaaacttccaaatcttacacatctaaacttaagtggaaataaactgaaagatatcagcaccctggaacctttgaaaaagttggaatgtctgaagagcctggatctgtttaactgtgaggttactaacctgaatgactaccgagagagtgtcttcaagctcctgccccagctgacctatctggatggctatgaccgagaggatcgtgaagccccagattcagatgccgaggtggatggtgtggatgaagaagaggatgatgaagaaggagaagatgaggataaggaggaggatgaagatggtgaggaagaagagtttgatgatgaagaggatgacgatgaagacgaagatgtagaaggggaggaggatgaagatgaagtcagtggggaggaagaagaatttggacatgatggagaagttgatgaagatgatgaagatgaggatgaagacgaagatgaagatgaagaagaggaagaaagcgggaaaggtgaaaagaggaagagagaaacagatgatgaaggagaagatgattaataagaccccaataacctgcaaaaaaaaaaaaaaaa

SEQ ID NO: 13 - DNA, 2049 bp, Ovis aries
  source 1..2049: mol_type = other DNA
agtgtgcgccgcgggtgctgggggctcgagaaccgagcggagctggttgagccttcaaagtcctaaaacgcgcggccgtgggttcggggtttattgattgaattccgccggcgcgggagcctctgcagagagagagcgcgagagatggacatggacaaacggattcatttagagctgcggaacaggacgccctctgatgtgaaagagctcgtcctggacaactgtcggtcgaatgaaggcaaaatcgaaggtctcacagatgaatttgaagaactggagttcttgagtacaatcaacgtcggcctcacctcagtcgcaaacttaccaaagttaaacaaacttaagaagcttgaactaagtgataacagaatctcagggggcctggaagtattggcagaaaagtgtccaaacctcacgcatctaaatttaagtggcaacaaaattaaagacctcagcacaatagagccactgaaaaagttagaaaacctcaagagcttagaccttttcaattgtgaggtgaccaacctgaacgactaccgagaaaacgtgttcaagctccttccgcagctcacgtatcttgatggctacgaccgggacgacaaggaggcccccgactcagatgccgagggctacgtggagggcctggacgacgacgaagaggacgaggatgaggaagagtacgatgaagacgctcaggtagtggaagacgaggaggacgaggaggaggaggaggaaggggaagaagaggacgtgagtggagaggaagaggaggatgaggaaggttataacgacggggaagtagacgatgaggaagatgaagaggagcttggtgaagaagaaaggggtcagaagcgaaaacgagaacctgaagatgagggagaagacgatgactaagcggaataacctattttgaaaaattcctattgtgatttgactgtttttacccgtacccccccaaatcccgccccctgaaacttatttttttctgattgtaacgttgctgtgggaacgagaggggaaaagtgtactgggggttgtggggggagggagggtgggagggggtggaataaaatactatttttacggccactctttatttttttcccctaatttttctttgtgtctggttctgtccctgtaaacgcaatagctgagtacacacaagtgaacgtttgttccttaccctcaaagaggacgatttggagatctctgacatttcctgatacttcccaagtctcaagttgattggaaggccagggctggcctcagtttggtttctcaaagcccaggagggctgagcaccagccgcgtcttacctcttgttccggatcccttgattttcacgtttcttgggccggctagaggcagcaagcgccttgattgtaaatagcaacctaaaggcgaatcttggcactggtctggggaccttccccgtcttccatcccctttctctcttcccagacggtggtgggctttgctctcctgagaagactctgatgtcactcttgagtgatgagccagagagctggaaacagcaggcttcagagtttaggagtaaaattgattcagtaattctaactaaaacaaaagaagcaaaccctcaaacttcaacctctttcaaagatacgaaaaaacaaaactgtccctatcttgtgtaaaatattagaacgctttgttttacaaaatgacaggagaattctgcgtgtcctgctcggcctagaccatttttacagacctaggcacaggacacactgctgcatgtcggctgggtttttttttcatacacccgagcacggaaaaacttaacgcaaaattgtattttcttacctagtggaaatctgaaatgactgcaaatccctagtgaatgtacaggtttgcttttctgtccctcttctgattgctttccaagtggcgtgtgacttcgtagcccatgtttcatctggataaaagaacatctgcaccggctttcctccccctcagaagagccaaacttttgagttttatgtctgtttgtcattgataaatttcaataaatctttttatacaatt

SEQ ID NO: 14 - DNA, 1407 bp, Ovis aries
  source 1..1407: mol_type = other DNA
cttttccctccatggtttctctccgctcccgcgagtaacttggctccgggggctccgctcgcctgcccgcacgccgcccgccacccaggaccgcgccgtcggcctgtgcctcgagcagaccccttccgacggccctcgctgcgcaggctgggacgcctctcccccctccgcccccgccgcggaaagttaagtttgaagaggggggaagagagagaaacatggacatgaagaggaggatccacctggagctgaggaaccggaccccggcagctgttcgagaacttgtcctggacaattgcaaatcaaatgatgggaaaattgagggcttaacagctgaatttgtgaacttagagttcctcagtttaataaatgtaggcttgatttcagtttcaaatcttcccaaactacctaaattgaaaaagcttgagctcagtgacaatagaatctgtggaggtctggatatgttagcagaaaaacttccaaatcttacacatctaaacttaagtggaaataaactgaaagatatcagcaccctggaacctttgaaaaagttggaatgtctaaagagcctggatctgtttaactgtgaggttactaacctgaatgactaccgagagagtgtcttcaagctcctgccccagctgacctatctggatggctatgaccgagaggatcgtgaagccccagattcagatgccgaggtggatggtgtggatgaagaagaggatgatgaagaaggagaagatgaggataaggaggaggatgaagatggtgaggaagaagagtttgatgatgaagaggatgatgatgaagacgaagatgtagaaggggaggaggatgaagacgaagtcagtggggaggaagaagaatttggacatgatggagaagttgatgaagatgatgaagatgaggatgaggacgaagatgaagatgaagaagaggaagaaagcgggaaaggtgaaaagaggaagagagaaacagatgatgaaggagaagatgattaataagaccccaataacctgcaaaaaaaagaactgttcagtattggttggactgctgacatggatttggtagcttttttttttttttttttttttttaagaaaaataaaagggtagctgtgatacaaaccccaggacacccacccacccaaagagccaaagaatagttcctgtgacattccaccttcctccatttagtccctcttggaaatccaccaccaagcttggggacttcaccccaacaaaattgtaagcgttgttaggtttttgtgtaagacttgctgtagcgtgtgtagctgtgattggtgaggcaactgtccgtggctaccagttacaccaagattgtaacagcatttttactttctgtacaacaaagaagctttgtaaataaaatcttaacattttg

SEQ ID NO: 15 - DNA, 3653 bp, Sus scrofa
  source 1..3653: mol_type = other DNA
caggttctggtgggcagtgtcggtggctggcctggctcttcgtcccagggaggaggctctgagccggctggggttgagcgcccctgcggccgcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaatagtgcctggtccttcccgccacctggccccccgcacaagatgcccgcatgtgtccatcccagggcccacggcagggcccatgttcctgaagcccgaggcacctgcgtcctccctttagggacaaagcctgtggcccatgttatctccattctgccctggggcccaatccagtttctggtgaccagactgtccaggtgtctcccagccactgccccttgtgccaaccccaccctgggcctggcccagggcccttctcagggaagtccagctccaaggccagatgagcagaagcccttgatggatgatggatggccttgaagaactgccctctaccctctctcccctttttccataaagaccctgaacctgagaatcaacagggcatcagatctgtatatttttttcctaggagtaaatgtaaataaaggatttctagatgagc

SEQ ID NO: 16 - DNA, 3808 bp, Sus scrofa
  source 1..3808: mol_type = other DNA
tgggcagtgtcggtggctggcctggctcttcgtcccagggaggaggctctgagccggctggggttgagcgcccctgcggccgcagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaaggtaagcccctctccttggcattgccccagtggcctgagggccagctgctgccaggctgcaggagggactgctctgctgaagcctggggagaggagggccgggggtcaggtcagtccatggccttctcagcagcccctccccggctctgctccctccccacagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaatagtgcctggtccttcccgccacctggccccccgcacaagatgcccgcatgtgtccatcccagggcccacggcagggcccatgttcctgaagcccgaggcacctgcgtcctccctttagggacaaagcctgtggcccatgttatctccattctgccctggggcccaatccagtttctggtgaccagactgtccaggtgtctcccagccactgccccttgtgccaaccccaccctgggcctggcccagggcccttctcagggaagtccagctccaaggccagatgagcagaagcccttgatggatgatggatggccttgaagaactgccctctaccctctctcccctttttccataaagaccctgaacctgagaatcaacagggcatcagatctgtatatttttttcctaggagtaaatgtaaataaaggatttctagatgagc

SEQ ID NO: 17 - DNA, 3412 bp, Bos taurus
  source 1..3412: mol_type = other DNA
tgcccgttcctgagcctccccgagctccctccccattctcaccatggccaaaggattctacatttccaaggccctgggcatcctggccattctcctgggcgtggcggccgtggccaccatcatcgctctgtctgtggtgtacgcccaggagaagaacaagaatgccgagcgtggcaccgcggcccccacgagccccacaggcccgacaaccacctcggccaccaccttggaccagagcaagccgtggaaccgataccgcctccccacgaccctcttgcccgactcctatagggtgacgctgaggccctacctcactcccaacaataacggcctgtatatcttcacgggctcaagcactgtccgcttcacctgcaaggagcccacggacgtcatcatcatccacagcaagaagctcaactacacccagcacagtgggcacttggcggccctgaagggtgtgggggacacccaggcccccgaaatcgacaggaccgagctggtgttgctcacagagtacctggtggtgcacctcaagagctccctggaagcaggcaagacatacgagatggagaccacctttcagggggagctggccgatgacctggcgggcttctaccgcagcgagtacatggacggcaacgtcaaaaaggtcctggccaccacacagatgcagtcaacagatgcccggaagtccttcccctgctttgacgagcccgccatgaaggccacatttaacatcaccctcatccaccccaaggatctcacagccctgtccaacatgccacccaaaggtcccagtgtcccgtttgacggagattccaactggagcgtcactgagtttgaaaccacgccggtaatgtccacgtacctcctggcctacatcgtgagcgagttcacgagtgtggagtcagtggctcccaatgatgtccagatccggatctgggctcggccaaaagcaactgcagacaaccatggcctttacgccctgaatgtgacaggtcccatcctgaacttctttgccaatcattacaatacagcctacccactccccaaatctgaccagattgccctgcccgacttcaacgccggtgccatggagaactgggggctggtgacctaccgggagaacgcgctgctctatgaccctcagtcttcctccagcagcaacaaagagcgagtagtcactgtgattgctcatgagctggcccaccagtggtttgggaacctggtgaccctggcctggtggaatgacctgtggctgaacgagggctttgcctcctatgtggagtacctgggagctgactatgcagagcccacctggaatctgaaagacctcatggtgccgaatgacgtgtacagtgtgatggccgtggacgcgctggtcacctctcacccactgaccacccctgccaacgaggtcaacacccctgcccagatcagcgagatgttcgataccatctcctacagcaagggagcctcggtcatccggatgctctcgaatttcctgactgaggacctgttcaagaagggcctggcgtcctacttgcagacctttgcctatcagaacaccacctacctgaacctgtgggagcacctgcagatggctgtggaaaatcagttgtccatcaggctgccagacaccgtgagtgccatcatggaccgctggaccctacagatgggcttccctgtcatcaccgtggatactaacacagggaccatctcccagaagcacttcctccttgatcccaactccaccgtcacccgcccctcacagttcaactatctgtggattgttcccatctcatccatcagaaatggccagccgcaagaacactactggcttcggggcgaagagagaaaccagaatgaactcttcaaagccgcagcagacgactgggtcctgctgaacatcaacgtgacgggctattaccaggtgaactacgacgagaacaactggaagaagattcagaatcagctgatgtcaaggcgggagaacatccctgtcatcaaccgggcacaggtcatctacgacagcttcaacctggccagtgcccacatggtccctgtcaccctggcgctgaacaacaccctcttcctgaaaaatgagatggagtacatgccctggcaggccgccgtgagcagcctgaactacttcaagctcatgttcgaccgcacagaggtctatggccccatgcagaattacctcaagaaccaggttgaacccatcttcctatattttgaaaatctcaccaaaaactggactgagatcccggaaaacctgatggaccaatacagcgagattaacgccatcagcactgcctgctccaatgggttgcctaagtgtgaggagctggccaaaacacttttcaaccagtggatgaataaccctaacgttaacccgatcgaccccaacctgcggtccaccatctactgcaacgccatcgcccagggtggccaggaagagtgggactttgcttggaatcagttacaacaagcagagctggttaatgaagctgacaaactccgctcagcgctggcctgcaccaaccacgtctggctcctgaacaggtacctgagttacaccctgaaccccgacctcatccggaagcaggacgccacctccaccattaccagcattgccagcaatgtcatcgggcagtctctggcctgggacttcatccggagcaactggaagaaactctttgaggattatggtggtggttccttttccttctccaacctcatccagggtgtgacccgaagattctccactgagtttgagctccagcagctggaggagttcaaggagaacaacatggacgtcggcttcggctcaggaacacgggctctggagcaagccctggagaagactaaagccaacatcaactgggtgaaggagaacaaggaggtggtgctaaactggttcaaagaccacagctaaacagtctctggtccttacattcacctggtccctatgtgagacgcccacctgcctctgtcccagggacccatggcagggcccttgtttctgaagcctgaggcaccagcgtcctccccttggggacagagtctctggcccatgttcactccactcttcccaggggaccactccagcttctgatgcccagactgtccaagtgcctcccagctcctgcccctcatgccaatggcaccccaggcttggcatggctcctgtcacccaaggctctgggtctgatctcagggagtccagctccagggtcagatgagaagccctcagtggatgatggatggccttgggggggctgccctgtgccgtctcctatcctcccctaaagaccctggaactgaaaagtctacagagcaccagatctgtatatttttttctaagataaaatgtaaataaaagatttctagaaaaaaaaaaaaaaaa

SEQ ID NO: 18 - DNA, 3118 bp, Sus scrofa
  source 1..3118: mol_type = other DNA
ggggaggatcggaactccgagctgggccgaggaagccgcgagcttcggaggaggcggaggagaaggaagagcgaggggcggggagcgcgggacccggagcgcggcaggtcaacttgaaagttccaaatacttacctgtacctgatgctgctgataaaatcaggatggctttaaactcaggatcgcggccaggtgtcgggccttactatgaaaaccacggataccaacctgaaagtgtctaccccccgcagccccccggggcccacaggccctatggggcgtatccggctcagtaccacccgccctcggtcccccagtatgccccgagggtgcagacgcacgcttccacacccgccgtcgtcgtcagcaggcagcccaaaccccgatccaggacgatgtgctcttcaaagaccaagaaagcgctgtgcatcacctttgctctgggggccatcctggcaggagcagtgctggccactgtcctgctctggaagttcatggagaagaagaggtgctccacgcccgagatggagtgcggctcttctgggacctgcatcagcccctctcactggtgcgatggcatcctgcactgtcccggcggcgaggacgagaaccagtgtgttcgcctgtacggacccaacttcattcttcaggtgtactcggctcagaggaagtcctggtacccagtgtgccaggacgactggactgagaactacgggagggcggcgtgccaggacatgggctaccggaatagctttttttctagccaaggaatcgccgatgacagcggggccacgagctttatgaagctgaacaaaagtgccaacaatatggatctctataaaaagctgtaccacagtgacgtctgtacttcgaatacggtggtttctttacgctgtatagagtgcggagtctccgggaagatgagcaaccgccagagccggatcgtgggcgggtccagcgcagcgctgggcgactggccctggcaggtcagcctgcacgtccagggcatccacatctgcggaggctccatcatcacccccgactggattgtgacggccgcccactgcgtggaggaacctctgaacaatcccaagatctggacagcctttgcaggaattttgagacaatcgttcatgttttacggcagcggataccgagtggccaaagtgatttcccacccgaactatgaccccaagaccaaaaacaacgacattgctctgatgaagctgcagacgccaatgaccttcaacgacaaagtgaagccggtgtgtctgcccaacccaggcatgatgctggagccgacacagtcctgctggatttccgggtggggggccacctacgagaaagggaaaacctcggaggtgctgaacgccgctatggtgcgcctcatcgagccctggagctgcaacagcaagcaagtgtacaacaacctgatcacgcccgccatgatctgcgcagggtacctgcagggatccgtcgactcctgccagggtgacagcggaggtcctctggtcactctgaagagcagcatctggtggctgattggggacaccagctgggggtccggctgtgccaaggcttaccgaccgggagtgtacgcgaacgtgacattatttacagactggatttatcgacaaatgagggcaaacagctaatccctcgagccttggcccttcgcaccactccgcgagaaaacgaaggcactgatttgtcatccccgtgcgtaacctattttcaggaatgatccccaggtcctctggtgtttatgaaatagtgaacccgtctgatttggcgctctccagggtcctgtgctggctgcgcgttccttcccggcccctcatctgaatagtgaggctggccggcggtggccacccaaggccagtgcagagcagggggtggggtctggcccactggggtcaccctttggaaccccttcccggggggccactcttgcacgccctgggggctggtgacttctcagctgctggacggctggagatggaagagagaaagccaggcagacactgcgggggccaccctcgcgccccccgggacctctcttcccccgagagagtttaatcattctttttaagtgccttcttcgcagccccggctggtgcctggaaataaagggaccagctcttgccggcttatgactccatagccacttgcaaggggcacggaaacctggtgagggtgtagacggcgccctcagagcaggtgcccccccccgcccccatcctgagcactgctgggactgggggccctgtgtgttgggtggggctccacccccggagaccagccttacaccccgcctcctcccagtccctctccccagcctcccgggagactgtgggacaccccccccaacactgtggtcctggggggctggtgctgctgacccttttaagacctgttaatcacgtggaaattgaggtcccccggggaaatcagggatgctgattaaacgacactgtttcctactgtaaaatttctacacgttgctacctcagtgctcacggaatctgggccgtgccccctaaggcgttcctccccttggttctttgaaaccactgtggtcctttgccacgtgtgagcagtggccggcgtgagctgcttttacaaagcgactggctcctcattcaatgttctctacgggacgcactgaagcacggcactgtggcaaagaagatgggggcctcggggctccctgtggccccttctggcccctgaggttccccagagggaaggttgggcctgaattgcggagtgtgacgtgtcaacgcagagatcctcgcagacacactgagaacgaagcgagcagttctgcgctgggacctaagcttggatggaggcagtacgatcccctggcaggatgcatccgtgccacgcacgtcttgggccacagcatccccagggaggggccgtgcagacagacgtgtctgctcccagagccagattttaaaaaaagcgttgtcgcggggtgaaggcatcccttctttagggctccttttaatttatgtgacgtctgttctggtctctgttttgtatctttttttaactgtaaaattcagttgtgaaaaattgtgcaaataaattatacattttttttttccaaa

SEQ ID NO: 19 - DNA, 2413 bp, Bos taurus
  source 1..2413: mol_type = other DNA
gtcaacttgaacgttccaaatacctccctttacccgatgctgctgataacatcaggatggctttaaactcaggatcatcaccaggtgtcgggccttactatgagaaccatggataccagccggaaagcttgtaccctcagcagccgcccacggctccccctgctttcgaggtgtaccccgctcagtactacccgcccgtggtgccccagtacaccccaagggttcagacgcacgcctcgacacctgtcatctacaggcagcccaaacctccatccaggacagcgtgcacctcaaagactaagaaagccctgtgcatcaccttcaccctggtggtcctgctggcaggagcaatccttgccgccgtcctcctctggaagtacatggaggacgagtgttcagggatggagtgcagctcctccgggacctgtgttagcccctctctctggtgcgatggcatcctgcactgccccagtggcgaggacgagaaccgctgcgttcgcctctatggaccgaacttcatccttcaggtgtactcagcacagaggaagtcctggcaccccgtgtgccaggacgactggagtgagagctacgggagggcggcctgccaggacatgggctaccggaacagtttttattctagccaaggaatagcagatgacagcggagccacaagctttatgaagctgaacataagtgccaatgatatcgatctctataaaaaactctaccacagcgatgtttgttcttcaaaaacagtggtttctttacgctgtatagaatgcggggtctcggtgaagacgagccgccagagccggatcgtgggcggatcgaatgcttactcgggcgaatggccctggcaggttagcctgcacgtgcagggcatccacgtctgtgggggctccatcatcaccccggagtggatcgtgacagccgcgcactgcgtagaggaacctcttaacaatcccaagatctgggtggcctttgcaggaatcttgaaacaatcttacatgttctatggaagtggataccgagtagcaaaagtgatttcccacccaaattatgattccaagaccaagaacaatgacattgcgctaatgaagctgcagacgcctctgactttcaacgacaaagtgaagccagtgtgtctacccaatccaggcatgatgctggagccaactcagtcctgctggatttccgggtggggggccacctacgagaaagggaagacctcagatgacctgaacgcggccaaggtgcacctcattgagccccggaagtgcaacagcaagtacatgtatgacaacctcatcacgcccgccatgatctgtgcaggctacctgcggggcaccgtcgactcctgccagggtgacagtggaggtcctctggtcacactgaagagcagtgtctggtggctgattggagacacgagctgggggtcaggctgtgccaaggcgtaccgaccgggagtgtacgggaacgtgacagtgttcacagactggatctatcaacaaatgagggcaaacagctaatctgtatggcctgggtccttgacaacattgcacaagaaaaagaaggggctgattgttaatccccacgcattatgtattttttgaagtgattccaggtccttttgtttttatgcgatcatgatcctgtatgtcttggccctttccacagttctgtgcagaccccgtggctcccctacccccgggcccacccttccagacccctcatctgaccggggaggctggctggccatggacagcagagccagcgcagagcaaggggacggggtctgccccgcggggacctcccctctgagtcccactgcagggactcctcggattcacagggggctggcgacttctcagctgctggacggcttgagaaaggaaggcgaaaagcctggcagaacctgcaggggccagcctctggggccactgctggtgagaccacttagggtagccccagggcctgtctctccacgagggaatttgatgaacattcttgagtgccttcttagcagcccagagtgctggctggaaataaaggggccagctcttcatgggtgatgacttgatagacacttgaaaggggacccaaacctttttctttcggggggtgtgtagacagagccctgggaacatatgtgatcacccccagcattgctgggggcccccacccagggagacccgccttattctccagtctctctctggcctcccttgaagatggtgcaatgccccgggcccccactgaggtcttcaccccgagtagggtgcctgctgaccctcttaacgcctgctaatcattcacctggaaatcgagctcacccagggaaatcagggatactaattaaagaaacactgtttcctgtaaaaaaaaaaaaaaaaaaaaaaaaaaaaaaaaaaaa

SEQ ID NO: 20 - DNA, 1897 bp, Sus scrofa
  source 1..1897: mol_type = other DNA
gcgtcctttccgtccggcccgcgccggcggcggggaggcggcgcgcgcggcccgcagcccgcccatggaggctttcccctgggcgccccgctcgccccgccgcggccgcgtccccccgcccatggcgctcgtgcccagcgcccgctacgtgagcgcccagggcccggcgcacccgcaacccttcagctcgtggaacgactacctgggactcgccacgctcatcaccaaggcggtggacggcgagccgcgctttggctgcgcccgcggcgaggacggcggcggcggcggcggctccccaccctcctcttcctcctcgtcgtgctgctccccccacgcgggggccgggcctggggcgctggggcccgcgctggggccacccgactacgacgaggacgacgacgacgacagcgacgagccggggtcccggggccgctacctggggggcacgctggagctgcgcgcgctggagctgtgcgcggacccctcggaggccgggctgctggaggagcgcttcgccgagctgagcccgttcgcgggtcgcgccaccgctgtgctgctgggctgcgcacccgccactgccgccaccgccgaggcggcaccgcgcgaggagcgggccccggcgtgggcggccgagccccggctgcacgcagcctctggggcggccggcgcccggctgctcaagcccgagctgcaggtgtgtgtgttttgccggaacaacaaggaggcggtggcgctctacaccacccacatcttgaagggacccgacgggcgagtgctgtgccccgtgctgcgccgctacacgtgtcccctgtgcggcgccagcggcgacaacgcgcacaccatcaagtactgcccgctctccaaagtgccgccgccgcgcagcgtcagggacggcctgcccggcaagaagctgcgctgagggcccggactccggtctgctactgccacctgacgccaccagggtcgccgcctgcccaatgtctagtttggcctgcgcaccatctctctctctcgctgctgaggagcgtggagctcagctgttggttgaacttgagatgtactgattttttttttttttttttcaaaagaacccggcggtactgagtcctttcctgtcgaagagcgcttaagactagaagctaaaatcttgatttgtttatctctagtttgtgcacatccagacggtgaaggctgggtgttcgttccactaactgaaatgtggcaacttagaagtgtttatttactctatacgtcaacctattttagatgcgcatcagtatatgaaattgtctcaatctaatcttggatgtttaattttatgaatggaggcactttactaggcctagaatatttttttaaaagcctctaaactgaacttaactggcgattttatggaatgtcagcaaaatgacttttattgtttgaaacaagtaataatatttctgttgtccttaatcagttattctaattccaggtgaagcaaccctcacctgcctggtagcatcattaagtgaaggcttagtaaactttccagtgttagtttgggtgggtgttccccccgtggcttgtttctgtcctagctggaggtgtaaagatgtacaatctgtggcaggtagaatacagctccttatccttttatgtaccacatcttttattactgaacgagcaactagcgtttcccatctttcaaagtcgtgccaggttatataatattgtgtatacacttggaaatggtgctgtttaaaagaatttgtgtatttatacagtaacagtatatgaattcattaatcttgtctttcactctggtacttggccttttgtctgtatacctccacctcgccatttctagttcaaaaatatttgattattgaagatcaaatggagccgtatgcactct

SEQ ID NO: 21 - DNA, 946 bp, Sus scrofa
  source 1..946: mol_type = other DNA
atgcagctgccaccctttgacatgtggaaggactacttcaacctgagccaggtggtgttgggactgatccagaatcgtcgacaagggccagaggccccgggcaccggggagccaagacctgagcccccactggagcaggaccagggcccgggagagcggggggccagcggggggctggccaccctgtgcaacttttgcaaacacaatggggaatctcgccacgtgtactcctcgcaccagctgaagacaccggagggcgtggtggtgtgtcccatcctacgacactatgtgtgtcccctgtgcggggccaccggtgaccaggctcacacactcaagtactgcccgctcaacggcggccagcagtctctctatcgccgcagtgggcgcaattcagccggccgcaaggtcaagcgctgaggaccgcaaggtgcccacccctgcacacccagcctctgggctcagtcctaccgagtccctggactcctctccattcccggactcaccccgccttctggaagatctgacccccaggatgccacccctgctggatcctgacaccatggaggctgtggggagccaggccagccctgccccttggacctctcagcccggggggttctgtactgggctgcttgtgaattcttggtgcggcgggacccctgccccttggaccacacagccctatggcttcttctggttcctggatcttttagtccttctgggacactagccctgccccctttgacaactcaggatcccactgtgctctcccaggcccacccctcagaatctggggagcgtgaacaggcaggcctgcgccctccagcccaagaacttcaagatgggattctgactcaagccagccgtcaccttcttcagccaccggcgacccaccggctcaccatggacgccaccggctgggtccctccctgccctcagcggcccg

SEQ ID NO: 22 - DNA, 2395 bp, Bos taurus
  source 1..2395: mol_type = other DNA
agcagcgcgcgctcactgcccgcccgccccccgcgcgtccgcctccgctccgcctccgtcccgggcgcgggcgaggtggcccgggcgggggagggcagggggcagggccgcggcggagaagccgcggggcggggaggagcgggtcccgataaaaggcggcgaggcagccccaccccgaggcaggccggcgggcaggctcggctcgtcccttccgtccggcccgcgccggcggcggggaggcggcgcgcgcggcccgcaacccgcccatggaggctttcccctgggcgccccgctcgccccgccgcggccgcgcccccccgcccatggcgctcgtgcccagcgcccgctacgtgagcacccagggcccggcgcacccgcagcccttcagctcgtggaacgactatctgggactcgccacgctcatcaccaaggcggtggacggcgagccgcgcttcggctgcgcccgcggcggggacggcggcggggacggctccccgccttcttcttcctcctcgtcgtgctgctccccccacgtgggggccgggcctggggcgctggggcccgcgctggggccgcccgactacgacgaggacgacgacgacgacgacagcgacgatccggggtcccggagccgctacctggggggcgcgctggagctgcgcgcgctggagctgtgcgcggaccctgccgaggccgggctgctggaggagcgtttcgctgagctgagcccgttcgctggtcgcgccgctgccgtgcttctgggctgcgcacccgccgccgccgccgcggccaccgccgaagcagcaccgcgagaggagcgggccccggcgtgggcggccgagcccaagctgcacgccgcctccggggcggccgccgcccggctgctcaagcccgagctgcaggtgtgcgtgttttgccggaacaacaaggaggcggtggcgctctacaccacccacatcctgaagggacccgacgggcgggtgctgtgccccgtgctgcgccggtacacgtgtcccctgtgcggtgccagcggcgacaacgcgcacaccatcaagtactgcccgctttccaaagtgccgccgccgcctgcagcccgcccgccgccgcgcagcgcccgggacggcctgcccggcaagaagctgcgctaagggcccggaccccggtctgctgctgccacctgatgccactggggtagccgcccgcccactctcgtgtttggtctgcgcaccatctcttcctcgctgccggggagtgtggagctcgtcttggtttttccagaggaagccgacggtaccgagtattttcctaacgaagagcagttgagactagacgttaaaattttgattaatgtttctagtttgtgcacatccagatggtgaaggctgggtattccactaactgaaatgtggcaacttagaggcgctgtggtttatttatacgtcgacctattttagatgcgcatcagtatgaaattgtctcagtctaatcttggatgtttaattttatgaatggaggcactttactaggtctagaatatttttttaaaagcctctcaactgaacttaaaactggcgattttatggagtgtcagcaaaatgactattttattgtctgaaacaatatttctgttgtccttacccagttgtaattccaggtgaagccctgcgtggtagcatcattaagtgaagacttggtatgctttacagtgttagtttgggtgggtgttccctccttgtggcttgtttttgtcctagctggagatgtataaaatgtacaatttgtaggtagcaggtagaatacagctcatgtaccagatctttttattactgaacgagcaactactaccgtttttcccctttaaaaatagtgccaagttataatcatattgtgtatacacttgaaaatggtgctgtttaaaaaaattgtgtatttatacagtaacagtatatgaattcattaaccttgcctttaactctacttggctttttctttatgccccttcctattccagttcttcaaaaatatgtgatacttaagatcaaacgggtgcaataactcattcactctgaattgctccatttcagggtctctaaatagtggaaatctcattccagctgttgcctctcagactaaatgtaagatggaatcctttgagctctggaaggttaatgaaacaactggtgttcaggaaggttccactctggactgtgtcagctttaaaccatcacagaagtcctcaaaccagtataagtaccaattaaaggaactgactgggtgtagggggggtaacacaaggaacacagcctccatctattgtgttcccattctcattagaagacaacccttctggaatcccaccagttattttcatcggtgagattaaatctaatcttgg

SEQ ID NO: 23 - DNA, 2113 bp, Sus scrofa
  source 1..2113: mol_type = other DNA
tcacagactccccaggacttggattggtcagaaagaagcagaggaggagccactgtgcacattttttttttccccttcacacaccataaaaatcacagaggcaactaacactcacagcaaagcttcaggttgggaactgattctctctgcgaggcagctgatctgagcatgcgcacacagattcattcttctcccaatagcacagcagccgctaggaaaattattttgaaaagacctgaatgcattaagactaaagttaaagtggaagtgagaacaaaatatcaaacagcagactcgacagagaatgagcgtcttgaagcctaagatttcaaagtgatgctaatcagagccctacctgaaagagactaaaaactccatttcaagcttcggagcatgtgatatttattcacaacaggcattccaatttcagcctcataactttcagacagataaagacttggagaaaatcgctgaggctacctgacccaggagcttaaatcaggtcagaggggatctcaacccacctggcgcaggatgaactcaacccatcaccatggaatgcatacttctctccacttctggaaccgcagcacctacggactgcacagcaatgccagtgagccccttggaaaaggctactctgaaggaggatgctacgagcaactttttgtctctcctgaggtgtttgtgactctgggtgtcataagcctgttggagaacattctggtgattgtggccatagccaagaacaagaatctgcattcacccatgtactttttcatctgtagcctggctgtggctgatatgctggtgagcgtttccaatgggtcagaaaccattgtcatcaccctattaaacagcacggacacggacgcacagagtttcacagtgaatattgataatgtcattgactcagtgatctgtagctccttactcgcctcaatttgcagcctgctttcgattgcagtggacaggtattttactatcttttatgctctccagtaccataacattatgacagttaagcgggttggaatcatcatcagttgtatctgggcagtctgcacggtgtcgggtgttttgttcatcatttactcagatagcagtgctgttattatctgcctcataaccgtgttcttcaccatgctggctctcatggcttctctctatgtccacatgttcctcatggccagactccacattaagaggatcgccgtcctcccaggcactggcaccatccgccaaggtgccaacatgaagggggcaattaccctgaccatcttgattggggtctttgtggtctgctgggcccccttcttcctccacttaatattctatatctcctgcccccagaatccatactgtgtgtgcttcatgtctcactttaatttgtatctcatcctgatcatgtgtaattccatcatcgatcccctgatttatgcactccggagccaagaactgaggaaaaccttcaaagagatcatctgttgctatcccctgggtggcctctgtgatttgtctagcagatattaaatggggacagaggagacttataaatgcaagcataagagactttctccttacacagtctggacaatatgcttcaacaacagcattttcttgtaaggcatcagttgagacattctattgtataaatttaagttcgtgattctgctcagtctctgtgtatttttaaggtcttgctaccttttggctgtaaaatgtttatctatactacaggttataggcacaatggatttataaaaaagaaaaaagtccttatgaaaagttaattaatgtatcttgtcattcgaaaggatttgacacattgcttgttttagtaaaatggaaatcacagtttcattaaatatatcctaataaatggttgctaatattacactatacaacgctgaagtgtagaggtttgattctagcattgaggggagaaatactgaaacaagtgtttaatcattaaaaaataagctgaaatttcaactaatttaataaaacatgctcattctccctgtgcagaaggagaaatgaagcttctactgggagaaaaacagttactaaaaaaaagtggggggatattttgagtttgaaaactatgttt

SEQ ID NO: 24 - DNA, 1809 bp, Bos taurus
  source 1..1809: mol_type = other DNA
cagcctaagatttccaagtgatgctgaccagagccacacttgaaagagactgaaaacttcctttccagctccggagcatgggacatttattcacagcaggcatgccactctccgccgcctaactttcgtttggggcaagtcaagactggagaaaggtgctgaggctgccagatccaggaggttcagtcagtccagaggggacctgaatccaaaatgaactctacccagccccttgggatgcacacctctctccactcctggaaccgcagcgcccacggaatgcccaccaatgtcagtgagtccctggcaaaaggctactcggacggggggtgctatgagcagctctttgtctctcccgaggtgtttgtgactctgggggtcatcagcttgttggagaatattctggtgatcgtggccatagccaagaacaagaatctgcactcacccatgtactttttcatctgcagcctggctgtggctgacatgttggtgagcgtttccaacgggtcggaaaccattgtcatcaccctgctgaacagcacggacacggacgcgcagagcttcacggtggatattgacaatgtcattgactcggtgatctgtagctccttgcttgcctccatctgcagcttgctgtcgatcgcggtggacaggtacttcactatcttctatgcgctccagtaccataacatcatgacggtgaagcgggtggcgatcaccatcagcgccatctgggcagcctgcacggtgtcgggcgtcttgttcatcatttactcagacagcagtgctgttatcatctgcctcatcaccgtgttcttcaccatgctggctctcatggcgtctctctatgtccacatgttcctcatggccagactccacattaagaggatcgcggtcctgccaggtagcggcaccatccgccagggcgccaacatgaagggggcgattaccctgaccatactgatcggggtctttgttgtctgctgggcccccttcttcctgcacctgatattctacatctcttgtccccagaacccatactgtgtgtgtttcatgtctcactttaacctgtacctcatcctcatcatgtgcaattccatcattgaccctctgatttatgccctgcggagccaagaactgaggaaaaccttcaaagagatcatttgttgctctcctctaggtggcctctgtgatttgtctagcagatattaaatggggacaaacgcgatgctaaacacaagcttaagagactttctccttctcatatgtacaacctgaacagtctgtatcagccacagctttttcttctgtgtagggcatggagtgaaaatttctattgtatcagttgaagtttgtgatttttttctgatgtgaaacagtgcccagtcttggtgtatttttaatgtcatgctactttctggctgtaaaatgtgaatccacatcacaggttataggcactatgcatttataaaaaaagaagaaaaaaagtccttatgaggagtttaacagtgtttccttcttgttatttacaaggatgtgacactttgcttgcttttgtaacatggaaatcacagcttcattaagtatatcctcataagtggtttttttatgttatactttacaacactgaagtgtaaaaatttgattctagcatttaggggagaaatattgagaacatattgcttaatcataaaaaacaagctgaaatttcaggtaatttaataagactttctcattcattcttcctgtgcagaagttgaaatgaagcttgtattgggagaaaaacagttacttaaaaaaaaaaaaaaa

SEQ ID NO: 25 - DNA, 3869 bp, Ovis aries
  source 1..3869: mol_type = other DNA
ccggcacctctgcgaggcagctgatgagagcatgcgcacacagagccggctcccagccgtagcgcagccaccaggcaaatcattctgcaaagacctgcatgcctccgactcaagttgaagtggaagggagaacaaaaaagccagcggcaggctcgacggagaaggagtatcttgcagcctaagatttccaagtgatgccgaccagagccgcacctgaaagagactgaaaacttcctctccagctccggagctcgggacgtttattcacagcaggcatgccactgtcggccacctaactttcgtttggggcaagtcaagactggagaaaggcgctgaggctgccggacccgggaggttcagtcagtccagaggggacctgaatccaagatgaactccacccagccccatggaatgcacacctctctccactcctggaaccgcagcggccacgggctgcccaccaatgtcagtgagtccccggcaaaaggctactcggacggggggtgctatgagcagctctttgtctctcccgaggtgtttgtgactctgggggtcatcagcttgttggagaatattctggtgattgtggccatcgccaagaataagaatctgcactcacccatgtactttttcatctgcagcctggctgtggctgacatgttggtgagcgtttccaacgggtccgaaaccattgtcatcaccctgctgaacagcacagacacggacgcgcagagcttcacggtgaatattgacaacgtcatcgactcggtgatctgcagctccttgctcgcctccatctgcagcttgctgtcgatcgcggtggacaggtacttcaccatcttctatgcgctccagtaccatagcatcatgacggtgcggcgggtggcgatcaccatcagtgccatctgggcggcctgcacggtgtcgggcgtcttgttcatcatttactcagacagcagcgctgtcatcatctgcctcatcaccgtgttcttcaccatgctggctctcatggcgtctctctacgtccacatgttcctcatggccaggctccacattaagaggatcgcggtcctgccaggcaccggcgccatccgccagggcgccaacatgaagggggcgatcacgctgaccatcctgatcggggtctttgttgtctgctgggcccccttcttcctgcacctgatattctacatctcctgcccccagaacccctactgcgtgtgcttcatgtctcactttaacctgtatctcatcctgatcatgtgtaattctgtcatcgaccctctgatctacgccctgcggagccaggaactgaggaaaaccttcaaagagatcatttgctgctctcctctaggtggcctctgtgatttgtctagcagatattaaatggggacaaactgcggtgccaaacacaagcttaagagaccttctccttctcatgtgtacaatctgaacagtctgtatgagccacagctttttcttctgtgtagggcatggagtgaaaaattctcttgtatcagttgaagtttgtgatttttttctgatgtgaagcagtgcccagtcttgctgtatttttaatatcatgctactttctggctgtaaaatgtgaatcctcatcacaggttataggcactatggatttataaaaaaaaaagaaacaaagtccttatgaggagtttaacagtgtttccttcttgtgatttacaagggtgtgacagtttgcttgtttttgtaacatggaaatcacagcttccttaagtatattctcatgagtggatttaatgttatactttacaacactggagtataaaatttgattctagtatttaggggagaaatattgagaacatattgcttaatcataaaaaaaaaaaccaagctgaaatttcaggtaatttaataagacttgctcattcattcttctgtgccgaagttgaaatgaaacttctactgggacaaaaacggtgactttaaaacaaaaaaaaaaaaaaaaggaggggggatattttgagtcagaaaactctgtgtgtttctcagagacagagagattaatttgctcaagcaaagagactgatatttctcacaggcaattcagtttgggattaaaaaaaaaaaatagcagctatgaggatccaagtgttctgggaaatctaaattaacaaggcaagctaagcttcctgatatcaaactgtgtacaactgtccctaataaagacagagaacaaggtcttcctgcccctttaaccagggccccagagcctgctgaaatgcctagaacaggtgatgccccactgagatccggcttctgctccctaaagaactacacaagtcatgctgagttctttcactttccacatgcaagtgacttactatttccctattacttattttttgaactggactttacgtactcattagcaaggattttagctttgaagactaaatctatagcataaggattttaggcaatatttgtaaaaatctaaaagaagggaactcttggtgctagaatgttataaatacggaaaaaagcctgacctgaaccccagaggacatcatgacataaactagccttccacaactatattttagagagaaatacttgctttgctccaaaataatgccacagatttgtctactgaacaataaaatctgtactttccaatttgcatgtattttgagaaatgtaccagtgaattatcttttgatttactttagagtctcctcagatgctcatgagaagagaaaacatgacaaggtactgcttaattcaaatttctaaatgttttgcatattgacagcaagtcctgagaagcactgtcagtactgatgaggaaacactactttaaaatgtaacagtaagattaaagcttacacatgaaagctgatactgaagcctgattcactgccatgcctctattttgacatagaaatgtgcgaggctttaaattatatttaaagagaccccacgttgtggcaggacacggtaggtgtttctgtctttgctactcttcctgtccattctttcacgtgattgcgaggaaccccggaaactggagacatgggggagagctagaggcaccccaccttctgcgggggctgtccttccagtcccctctgcttctccagcaggggtcatttcagacgctgcccggtccactgaggaccagcccacttctgccactgacaggaaatcagcctcctgtatcttgcctccagcacttcctcctggagcagtttacagggatggggggagtcttagtcaagagccgtgtgcccagcccccctcccctacacgcccagggcccagtatcaggcttctgtttctttaagtgctaagaccatgcctttctcattttttcctgttctccaaacattcctctgtgacgtttcttcttctttctccagtaaaaccaagattctcccaaagggatacatcattatcactcaaagtccatagattacatgagggttcattctcaagtgttgtatatcccatgtgctttggcagacaatgtcagtggcaattgtttcactgcctgaagaatcctctgtgttccacctgttcccccctcttttcctcctacccgctggctacctgtgacctttttactgttgcaatagttttgccttttccagaatgtcctatagttgtgaccatacactatataaccttcagactggcttctttcactggataatatgcatttaagagtgcttcagatatgtttatggtttggtagctgacttctttttggtattgaatagtatttagttgtatgactgtcatatagtttatctgtttgcctactgaagaacatcttggttgcttccagattttggcaattgtgaataaagctgctacaaaca

SEQ ID NO: 26 - DNA, 2387 bp, Bos taurus
  source 1..2387: mol_type = other DNA
ggcaaatgctgaggatactttccaagtgaaccctgagtgaacctctaatatatttatttcctgtggaaagaggaaggagccaacatgaaggaaaatgcagcatctagagtggttttcattttgctactttttctcagtgtcagccttctgaatggacagtcacctcctgaaaaacccaagctcgttaaatgtcggtctcctggaaaggaaacattcacctgctggtgggagcctggggcagatggaggacttcctaccaattacacgctgacttaccacaaggaaggagaaacactcatccatgaatgtccagactacaaaaccgggggccccaactcctgctactttagcaagaagcacacctccatatggaagatgtacgtcatcacagtaaacgccatcaaccagatgggaatcagttcctcggatccactttatgtgcacgtgacttacatagttgaaccagagcctcctgcaaacctgactttggaattaaaacatccagaagatagaaaaccatatctatggataaaatggtctccacccaccatgactgatgtaaaatctggttggttcattatccagtacgaaattcgattaaaacctgagaaagcaactgattgggagactcattttactctgaagcaaactcagcttaagattttcaacttatatccaggacaaaaataccttgtgcagattcgctgcaagccagaccatggatactggagtgagtggagcccagagagctccatccagatacctaatgacttcccagtgaaggacacaagcatgtggatctttgtggccatcctttctgctgtcatctgtttgattatggtctgggcagtggctttgaagggctatagcatggtgacctgcatcctcccaccagttccagggccaaaaataaaaggatttgatgttcatctgctggagaagggcaagtccgaagaacttctgcgagctctggaaagccaagacttcccccccacttctgactgcgaggacttgctgatggagttcatagaggtagatgactgtgaggaccagcagctgatgccacgcccctccaaagaacacacggagcaaggcgtgaagcccatgcacctggatcttgacagtgactctggccggggcagctgcgacagcccttcgctcttgtctgaaaagtgtgatgaacctcaggcccatccctccaagttccatactcccgagggccctgagaagctggagaatccggaaacaaaccttacatgtctccaggcccctcagagcacaagcgtggaaggcaaaatcccctattttctggccaatggacccaaatcttccacatggcctttcccgcagccccccagcctatacagccccagatattcttaccacaacattgctgacgtgtgtgagctggccctgggcatggccggcaccacagccacttcgctggaccaaacagaccaacatgctttaaaagcctcaaaaaccattgaaactggcagggaaggaaaggcaaccaagcagagggagtcagaaggctgcagttccaagcctgaccaagacacggtgtggccacgaccccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatagaatccagccaaggccgacctggctatctcccccacaaccccaggcaactgcagactccagttgggctggggactgggtcccgcaggttttatgcactcttgcagtgagagttatggaaggatgggttcaattgtgattttccttcagggaacactacagagtacgtgaaatgcactctaccagagagggctcaagaacagggttagaatgacactacccaactcccagttcactcttaattctctattttcaaccagttgcctctttgtccaacagctgattccagaacaaatcgttccatcttgtgtgatttgtagatttacttttttgctattagttgtcagattatatgttcaaagatataaaagcacattgcctagtattcttaagagacagtgccaataggtatataatctggaaaaggccttcatggtttcgtatgtgacagaggggtataagtcagtcaaaattgtttaccatgggaagatggtagataggagagaaatgccatgaaaaccactttgaagaccagttgcttaacctttgcactcctcttt

SEQ ID NO: 27 - DNA, 2426 bp, Bos taurus
  source 1..2426: mol_type = other DNA
ggcaaatgctgaggatactttccaagtgaaccctgagtgaacctctaatatatttatttcctgtggaaagaggaaggagccaacatgaaggaaaatgcagcatctagagtggttttcattttgctactttttctcagtgtcagccttctgaatggacagtcacctcctgaaaaacccaagctcgttaaatgtcggtctcctggaaaggaaacattcacctgctggtgggagcctggggcagatggaggacttcctaccaattacacgctgacttaccacaaggaaggagaaacactcatccatgaatgtccagactacaaaaccgggggccccaactcctgctactttagcaagaagcacacctccatatggaagatgtacgtcatcacagtaaacgccatcaaccagatgggaatcagttcctcggatccactttatgtgcacgtgacttacatagttgaaccagagcctcctgcaaacctgactttggaattaaaacatccagaagatagaaaaccatatctatggataaaatggtctccacccaccatgactgatgtaaaatctggttggttcattatccagtacgaaattcgattaaaacctgagaaagcaactgattgggagactcattttactctgaagcaaactcagcttaagattttcaacttatatccaggacaaaaataccttgtgcagattcgctgcaagccagaccatggatactggagtgagtggagcccagagagctccatccagatacctaatgacttcccagtgaaggacacaagcatgtggatctttgtggccatcctttctgctgtcatctgtttgattatggtctgggcagtggctttgaagggctatagcatggtgacctgcatcctcccaccagttccagggccaaaaataaaaggatttgatgttcatctgctggagatatcacagccttctcgccttgtgtctatgttttaataggagggcaagtccgaagaacttctgcgagctctggaaagccaagacttcccccccacttctgactgcgaggacttgctgatggagttcatagaggtagatgactgtgaggaccagcagctgatgccacgcccctccaaagaacacacggagcaaggcgtgaagcccatgcacctggatcttgacagtgactctggccggggcagctgcgacagcccttcgctcttgtctgaaaagtgtgatgaacctcaggcccatccctccaagttccatactcccgagggccctgagaagctggagaatccggaaacaaaccttacatgtctccaggcccctcagagcacaagcggggaaggcaaaatcccctattttctggccaatggacccaaatcttccacatggcctttcccgcagccccccagcctatacagccccagatattcttaccacaacattgctgacgtgtgtgagctggccctgggcatggccggcaccacagccacttcgctggaccaaacagaccaacatgctttaaaagcctcaaaaaccattgaaactggcagggaaggaaaggcaaccaagcagagggagtcagaaggctgcagttccaagcctgaccaagacacggtgtggccacgaccccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatagaatccagccaaggccgacctggctatctcccccacaaccccaggcaactgcagactccagttgggctggggactgggtcccgcaggttttatgcactcttgcagtgagagttatggaaggatgggttcaattgtgattttccttcagggaacactacagagtacgtgaaatgcactctaccagagagggctcaagaacagggttagaatgacactacccaactcccagttcactcttaattctctattttcaaccagttgcctctttgtccaacagctgattccagaacaaatcgttccatcttgtgtgatttgtagatttacttttttgctattagttgtcagattatatgttcaaagatataaaagcacattgcctagtattcttaagagacagtgccaataggtatataatctggaaaaggccttcatggtttcgtatgtgacagaggggtataagtcagtcaaaattgtttaccatgggaagatggtagataggagagaaatgccatgaaaaccactttgaagaccagttgcttaacctttgcactcctcttt

SEQ ID NO: 28 - DNA, 1746 bp, Capra hircus
  source 1..1746: mol_type = other DNA
atgaaggaaaatgcagcatctagagtggttttcattttgctactttttctctacgccagccttctgaatggacagtcacctcctgaaaaacccaagcttattaaatgtcggtctcctggaaaggaaacgttcacctgctggtgggagcccggggcagatggaggacttcctaccaattacacactgacttaccacaaggaaggagaaacactcatccatgaatgtccagactacaaaaccgggggccccaactcctgctactttagcaagaagtacacctccatatggaagatgtatgtcatcacagtaagcgccatcaaccagatgggaatcagtccctcagatccactttatgtggatgtgacttacatagttgaaccagagcctcctatgaacctgactttggaattaaaacatccagaagatagaaaaccatatctatggatcaaatggtctccacccaccctgactgatgtaaaatctggttggttcagtatccagcatgaaattcgattaaaacctgagaaagcaactgactgggagactcattttgctccaaagctgactcagcttaagattttcaacttatatccaggacagaaataccttgtgcagattcgctgcaagccagaccatggatactggagtgagtggagcccagagagctccatccagatacctaatgacttcccagtgaaggatacaagcatgtggatctttgtgggcgtcctttctgctgtcatctgtttgattatggtctgggcagtggctttgaagggctatagcatggtgacctgtatcctcccaccagttccagggccaaaaataaaaggatttgatattcatctgctggagaagggcaagtccgaagaatttctgagagctctggaaagccaagacttccttcccacttctgactgcgaggatttgctgatggaattcatagaggtagatgacagtgaggaccaacacctgatgccacgcccctccaaagaacacatggagcaaggcgtgaagcccatgcacctggatcctgacactgactctggccggggcagctgtgacagcccttcactcttgtctgaaaagtgtgatgaacctcaggcctatccctccaagttccacattccggagggccctgagaagctggagaatcccgaaacaaatcttacatgtcttcaggcccctcagagcacaaacggggaaggcaaaatcccctattttctggccaacggacccaaatcttccacatggcctttcccgcagccccccagcctgtacagccccagatattcttaccacaacattgctgacgtgtgtgagctggccctgggcatggcaggcaccacagccactctgctggaccaaacagaccaacatgcttttacaccctcaaaaaccattgagactggcggggaaggaaaggcagccaaacagagggagtcagaagcctacagttccgagcctgaccaagacatggcatggccactgctccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatag

SEQ ID NO: 29 - DNA, 1878 bp, Sus scrofa
  source 1..1878: mol_type = other DNA
atgaaggaaaatgtgccatccacagtggttttcattttgctactttttctcaacatcagccttctgaatggacagtcacctccgggaaaacctgagatcttcaaatgtcgttctcccgaaaaggaaacattcacctgctggtggaagccgggggcggatggaggacttcctaccaactacacgctgacttaccacaaggaaggagaaacattcacccatgaatgtccagactacaaaactggcggccctaactcctgctacttcaacaagaagcacacctccatatggacaatatacatcatcacagtgaacgccacgaaccagatgggaagcagttcctcggatccacgttacgtggatgtgacttacatagttgaaccagaccctcctgtgaacctgactttggaattaaaaaagcccgaagaccaaaagccatatctgtggatcaaatggcttccacccaccctggttgacgtaagatctggttggctcacactccagtatgaaattcgattacaacctgagaaaacagctgagtgggagactcatttcgctgggcagcagactcagtttaagattctcagcttgtatccaggacagaaataccttgtccaggttcgctgcaagccggaccatggattctggagtgagtggagcccagagagctccatccagatacctaatgacttctcaatgaaagatacaaccatgtggatcttcgtggctattctttctgctgtcgtctgtttgattatgatctgggcagtggctttgaagggctatagcatggtggcctgcatccttccgccagttccagggccaaaaataaaaggatttgacacccatctgttggagaaaggcaagtccgaagagcttctgagcgccctgggatgccaagacttcccccccacttccgactgcgaggacctgctggtggagttcctggaggtggatgacagcgaggaccagcagctgatgccagcccactccaaggaacacccgagccaaggccggaagcccacacacctggatcccgacagcgattccggccggggcagctgtgacagcccttcccttttgtccgaaaagtgcgatgaaccgcgggccaatccccccaagtttcacactcccgagggcatcgagaagccaggggatcctgaaacaaaccttccccgtccccaggaccctcagagcacaagcgtggaaagcaagctgctctattttcacgcggatggatccaagtcttcgacttggcctttgccgcagcccccgatcctgcacgatccgggatcttcttaccacaaccttgccgacgtgggtcagctggtcctgggcaccgcaggggccaccgccgctttgctggaaaaaacggaccgacatgctttcaaccctccaaaaaccaccgagactggcggggaaggaagggcaaccgagcagaaggagtcggagagcttccactccaagactgaccaaggcacagtgtggctgctgccccaggacaaggggccctttgtctcccctaagcccatggagtacgtggagatacacaaggtcagcaaagatggagcactggcgttgctcccaaaacagcaggagaacggcgaccggccggagaaggctggcgcccctgaaaccagcaaggaatacgcccaggtgtcccgggtgatggataaccacatcctggtgttagtgcaggatccgcgagctcgaaacgtggctccgtttgaagaaccaaccaaggagaccccgccatcccggccgcagaatccagctgcgaaagacctggccggcttcaccacggccccgggccactgcagacacccgctgggtgggctggattacctcgatcccgcaggctttatgcactcctttcagtga

SEQ ID NO: 30 - DNA, 1244 bp, Sus scrofa
  source 1..1244: mol_type = other DNA
cgcacactcatcccgcttgaatcctggggcagggactcagaaaacttccagcccgggcagcgcgcgcttggtgcagaacgcaggagcaaccggtccgtccccctccaactctccggtgccgctgccgcctgctcccgccatccgaggaggcgcggtgccacccaccgctccgtcccctgcctgcgctcagtgcgcgacccgatccccgcagaatctccccatcctccctttgctttctgactgcccaaggcgctatttgttttctctctttctctttcttgctctgcgagaggggaggggggggaagaggaggaggaattctttccccgcctaacgtttcaagggacacaatccactccaagtctcttccctttccaagccgcttccgaagtgctcccggtgatcgcaacttttgatcccaaaccgagaggggagtctcccaacttcaaatcctcttttccttctccttcgctttcctcctctcactacctccaccgccaccgccacctccacctccggcacccacccaccgctgccgccgccaccagcagcgcctccttctctcctcctcctcctcccctcttctctttttggcagccgctggacgtccggtgttgatggtggcagcggcggcagcctaagcagcagcagccctcgccgcacgccagctctagcccaccctgccttattctctagtcctacctcccaatctcccgaaaggtgctgggcagattcggggcggctgaggcgaagcggcagcggtaggagcggcggcggcagcggtagcggcggcggcgggaggcaggatgagcgcacgcggtgaaggcgccgggcagccgtccacttcagcccagggacaacctgccgccccggtgcctcagaagagaggacgcggccgacccaggaagcagcagcaagagccaaccggtgagccctctcctaagagaccaaggggaagacccaaaggcagcaaaaacaagagtccctccaaagcagctcaaaagaaagcagaagccactggagaaaaacggccaagaggcagacctaggaaatggccacagcaagtcgttcagaagaagcctgctcaggaggagactgaagagacatcctcacaggagtctgcggaagaggattagggggcgccaacattcaatttctacctcagcagcagttgaatcttttgaagggagaagacactgcagtgaccacttttactctctatcgccatggtctttccactttca

SEQ ID NO: 31 - DNA, 1194 bp, Sus scrofa
  source 1..1194: mol_type = other DNA
cgcacactcatcccgcttgaatcctggggcagggactcagaaaacttccagcccgggcagcgcgcgcttggtgcagaacgcaggagcaaccggtccgtccccctccaactctccggtgccgctgccgcctgctcccgccatccgaggaggcgcggtgccacccaccgctccgtcccctgcctgcgctcagtgcgcgacccgatccccgcagaatctccccatcctccctttgctttctgactgcccaaggcgctatttgttttctctctttctctttcttgctctgcgagaggggaggggggggaagaggaggaggaattctttccccgcctaacgtttcaagggacacaatccactccaagtctcttccctttccaagccgcttccgaagtgctcccggtgatcgcaacttttgatcccaaaccgagaggggagtctcccaacttcaaatcctcttttccttctccttcgctttcctcctctcactacctccaccgccaccgccacctccacctccggcacccacccaccgctgccgccgccaccagcagcgcctccttctctcctcctcctcctcccctcttctctttttggcagccgctggacgtccggtgttgatggtggcagcggcggcagcctaagcagcagcagccctcgccgcacgccagctctagcccaccctgccttattctctagtcctacctcccaatctcccgaaaggtgctgggcagattcggggcggctgaggcgaagcggcagcggtaggagcggcggcggcagcggtagcggcggcggcgggaggcaggatgagcgcacgcggtgaaggcgccgggcagccgtccacttcagcccagggacaacctgccgccccggtgcctcagaagagaggacgcggccgacccaggaagcagcagcaagagccaaccggtgagccctctcctaagagaccaaggggaagacccaaaggcagcaaaaacaagagtccctccaaagcagctcaaaagaaagcagaagccactggagaaaaacggccaagaggcagacctaggaaatgggtgagtaataagataaaatttttgttcttttttttttttttctttggtaatgaaaactttgtctctctttttcttttttttaaaatgaaatgaattctattttatgaattacacgaatttcttttaacttccggtttga

SEQ ID NO: 32 - DNA, 1437 bp, Bos taurus
  source 1..1437: mol_type = other DNA
gcctctggcccgcggccaggcgccttggctcggcggtctgcctggcctccctcctcctcatacttttcctccagcgcagccccctcccctttatccgcccacgattagaggtgggcactccccccatctcgccgccccctccccaagcacacacacactcaacgcgctcgaattctggggtagaaactcagaaaacttccagcctgggcagcgcgcgcttggtgcaggacgcaggagtaatcagcccgtccccctccaactctccggtgccgctgccgcccgctcccgccacccgagaaggaacggtgccacccaccgctccgtcccctgcctgcgcccagtgcccgaccggatccccgcagaatctccccatcctcccttcgctttccgactgcccaaggcgttatttgttttctctctctttctctttcttgctctgcgagcgcggagcgggggggaagaggaggaggaattctttccccgcctaacatttcaagggacacaattcactccaagtctcttccctttccaagccgcttccgaagtgctcccggtgaccgcaacttctgatcccaaactgagaggggagcctcccaacttcagttcctcttttccttcttcgctttcctcctcctctcactacctccacctccaccgccacctccacctccggcacccacccaccgctgccgccgccaccagcagcgcctccttctctcctcctcctcctcccctcttctctctttttggcagccgctggacgtccggtgttgatggtggcagcggcggcagcctaagcagcagcagccctcgccgcacgccagctctcgcccaccccgcctcattctccagccctacctcgcaatctcccgaaaggtgccgggctgattccgggcggcagaggcgaagcggctgcagcggcggtaggagcggcggcggcagcggtagcggcggcgggaggcaggatgagcgcacgcggtgaaggcgccgggcagccgtccacttcagcccagggacaacctgccgccccggcgcctcagaagagaggacgcggccgacccaggaagcagcagcaagagccaaccggtgagccctctcctaagagaccaaggggaagacccaaaggcagcaaaaacaagagtccctccaaagcagctcaaaagaaagcagaagccactggagaaaaacggccaagaggcagacctaggaaatggccacaacaagttgttcagaagaagcctgctcaggaggagactgaagagacatcctcacaagagtctgcggaagaggattagggggcaccaacattcaatttctacctcagcagcagttgaatcttttgaagggagaagacactgcagtgaccacttactctctatcgccatggtctttccactttcatctggggtcgggggtgagggggtggggcgggggcgg

SEQ ID NO: 33 - DNA, 1225 bp, Sus scrofa
  source 1..1225: mol_type = other DNA
cggggcgggtggccggccggacactggacccggaaggggggtaggcggctgggatgagtggcgagctgtccatgggagcacccagcggccccattggcaccagtacaggcaggggcacctgcagcagctgaggggccgaagagtcacccctgaacttgaggacgagcagccggattccagcccccagccccagggccccacatctcctcgggctcagccgcgcgccccagctgccccccagcctgagctgcagcaggccagggctgcccgagaccccagcccccaggtgagctgctgcagcctgtggcccaggagatctccgccggctcagaactgaggcgggcagcccacccagcccacagcgattccaatggggatcccgatgaggaagccgctgctcgtgctgctcgtcttcttggccttggcctcgtgctgctatgctgcttaccgccccagtgagactctgtgcggcggggagctggtggacaccctccagtttgtctgcggggaccgcggcttctacttcagcaggccggcaagccgcgtgaaccgccgcagccgtggcatcgtggaagagtgctgcttccgtagctgcgacctggccctgctggagacctactgcgccacccccgccaagtccgagagggacgtgtcgacccctccgaccgtgcttccggacaacttccccagataccccgtgggcaagttcttccgctatgacacctggaagcagtccgcccaacgcctgcgcaggggcctgccggccctcctgcgcgcccgccggggtcgcacgctcgccaaggagctggaggcggtcagagaggccaagcgtcaccgacccctgaccgcccgtcccacccgagaccccgccgcccacgggggcgcctctcccgaggcgtccggccatcggaagtgagccaaattgtcgtaattctgcggtgccaccatccacctcgtgacctcctctcgaccgggaccgcttccatcaggtcccccttctgagatctctgtacccttctgtctgcgggcatctccgccccgggccccgtgccccaacctccccatgtcaggctagtctctcctcggccccttccatcgggccgagggcatccaaaccacaaacccaattggcttggtctgtatctccccccaaattatgcccccaattatccccaagttacataccaaaaattgaacccctcaaccacacccacataccatcacg

SEQ ID NO: 34 - DNA, 3622 bp, Bos taurus
  source 1..3622: mol_type = other DNA
gcggggggctgggctgatcagtgatctgtccgtgggctcacacgggcggccctgtgcgtgcaggtcggggcaggggtgccccgcaggagctgagacatcaatggggatcacagcaggaaagtcggtgctggtgcttcttgccttcttggccttcgcctcgtgctgctatgctgcttaccgccccagcgagactctgtgcggcggggagctggtggacaccctccagtttgtctgtggggaccgcggcttctacttcagccgaccatccagccgcataaaccgacgcagccgtggcatcgtggaagagtgttgcttccgaagctgcgacctggccctgctggagacttactgtgccacccccgccaagtccgagagggatgtgtctgcctctacgaccgtgcttccggacgacgtcaccgcataccccgtgggcaagttcttccaatatgacatctggaagcagtccacccagcgcctgcgcaggggcctgcccgccttcctgcgagcacgccggggtcgcacgctcgccaaggagctggaggcgctcagagaggccaagagtcaccgtccgctgatcgccctgcccacccaggaccctgccacccacgggggcgcctcttccaaggcatccagcgattagaagtgagccaaagtgtcgtaattctgccaagtggcaccatctacctcgcgccgacctcctgaccgggaccgccccactaggtctctctctgaaatccctgtaccgtcctgtctgcgggctcccctgccccggcctctgtgccccaacctccccacgtcaggcgaatccccctcggccccctccatctggctgaggggatcagaacaacatctctaaaaatgtacaaaaccaattggctttaaatatccccccaaattatcaccccccaaattacccccaaattacacaaccaaaattgcaatcatgaacccctcaatcagcccccttgaaacgaattggctttttagcaacaccagaaaagcaaactagctttccaaaaacttcttaaaaaaaaaaaaatcaattggctgaaaaaaatactaaaaataaattggcttaaaaacaattggcaaaataaaagaattcggcccccccccccttccttctcgttccttttggatctggacttaaattggctttgatctaatcatctaagagaaaggaaggggccaaatttgcaggtaggcttgtccttgctggccagccatctccccccgaccgtcgccaggccctcctcgggcactggcagtgtgacaccaaagacccaaacctgctcctcctccaagtttccatcactgggaccacagagcagtgaggggacgctgtgagaccagaggaggggagcacggagcagaaaccccggatcgcgcagataaagttgaattggcaaaacacccccacactccaaatctgacatcacaatcctaccttggaaagcacatgcaaccctgcacacacacacacacacacacacacacatccatgcacgcacacacacacacacgcatccatgcacgcacacacccacatgcatgcatgcacacacacacacacagcctcacatgcatccatgcacacacacacacacacacacacatccatgcacgcatgcacgcacacacacacacacacacacacacacacacacacacacactctctagggacacccgtgcccagccttgtgcagctgagaccgaattccatcccaaattattctctgagctgccccgcgatgcggttttcctgttccgtgctcccctctgatcccctgggatgcaccattgtgggaaggtgtgtctcgggctcaggcaggtggttccatctttccgagaagagggtgatccccacctcttctcggcacctgttctgaccccacagcccacagaatgtaagtcctgggtgcccctagaccccagaggcatccacgcaccgaccatcgtgcagagtgaggaacggtttggccaggcgggagatggctgcagccaagcgagacgagcccaaccacgtagcctgaccccctgggtgtgcttctgcaagatctggggagccctctcccccgagcacacctagggatcatctttgccagcctcctggggaagcctctcagaaatgagggcccccccggaggctggcaaaggtgacggggagtgtgggaagtttggaggatggcggggtgggtggggggcagtgggggctgggtggggggacattccgaggcaggaagtggtccagcaagatttcggagagaaacgttcggggggaaggaacagcgagacacttgcagaattacagacggaaatcagagcccaaattgacgccaattgatctatctcccctctttaattactcctggggcatctttccttttttttttttatccctccttagctttttacgcgctcttttatcaatttgctccctaccccacgtaacaggggggcagtgaccaaggacgaggaacacacagagtcaccaccgtcacccgcggctccgccgccatcctgccctccgccatttatcgccctgtttttaattttttttttatctgtccctgtcgcttgggttgagtggagagtggagcctctgtgggggtgtcggccactgtgccccccagagaagtcaggggaatggagacggccgctgcctggcctggcccccgggggacagtggctggtccccggggggtcctgaggggcggaggagggggcggggaggcgtctctccaggtgtcaagaaggtgctcggtggccaccaaggtggggcccctggccggcctggccggccaggggggaaggggctacagatgtgtgagcgaagggctccaacgggcgggagcaggtggccgccttctgcgcacttggggaggctccccccgtacccctcgggaacagcttgcccgcctcctcagcgccctgtcttgcccccaggaagctcagagctccgcagggcttcctggcgtccaggcggggtgaggtcgggtggtaggagaggccgggagaaccgggccctgcagagcagaagagctgcgtagttttctgaccgctgggtcgcttgggccccgaggccctcgggtctggtgacgcgacgcagccaccactctcggcacctcgggctgtggcagggacccgggcggctgggggcttacctcacccccgcctctgcccccagcgcagtccccctgcacggcagcccgactagtgagctagaggcccgacacctcgggtccccggtgacggggctgacacgacccagggggcggttgtaccagtccaccccagacacagcgggcagctctccgtgggtcagctccatccgacccggggccccttccgacctccgcgcccagctcgtggccctccatcttgtctcttccccgagtccccccagctgtcttcagcgggaccccctctcgggtctctgagtcatcacgcgaggacccccacgcgcagaacaccaagtcatcgtgtccgcgccccgtccaccgcgcggctgtgttctcctcctcttgcccgtaacgtgctgtttctggcagaatccacagcctgggttttgtctttaacctttatcgctcgcaatcccaataaagcatttaaaattagtt

SEQ ID NO: 35 - DNA, 3494 bp, Sus scrofa
  source 1..3494: mol_type = other DNA
taactccagataaatccaggaacacggtgtttacgaggtgcccttaaaggcgggcagagaacaggagagggtgacaggagagggtgacatcagggccctgagcagaggcactagcacgggggtgggcggtggggcaggtcccaggccttggtggccgcaaacctggacggcaggcgtcccagcctgcatcccgcgctccccccacagctcggctgcataaattccaagggacgccgcagggccctcggtaaatatttgtccaaggcgcacaggagtaaaggcaggagcgggcagggtgtgaccgtccatccgagctcttcccggcagccgcacctcgggggccggtcccgctcggataaaactctccgccgccagcgagggtcgccgcgtcgcagccatgcccaggtacacggtgcacgtgcgcggggagtggctggccgtgccctgcggggacgcgcagctcacggtgggctggctgggccgcgaggccgtgcggcgctacatcaggaacaagcccgacaacggtggcttcgcctcggtggacgatgtgcgcttcctcgtgcgccgctgcaagggcctgggcctgctggaccacgaggacctgctggaggtggccctggaggacaatgagttcgtggaagtggttatagaaggggatgccatgtctccagacttcattccatcgcaaccagaaggcgtttacctttacagcaaataccgggaacccgaaaagtacatcgccttagatggggacagcctgaccacagaggacctggtcagcctgggaaagggacactacaaaatcaagctcacccccactgctgaaaagaaggtgcaaaaatccagggaagtcatagacagcatcgtgaaagaaaaaaccgttgtttatggcattactacaggttttggaaaatttgccagaaccgtaattcctgtcagtaaactagaggagctccaggtcaacctggtgcgctcacattcttcaggcgttgggaaaccgctaatccctgagagatgccggatgctcttggctttaaggatcaatgtcttagccaaaggatacagtggcatttccctggagactctcaaacaagttatcgaagtatttaatgcctcctgcctgccctatgttccagagaaaggaactgttggtgccagcggagaccttgccccgctctctcacctggcactcggtctcatcggggaagggaagatgtggtctccgaagagtggctgggccgacgcgaaatatgtcctggcagctcatggattgaaaccaattgtcttgaaaccaaaagagggcctggcgctcatcaacgggacccagatgatcacctctctgggctgcgaagctgtggaaagagccagtgctattgctcggcaggctgacatcgtggcggccttgaccctggaggtgttgaagggcaccaccaaagccttcgataccgacatccacgcggtccgccctcaccgtgggcaagtcgaagtcgcctttcgctttcgctccctcttggactcggatcaccacccgtctgagatagcagaaagtcacaggttctgcgatcgggttcaggacgcctacaccctgcgctgctgtcctcaggtccacggtgtggcgaatgacacgatcgcatttgtaaagcacatcattaccacagaaattaacagtgccacggataaccctatggtctttgccagcaggggagagactatttctggaggaaacttccatggtgaatatccagccaaagccctggactatctggccattggcatccacgagcttgctgccatcagtgaacgacgaattgaaaggctctgtaacccctccctcagcgagctgcctgccttcctggtggctgaagggggtctcaactccggattcatgatcgcccactgcacagccgcagcccttgtttctgagaacaaggctctgtgccacccctcgtccgtggactctctctccaccagcgctgctacggaggatcacgtctccatgggaggatgggcagccaggaaggcgctcagggtcgtcgagcacgtggagcaagtgctggccatcgagcttcttgccgcctgccagggcatagagtttctgcgccccttgaaaaccaccactcccctggagaaggtctatgacctggtgcgctctgtcgtcaggccctggattaaagaccgcttcatggccccagacatcgaggcggcacataggctgctcctagagcagaaggtttgggaagtggctgcaccatatattgagaaatacagaatggagcatattccagaatcaagacctgtctctccaacagcattttcactggaatttctgcacaagaagtcccccaaaatcccagagtccgaggatctttaatgcgttccgccgtggattagcagatgtgatggtacttagtttcacagcaagcaagcagtatcttgctgaagaagagacctgaggactttcttgtggatcaatctattctatagtccaggtctctaatacctgctctagttaggctggtgacagtattacatttactaaatctagtccgcttcttgttgagctggttagagacacccctggtgttttcagatgataggcgttttcttgtttcttcaccatatcaaaaggccactcactcttaagagtgagaagagtaaccaccactgcatggttctttaattctttttttttttttctgtctttttgcctttttgccttttgagggccgctcccgcagcacatggaggttcccaggccaggggtccaatcggagctatagctactggcctacgcctgagccacagcaatgccagatccttaacccactgagtaaggccagggatcgaaccgcaacctcatggttcctagtcagattcgtcagccaccgagccacgacaggaactccaggttctttaattcttaacatcagatgaactcaaatctagacggcattattttccaaaaacattgtactttcgtcgatctttttggggtttttttttgtttgttttttttgcttgacaagctctccactagctaactttgactgactgacccatgccaagatgtgacaaatgttccttactgtgtcttcataaaatatccttttcaggtcataatttggaagcctttctttctttattaattgaaccctagtgaagaataatcaagggagttccggttgtggctcagcaggaacaagcccgactagtatccatgaggatgtgggtttgatccctggccttgcttagtgggttaaggatcctgtgttgccatgaacggtggtgtaggtggcagacgtggctgtgttgctgtggctgtggtgtaggccagtagttgcagctgatttgactcctagcctgggaatttccatatgctgtgggttcagccctaaaatgagaaaaaaaaaaaaaaattggtcaattggtcaataggagatgtttgcataaaggagtgaaggaatgaatataaagattgctattggagtggctct

SEQ ID NO: 36 - DNA, 2625 bp, Sus scrofa
  source 1..2625: mol_type = other DNA
ggggctcgcgcggcagaaaagcggaggagcagccagaccccggcgctggtcaccgcaccggggacggtaagtgtgggagaacagccctgcatttctgctgacgggtcaacgtcacagcgtcaaagaaaaggaaggtacatttcagctgaactgatcaaggaagaagatggtttattccaactgtgaaagtaaagaacctgattcagtttctgcatccaatcacctgttactaaatgggaatgatgaattggtggagaaaagtcacaaaacagggcctgagaacaacctgtacagccagtacgaggagaaagtgcggccctgcatcgacctcatcgactcactgcgggccctgggcgtggagcaggacctggccctgcccgccatcgccgtcatcggggaccagagttcgggcaagagctccgtgctggaggccctgtcaggggtcgctctccccagaggcagcggaattgtgacaagatgccctcttgtgctgaaattgaaaaaactcgtgaacgaagaagacgaatggaagggcaaagtcagttaccgggacagcgagattgagctttcagatgcttcgcaggtggaaaaggaagtcagcgcagcccagattgccatcgctggggaaggcgtgggaatcagtcatgagctaatcagtctggaggtcagctcccctcacgtcccagatctgaccctcatagaccttcctggcatcaccagggtagctgtaggcaatcagccatacgacatcgaataccagatcaagtctctgatcaagaagtacatctgtaagcaggagaccatcaacttggtggtggtcccctgcaacgtggacattgccaccacggaggcgctgcgcatggcccaggaggtggaccccgaaggagacaggaccatcgggatcttgacgaagccggatctggtggacaaaggcactgaggacaagatagtggacgtggcgagaaacctggtcttccacctgaagaagggctacatgattgtcaagtgcaggggccagcaggacatccaggaccagctgagcctggccaaggccctgcagaaggagcaggccttctttgaaaaccacgaacatttcagggatcttctggaggaagggcgggccacgatcccctgcctggcagaaagactgacctctgaactcatcatgcacatctgtaaaactctgcccctgttagaaaaccaaataaaagagagtcaccagaaaataacagaggagttacagaagtatggctccgatattccagaggatgaaagcgggaagatgttttttctgatagataaaatcgatgcatttaatagtgatatcactgctttgatacaaggagaggaactggtggtggagtacgagtgtcggctgtttaccaagatgcgaaatgagttctgcaaatggagtgctgtggttgaaaagaatttcaaaaatggttatgacgccatatgtaaacaaatccagctcttcgaaaatcagtaccgggggagagagttgccagggtttgtgaattataagacatttgaaaccatcattaagaagcaggtcagtgtcctggaagagccagccgtggacatgctgcacacagtgactgatttagtccggctcgccttcacagatgtttcagaaacaaattttaatgaatttttcaacctccacagaactgccaagtccaaaattgaagacattaaattagaacaagaaaaagaagctgagacgtcgatccggctccacttccaaatggagcagatcgtgtactgccaggaccaggtctatcggggcgcgctgcagaaggtcagagagaaggaggcggaagaagaaaagaacagaaaatcaaaccagtactttctgtcgtcgccggccccctcctcagacccctccatagccgagatctttcagcacctgattgcctaccatcaggaggtcggcaagcgcatctccagccacatccctctgatcatccagttcttcatcctccggacctttgggcagcagctgcagaagagcatgctgcagctgctgcagaacaaggaccaatacgactggctcctgagggagcgcagtgacaccagcgacaagaggaagttcctgaaggagcggctgatgcggctgacccaggctcggcgccggctcgccaagttcccaggctgaaccgggctctccaggcggcccggggtctccagggcacgtctccaggcaacgaggaccaacctccttccctaacagactagcatcatgagctcctgtttcgcacatcctcctgtggttagtagactctaaagccaccgtccctgctgttagtggctgaggacttagcaagaagctgtgataagcacgctggctgcaagcatcaggccatttacttgaatgagccccgccaacgcttccgcctcccgcgcctctctccatccctctctccatccttctctccatccctgtataggatactggtccccgcatagcatcatagaagggtcattctggtttctgtacaagcctttcacgcccaatgtcttaggggcattacagccacctgtgtggatggatgcacatagaagcctatttcttttatttgtaataaacttggttctaccagccgttaaaaaaaaaaaaaaaaa

SEQ ID NO: 37 - DNA, 6609 bp, Sus scrofa
  source 1..6609: mol_type = other DNA
gtgcctgcaggacccaacgtactcaatgagctttccagcgcaatgtccgatcgctcggggccgactgccaaggggaaggatggaaagaagtattcctcgctcaacctgtttgatacgtataagggcaagtccttagagatccagaaacccgctgttgcccctcgccatggcctgcagagtctcgggaaagttgctattgctcggcgtatgccacccccagccaaccttccaagcctgaaagccgagaacaaaggcaatgaccccaacgtctcactagtgccgaaagacggaacaggatgggcaagcaaacaggagcagtccgaccccaagagttccgatgcctcaaccgctcagccgccggaatcgcagccactgccggcttcacagacgcctgcctccaaccaaccgaaacgacccccagcagccccagagaacactccttcggttccaagcggggtaaagtcctgggcacaagccagcgtcacccatggagcacatggagatggtggaagggcatcaagcctactgtcacgattctctcgagaggaatttccgaccctgcaggcggctggcgaccaggacaaggctgccaaggaaagggagtctgccgaacagtcgtctgggcccggaccaagcctccgcccccaaaattccacaacttggagggacggaggtgggcgtggccctgatgagctggagggcccggactccaaacttcatcacggccatgatccccggggtgggctgcagccttcaggcccaccccagttccctccctaccgcggaatgatgccgccctttatgtatcccccatatctcccattccctccgccctatggaccccaggggccttaccgataccccgctcctgatgggcctagccgtttcccccgtgtggcaggcccccgaggctcagggccgccaatgcgcctcgtagagcctgtgggtcggccttccattcttaaagaagataatctcaaagagtttgaccagttggaccaggagaatgatgatggttgggcaggggcccatgaggaggtcgactacactgagaagctcaagttcagcgatgaggaagacgggcgagactctgatgaggagggagctgagggccacaaggagtcccaggcagctgccggtgaggaacggccccctgaagcagatggcaaaaagggcaactcccccagcagcgaaccgccccctcccaagacggcctgggcagagacctctcggcctccagagacagagccggggcctcctgccccaaagcctcccccacccccacctcaccggggccccgccgggaactggggcccccctggggactacccagatcgagggggccctccctgcaagcccccagcacctgaagacgaggatgaggcatggcgacagcggcggaagcagtcttcatctgagatctccctggctgtggagcgggcccggcggcggcgagaggaggaggagcgacgcatgcaagaggagcgcagggctgcctgtgccgagaagctcaagcgacttgatgagaagtttggggcacccgacaagcggctcaaggcagagcctgcggcaccacccgctgtccctcctgcccccgctccaccacctgcagtccccaaagaactccccacacctccagcaccttcaccggcaccagtcccaacacctgaaaaagaaccagaagagccggcacaggcccctcctgcccagtccacccccactccaggtgtggctccagctcccaccctggtgagtggtggtggcagtaccagtagcaccagcagtggcagcttcgaagccagcccagtggaaccccagctgccctcaaaagagggtcctgaatcaccagaagaggttcctgcacctgccacaccgccagcaccaaaggtggaacccaagggtgatgggattggtcctacccggcagccccccagccaaggcttaggctaccctaaatatcagaagtcattgccgcctcgtttccagcggcaacagcaggagcagctcttaaagcagcaacaacagcagcagtggcagcagcatcaacagggctctgccccacctgccccagtacccccgtcaccaccacagcctgtgactatgggggctgtgccggctccacaggctgctccaccacccccaaaggccctgtaccctggtgctctgggccggcccccgcccatgcccccaatgaactttgatccccgatggatgatgattcctccttatgtggacccccggctcctccagggcaggccccctctggacttctacccccccggtgtgcatccctctggcctagttccccgggagcgctcagacagtggaggctcaagttcagagccatttgagcgccatgcagcccccctcttacgagatcggggcaccccaccagtggatccaaagttggcctgggtaggagatgtcttcaccaccacagccactgactcccgcccccttacctcaccactgcgccaggctaccgacgaggaggacaaggggatgaggagcgagacccctccagtacccccaccgccctatctggccagttatccaggctttcccgaaaatggagcccccggacccccgattcctcgcttccctctggaggagccagctcccccagggccccgtcccctcccctggcccacaggcactgatgaggcagccaagatacaagctccaccacccaagaaggaaactcccgaggaggagacaccacagctgacaggcccagaagctggtcgaaagcctacccgtggagtcggaggccaaggccccccaccgcctcgaagagagagccgcactgagacgcgctggggtcctcgtccgggcagcagtcgtcggggaatccctccagaggagccaggggcccctccccgccgggctgggcctatcaagaagcccccactagctacaaaagttgaagagctgccccccaagcccctggaacagggggatgaaacccccaaggttccaaagccagacacactgaagacggccaaggggaagatagggggccccaaggagaccccacccagtgggaatctttcccctgccccgaggcttcggagggactattcctacgaaagagtgggtcctacctcttgccggggtcgtggccgaggggagtactttgccagagggaggggttttcggggaacctatgggggacgagggcggggagcccgaagccgagagttccgtagttaccgagagttccgaggagatgatggtcgtggaggagggacagggggaccaaaccatccgcctgctccccgaggccgcactgccagtgagacacgaagtgagggctcagagtatgaagagatccccaagcggcgtcggcagcggggctcagaaacaggcagtgagacccatgagagtgatctggccccctcagacaaagaggcccccacacccaaggaaggggtactcacccaggtccctcttgctcccccaccatcaggagcgcccccttcgccagccccagcccgtttttccactgcccgaggtgggcgagtcttcactcccagaggggtgccgtcacgccggggccggggcggcgggcggcctcctccccccatctgcccaggctggagccctccaaccaagtccctggctccgaagaagcctccaacgggtcctttgccaccaagtaaggagcctttgaaagagaagctgctcacagggcctctgtcccctgtggcccgcggaggcagcagtggaggcagtaacatgggcctgggtgtggaagatggggagcggcccagaaggaggcgacacgggagggctcagcagcaggacaaaccacctcgcttccggaggctgaagcaggaacgggagaatgctgcgaggggggccgatggcaagccctcttccctatccctgtcaacttcggctccgggaccggaggaggccctccccacggtagcagtgcccccaccgcctcgccgggcagctgccaagtctcccgacctgtcaaaccagaaatcagaccaggccaatgaggaatgggagactgcgtcagagagcagcgactttgccagtgaacgccgaggggacaaagaggctccaccgccagcactactaacttccaaagccgtggggactcccgggggcggtggaggtggaggcggaccaggcctttcaaacatgtcccgtggagatttgagccagagagccaaggatctgagcaagcggagcttctcgagtcagcggcctggcatggaaaggcagaaccggcgcccgggtccaagcagcaaggccagcagtggtggcggcaacagtggaggaggaggtggggggcctggaggaaggacaggcccagggcgaggggacaagagaagctggccatctcccaagaaccgaagccgtcctccggaagagcgggccccagggctgccgctgcctcccccgccccccagcagttctgctgtcttccgcctggaccaagttatccacagcaaccctgctggcatccaacaggctctggcccagctcagcagccgccaaggaaatggggctgcacctggggggcacccaaggcccaaaactgggccttcccaaacccctcaaggaccgccgccgcctcggcccccagcccggtatgaccctcagagggccaacagcaacagtgtcacttccgacccccactttgaggagccggggccgatggtgaggggggtgggcggaacaccccaggactctgccggggttaatcccttcccccccaaacgtcgagaacggcctcccagaaaaccggagctgctgcaggaggaatctttgccatcttctcacacctctggattcttgggccctaaacccgagggcccgggccctcagggagagtccagagatgcaggaacagaggccctaagccctcacatctggaatcgtttacatgccgccgctggccgaaagcgttaccagcctggctccatggagccgtggatggaacccctgagtccgtttgaagatgtggctggcacagagatgagtcagtctgacagtggggtggacctgagtggggattctcaggtgtcatcaggtccctgcagccagcgaagttcccctgatggaggactcaagggggcagcagacgggctccccaagcggcctggaggcccctcacccctgaatgctgttcctggtgagggtccacctggctctgaaccctctgaacctcctaggagacggccacctgccccccatgatggggacagaaaggagctgccacgggagcagcctctgccccctgggcccattggcacagagcgattgcagcgtacagaccgaggcccagagcctagctccctccggtcggcccatcgacctggtcccccagtccaatttggcaccagtgacaaggactcggatttgtgcctagtggttggagacagtttaaaagcagagaaggagctaacggcatcagtcgcggaggccattcccatcgctcgagactgggagctgctccccagtgcttctgcttcagctgagcctcagtccaagagcctgggttctgcacactgtggcccagagcccagctcctcaggccaacgcttgtaccctgaggtcttctatggcagccctgggcctcccagttctcaggtctcggggggagccatagactctcagttgcatcccaacaatggaggcttccgccctgggacaccctcactgcacccttacagatcacagcccctatatctgcccccgggcccagccccaccctcagcactgctctctggggtagccgtcaagggccagtttctggatttctcagcactgcaagcagcagaactggggaagttgccagccggaggagttctcttccctccaccttccttcctctactctccagctttctgccctagccctttgccagatccacccttgctgcaggtgcgccaggatctgccatccccctcagatttttattctactcctctgcaacctggtggccagagtggtttcctcccctcgggggctcctgcccagcagatgcttctacccatggtggactcacagctgcctgtggtaaactttggctccctgccaccagcaccacctcctgctccaccgcccctttctctgttacctgtgggtcctgctctgcagccccccagcctggctgtgcggcctccgcctgctcctgctgctcgggtgctgccttcaccggccaggccctttggccctagcttggggcgagcagagctgcaccctgtagaactaaagccgttccaggattaccgaaaactgaacagcaaccttgggggacctgggtcgtcacgtactcccccagctggaaggtctttctctggcctcaattcccgtctcaaggccccaccttccacctacagtggagttttccgcacccagcgcatcgacctctaccagcaggcctccccaccagatgccctgcgctggatgccgaagccttgggagcggacagggccaccatctcgagaagggccatcccgaagggcagaggagcccgggtcccgaggggacaaggagcctgggttgcccccaccccgctgagggagttccctttgccccccaccccccggggcttgttatatagattataaatatataagggggaaa

SEQ ID NO: 38 - DNA, 3869 bp, Sus scrofa
  source 1..3869: mol_type = other DNA
agcggcgatggcggcggcggcgggagctgcagcggcggcggccgccgagggggaggcccccgctgagatgggggcgctggtgctggagaaggagcccagaggagctaccgagagagttcatggctctttgggggacacccctcgtagtgaggaggccctccccaaggccaaccctgactccctggaacctgctggcccctcatccccagcctctgtcacagtcaccgtcggcgatgagggagctgacactcctataggggccacacctctcattggggatgaacccgagaatctcgagggagatggggacctccatgggggacgcatcctgctgggccatgccacaaagtcattcccgtcttcccctagcaaggggggtgcctgtcccagccgagccaaaatgtcaatgactggggctggaaaatcacccccatcagtccagagtttggctatgaggctgctgagtatgccgggggcccagggggcagccacagcagcaatacctgaaccccctccagccacagccagcccagaggggcagcccaaggtccatcgagccaggaagaccatgtctaaacccggaaacggacagcccccagtccccgagaagcggccccctgaagtgcagcatttccgcatgagcgatgacgtgcactcgctggggaaggtgacctcagacgtggccaaaaggaggaagctgaactcaggaggtggcctgtcggaggagttgggttctgcacggggttcaggagatgtgtccctggagaagggcgaccccgggtccctggaggagtgggagacggtggtgggggatgacttcagcctctactacgattcctactctgtggatgagcgagtggactcggacagcaagtctgaggttgaagctctggctgaacagctgagtgaggaggaggaagaagaggaggaggaagaggaggaagaggaggaggaggaggaagaagaggaggaggaggaggaagatgaagagtcagggaatcagtcggacaggagcggttccagcggccggcgcaaagccaagaagaagtggcggaaggacagtccgtgggtgaagccgtcacggaaacggcggaagcgggagcctccgagggccaaagagccacgaggcgtgtccaacgacacatcctccctggagacagagcgagggtttgaggagttgcccctctgcagctgccgcatggaggcacccaagatagaccgcatcagtgagagagcggggcacaagtgcatggccacggagagtgtggatggagagctatcgggctgcaacgccgcgatcctcaagcgggagaccatgagaccgtcgagccgcgtggccctgatggtgctctgtgagacccaccgcgcccgcatggtcaaacaccactgctgcccaggctgtggctacttctgtacggcgggcaccttcctggagtgccaccccgacttccgagtggcccaccgcttccacaaggcctgcgtgtcccagctcaacgggatggtcttctgtccccactgtggggaggacgcatctgaggcccaagaggtgaccatcccccggggggatggggtgaccccaccagcgggcactgcagcccccgcccccccacccctggcccaggatgcccccgggagagcggacacttcccagcccagcgcccggatgcgaggtcacggggagccccggcggcccccctgcgaccccctggctgacaccatcgacagctcggggccctccctcaccctgcccagtggaggctgcctctcagctgtggggctgccacctgggccaggccgggaggccctggagaaggcactggtcatccaggagtcggagaggcggaagaaactccgtttccacccccgacagctgtacctgtcagtgaagcagggcgagctgcagaaggtgatcctgatgctgttggacaacctggaccccaacttccagagcgaccagcagagcaagcgcacgcccctgcacgcggccgcccagaagggctcagtggagatctgccacgtgctgctgcaggctggagccaacatcaacgcagtggacaagcagcagcggacgccgctgatggaggctgtggtgaacaaccacctggaggtggctcgttacatggtgcagcgcggcggctgcgtctacagcaaggaggaagacggctccacctgcctccaccacgcagccaaaattgggaatctggagatggtgagcctgctgctcagcacgggtcaggtggacgtcaacgcccaggacagcgggggctggacgcccatcatctgggctgcggagcacaagcacatcgaggtgatccgcatgctgctgacacggggcgccgacgtcaccctcacagacaacgaggagaacatctgcctgcactgggcctccttcaccggcagcgccgccatcgccgaggtcctcctgaacgcccgctgtgacctccatgctgtcaactaccacggggacacgccgctgcacatcgcagcccgggagagctaccacgactgcgtgctgttgttcctgtctcgaggggccaaccctgagctgcggaacaaggagggggacacggcgtgggacctgacccctgagcgctccgacgtgtggtttgcactccagctcaaccgcaagcttcggctcggggtgggaaaccgggccattcgcactgagaagatcatctgccgggacgtggcccggggctatgagaacgtgcccattccctgtgtcaacggggtggacagcgagccgtgccccgaggattacaagtacatctcggagaactgcgagacgtccaccatgaacatcgaccgcaacatcacgcacctgcagcactgcacgtgtgtggacgactgctccagctccaactgcctgtgcggccagctcagcatccgctgctggtatgacaaggatgggcggctgctgcaggaatttaacaagatcgagcccccgctgatttttgagtgtaaccaggcgtgctcctgctggagaaactgcaagaatcgcgtagtgcagagcggcatcaaggtgcgactgcagctctaccgaacagccaagatgggctggggggtccgcgccctgcagaccattccccaggggacttttatatgcgagtatgtgggagagctgatctctgatgccgaggctgacgtgagagaggatgattcttatctcttcgacttggacaacaaggatggtgaagtctactgcatcgatgcccgttactacggcaacatcagccgcttcatcaaccacttgtgtgaccccaacatcatccccgtccgggtcttcatgctacaccaagacctgcgatttccgcgcattgccttcttcagttcacgagacatccggaccggggaggagctggggtttgactatggtgaccgcttctgggacatcaaaagcaaatatttcacctgccagtgtggctctgagaagtgcaagcactcagcggaggccatcgccctggagcagagccgcttggcccgcctggacccccaccccgagctgctgcccgagctcagctccttgccccccgtcaacccctgagagtggaccacacacacccctccctaccccaggacggccaccaactcagcagccacctctgccgcttgctgcccacagcccacacctgggggtgccacagtctcctgtccctgcacccctccacacattcctcagccagaaatccccgccaggccctggaggtctgacagcccctctctcccagagctggttcctccctgggagagtgacttcagggctggccacccccctgttccccatccttagctgaagtttgatgaattgaaattggggccctatgctaactggtttcctttgttctcaataaatgttgggtttgtaataa

SEQ ID NO: 39 - DNA, 3980 bp, Bos taurus
  source 1..3980: mol_type = other DNA
atggcggcggcggcgggagctgcagcggcggcggccgccgagggggaggcccccgctgagatgggggcgctggtgctggagaaggagcccagaggagccaccgagagagttcatggctctttgggggacacctctcgtagtgaggagaccctgcccaaggccaaccccgattccctggagactgctggcccctcatccccagcctctgtcacagtcactgtcggcgatgagggggctgacacccctgtaggggccacaccactcattggggatgaacccgagaatctcgagggagatggggacctccatgggggtcgcatcctgctgggccatgccacaaagtcgttcccgtcttcccccagcaaggggggtgcctgtcccagccgagccaagatgtcaatgacaggggccggaaaatcacccccatcagttcagagtttggctatgaggctgctaagtatgccgggcgcccaaggggcgccagcagcagggcctgaaccccctccggccacagccagcccagaggggcagcccaaggttcatcgagccaggaaaaccatgtccaaaccaggaaatggacagcccccagtccctgagaagcggccccctgaagtgcagcatttccgcatgagtgatgacgtgcactcgctggggaaggtgacctcagatgtggccaaaaggaggaagctgaactcaggaggtggcttgtcagaagagttggcttctacgcggagttcgggagaagtgaccctggagaagggggaccccgggtccctggaggagtgggaaacggtggtaggggatgacttcagcctctactatgattcctactctgtggatgagcgggtggactctgacagcaagtctgaggttgaagctctggctgaacaactgagtgaggaagaggaagaggaggaggaggaagaggaggaagaagaagaggaggaggaggaggaggaagaagaagaggaagatgaagagtctggcaatcagtctgacaggagtggttccagcggccgccgcaaagccaagaagaaatggcggaaggacagcccatgggtgaagccatcacggaaacggcggaaacgagagcctccgagggccaaggagccacgaggagtgaatggtgtgggctcctcaggccccagtgagtacatggaggtccctctggggtctctggagctgcccagcgaggggaccctctctcccaaccacgctggggtgtccaatgacacatcttcgctggagacagagcgcgggtttgaggagctgcccctctgcagctgccgcatggaggcccccaagatagaccgcatcagcgagagagcagggcacaagtgcatggccacagagagcgtggacggagagctgtccggctgcaatgctgccatcctcaagcgggagaccatgagaccgtccagccgtgtggccctgatggtgctctgtgagacccaccgtgcgcgcatggtcaaacatcactgctgccccggctgtggctacttctgtacggcgggcaccttcctggagtgtcaccccgacttccgtgtggcccaccgcttccacaaggcctgtgtgtcccagctgaacgggatggtcttctgtccccactgtggggaggacgcatctgaggcccaggaggtgaccatcccccggggggatggggtgaccccaccggctggcactgcagcacccgctcccccacccctggcccaggatgcccccgggagagcggacacttcccagcccagtgcccggatgcgggggcagggggagcctcggcgtccaccctgcgaccctctggccgacaccatcgacagctcagggccctccctaaccctgcccaatgggggctgcctctcagctgtagggctgccaccagggccaggccgggaggccctggagaaggccctggtcatccaggagtcagagaggcggaaaaagctccggttccaccctcggcagttgtacctgtccgtaaagcaaggggagctgcagaaggtgatcctgatgctgttggacaacctggaccccaacttccagagtgaccagcagagcaagcgcacacccctgcacgcggctgcccagaagggctctgtggagatctgccatgtgctgctgcaggctggagccaacatcaatgccgtggacaagcagcagcggacgccgctgatggaggccgtggtgaacaaccatctggaggtggctcgctacatggtgcagcgtgggggctgtgtctacagcaaggaggaagacggctccacctgcctccaccacgccgccaaaattgggaacctggagatggtcagcctgctgctgagcacgggacaggtggacgtcaatgcccaggacagcggcgggtggacccccatcatctgggctgcagagcacaagcacatagaagtgatccgcatgcttctgacacggggcgcggacgtcactctcaccgacaacgaggagaacatctgcctgcactgggcctccttcaccggcagcgccgccatcgccgaggtcctcctgaacgcccgctgcgacctccacgctgtcaattaccacggggacacacccctgcacatcgcggcccgggagagctaccacgactgcgtgctgttgttcctgtcacgcggggcgaaccctgagctgcggaacaaggagggggacacggcgtgggacctgacccccgagcggtccgacgtgtggtttgccctccagctcaaccgcaagctccggctcggggtggggaatcgggccatccgcacggagaagatcatctgccgggacgtggctcggggctatgagaacgtccccatcccctgtgtcaatggtgtggacggggagccctgccccgaggattacaagtacatctcagagaactgcgagacgtccaccatgaacattgaccgcaacattacccacctgcagcactgcacgtgtgtggatgactgctccagctccaactgcctgtgtggccagctcagcatccgctgttggtatgacaaggatgggcggctgctccaggaatttaacaagatcgagcccccgctgatttttgagtgtaaccaggcatgttcctgctggagaaactgcaagaacagagtagtgcagagcggcatcaaggtgcgactgcagctctaccgaacagccaagatgggctggggggtccgtgccctgcagaccattccccagggtaccttcatttgcgagtatgtcggagaactgatctctgatgccgaggctgatgtgagagaggacgattcttatctcttcgacttggacaacaaggatggagaggtgtactgcatcgatgcccgttactacggcaacatcagccgcttcatcaaccacttgtgtgaccccaacatcatccccgtccgagtcttcatgctgcaccaagacctgagatttccacgcatcgccttcttcagttcccgagacatccgggccggggaggagctggggtttgactatggtgaccgcttctgggacatcaaaagcaagtacttcacctgccagtgtggctctgagaagtgcaagcactcggctgaggccattgccctggagcagagccgcctggcccgcctggacccacaccctgagctgctgcctgagctcagctccctgccccccgtcaacccctgagatcggaccacagccactgttccacccctggacagccaccagctcagtagccacttctgccgcttgcccacagcctacgcctgggggcttttaccgtttcctctctcccttacttaccccttcacacattcctcagccagctcccagccaggccctggaggtctgacagcccctgtctcccagagctggttctttcctgggagggtgacttcagggctggccacccctccccccaagcgcctcctcccacccctgttccccaccctcaactgaagtttgatgaaattgagcctctgtgctaattggtttcctttgttctcaataaatgttgggtttataataaa

SEQ ID NO: 40 - DNA, 4924 bp, Bos taurus
  source 1..4924: mol_type = other DNA
atgttggatatttgcttggaaaaacgtgtgggtacgaccttggctgcccccaagtgtagctccagtgctgtgaaggttccagggttggcagaggggactgaagggaccatgaaaatggacatggaggacgcggatatgactctgtggacagaggctgagtttgaggagaagtgtacatacattgtgaatgaccacccctgggattctggtgtggaaggaggcacttcggttcaggcggaggcttccttaccgaggaatctgcttttcaaatatgccacaaacagcaaagagattactggagtggtgagtaaagaatacataccaaagggaacacgttttggacctctaataggtgaaatctacaccagtgatgcagttcccaagaacgccaacaggaaatatttttggcggatctattccagaggggagcttcaccacttcattgatggctttaatgaggagaagagcaactggctacgctacgtgaacccagcgcacactgcccgggagcagaacctggccgcctgtcagaacggcatgaacatctacttctacaccattaagcccatccctgccaaccaggagcttcttgtgtggtactgccgggactttgcagaaaggcttcactacccttattccggagagctgacaatgatgaatctcacacaaacccagagccgtccaaagcagcagagcactgagaaacatgaactgtgcccaaagagtgtcccgaagagagagtatagcgtcaaagagatcctaaaattggactcccacccttccaaagggaaggacttgtaccgctccaacatttcacccctcacttcagagaaggacgcggaagacttcaggaaaaacgggagcccggagatgcccttctacccgcgcgtggtgtacccgattcgcgccccgctcccggaagactttctgaaagcacccctgggatgctacgggatggagcggccgccctacatcactcactcccccatatcgtcctccacgacgcccagcccctcggcgcgcagcagcccggaccggagcctgcagagctgcagcccgcacagcagccccgggaacacggtgtcgccgcgggcccccggcccccaggaggcccgcgactcctacgcctacttgaacgccccctacggggccgccgccgaggggctgggctccctagccggctacgggccgcccacccacctgccgcccgccttcatcccctcctacaacgcgcactacccgaagttcctgctgcccccttacggcatgaactgcaacggcctgggcgccgccgtgagcaacatcaacggccttaacagcttcggcctcttcccgaggctgtacccggtgtacagcagcctcttgggcgggggcggcctgcctcccccggtgctggcgtccgcctcgctccccagctcgctgccctcggagggggcgcgccgcctgctgcagcccgagcacccccgggaggtgctggtgcccgcgccgcacagtgccttctccctccccggggcggccgccagcctgaaggacaaggcctgcacccccaccagcggctccccgacagccgggaccgccgccacggccgagcacgtggtgcagcccaaagctacctcagcggccgctgcggcccccggaggcgaggaggcggtgaatctcatcaagaacaaaaggaacatgactggctacaagacactcccctacccgctgaagaagcagaacggcaagatcaagtatgagtgcaacgtctgcgccaagactttcgggcagctctcaaacctgaaggtccacctcagagtgcacagtggagaacgacctttcaaatgccagacgtgcaacaagggcttcactcagctcgcccatctgcagaaacactacctggtacacacgggagagaagccacatgaatgccaggtttgccacaagcgatttagcagcaccagcaatctcaagacccacctgcgactccactccggagagaaaccttaccagtgcaaggtgtgccccgccaagttcacgcagtttgtgcacctgaaactccacaagcgcctacacacgcgggagcggccccacaagtgcgcccactgccacaagagctacatccatttctgcagcctcaaggtccacctgaaggggcactgtcctgtggccccggccactgggctgcctctggaggacctggccagaatcaacgaagagattgagaagtttgacatcagcgacaatgccgaccggctggaggacatggaggacaacatcgacatgacctctgtggtggaaaacgagattctggccgtggtcagaaaagagaaggaagaaactggcctgaaagcgtctttgcaaagaaacctgggaaatggactgctctcagggtataacctctatgagtcaacagagccgtccctcctgaagttgcctcacagcaacccactacctctgggacctgtaaaggtcaaagaagaaacagtcgaacctatggatccttaagattttcagaaaacaaaaggtgttttgtcttgtttcttaacttacgacttggcgagtcagggtgcctgtcgcaagcgcttgtacataattccaattctgcaaagctctccctgacggcagatggtttccctcacctctctggaattaaagaaggaaccccaaagttactgaaatctcagggcataaatgaggcaaagacaatatatatacctattatatatacttatttacaccccatctatatatttgaacctgttgtattttgaatatttgtgtgaatatgtttgcatagcctccccattgctaagactattgcctagccataattattttttcaatgataatccttcataatttattatacagtttatcattcaaaaagcaataattaaaaaaagtttacaatgactggaagaattccttgtaatttgagttaagtgttatatttttgtctggtggccattctttgtagataatttctgcacatctgttttaagtatcttaagatttaataaacagatacattacttcaagtcaacctccctctataataatggtttgaaagtgaggtttgagtattgccaaagttaggcagttgatgtgttcattcataggattatattatttgaacagccttgtatcatttgaacataatttgggggtatgtttgcagaattaaccagtaataggtggaagaaataaaaaccttgtatgtttctgtagagagttcagcattcctgcccccaatcacagatttaccagaaaggtgataaggcgttgccagtctgtctccctcttcaaaaaaacagggttgtcccatccccaaagtcatgtgaccatttgcagtggccatgagactttagcatcccaatgtattatctgaaaatgtgaagaagacaaatgccatgtttaaaaccactgcaaaatgtttccaaagcacaggtggttttgtatgggtgaggtctgggggcatgagtccaggtgtttttgttgttggtcttttgttattacgtcagaattgcacaagcatggtgctccaccaggaaggatttgagttagataggctcaggccacactttaaaaaccaaaagccagaaaacgggtattctcgttgtcttagggtcaaagcgggtaatgaacattcctgtccccaacacatcagttgtatttgttttttctgtgcaactcaagacttttcctcagtatttgtgtttttacattttatggttaatttaattgaagaagaaagggcattgcaaagttgttcaacaacaattacctcattgagtgtgtccagtagtgcaggaagtgatggcatctttaatgatttgctactcaagaggagagagcacgtgtgcaccagagagatggaatgcatcccattctgcctcgtcctctgctaagcccaaagataacacgttggcattcaaggtgtagcggacaatgatgtatatttataaatctatttataccactataccatgtatatagaatatatttataaccacttaaaattgtgagccaaaacatgtaaaagatccttcttttcctaatggcaaaaaaaaaaaaaaaaaaatcaaaccctctctgactctttgcttaattagcactcgatgacctcacaattacattggtgagcttgggcacccccttggtattataagagatcctaaaattttggtgcaaaggtggggaccgcataacaaccagggagaaagagacataccttttttaactattaaggacaatctaagatggcttttttttttttttttttgtcacattctgatgtggtcacacccaagtcccagaaatagaaatcccatcaaccactgtaacttttctctcttcctctccttccttcttaaataactgatggatcactaatgaatctggtttatatattcagcattttggtctgtggcaactagtatcagcctacagaagagaacaacaaatgtaggataatccagggaaacctaagagccttactgatctgttcctttaaataatgaaactgacagttttttttaatattttttttaacagtgtttgacaaacagtccttgattataagtaaaccaaagcatcatactgacactgacatcaaatattttctattccccatcactcatttgttctcttttttccctctcatgctttctcattagtggaaatttttactgagacctgcacagtggcttgagaccccgtaattctctaccatctgactttccctctgaggcctcttacatgtgaatattgagcccacaatcaagagtggttttgtttttttctttcttctactcaaaactgaccaaagttactggctttttactttgctagaacaaactatcttatgtttacgtactggtttacattgttatttatgtgcaaattgtcaaaatgtaaattaaatataaatgttgatgcttta

SEQ ID NO: 41 - DNA, 4944 bp, Sus scrofa
  source 1..4944: mol_type = other DNA
gggtttctgagaggcgagctgtttggatcattcatgatttgctgccattgtgaacactcttaaggagagaactctgcctgagaaatgctgattaataaaggctgcccccaagtgtagctccagtgccgtgaagtttccagggttggcagtggggaccgaggggaccatgaaaatggacatggaggacgcggatatgactctgtggacagaagctgagtttgaggagaagtgtacatacattgtgaacgaccacccctgggattctggcgtcgatggagggatttcggttcaggcagaggcatccttaccaaggaatctgcttttcaaatatgccaccaacagcaaagaggttattggagtggtgagtaaagaatacataccaaagggaacacgttttggacctctaataggtgaaatctacaccaacgacacagttcccaagaacgccaacaggaaatatttttggcggatctattccagaggggagcttcaccacttcatcgacggctttaatgaggagaagagcaactggctgcgctacgtgaacccagcacactctgcccgggagcagaacctggccgcctgtcagaacggcatgaacatctacttctacaccattaagcccattcctgccaaccaggagcttcttgtgtggtattgtcgggactttgcggaaaggctccactacccttatcctggagagctgacaatgatgaatgacacacaaatccagagccatccaaagcagcagagcaccgagaaacatgaactttgccccaggaacgtcccaaagagagaatacagcgtgaaagaaatcctgaaattggactcccaccctcccaaaggaaaggacttgtaccgctccaacatttcacccctgacttcagaaaagggcccggacgacttcagaaaaaacgggagccccgacatgcccttctacccgcgggtggtgtaccccatccgggcccctctcccggaagactttgtgaaggcgtccctggcctacgggatggagaggcccacctacatcacgcactcccccatcccgtcctccacgccgcccagcccctcggcgcgcagcagccccgaccggagcttgcagagctgcagcccgcacagcagcccagggaacgccgtgtcgccgctggcgcccggcgcccaggagcaccgcgactcctacgcctacttgaacgctccgtacggcgccgctgaaggactgggctcctaccccggctatgcgccagcaccacacctgccgcccgccttcatcccgtcctacaacgcgcactaccccaagttcctgctgccgccctacggcatgaactgcaacagcctgggcgccgtgggcaacatcaacggcctcaacaacttcggcctcttccccaggctgtacccggtgtacggcggcctcctgggagggggcggcctgcctcccccgatgctggcgcccgcctccctccccagctcgctgccgtcggaggggacgcgccggctgctgcagcccgagcacccccgggaagtgctggtgcccgccccgtcgcacagcgccttctccctgcccggggcggccgccagcctgaaggacaaggcctgcagccccaccagcgggtccccgacggccggcaccgcggccacggccgaacacgtggtgcagcccaaagctacctcagcggccgcggccgctcccggcggcggcaacgacgaggccgtgaatctcatcaagaacaaaaggaacatgaccggctacaagacgctcccctacccgctgaagaagcagaacggcaagatcaagtatgagtgcaacgtctgtgccaagacctttggccagctctcaaatctgaaggtccacctccgggtgcacagtggagaacggcctttcaaatgccagacttgcaacaagggtttcacgcagctcgcccacctgcagaaacactacctggtacacacgggagagaagccacatgaatgccaggtttgccacaagcgatttagcagcaccagcaatctcaagacccacctgcgactccactcgggagagaaaccttaccagtgcaaggtgtgccccgccaagttcacccagtttgtgcacctgaaactccacaagcgcctacacacccgggagcggccccacaagtgcgcccactgccacaagagctacatccacctctgcagcctcaaggtccatctgaaggggcactgtcctgcggccccggccactgggctgcctctggaggacctgaccagaatcaatgaagaaatcgagaagtttgacatcagcgacaatgctgaccggctggaggacatggaggacaacatcgatgtgacttcagtggtggaaaaggaaattctggccgtggtcagaaaagagaaagaagaaactggcctgaaagtgtctttgcaaagaaacgtggggaatggactgctctcagggtgtagcctctatgagtcaacagacccgtccctcctgaagttgcctcacagcaacccactacctctaggacctgtaaaggtcaaacaagaaacagtcgaaccaatggatccttaagattttcagaaaacaaaaggtgttttgtcttgtttcttaacttatgacttggcgagtcagggtggctgtaacaaatgcttgtacataattccagttctgcaaagctctcctgacggcacatggtttcccctcacctctctggaattaaagaaggagccccaaagttactgaaatctcagggtataaatgaggcaaagacaatatatatatacatattatatatacttatttacaccccatctatatatttgaacctgtgtattttgaatatttgtgtggatatgtttgcatagccttcccattgctaagactattgcctagccataattattttttcaatgataatccttcataatttattatacaatttatcattcaaaaagcaataattaaaaaaaagtttacaatgactggaagaattccttgtaatttgagttaaatgttgtatttttgtctggtggccattctttgtagataatttctgcacatctgttttaagtacctaagttctagtaaacaaacattacttcagtcaacctccctctataataatggtttggaaatgaagttttggtattgccaaagttaggcagttgatgtgttcattcataggattgtatcatttgaacagcattgtataacttggggggtatgtgtgcagaactatccagtaataacttcagtagaagaaatgaaagagaatcttgtatgtttctgtagatagctcagattccccccccacacaccccagccacagatttaccagaaaggtgacaagaacgctttgccaatctgtctccctccaaaaaaaacaatcagggtcaccccaccccaaagtcatgtgaccattcagagtggccaggaaacttttacatcccagagctttatctgaaaatgtgaagaagacaaaataccacatttaaaaccactgcaaaacattccaaaagcacaggtagttttgtatgtgtgaggtcttggggcatgacatgagtctgggtgttttttttgttggtcttttttgttgttatgtcacaatggcacaaacatggtgctctaccaggaaggattggagttagtcaggctcaggccaccctttttaaaaaaaaaaaaaagccccaaaccggaaaacgggtattctcattgtcttagggtcaaagcgggtaatgaacattcctatccccgacacatcaattgtattttttttctgtacaactcagactttcctcagtatttgtgtttttacattttatggttaacttaattgaagaagaaagggcattgcaaagttgcaacaacaattacctcattgagtgtgtccagtagtggaggaagtgatggcttctctaatttgctaccagaggagaaagctagtgtgcgccagaaagctagaatgcatgccattctgtctcctctgctaagcccaagatgacttgtcggcattcaaggtgtaggggagaacgatgtatatttataaatctatttataccactattccatgtgtatatataatatatttataaccacttcaattgtgagccaaaccatgtaaaagatcctactttttttcctaacggccaaaaaaaaaaaaaaaaaaaaaaaacctttctaagactttgcttaattagcactcaatgacctcgcaatcacattggtgagcctgggcacccccttctcttgtaagagaccctaaatccttggaacaaaggtggagaccgagtaacaagcagggagaaagagagacatcacttttttactattaaaggacaatccaagatggctttatttaattttggggtgggggccacattctgacaatgtggtcaaacccaagtcccagaaataaaaatcatattgtccactgtaacttttccctctggttgtttgtctccctcctttctgagtaactgatcctgcagaagagaatcctgcagattgagggtaatccagggaacaccaagagccttactgatctgttcctttgatgaaactgacaattttgtatgtctcagtgtttgacaaaaacagtccttaatcgcaggtaaaccaaagcatcagagacaccaaatactttctattcccaaccactcatttgttctcttttccctttgagctttctcctacatggggattttttaccgagacctgaccagtggcttgagactgcttgattctctttatcatctgactttccctctgaggcctcttacatgtgaatgttgagcccacaatcagcagtggttttattattttttccttctactcaaagttaaaactgaccaaagttattggctttttactttgctagaacaacaaattatcttatgtttacgtactggtttacattgttatttatgtgcaaattgtcaaaatgtaaattaaatataaatgttcatgcttta

SEQ ID NO: 42 - DNA, 1716 bp, Bos taurus
  source 1..1716: mol_type = other DNA
atggccctgcggccgccgggggaggccggcccccaggacaagggatgctaccctcccgactggctccagggcagcccccaacacctggccgcctactacacgcctttcccgccctacggccactacgggaacaccctgaccgcggcagaggagggcttccagcctttccggcccctggaagccctggcagcgtccccggctctgccccccttcggcttccggaccgcgcctcccttgctaagccagggcctggccctcccgagggagccgctatgcgacctgccctggtacagcaagctgccgacctggtaccctgttccccacctccccagggaggtgccgcacttccagaacagcagtcgtgagtttaccggcgccgccagcgaggacttggcacacaccgccggccggagcgacggtggccagtgttggggacctgagattttcgttccgccgccccccgtggatgcttcgttgttacctgagaggccgaagccctcgcagctgttatcggggtcccccagcgagccctctgaggatggccccaaactcgtgaaccgaaaagggaaggcttctcgccgctaccacttcacccaagaagacctgcacttggttctgtacggggtcatccccagcccgcagcactcagcccgcctgcagcacgcgatctcgggcctcctgatccccaccgatagcgccggggctgattctcttcctcagactctggataacgactcccttcagctaccagaaggtctctgcctccagcagacgaagtttggtgaagtcgcacattttggggtgttctgcagtagtcttgtggccaaaggagtcaggtttgggccctttcagggtaaagtggtcaacaccagcgaagtcaagacacacggagacaattccctgatgtgggagatctttgaagatggtcagttgagccactttattgatggaaaaggaggtgcagggaactggatgtcctatgtcaactgtgcccgtttcccgtcggagcagaacctggtggcagtacaatgtcaagggcagatcttctacgagagctgcaaggagatctaccagaaccaggagctcctcgtgtggtacggagattgctatgagaagtttctggacattcccgtgagcctgcaggttctcaagcaggggaagcaggcatctgggtcctctgaagagtctgcggaagggtacaggtgtgaaagatgtggcaaagtatttacctacaaatattacagagacaagcatctaaagtacacgccttgtgtggacaagggtgacaggaaatttccctgttctttatgcaaacgatcctttgagaagagagaccggctccggatccacatccttcacgttcatgagaagcatcggcctcacaagtgttctacatgcgggaaatgtttctctcagtcttccagtctaaacaaacacatgagagtccactctggagacagaccctaccactgtgtgtattgtactaagaaattcactgcctccagtatactccgcacgcatatcaggcagcactcgggggagaagcccttcaaatgcaagtactgtggtaaatcttttgcatcgcacgctgctcacgacagccatgtccggcgctcgcacagggaggatgacggctctccctgtaacacctgtgggaagaccttctcagatcaagaagccttccactcccacatgaagtctcatgaagactgctag

SEQ ID NO: 43 - DNA, 1915 bp, Sus scrofa
  source 1..1915: mol_type = other DNA
tcgcggtggcccaggatggctctgcggctgccaggtgagaccggtccccaggacaaggagtgctactcgcccgacaggcttcagagcagcccccaacacctggccgcctactacacccctttcccgcactatggccattacgggaacaccctcaccacggcggaggagggtttccaacctttccggcagctggaggccctgtcggcgtccccggctctgccccccttcgccttccggacagcgcctcccttgctgagccccgggctggcgctgccgagggagccgctctatgatgtgccctggtacagcaagctgccgacgtggtacccgatcccccacctgcccagggaggtgccacacttcctgaacagcccgcgcgagttcacgggcgcccccgccgaaaacttgggccaggtcggcggccggagcgatggtggccagtgttggggacctgaagttttccttccgccgcccccagtggatgcttctctgctacccgagaggccgaagccctcccagttgttacctggctcccccggcgagcgctctgaggatggccccaaactcgtgaaccaagagagaaaggcatcgccccgttaccacttcacccaggaagacctgcacttagttctgtacggggtcattcccagccctcagcacccagcccgcttgcaccacgcgatgtcgggcctcctggtccccaccaacagctctgggtctgattctcttcctcagactctggataacagctcccttcagctcccggaaggtctctgcctgcggcagacgatgtttggtgaggtcgcacattttggtgtgttctgcaatagcctcgtggccaaaggagtcaggtttggaccctttcagggtaaagtggtcaatgccagtgaagtcaagacccacggagacaattctctgatgtgggagatttttgaagatggtcatttgagccactttattgatggaaaaggaggtgctgggaactggatgtcctatgtcaactgtgcccgtttccccacggagcagaacctagttgcggtgcaatgtcaagggcagatattctatgaaagctgcaaggagatccaccagaaccaagagctcctggtgtggtatggagattgctacgaaaagtttttggacattcctgtgagcctgcaggtcataaagcaggggaagcaggcatctgggccctcagaagagtctgcagaagggtacagatgtgaaagatgtggaaaagtatttacctacaaatattacagagacaagcatctcaagtacaccccctgtgtggacaagggtgacaggaaatttccctgtaatttgtgcagacgatcctttgagaagagagaccggctccggatccatatccttcacgtccatgagaagcaccgaccccacaagtgttctacgtgtgggaaatgtttctctcaatcttccagcctaaacaagcacatgagagtccactctggagacagaccctaccagtgtgtgtattgtacaaagaagttcactgcctctagtatactccgcacccacatcaggcagcactccggggagaagcccttcaaatgcaagtactgtggtaaatcttttgcgtcccacgccgcgcacgacagccatgtccggcgttcacacaaggaggatgacagctgttcctgtagcatctgtggcaagaccctccccaatcaagacgctttctatgcccacatgaagtttcatgaagactactaactagcctttagagattttgaggatatttcttgtttatgggtgtgtcttgtggggaaaagtgatgcttatattgagatgagacacagagtagtggaactgaccaattagcagaatttgccagagttcttggttgtggtattaacatcctttgcagaaatgttttttgttttttgtttttgtcttt

SEQ ID NO: 44 - DNA, 1788 bp, Sus scrofa
  source 1..1788: mol_type = other DNA
atgaccatgaccctacacaccaaagcgtccggcatggccctgctgcaccagatccaagccaacgagctggagcccctgaaccgcccgcagctcaagatccccctggagcggcccctgggcgaggtgtacgtggacagcagcaagcccgccgtgtataactaccccgagggcgccgcgtacgacttcaacgccgcggccgccgcctccgcgcccgtctacggccagtcgggcctcgcctacggcccggggtcggaggcggcggcgttcggcgccaacggcttggggggcttccagccgctcaacagcgtgtctccgagcccgctggtgctgctgcacccgccgccgcagctctcgcccttcctgcacccccacggccaacaggtgccctattacctggagaatgagccgagcggctatgcggtgcgcgaggccggccctcccgccttctacaggccaaattcagataatcggcgccagggtggcagagagagattggccagcaccagtgacaagggaagcatggccatggaatctgccaaggagactcgctactgtgcagtgtgcaatgactatgcctcaggctaccattatggagtttggtcttgcgagggctgtaaggccttcttcaagagaagtattcaaggacataatgactacatgtgtccagctaccaaccagtgcacaattgataagaacaggaggaagagctgtcaggcctgccggctacgcaagtgctacgaagtgggcatgatgaaaggggggatacggaaagaccggagaggagggagaatgttgaagcacaagcgccagagagatgatggagagggcaggaatgaagcggtgccccctggagacatgagatctgccaacctttggccaagccctctcttgattaaacacactaagaagaacagcccggtcttgtccctgacagccgaccagatgatcagtgccttgttggaggctgagccccccataatctattccgagtatgatcctaccagacccctcagtgaggcttcaatgatgggcttgctgaccaacctcgcagacagggagctggtacacatgatcaactgggcaaagagggtgccaggatttttggatttaagcctccatgatcaagtgcatcttctggaatgtgcctggctagagatcctcatgattggtcttgtctggcgctccatggagcacccagggaagctcctgtttgctcctaacttgctcctggacaggaaccagggcaagtgtgtcgagggaatggtggagatctttgacatgttgctggctacatcatctcgcttccgtatgatgaatctccagggagaggagtttgtgtgcctcaaatccatcattttgcttaattctggagtgtacacgtttctgtccagcaccctgaagtctctggaagagaaggaccatatccaccgtgtcctggacaagatcacagacaccttgatccacctgatggccaaagcgggcctgactctgcagcagcagcaccggcgtctcgcgcagctcctcctcatcctgtctcacttcaggcacatgagtaacaaaggcatggagcatctgtacaacatgaagtgcaagaacgtggtgcccctctatgacctgctgctggagatgctggacgcccaccgcctgcacgccccaaccaaccttgggggcccacccccggaggacatgagccagagccagctggccacctcgggctcaactccatcgcattccttgcaaatgtattacatcacaggggaggcggagaacttccccaccacaatctga

SEQ ID NO: 45 - DNA, 4092 bp, Bos taurus
  source 1..4092: mol_type = other DNA
atggatgcaaaagctcgaaattgtttgcttcaacatagagaagccttggaaagggacatcaaaacatcctacattatggatcacatgattagtaatggagttttaacagtgtcagaggaggaaaaagtgaaaaatgagcccacccaacggcaacgagcagctatgctaattaaaatgatacttaaaaaagataatcattcctatatatcgttctacaatgctctacttcatgaaggttacaaagatcttgctgcccttctgcatggtggcattcctgttatctcttcttccgatgtaaggacagtcctgtgtgaaggtggagtaccacagaggcccgttgtttttgttactaggaagaagctagtgaatgcaattcagcagaagctctccaaactgaatggtgaaccaggatgggtcaccatatatgggatggcaggttgtgggaagtctgtattagctgcagaagctgttcgagatcattctttcctagaagattgtttcccaggaggcgtgcattgggtgtcagttgggaaacaggacaaatccgggcttctgatgaaactacagaacctttgtgcacgattggatcaggatgagagcttctcccagaggcttccacttaatattgaagaggctaaagaccgtctccgcattctgatgctgcgcaaacatccaaggtctttattgatcttggatgatatttgggatccttgggtgttaaaagcttttgacaatcagtgtcaaattcttctcacaactagagataagagtgttacagattcagtgatgggtcctaaatatgtagtgcctgtggagagtggtttaggaaaagaaaaaggacttgaaattttatccctttttgttaatatgaagaaagcagatttgccagaacaagctcacagtattatacaagaatgtaaaggttcccctcttgtagtatctttaattggtgcacttttacgagattttcccaaccgctgggagtactacctcaggcaacttcagaataagcaatttaagaggataagaaaatcttcatcttatgactatgaggctctagatgaagccatgtctataagtgttgaaatgctcagagaagacatcaaagattattacacagatctttccatcttacagaaagatgttaaggtgcctacaaaggtgttatgtatcctctgggacttggaaacggaagaagttgaagacatattacaggaatttgttaataagtctctcttgttctgtgaccggaatggaaaatcatttctttattatttacatgatctgcaagtagatttccttgctgaaaagaatcgcagccagcttcaggatctccacaagaagatggtcactcagttccagagacatcaccagctatattctctttcaccagatcaggaggattgcatgtattggtacagttttctggcctatcacatggccagtgctaatatgcacaaagaactgtgtgctttaatgttttccctggattggattaaagcaaaaacggaacttgtaggccctgctcatctgattcatgaatttgtggaatacaggcatatattggatgaaaaggatggtatagactgtgagaatttccaggagtttttatctttaaatggacatcttcttggacgacagccatttcctaatattgtgcaactgggcctctgtgaactggaaacttcagaggtttatcggcaagctaagctgcaggccaagcaggaggtcgataacggaatgctttacctggagtgggtaaacaaaaaaaacatgaagaacctctcccgcttggttgtccgcccccatacagatgctgtttaccatgcttgcttttctgaagatggtcaaagaatagcgtcttgtggagctgataaaactttacaggtattcaaagctgaaacaggagagaaacttctagaaatcaaggctcatgaggatgaagtgctttgctgtgcattctctgcagatgacaaatttatagcaacctgctcagtggataaaaaagtgaagatttggaattcaatgactggggaactagtgcgtatctatgatgagcactcagagcaagtcaattgctgccacttcaccaacaatagtaatcaccttctcttagccactggttcaaatgactacttccttaaactctgggatctgaatcaagaagaatgtcgaaataccatgtttggccatacaaattcggtcagtcactgcagattttcaccagatgatcaggttttggccagttgttcagctgatggaacattaaagctttgggatgtgaaatctgcaaatgagaggaaaagcattaatgtgaagcaattcttcctgaattcagaggagcctcaggaggatctggaagtgatagtgaaatgctgttcgtggtctgctgatggtgctaagataatggtggctgcgaaaaataaagtctttctttttgacattcactccagtggcctgttggcagaaatgcacatgggccatcatagcgctgtccagtactgtgacttctccccccacaaccatttggcagtggtggctttgtcccagtactgtgtggagttgtggagtatggactcaggtttaaaagtagctgattgcagaggacatttaagttgggtccattgtgtgatgttttctcctgatggatcatcgtttttgacatcttctgatgaccagacaatcaggctctgggagacaaagaaagtatgtaagaactctgctacagtgttaaagcaagaaatagatgttgtgtttcaagaaaatgaagtgacggttcttgcagttgacaatataagacgtctgcaactcattaatggaaacacaggtcagattgattacctaactgaagctcaagtgagttgctgttgtttaagtccacatcttcagtaccttgcattcggaggtgaagatggagccattcagattttagaactcctgaacaacagaatcttccaatccagaaatgggcacaagaccactgttcggcacatccagttcacagctgatggaaagactcttatctcaagctctgatgattctgcagttcagatatggaactggcaatcagaggaccctttctttctccaagcccaccaggaaacagtgaaagacttcagactcttgaaaaattcaagacttctttcctggtcatttgatggaacagtaaaggtatggaatattattaccagaagaatagaaaaagatttcgtgtgtcaccaggatacagttctttcttgtgatatttcctctgatgctactaagttttcttctacctctgctgacaagactgcaaagatttggagttttgaactcctttctcctcttcatgaattgagaggccacaaaggctgtgtgcgctgctcttccttctctgtgaacaataccttgttggcgactggagacgacaatggagaaatcaggatatggaatgtctcaaacggtgagcttcttcatttgtgtgctccgatttcagtagaagaaggagctgctactcatggaggctgggtgaccgacctttgcttttctccagacaccaaaatgcttgtttctgctggaggatatctcaagtggtggaatgttgtcactggggaatcctcacagaccttctacacaaacggaaccaatcttaagaaaatacacgtgtcccctgacttcaaaacatatgtgactgttgataatcttggtattttatatattctaaagattttagagtaaaatagttaagtggtgatgtaagttgacctcttttaattttgaattggaaaaaaatcctaatgaaactctgtactttaacttttataaagctgtgaattgttttgtagtattgcattcattacaaaagtgtttgtggttggatgaataatattaatgtaatttttcccaaatggacatgcctttaatcttgtttttcataatcaccatcattttttaacagttcgtccttaagatgcaaatgaaaatgtaaatatgtacctagttataccattggtaaaattctcccttgctacattcaaattggttgacataataatgaaaataattggaaggaaatatatatattttaatactatcattggtgcaggccatgcc

SEQ ID NO: 46 - DNA, 5998 bp, Sus scrofa
  source 1..5998: mol_type = other DNA
ctttcagacttggcaccgcccagaagccagccccctccctcgcccggtcatccccgcggttccactgcgactcctatccacctgcctttctagttcctccagctcatggttgacagctcagagaaagaaaaatttgaggaaggatggatgcaaaagctcgaaattgtttgcttcaacatagagaagccctggaaagggacatcaaaacatcctacatcatggatcacatgattagtaatggcgttttaacactttcagaggaggaaaaagtgaaaaatgagcctactcaatgccaacgagcagctctgctaattaaaatgatacttaaaaaagataattatgcctacatatcattctacaatgctctgctacatgaagggtataaagatcttgctgcccttctccatggtggccttcctgtcgtctcttcttccaacagcaaagattcagttgctggaataacttcctatgtaaggacagtcctgtgtgaagggggagtgccacagaggcccgtcgtctttgttatgaggaagaagctcgtgaatgcagttcagcagaagctcttcaaactgaatggtgaaccgggatgggtcaccatatatggaatggcgggttgtgggaagtctgtgttagctgcagaggctgttcgagatcattctctcttagaagtttgtttcccagggggtgtgcattgggtttcaatagggaaacaagacaaatctgggcttctgatgaaactgcagaatctttgtgcacggttggatcaggatgagagtttctcccagaggcttccacttaatattgaagaggctaaagaccgtctccgaattctgatgctgcgcaaacacccaaggtctctgttgatcttggatgatatttgggatccttgggtattaaaagcttttgacaatcagtgtcagattcttcttacaactagagacaagagtgttacagattcagtaatgggtcctaaatatgtaatacctgtggagagtggcttaggaaaagaaaaaggacttgaaattttatccctttttgttaatatgaagaaagcagatttgccagaacaagctcacagtattataaaagaatgtaaaggttctcctcttgtagtgtctttaattggtgcacttttacgtgattttcccaaccgctgggattactacctcaggcaacttcagaataagcaatttaagaggataaagaaatcttcatcttatgattatgaagctctggatgaagccatgtcgataagtgttgaaatgctccgagaagacatcaaagattattacacagatctttccatccttcagaaagatgttaaggtgcctacaaaggtgttatgtattctctgggacatggaaaccgaggaagtcgaagacatactgcaggagtttgttaataagtctctcttattctgtgatcggaatggaaagtcatttctttattatttacatgaccttcaagtagatttccttacagagaagaatcgcagccagcttcaggatctgcacaagaagatgatcactcagttccagagacatcaccggctacacactctttcaccagatcaggaggactgcatgtattggtacaattttctggcttatcacatggccagtgctaatatgcacaaagaactgtgtgctttaatgttctccctggattggattaaagcaaaaacggaacttgtaggccctgctcatctgattcatgagtttgtggaatataggcatattttggatgaaaaggatggcacagtctgtgagaatttccaggagtttttatctttaaatggacatcttcttggacgacagccgtttcctaatattgtgcaactgggtctctgtgaactggaaacttcagaagtgtatcggcaagctaagctgcaggccaagcaggaggtcgataacggaatgctttacctggagtggataaacaaaaaaaacatcaagaacctctcccgcttagttgtccgcccccatacagatgctgtttaccatgcttgcttttctgaggatggtcaaagaatagcttcttgtggagctgataaaaccttacaggtgttcaaagctgaaacaggagaaaaacttctagaaatcaaggctcacgaggatgaagtgctttgctgtgcattctctgcagatgacagatttatagcaacttgctcagtggataaaaaagtgaagatttggaattctgtgaccggggaactggtacaaagctatgatgagcactcagagcaagtcaactgctgccacttcaccaacaacagtaatcaccttctcttagccactggctcaagtgactacttcctcaaactctgggatctgaatcaaaaagaatgtcgaaataccatgtttggtcatacaaattcagtcagtcactgccgattttcaccagatgataagcttttggctagttgttcagctgatggaacattaaagctttgggatgtgaagtcagcaaatgagaggaaaagcattaatgtgaagcagttcttcctaaattcagaggagtctcaagaggatatggaagtgatggtgaaatgctgttcgtggtcagctgacggtgccagcttagtggtggcagcaaaaaataaagtcctgctttttaacattcatacaggtggcctgttggcagaaatccacacaggccatcacagcaccatccagtactgtgacttctcccccagtaaccatttggcagtagtggctttgtcccagtactgtgtggagttgtggaatatggattcatgtttaaaagtagctgactgcagaggacatttaagttgggttcattgtgcaatgttttctcctgatggatcatcgtttttgacatcttctgatgacgagacaatcaggctctgggagacaaagaaagtgtgtaagaactctgcgatagtgttaaaacaagaaatagatgttgtgtttcaagaaaatgaagtgatggttcttgcagttgacaacataagacgtctgcaagtcattaatggaaaaacaggtcagattgattatctgacggaagcccaagtgagttgctgctgcttaagtccacatcttcagtaccttgcgtttggaagcgatgatggagccattcaggttttagaactcctaaacaacagactcttccagtccaggactaggcacaagaacactgtacggcatatccagttcactactgatggaaagaatgttatctcaagctctgatgattctgcaattcaggtgtggaattggcagtcagaggagactgtctctctgcaagcccatcaggaaacagtgaaagactttagactcttgaaaaattcgagactgctttcctggtcatttgatggaacagtaaaggtatggaatattataactggaagaatagaaaaggactttgtctgtcaccaggatacagtactttcttgtgatatttctcctgatgctaccaaattttcttctacctctgctgacaaaactgcaaagatttggagttttgacctcctttcccctcttcatgaattgagaggccacaaaggctgcgtgcgctgctctgccttctccgtggacagtaccctgttggcgactggagatgacaacggagaaatcaggatatggaatgtctcaaacggtgagcttcttcattcgtgtgctccagtcttagtagaaggaggagctgctacccacggaggctgggtgaccgaccttcgcttttctccagatggcaaaatgcttgtttctgctggagggtatcttaagtggtggaatgttgactctgggaaatccatacagacgttctacacaaatggaaccaatcttaagaaaatacatgtgtcccctgacttcaaaacatatgtgactgtggataatcttggtattctgtatgttttaaagattttagagtaaaatagttgagcagtgatgcaagttgaactcttaattttgaattggaaaaaatcgtaatgaaactctgtatttcaactttttttataaagctgtgaattgttttgcagtattgcattcattacaaaagtatttgtggttggatgaataatattaatgtagcttttcccaaatgaacacacttttaatcttgtttttcataatcatcatcattatttttaacagtttgtcctgaagatgcaaatggaaatgtaaatatgtacctcgttatactgttggtaaaattctcccttgatgcactcaaattggttgacataattaataagaataatttgaaggaaatgtgtattttaatattatcattatgcaggctgtgctacagggtagtagtggcttggctttatgaaccacacttatcccaagagggtatagttcccccaaatatcatctcagaccttatttgtacccctgtaatttgcttgaaaaatattgtgtctgtgtacacaatgtaaaaaatgtcctttaatgtacttgataagtgagtcttagattccaaaagatctttctgtgagtcaaaatttatgcacgttggagagagtagattttgttggtctaccctttctcatccactgatggcagcccgggctggacctggactcaagctgtcagtctttaagttggtccgtgtcctgctctctgcttttttatataccagacttggtatacttgactgaccttgacctttcttaatcctcacttcagtacttgctccacagctaactgttagtaaatcagagtaatcagggaagagccaattttaatcataagttgaaacaacagaatttttctaagatagggaaaatagtaaaaaataatttttgtctaattccatttacttgtagatgaatagcattgctaatgctgttagtgaatttcaagagaattctatttggttttctgagcaatgctggaagagtcatgtgactctggaagatgtcacctttaaccttccagcctgtcgctttctatccccttctctccctccgcatttccttcctttccctccctccttcattctaagcttcatcaggaaaaataatttttgataaggaagaggcgtatcaatatcagtaactagctttttttacctcctggtcaggtacttacattgcagcttaactctaaacatttcccctgaagtatactgttaagggaaatgtgataaatataaaattaataaaattatttatgtaaaagacaaccaaaattcagaagtttttgctgctgcaagagcgaagtcagattagtgttactagtaggtaagcattgagctagtttctgttgttgatacatgtgaggtgggtacggggaatagaccactccagatgtgctttcagatgttcctctttagtatttgagggaaccttaagagtgaataaagtctttcacattgttaaggaggcttgttttttgttttgtcgcatagtgggttgaggagctcatttataggttcactgtgacttaagcaaatgtgatgtaaaaaatcttacttttataaaagaatttctataaatagtatgttctacttagagtactcaaatcataatttgattggtttacaatgtttaggaactcatagctcagagtataaagtattaacttttagaattaaatgtcacttgactgtaaaagtatgacaacaaacatcagcttttaaaaagccttgattggcccttttatcttaccaataaattaaaaagaaaaaatgagaagagagactgggtaacatgaggaaagaagagaagcctttcatggcagtgtaagtggccagtcaagggggacggttttgttttgttctttggggtgagtgtttttcaaggctttccccctgctttattttttaattttttttttgtattgaacatacactgcattttgtgtgtataaaggtttttttgttttttgtattggatatgtgttatgatgttttggaaacagttttgtatttatgatacagcttacatttttttttttttgccttggtatacacttaaatacatgaattaaaaaattttataaaaataaaattcctaaaatatttttgaaaa

SEQ ID NO: 47 - DNA, 5454 bp, Sus scrofa
  source 1..5454: mol_type = other DNA
gccagtgtccactgccagcgaggggcggagccagaacgcaacccacagtcctcagttcgttcagcacctccccagccccagcaaggagcagcctccatttttgacgaatctgagccaccccagggtgatgcttctaaagcagaaagaggacaccgaactgcattgtggcgtgtttggtttctagcagcgatttacctttccaagtcagtagtgaaaatgcacattaagtcaatcattcttgagggattcaagtcctatgcacagaggactgaagtcaatggttttgaccccctcttcaatgctatcactggtttaaacggtagtgggaaatccaacattttggactccatctgctttttgctgggcatctccaatttgtctcaggttcgggcttctaatttacaagatttagtttacaaaaatggacaggctggtattaccaaagcctcagtgtcaatcacctttgataattctgacaaaaagcaaagtcctttaggatttgaggttcatgatgaaattacagtaacaaggcaggtggttattggtggcagaaataaatatttaatcaatggagtaaatgcaaataataccagagtacaggatctcttctgttctgttggccttaacgttaacaaccctcactttctcatcatgcagggccgaattacaaaggtgttgaatatgaaacctccagagattttgtccatgatagaagaagcagctggaactaggatgtatgaatacaaaaaaatagctgcccagaagacaatagagaaaaaggaggctaagctaaaagaaattaagacgattcttgaagaagaaattactccaaccattcaaaaattaaaagaggaaagatcttcctatttggagtaccaaaaagtaatgagagaaatagaacatttgagtcatttatacattgcttatcagtttttgctggctgaagataccaaagaacgctcagctgaggagttaaaagaaatgcaagataaaattctaaagcttcaagaagaactgtctgagaatgataaaaaaataaaagcacttagtcatgaaatagaagagttggaaaaaagaaaagataaggaaactggaggtatacttcgatctttagaagacgctcttgcagaagctcagcgaattaatactaaatctcaaagtgcctttgatctcaagaagaagaatctggcaagtgaagagaacaaacgcaaagagctggaaaaaaatatggttgaagattctaaaacgttagcagccaaggaaaaagaggttaaaaagataacagatggactgaatgctcttcaagaagcaagcaataaagatgctgaagctttggctgccgcacaacagcacttcaatgctgtttctgctggcctgtccagtaatgaagatggagcagaagcaactcttgctggtcaaatgatggcctgtaaaaatgatataagtaaagctcagacagaagccaaacaggctcaaatgaagttgaaacatgcccagcaagaattaaagactaaacaaaccgaagttaagaagatggatagtggctacaggaaagatcaagaagcattagaagctgtgaagaaacttaaagaaaaacttgaagctgaaatgaaaaagctaaattatgaagaaaacaaagaggaaagccttctggaaaagcgcaggcagctgtctcgtgatatcagtagattgaaagaaacatatgaagctctcttggccagatttcccaatcttcgatttgcctatagggatccagagaagaactggaatagaaattgtgtgaaaggacttgtagcttcactgattagtgtgaaagatatttctgcaaccacagctttagaattggtggctggggacagactctataatgttgtagtcgacacggaggttactggaaaaaagctactagaaaagggagagttgaagcgtcgatatactataattccactcaataaaatttcagccagatgtattgctccagaaactctgaaaattgctcagactcttgttggccctaataatgttcatgtggctctttctctggttgagtataaaccagaacttcagaaagcaatggaatttgtatttggaacaacatttgtttgtgacaatatggataatgcaaaaaaaatagcttttgataaaagaataatgactagaactgtaactctgggaggtgatgtatttgatcctcatggaacattgagtggaggtgctcgatcccaggcagcttctattttaacgaagtttcaagaactcaaagatgttcaagatgagctgagaatcaaggagaatgagctacaggctctagaagaggaattggcacatcttaaaaacactgctgaaaagtatcgccaactaaaacagcagtgggagatgaaaactgaggaggcagatttattacaaaccaagcttcagcaaagctcctatcataagcaacaagaagaattagatgctcttaaaaaaatcattgaggaaagtgaggagaccttaaaaaacaccaaagaaattcaaaaaaaagcagaagaaaaatatgaagtattggaaaataaaatgaaaaacgcagaagctgaaagagagagagagctaaaggatgctcagagaaaactagatagtgccaaaactaaagcagatgcttctagcaagaaaatgaaagagaaacagcaggaagttgaagctattaccctggagctggaagagctcaagagagagcatgcatcctgtgaacaacagcttgaagctgtaaatgaagctatcaaatcctatgaaggccagattgcagtagtggcagcagaggtggctaaaaataaggagtcagtaaataaagcccaagaagaggtgaccaagcaaaaagaagtaataacagcccaagacaacatagttaaagctaaatatgcagaagtagcaaaacataaggagcaaaacaatgaatatcagcttaaaattaaggaactggatcacaacatcaacaaacataaacgggaagctgaagatgctgctgcaaaggtgtccaaaatgttgaaagattatgactggattaatacagacaaacacctctttgggcaacccaatactgcctatgatttcaaaactaccaacccaaaagaagctagtcagaggcttcagaagttggaagaaatgaaggaaaaactaggaagaaatgttaacatgagagctatgaatgtactgacagaagctgaagagcggtataatgatttgatgaagaagaagagaattgtagaaaatgacaaatctaaaatcctggcaactatagaagatcttgaccagaagaaaaaccaagccctaaatattgcttggcaaaaggtgaacaaggactttggatctattttttctactcttttgcctggtgctaatgctatgcttgcaccaccagaaggacaaactgtattggatggtctggagtttaaggttgccttaggaaatacttggaaagaaaacttgactgaacttagtggtggtcagaggtctttagtggctttgtcattaatactgtccatgcttctcttcaaacctgctccaatttatatcctggatgaggtagatgcagccttagatctttcccatacccagaatattggacagatgctacgtactcatttcacacattctcagttcattgtggtgtccctaaaggaaggtatgttcaacaatgccaacgtccttttcaaaaccaagtttgtggatggtgtttctacagtagccagatttactcagtgtcaaaatggaaaagttccaaaggaaacaaaatctaagacaaaaggacccaaatgatcacatatggaagttaaaagtacagatttattccattccttcatcttgacctgttttttaaacataaacttttttaagaatttggggttcattattttgttacttaaccacttatttaatcacttataaatgagcctaaattcctcgtctgaattagtgttaactatggtccaattactgtgatagtgattttctgtatgttatcaactgttttttcttacacaagtcttttatatatcagggagcctgagacatatagttggtggtaaattttgtatgtaaaagcaaagtataaaaagatgaaattaaattataggataaatatagctgatagtcagcttttaataattagttaattcctaaggtaatattactgtgttttctattactaataatatcattcgggtaaggaagaattataaagcaagcaagtaggtggaccaacaaagggaacacaaagaaaactattagacatgcactaagctttttgtgatttagttcttcgaggacataatgccagatccctgatttatactcacatgagaaacagatcttgaaaggtttctcttgttttaatgatgtttgaatatcatgtttctatatatttagtaattggtctgtgcaatcttaaattttttaaaaagtctatgaatatttaataaatgcttgggagaaggtatgttgcatgttataagtttatccaaagtctcctaatatttttttttaaagatttttgtcagccttttcataatccagtcaataacaatctattggtgagtgaaaatgtaggagccagcagaccaaaccaaattatctttttcctttagttctgatatatatgtcatatgtatatatatatatatatatatatagatccatttgttctatcaaaagggctcagaattcattgattattatatatgttagtgtccctgctttccattgcaaaaacatgaatttctgttaagaactcaaaagctaggcctaacaaacaccataagaatactatatttggccccttagtaccttagtatctcttagtgtgtcactagaaccatgtctccaaactctaaatcttgaaagttctgacatctaatctgaccttggatgtgttattttggctccacgttgtttcttagttcctttgtggtatccttttcttaacatatgctcatattccctggccctaggtttattttttctcatccttcatcctcatgcatatgtgaggatgtcaggtaactgatactctggggcacattgtaaaggattgaatgtggaattagttcagtctaccgtaagaggaggagacaaagctgtaaatgctgttatataggctgtccattatcacatgcaatagatgtaagtttatttcttttcaagtatattgcagattgtttgtcaaaggaattaatcagttcacactgtcaccaatatagtatgttgaccacaacctacacagcactggctgttatttgtaatgcttctgatttactagttataaaacagtatttaattgctatttttaattatcattttataagtaacttgtgggattgaatattttgccatattattaattttctttcctcagccaacaattctgtttgctttcctaatttttgttacggaaatcctcaaacattaaatttttgtgagtaa

SEQ ID NO: 48 - DNA, 4944 bp, Bos taurus
  source 1..4944: mol_type = other DNA
gaagttcgttcggcgccgccccagccgcaacaaggtgcagcttccatttagaggaccctggcccagcccagagggattctgaggcagaacgaggagaccgacacgaagccatcctgcttccggtacttggagaccacagaggcctgccccactgaatcccgccgggcctgagaggaggcggggcttcccgggcgcgatgacggccagacccggcctgcggcagggcggggccggcgagcgcgaggcccccaggctgaggtgcggggctgcccgaggtgtgccggataccgagctcaacaattgccgccagcttctttagtcctcagaactgctttgtggcttgatttctagcagacctgtccaagtcagtagtgaaaatgcacattaagtcagtcattcttgagggattcaagtcctatgctcagaggactgaagtcaatggctttgaccctctgttcaatgctatcactggtttaaatggtagtggaaaatccaacatattggactccatctgctttttgctgggcatctccaacctgtctcaggttcgggcttctaacttacaagatttagtttacaaaaacgggcaagctggtattacaaaagccttggtgtcaatcacctttgataattctgacaaaaagcagagtcctttaggatttgaggttcatgatgaaattacagtaacaaggcaggtagttattggtggcagaaataaatatttaatcaatggagtaaatgcaaacaataccagagtatacgatctcttctgttctgttggccttaatgttaacaaccctcactttctcatcatgcagggccgaattaccaaagtgatgaatatgaaacctccagagattttgtccatgatagaagaagcagctggaaccaggatgtatgaatacaaaaaacaaaatgctcagaggactatagaaaaaaaggaggctaagctgagagaaattaagacgatacttgaagaagaaattactccaaccattcaaaaattaaaagaggaaagatcttcctacttggagtaccaaaagctcttgagagaaatagaacatctgagtcgtttatatatagcttatcagtttttgctggctgaagatactaaagaacgctcagctgaggcattaaaagaaatgcaggataaaattaagaagcttcaagaagaagtgtatgagaatgataaaaaaataaaagcacttagtcatgaaatagaagagttggaaaaaagaaaagataaggaagttggaggtatacttcgatctttagaagatgctcttacagaagctcagcgagttaatactaaatctcaaagtgcctttgatttcaagaagaaaaatctggcgagtgaagagaacaaacgcaaagaactggagaaaaatatggtagaggattctaaaactttagcagcaaaggaaaaagaggttaaaaaaataacagatggactaagtgcccttcaagaagcaagtaataaagatgctgaagctttggccgttgcacagcagcactttaatgctgtttccgctggtctgtccagtaatgaagatggagcagaagcaactcttgctggtcagatgatggcctgtaaaaatgaaataagtaaaactcagacagaagccaaacaggctcagatgaagttgaaacatgcccaacaagaattaaagactaaacaaacagaaattaagaagatggatagtggctataggaaggatcaagaagcattagaagctgtgaaaaaacttaaagaaaaacttgaaactgaaatgaaaaggctaaattatgaagaaaacaaggaggaaagccttctggaaaggcgaaggcagctatctcgtgatatcagtagactgaaagaaacagaagaagctctcttggccagatttcccaatcttcgatttgcctatagggatccagagaagaactggaatagaaattgtgtgaaaggacttgtagcttcactgatcagtgtaaaagatagttctgcaaccactgctttagaattagtggccggagaccgactctacaatgttgtagtagacacagaggttactggaaaaaagctactagaaaagggggaattgaagcgtcgatacactataattccactcaataaaatttcatccagatgtattgctccagaaactctgaggattgctcagaatcttgttggccctaataatgttcatgtggctctttccctggttgaatacaaaccagaacttcagaaagcaatggaatttgtctttggaatgacatttgtttgtgacaatatggataatgcaaaaaaagtagcttttgataaaaggataatgactagaactgtaactctaggtggcgatgtgtttgatcctcatgggacgttgagtggaggtgctcgatcccaggcagcttccattttaatgaagtttcaagaactcaaagatgttcaagatgagctcagaaccaaggagaatgagctacgggctctagaagaggaattagccaatcttaaaaacactgctgaaaagtatcgccacctaaaacagcagtgggagatgaaagctgaggaggtagatttattacaaaccaagcttcagcaaagctcgtatcacaagcaacaggaagaattagatgctcttaaaaaaatcattgaggaaagtgaggagaccttaaaaaacaccaaagaaattcagaaaaaagccgaagaaaaatatgaggtgttggagaataaaatgaaaaatgctgaagctgaaagagagagagaactgaaggatgcacagaaaaaactagattgtgccaaaactaaagcagatgcttctagcaaaaaaatgaaagaaaaacaacaggaagttgaagctatcaccctggagctggaggagctcaggagagagcatgcatcctatgaacaacagcttgaagctgtaaaggaagctatcatatcctacgagggtcagattgcagtgatggcctctgaggtggctaaaaatgaggagttagtaaataaagctcaagaagaggtaagcaagcaaaaagaagtgataacagcccaagacaacataattaaagctaaatatgcagaaatagcacaacacaaggagcaaaacaatgaatctcagcttaaaattaaggaactggaccataacatcagcaaacataagcgggaagctgaagatgctgctgcaaaggtatccaaaatgttgaaagactatgactggattactgtagaaaaacacctctttgggcaacccaatagcacctatgatttcaaagctaacaatccaaaagaagctggtgagagacttcagaagttgcaagaaatgaaggagaaactaggaagaaatgtcaacatgagagccatgaatgtactgactgaagctgaagagcgttataatgacttgatgaagaagaagagaattgtggaaaacgacaaatccaaaatcctggcaactatagaagaccttgaccagaagaaaaaccaagccataaatattgcttggcaaaaggtgaacaaggactttggatctattttttctactcttttgcctggtgctaatgctatgcttgcaccaccagaagggcagactgtgttggatggtctggagtttaaggttgccttaggaaatacttggaaagaaaacctaactgaacttagtggtggtcagaggtctttagtggctctgtcattaatcctgtccatgctccttttcaaacctgccccaatctacatcctggatgaggtcgatgcagccctggatctttctcatactcagaatattggacatatgctacgtactcatttcacacattctcagttcattgtggtatccctaaaagaaggtatgttcaacaacgcaaacgtccttttcaaaaccaagtttgtggatggtgtttctacagtagccagatttactcagtgtcaaaacggaaaagttccaaaggaaaacgaatccaagtcaaaaagacccaaatgatcacacatgaagttaaaagtaaagattcactccttcaccttgacctgttttttaaatatacactttataagaacttgggattcatgttgtttgtataaaaatgtaatattgttacctaacccattttctcttccttatataatcacttataactgagcataaattcctcttttctgaatcagtgttaactatggtccaattactgtaattgtgatttcatgtatgtcaccaagtgttttttcttacacaagtcttttctatattagggagcctgagatacatagttggtggtaaattttgtatgtaagagcaaaattaccaagaagcagatgaaattaaattacagaataaatatagctgataatcagcttttaatagctaattcctaaggtaatattagtgttttctgttgctaataatgtcgttcaggtaaggaagaatcaagtagcaagtagctggaccaacagagggaacacaaagaaagctgttaggcatgccctgagctatttgtgtttagttccttgaggacagtgccaggtctctgatttagactcaaatagaaagcagatcttaagaagattcctgttattttaatgatgtttgaataccatattatgcttagtaattggtctgtgcagtcttcttttttttttttgatctttatgagtattttaataagagcttaggacaaaggtatcctctgcgtgctgctctctgctgcaggtttatctgaagtctcctaatatggtcagagattttgttagccttttcataattcaatcaataacagtctgttggtgagtgagaatataggagccagcagactaaaccaaattaatctttttcttttagtcatatacatatgtaaatagattcatctgttttaccaaaaggacttaaaatttcatgactatttcttagtgtcctgttttccaattcaaaaccatgaatttccttttaagatttcaaaagatggcgctaacaatcagca

SEQ ID NO: 49 - DNA, 3788 bp, Bos taurus
  source 1..3788: mol_type = other DNA
cgctcggcgaaggtctacgcgccatccccaaggtcccccctggatcgccggctttcatcttcctgctgaattcctgctttgcagatagcgcaatggcagcccgagtacttgtcattggcaatggaggacgagaacacacgctggcctggaaacttgcacagtctactcatgtcaaacaagtgttggttaccccaggaaatgcaggcactgcctgctctgagaagatttcaaatactgacatctcaatcagtgatcatactgcccttgcgcagttctgcaaagatgagaaaattgaattcgtagttgttggaccagaggcacctctagctgctggaattgttgggaacctgaactctgtgggagttcggtgctttggccccacagcacaagcagctcagttagagtccagcaagagatttgccaaagagtttatggaccgacatggaatctcaactgcaagatggagagctttcactaaacctaaagaagcctgtgacttcattatgagtgcagacttccctgctttggttgtgaaggccagtggtcttgcagctgggaaaggggtaattgttgccaagagcaaagaggaggcctgtgaagctgtcagagagattatgcagggtaaagctttcggagaagcaggagaaacagttgtcattgaagaacttcttgaaggagaagaggtgtcttgtctgtgcttcactgatggacggactgtggcccccatgcccccagcacaggaccataagcgattgctggagggcgatgagggccccaacacgggaggaatgggagcctattgtccagcccctcaggtttctaaggatttgttgctaaaaattaaaaataatattcttcagaggacagtggatggcatgcaggaggagggtatgccatacacaggtgtcctctatgctggtataatgctgaccaagaacggccccaaagttctggaatttaattgccgtttcggtgatccagagtgccaagtgatcctcccacttcttaaaagtgatctttatgaagtgattcagtctatcttagatggcttgctctgcacttctctgcctgtttggctggacaactgtgctgccgtaactgtggtcatggcaagtaaaggttatccaggagactacaccaagggtgtagagataacagggtttcctgaggctcaggctttaggactggaggtgttccaagcaggcactgccctaaaagatggcaaggtggtgactaacgggggtagggtccttactgtcacggccatcagggaaaatctcatctcagctcttgaagaagccaggaaaggacttgccgccataaagtttgagggagctgtttataggaaagacatcggctttcgagccatagctttcctccagcagcccaggggcctgacttacaaggaatctggggtagacattgcagctggtaatatgctggtccagaaaattaaacctttagcgaaggccacctccagaccaggctgtgatgttgatcttggaggttttgctggtctttttgatttgaaagcagctggtttcactgatcctcttctggcttgtggaacagatggtgttgggacgaaactgaagattgcccagcagtgcagtaaacatgacaccattggtcaggatttggttgcgatgtgtgtaaatgacattctggcacaaggagcagagcccctctttttcctcgattacttttcctgtggaaaacttgacctccgtactactgaagctgttatcactgggatcgccaaagcttgtaaaaaggctggatgtgctctcctcggaggtgaaacggcagagatgcctgacatgtaccctcctggagagtatgatctagctggttttgccgttggcgccatggagcgggatcagaaacttcctcaactggaaagaatcacggaaggagacgctgttattggaatcgcttcatctggtcttcacagcaacggattcagccttgtgaggaaaattgtagcaaaatcttctctcgagtactcttctccagcacctggtggctgtggtgaccagaccttaggtgacttacttctaaccccaaccaaaatctacagccgctcactgttacctgtcctgcgttcaggacgtgttaaggccgttgcacatattactggtggaggattactggaaaacatccccagagtcctccctcagaaattgggggtgaatttagatgcccagacctggagggtccccaggatcttctcatggttacagcaggaaggccacctctctgaagaggagatggccagaacatttaactgtgggattggggctgccctcgtggtatcagaggacctggtgaagcagactctgcaggatattgagcagcaccaggaagaagcctgcgtgatcggcagagtggttgcatgtcccaagggttctcctcgtgtgaaagtcgagcatctgattgaaaccatgcaaataaatggatcagtattggagaatggaaccctgagaaatcatttctctgttcaaccaaaaaaggcaagagtagctgtcttaatttctgggacaggatcaaacctccaagctctcatagacagcactagggagccaagtagcttggcacacattgtcattgtgatctccaacaaagctgcagtggctgggttagataaagcagaaaaagccgggattcccacgagagtaattaatcataaattatataaaaatcgtgcagcatttgacactgcaattgacgaagtccttgaagagttctccacagacatagtctgtcttgcaggattcatgagaattttgtctggccccttcgtcagaaaatggaatgggaaaatgctcaatatccacccatccttgctcccttcgtttaagggttcaaatgcccatgaacaagtcctggatgctggggtcacagtcaccgggtgcaccgtgcactttgtagctgaagatgtagatgccggtcaaattatcttacaggaagccgttccagtgaagaggggtgacactgttgaaactctatctgagagagtaaaattagcagaacataaaatatttccctcagccctccagctggtggccagtggagctgtacggcttggagaaaatggcagaatctgttgggtcacagaggactgaagcgtcatcccccgggaatggagacagctgagagcattgtaggtgctgcagggggacttttatcctggactccacctaaaagatgtgtgagacatctgtgtgttgcctccagtggtctttcaggttttacctgtctggggccttcttgttcataatcaaactaggccttcagaattccttttactttcagccaagtattataccaaaacttagtcttttctttttcaaagagctaacaagtctaaaaagttagtatcttgtcctcatattgtatatctgtactaatttgaattgagacttctcgatttattctgatcataagttgttatcataaaatttctgggtagttcccattgagggatagtgttctctgtgctttgaagaatcattacactttcctgtaataattgacgatttctataatacccagatttcagcttcacagtttgggaggatacaaggcaatctactcagaccagtaaagcgtttattttttttttttatgttatttaattattgcaatagtaattttctgtttttcataacatgtacagattccacagtgttatggattaaacagtcttctgtctaaaaaaaaaaaaaaaaaaaaaaaaaaaaaaaaaaaaaaaaaaaaaaaaaaaaaaaaaaaaaaaaaaaaaaaaa

SEQ ID NO: 50 - DNA, 4609 bp, Sus scrofa
  source 1..4609: mol_type = other DNA
acagacagcaatggcagcccaagtacttgtaattggcaatggaggaagggaacatacactggcctggaaacttgcacagtctaatcatgtcaaacaagtgttggttaccccgggaaatgccggcactgcctcctccgaaaagatttcaaatactgatgtctccgtcagtgatcatgctgcccttgctcagttctgcaaagaggagaaaattgagtttgtagttgttggaccagaggcacctctggctgctggaattgttgggaacctgacatctgcaggagtacggtgctttggccccacagcagaagcagctcagttagagtccagcaaaagatttgccaaagagtttatggatcgacatggaatcccaacggcacaatggagagctttcaccaaacctgaagaagcctgtagcttcattatgagtgcagatttccctgctttggttgtgaaggccagtggtcttgcagcagggaaaggggtgattattgcggagagcaaagaagaggcctgcaaagctgtacaggagatcatgcaggatagggctttcggagaagctggagaaacaattgtcattgaagaacttcttgaaggagaagaggtgtcttgcctgtgcttcactgatgggaggactgtggcccccatgcccccagcacaggaccataagcgattgctggatggagatcatggccccaacacggggggaatgggagcctattgtccagcacctcaggtttctaaggatttgttgctaaaaattaaagatacgattcttcagaagacagtggctggcatgcagcaggagggtgtcccatacacaggtattctctatgctggtataatgctgaccaagaatggtccaaaagttttggagtttaactgccgtttcggtgatccagagtgccaagtgatcctcccgcttctgaaaagtgatctttatgaagtgatccaggctaccttagatggtagactgtgcacatctctgcctgtttggcatgacaaccgcgctgccgtaactgtggtcatggcaagtaaaggttatccaggagactacaccaagggtgtagagataacagggtttcatgaggctcaagctttaggactggaggtgtttcaagcaggcactgccctaaaagatggcaaagtggtgactaatgggggtagggtccttacggtaacggccatccaggaaaatctcatctcagccctggaagaagccaagaaaggactcgctgctataaagtttgagggagccatttataggaaggacattggctgtcgtgccatagctttcctccagcagcccaggggcctgacgtacaaggaatctggggtagacattgcagctggaaatatgctggtcaaaaaaattaagccattagcaaaagccacttccagaccaggttgtgatgttgatcttggaggttttgctggtctttttgatttgaaagcagctggtttcagtgatcctcttctagcctgtgggacagacggcgttggaaccaaactgaagattgcccagcagtgccataaacatgataccattggtcaagatttggttgcaatgtgtgtaaatgatattttggcacaaggagccgagcccctctttttcctcgattacttttcctgtggaaaacttgacctcaatacgacggaagctgttgtggctggaatcgctgaagcttgtaaaaaagccggatgtgctctcctcggtatgatcatccattttaatcacacggggtgaaacgcagaaatgcccgacatgtaccctcctggagagtatgacttagccggttttgccgttggtgccatggaacgagatcagaaactccctcatctggaaagaatcacggaaggggacgctgttattggagtggcttcatctggtcttcacagcaatggatttagccttgtgaggaaaatcatagcaaaatcttccctccagtactcctctccagcacctgatggctgtggtgaccagaccttgggggacttacttctgactccgaccagaatctacagccattcgctgttacctgtactacgctcgggacatgttaaggcctttgcccatattactggtggaggattgctggaaaacatccccagagtcctccctcagaaatttggggtggatttagacgcccggacctggaggatccccaggatcttctcctggttacagcagctaggacatctctcagaagaagagatggccagaacatttaactgtgggattggggctgccctcgtggtgtcaaaggatctgacagagcagattctgcaagatattgagcggcataaggaagaagcctgggtgattggcaaggtggtagcgtgtcctgaaggttctcctcgtgtgaaagtcaagcatctgattgaaagcatgcaaataaatggatcagtgttggagaatggcaccctgaaaaatcatttctctgttcaaccgaaaaaggcaagagtagctgtcttaatatctggcacaggatcgaacctccaagccctcatcgagagcactcaggcaccaagtagctctgcacacatcattgttgtgatctccaacaaagcagcagtggccggcttagataaagcagcaagagccggcatccccaccagggtaattaatcacaaattatataaagatcgtgtagcattcgacatggcagttgaccaagtccttgaagagttctccacagacatagtctgtcttgcaggattcatgagaattttatctggcccctttgttagaaaatggaatgggaaaatgctcaacatccacccatccttgctcccttcatttaagggttcaaatgcccatgagcaagtcctggatgccggggtcacggtcactgggtgcactgtgcatttcgtagctgaggatgtcgatgctggacaaattgttttacaagaagctgttccagtgaagaggggagacaccattgaaactctctctgaaagagtaaaattagcagagcataaaatatttccctcagccctccagctggtggccagtggcactgtacagcttggagaaaatggcaagatccgttgggtcagagaggaatgaaatgtccttgtccagggatggagccagctttgaacaaattctgaccactgcagagtggctttcctcatggactcagccctaaaagatccaaagcataccggtatagtctccagtggtcttgcaggttttacctgcgtggggccttcttggttacagtcaaactggtcattcagaattccttttgctctcagccaagcaggataccaaaacttaatcttatgttctttctcatagagttagttaacaagtctaggaagttaatagctcttcccccatgttgagtatatctatgccgatttgaattgagatttcgctaattattctcatcataagttggtgtcataaaacttctggggagctcccacttctgagggtagtatgctttgaagtatcattaaactttcctctaataattgacaatttctaccatacccatattttagcttcatagtttgagaggatggaaggcaatctacccagactagtaaagagtatcatgagactgcgtgcatttcccactctagccagtatttccaatttggatattcagttcgggaaccaggaggtctgggttgacatactgggcacatgcacttgaccctacacagaggaccaaccacatttctttctttaatagctgagcgaacaggcaatggccacctggcccacagccaggcaaccagcccgcctgagactgccgagcctccttccgggtggcctaagagccctgcctgcccaacaggacgaccttcccgcaagggaacagcacagatgtacactagttacaaacagaagataatcaactcccaccttaagacaggagggagcacagagggtcccggggaagcagagtgccagaaaaaagaaacgagacctttgccaagagaccctcctcactgcctctggtccaagtaggtttccccaggcagctggcagcattaccctacctcttgttgaccgcagtcagcaactgccatcagatgggaagtccaggtacatcatttcacataggtgcctttgctgcaaaaacaactgattcactttttttctagagttcactcaacataagtaagcatctatacgccaaacactgtcctatgccatttttaggacgttagaagatacaaaggaacatgttactggttgataaaatcgcatattgtgataaaagtagaagctggtactaggatgtaaattagaaaaatcaatgtgaatttatgcttttagaaatatagttgtctagctcttaactttgacactagtctccacaaaaaggaacccaaactccacagaaggtagctaattcagtatcttgggataggaagaatcaaagtgtgtagaaactaggcgtttgcttaaagggtatcctactgtctagtttgtgataaactgaatgttatttgtacatgttgagtgtgaacagccattggcttataatactcaaagacaaaaggtcattgaagttatgaagagtcataatgcataaaagggtgaaaacaagatgttcatcgtttaataaatatagatgtttgtcttttatta

SEQ ID NO: 51 - DNA, 2317 bp, Bos taurus
  source 1..2317: mol_type = other DNA
gaggagccttggaggaacgtccggcgtgggatctgtacagccagttcagggttcatggcttcctccaggacgctcggcactttccgtctcccgccgttacccacgattcgagaaatcattaagttgtttagactgcaagccgtgaagcagctatcccagaattttcttctggacttaaggctgacagataaaattgtaaggaaagctggcaatctgacaaatgcttatgtatatgaagtgggccctgggcctgggggaatcacaagatccattctcaatgccggtgttgctgaacttctggtggttgaaaaggatagtcgatttattcctggattacagatgctttctgatgcagcacctggaaaactgagaattgttcatggagatgtcttgacgtttaagatagaaagggcttttccagaaagtctcaaaagacagtgggaggatgatcctccaaatgtacatattattggaaatctgccttttagtgtatcaactccactgattatcaaatggcttgaaaatgtttctcaaagaaatggaccatttgcatatggtagaacccggatgatgttgacttttcaaaaggaagtggcagagagacttacagctactactggaagcaaacagcgaagtcgcctctccatcatggcccagtacctctgcgacgttcagcacatcttgacaattccaggtcaggcttttgtgcccaaaccagagaatgttattccctgaagctcggcgattagaaagcacgggaaaactgttagagttggcagatgtggacccgactctccgacctacccaactcactgtctcacattttaagagcctctgcgatgtgtaccgaaaaatgtgtgatgaagacccacacctctttgcttacaatttcagagaagaactcaggcaaaagtcaaaaaaagaggatgacaagcagagctgcagactgtagctgctactcaaggggctgccagcctgccaggtgctgaacttctcatgtgtgcgcacgactgtgacttactccccttttacagcttggtaaagagaataaatatcaagtaagatagagtacaagctttaatatacatatatatactgcatagatgtgacatgtttagtataaacagcatctattacaaccttacaaaaatgaagtatctgcacattgtacccctaacccacattaaaataatttaaatatgctttaaataaagtgaaacctaatagctgaaagtattggagaaaaaatgaccttttaaaagctattaatcacaacttccccacctttttctacataaattagaatgcattcatcttaaggattctaaatcattttgatacctgctcaaaagggatataaactaagtaatttacaaattctgaccagtttaagctagttttctgatccacaataaattaaaataacttaagtctttacaactatttgtgggaaaatgtatcctgcacaaaacctaataatgaatagtgtttctgcaattttcattttgccaccagtttaaaagtgaaaccagcaatgtaagccgtatctgggttggactgtgctaggactttccatggctctctgtttcataaaagcactcttcgttgacaaccgatgttaagctctggacgctgaattctcgctcaaggacagagctgagatcaaggttggcgtttccaggatgactgtccagggactggtgacgcgtctgcgccttcagcagggtgcccctgtcccgcttggtgctgttggccttgcgtttgatggcgcagaagtgcccccgcaccagcttgggctgcccatccttgctgggcggctcttcagccgggtgagcatcggggtcctcagagatcctcagtctctggaactggatctcgatgtctttggtggggctgccctgcctccgagcctgggggtagtcgtcctcgtcgtcgctcagaatgtcgctctctttgatgagactgtcggagaagtcggccaggcccgcgtgggggtgctgctgggtgtcctgcccccttccggcaggggggcagccgctgctctgggtgctgctgctcaggctgccctcgtccgagtccagggagctgcccttgcccggctcccccggccactcgctgctggaggaattcttaaggacttttaaggacctggatgaagctgcaacgagaaaagcacacgttcccttatgacagaggaactgtttccacaatgtgtaacgttggccttggaggttatgagagttaacatcttaattaaaattttttcatctcactgaaattactaactgtcctgggacttgagcaaaaaaaaaaaaaa

SEQ ID NO: 52 - DNA, 1023 bp, Sus scrofa
  source 1..1023: mol_type = other DNA
atggctgtctccgggccgctcggcactttccgtctcccgccgttgcccacaattcgagaaatcattaggttgtttaaactgcaagcagtgaagcagctatcccagaattttctcttggacttgaggctaacagataaaattgtaaggaaagctggcagtctgacaaatgcttatgtttatgaagtgggccctgggcctggtggaatcacaagatccattctcaatgctggtattgctgaacttctggtggttgaaaaggatagtcggtttattcctggattacagatgctttctgatgcagcacctggaaaactgagaattgtccatggagatgtgttgacatttaagatagaaagggcttttccagaaagtctcaaaagacagtgggaggatgatcctccaaatgtacatattattggaaatctgccttttagtgtatcaactccactgattattaagtggcttgaaaacgtttctcatagaaatggaccttttgcttatggcagaacccagatgacactgacttttcagaaggaagtggcagagagacttatagccagtaaaggaagcaagcagcgtagtcgtctttccatcatggcccagtacctctgcggcgttcagcacgtcttgacaattccaggtcgggcttttgtccccaaaccagaggtggatgtgggggtggtgcactttacccccctgactcagcccaagatagagcagccgttcaagctggtggagaaagtggttcagaatgcatttcagttccgaaggaaatactgctatcgagggcttggaatgttattccctgaagctcagcgcatggaacgcactgggaaactgttagagtcggcagacgtggaccctactcttcgacccacccagctctcggtctctcatttccagagcctctgtgacgtgtaccggaaaatgtgtgatgaagacccacacctctttgcttacaacttcagagaagaactcaggcacacgtcagaaaagagagagagagatgatggggagagttag

SEQ ID NO: 53 - DNA, 3992 bp, Sus scrofa
  source 1..3992: mol_type = other DNA
agaggcagttgaaagatggcggacgaggcggcgctcgcccttcagcccggcggctccccctcggcggtggcggctgagagggaggccccgtcgccccccgccggggagccgctccgcaagaaaccgcggagagacggcccgggcgtcgggcggagcccgggcgagcctggtggggcggccttggagcgtgagctgccggcggcggcgggcggctgcccggcggcggcggtgctgtggcgggagaccgctgcgggcggggagcgggaggcccaggcggctgcggcggcaggagaaggaaacaatgggccgggcttacagggcctatcccgggaggcgcccccggccgacgacttctacgacgacgacgacgacgacgagggcgaggaggaggaagaggcggcggcagcggcggctattgggtaccgagataaccttctgtttggtgatgaaatcgtcaccaatggtttccattcttgtgaaagtgatgaggatgatagagcctcacatgccagttctagtgactggactccaagaccacggataggtccatatacttttgttcagcaacatctcatgattggcacagatcctcgaacaattcttaaagatttgctaccagaaacaattcctccacctgaattggatgatatgacactgtggcagattgttattaatatcctttcagaaccaccaaaaaggaaaaaaagaaaagatattaatacaattgaagatgctgtgaaattactacaagagtgcaaaaaaataatagttctaactggagctggggtttctgtttcttgtggaatacctgacttcaggtcaagagatggcatttatgcacgccttgcagtagactttccagaccttcctgatcctcaagcaatgtttgatattgaatatttcagaaaagatccaagaccattcttcaagtttgcaaaggaaatatatcctggacagttccagccatctctctgtcacaaattcatagccttgtcagataaggaaggaaaactgcttcgcaactatactcagaacatagatacactggaacaggttgcaggaatccagaggataatccagtgtcatggttcctttgcaacagcatcttgcctgatttgtaaatataaagttgactgtgaagctgtacgaggagatatttttaatcaggtggttcctcgatgtcctaggtgcccagctgatgaaccacttgctatcatgaaaccagagattgtcttttttggtgaaaatttaccagagcagtttcatagagccatgaagtatgacaaagatgaagttgatcttctcattgttattgggtcttccctgaaagtaagaccagtagcactaattccaagttccataccccatgaagtgcctcagatattaattaatagggaacctttgcctcacctgcattttgatgtagagcttcttggagactgtgatgtcattattaatgaattgtgtcataggttaggaggtgaatatgccaagctttgctgcaaccctgtaaagctttcagaaattactgaaaagcctccacgaacacaaaaagagttggctcacttgtcagagttaccacccacacctcttaatatttcagaaggctctagttcaccagaaagaacttcaccaccagcttcttcagtgactctccctcttttagaccaagcaacaaagagtaatgttgatgatctagatgtgtctgagtcaaaagactgtgttgaagagaaatcacaggaagtacagacttctaggagcattgaaagtgttaaagaaccgatggagagtccaggtttgaagaatgttgcctgcagtaatggagagaaaaatgaaagaacttcagttgccgaaacagtaagaaaatgctggccagctagacttgcaaaggagcagattagtaaacggcttgatggtaatcagtatctgtttttaccaccaaaccgttatattttccatggcgctgaggtatattcagactctgaagatgatgtcttatcctctagttcttgtggcagtaacagtgagagtggaacttgccagagtccaagtctagaagaacccatggaggatgaaagtgagattgaagaattttacaatggtttggaagatgatgctgatgttaatatgcgagctggaggaactggatttggagctgatggaagtgaccaagaggcagttaatgaagctatatccatgaaacaggaagcaacaggcgttaactatccatcaaacaaatcataatgtaataattgtccaggtacaggaattgttccaccagcattagggactttagcatgtcaaattgaacatttacttgtgaacctagtagcgcaaggaaaccagaaaggtgtaatatttataaactggtaaaagattttcttcccatgggtaatttttaacttcttgtttctgtgcttatatactcaaacactaactttttttttttttttttaaagaaaggtactaagtatctttaatcagctgttggacaagactttattttaaagattcatttgtatgatatatccccatgtatatataatttttttttttttgcctagtgagtttcaacattttaaagttttcaaaaagccattggaatgttaaattaatgtaaagggaacagcttatctagaccaaagaatggtactttcacacttttctttgtaacattgaatggtttaaaatcctcaaatctgtctgctgaacttttgattctttattagcacaattacctatttttaaacactggcatttttccaaaacttgtggcagctaactttttaaaaatctcatgacatgcagtgtgagtagaaagaagtcaacggtatgtgggagagcacttagttgtctttgctttttaaagtgagacttggtgctaatcgtttcaggaatattgtattgttcaaatgaagatggcttttgtacttcctgtggacatgtggtaatgtctatattgcctcataaaactaacctaaaaaacaaataaatgctttggaaatgtttcagttgctttagaaacagtagtgcctgccttggtccccttagttataagaatatttgctattgtttaaatacctatcactgtggtagagcttgcattgatccttttccacaagtattaaactgccaagatgtgaatttgcaaagtctttatgaaccaataataacggtacttcaacttggggagcgtgtaataatttggactgttgctcccccttacccctgcaccccccattaagtgagagcaacaggccctgatttaggttccaaagccagtaagtacatgtttcctgttgggaagatttggtgtaataaacaccaaactattagacggagacgaaggcgatgtgacttgtaataccagaatagctgatgaaactagttcctataaaaagcttagcctgccttaaaactagacatcaattttctcagctgcagaagcttctagcctttcaaaaaagttcatactttataaagttgcagagaagcatttattttccagtcctttccccaatgttacttccaaataaagtattcctatgttgctttagtatttattacaatttaacaaaaaagggtttgaaatgtagctgttctttaagtataaaatacccagcttggaccattactgccggagaagagttttattgaatggctatttccctacctaaaagaggtctcaaactgaatttattgggctacgctaaagaatgcagtatatttagttttccatttgcatgatgtgtgtttgtgctatagatgatattttaaattgaagaatttgttttaaattatttttacagtgaagactgttttcagctctttttatattgtacatagtcttttatgtaatctactggcatatgttttgtagactgtttaatgactggatatcttcctccaacttttgaaatacaaaaccagtgttttatacttgtacactgttttaaagtctattaaaattgacatttgacaaaaaaaaaaaa

SEQ ID NO: 54 - DNA, 3956 bp, Bos taurus
  source 1..3956: mol_type = other DNA
gcgagggaggagggccagagaggcggttgaaagatggcggacgaggcggcgctcgcccttcagcccggcggctctccctcggtagtggcggctgagagggaggccccgtcgccgcctgctggggagccgctccgcaagaggccgcgaagagacggccccggcgtcgggcggagctcgggcgagcccggtgggacggcccccgagcgggaactgccggcggcggccggcagttgcccggcggcggcggcggcgctgtggcgggaggcccaggcggcggcggcggcggcggccgaagaagaagacaatgggccgggcttacagggcctatccagggaggcgccaccggccgacgacttctacgacgacgacgacgagggcgaggaggaggaagaggcggcggccgctattgggtaccgagataaccttctgtttggtgatgaaatcatcaccaacggtttccattcgtgtgaaagtgacgaagatgatagagcctcgcatgcgagttctagtgactggactccaagacctcggataggtccatatacttttgttcagcaacatctcatgattggcacagatcctcgaacaattcttaaagatttactaccagaaacaattcctccacctgaattggatgatatgacactgtggcagattgttattaatatcctttcagaaccaccaaaaaggaaaaaaagaaaagatattaatacaattgaagatgctgtgaaattactgcaagagtgcaaaaaaataatagttctaactggagctggggtttctgtttcttgtggaatacctgacttcaggtcaagagacggtatttatgctcgccttgcaatagactttccagaccttccagatcctcaagcaatgtttgatattgaatatttcagaaaagacccaagaccattcttcaagtttgcaaaggaaatatatcctggacaattccaaccatctctttgtcacaaattcatagccttgtctgataaggaaggaaaactacttcgcaactatactcagaacatagatacactggagcaggttgcaggaatccaaaagataattcagtgtcatggttcctttgcaacagcatcttgcctgatttgtaaatataaagttgactgtgaagctgtacgaggagatatttttaatcaggtggttcctcgatgtcctagatgcccagctgatgaaccgcttgctatcatgaaaccagagattgtcttttttggtgaaaatttaccagaacagtttcatagagccatgaagtatgacaaagatgaagttgatcttctcattgttattggatcttccctgaaagtaagaccagtagcactaattccaagttccataccccatgaagtgcctcagatattaattaatagggaacctttgcctcacctgcattttgatgtagagcttcttggagactgtgacgtaattattaatgaactgtgtcataggttaggtggtgaatatgccaaactttgctgtaaccctgtgaagctttcagaaattactgaaaaacctcctcgaatacaaaaagagttggcacatttgtcagaattgccacccacacccctcaatatttcagaaggctcaagttcaccagaaagaacttcaccaccagattcttcagtgattgtcactcttttagaccaagaaacaaagagtaacgttgatgatccagatgtgtctgaatcaaaagaccatgtcacagaaaaatcacaggaagtacagacttctactaggagcattgaaagtgttaatgaacagttggagagtccagatttgaagaatgctgtctccaattctggtgagaaaaatgaaagaacttcagtagctgaaacagtgagaaaatgctggccagctagacttgcaaaggagcagattagtaaacgccttgatgataatcagtatctgtttttaccaccaaaccgttacattttccatggcgctgaggtatattcagactctgaagatgatgtcttatcctctagttcttgcggcagtaacagtgatagtggaacgtgccagagtccaagtttagaagaacccatggaggatgaaagtgagaatgaagaattttacaatggtttggaagatgatgctgatgttaatgagagagctggaggaactgtatttgaagctgatggaggtgatcaagaggcaattaatgaagctatatctgtgaaacaggaagcaacatgcattaactatccatcaaacaaatcataatatagtaattgtccaggtacagaaattgttccaccagcattagaatctttagcatgtcaaattgaatgtttacttgtgaactcaatagagcaaggaaaccagaaaggtataatatttatagactggtatcccctcaatgggtaatttttaacttccttatttctgtgcttatacactcaaacactaactttttttttttaaacaaaggtacgtaagtatctttagtcagctattggactagactttctgttaaaggttcatttgtatgatacatcgacatgtatatataattgtttttgcctagtgagtttcaacatttcagagtcttcaaaaagccattggaatgttaaattaatgtaaagggaacagcttatctagaccaaagaatggtattttcacacttttcgttttaacattgaatggtttgaaatcctcaaatctgttctgctaaacgtttgattctttattagcacaattacctatttttaaacactggcattttccaaaacttgtggcagctaactttttaaaaatctcatgacatgcaatgtgagaaggaagtcaacgtgggagagcactcagttgtttttgcttttttaaaatgaaacttggtgctaacagtttcagaaatgtatcgttcaaatgaagatcgcttttgtacttcctgtggacatgtagtaatgtctatattggctcataaaactaacttaaaaaataaatgctttggaaatgtttcagttgctttagaaacaatagtacctgcctgggtccccttagttttaagaatatttgccattgttgtttaaacacctatcactgtggtagagcttgcattgatcctttccacaagtattaaactgccaatgtcaatctgcaaagcctttatgatcaataataatggtactttaactggggagagtgtaatattttggactgttgccttttttgcccattcatgagagcagcagacccagattacggttccaaagccagaaagtacatgtttcctgttgggaggacttggtgtaagaaacaccaaacatactagcctagtattatggagatgaacgtgatgtaacttgtaatagcagaatagttaatgaaactagttcctataatgtctttatttaaaagcttagcctgccttaaaactagagaacaactttttcagctgcagaagcttctagcctttcaacaaaagtttgtactttataaaattgcacagtaacgatttatttctcagacctttccccaacattgttttcaaaaagtatttttatgttgctttagtatttattacagtttaacaagaagggtttgaaaaacagctgttcttaagcttaaaatatccagctaggaccattactgccagaggaaaaaattttattgaatggccatttccctacctaaaagatgtttcaatctgaatttatttgactacactaaagaatacagtatatttagttttccatttgcatgatctgtgtttgtgctatagatgatattttaaattgaaaaatttgttttaaattatttttacagtgaagactgttttcagctctttttatattgtacatagtcttttatgtaatctactggcatatgttttgtagactgtttaatgactggatatcttcctcaaacttttgaaatacaaaaccagtgttttatacttgtacactgttttaaaagtctattaaaattgtcatttgactttt

SEQ ID NO: 55 - DNA, 1826 bp, Sus scrofa
  source 1..1826: mol_type = other DNA
gctgctgggagttgtagttctctgccttgtctcggccccctcctgtttccggtgccgtcacgggacagagcagtcggtgacagtcccgagggcccccgccccgcgcccatggccgagccggatccctcagaccctgtggagacccagacagggaaggtgcaagaggctcaggactccgattccgattcggacactgaggaaggagcagctggtggagaagccgagatggacttcctgcggaatttcttctcccagaccctgggcctgggcacccagaaggagcgtctgctggacgagctcaccctggaaggggtgtcccgctacatgcagagtgagcgctgtcgcagggtcatctgtttggtgggagctggaatctccacccgcggcatccctgacttccgctccccgtccacgggcctctatgccaacctggagaagtaccgtctgccctacccggaggccatctttgagattggctacttcaagaaacatccagagcccttcttcgccctcgccaaggaactctatcctgggcagttcaagccgaccatctgtcactacttcatccgcctgctgaaggagaaagggctgctcctgcgctgctacacgcagaacatagacaccctggagcgagtggcgggactagagcccgaggacctggtggaggcccatggcaccttctacacgtcacactgcatcagccccctctgccggcaggagtacacgctaggctggatgaaagagaagatcttctccgaggtgacccccaagtgtgagaaatgtcagagcgtggtgaagcctgatatcgtgttcttcggtgagaacctcccagcgcgtttcttctcctgcatgcagtcagacttcctgaaggtggaccttctcatcgtcatgggcacctccctgcaggtgcagcccttcgcatccctcatcagcaaggcgcccctctccaccccacgcctgctcatcaacaaggagaagaccggccagactgacccgttcctcgggatgatgatgggcctcggaggaggcatggacttcgattcaaaaaaggcgtacagggacgtggcctggctgggtgactgcgaccagggctgcttggccctcgctgatctcctcggatggaagaaggagctggaggaccttgtccggaaagagcatgccagcatagatgcccagtcgggatcgggcacccccaatcccaccacttcagcttcccccaggaagtctccgcctcctgccaaggcagaggccaggacctcggagggagagaaaccccagtgacagctgcgtctcccaggcaggacgcccagctttgggacagctgagtcccaaccagccctggcctcctctaacttacagctcttttctggggagctcagaacgtttccgcaatcttttaactactccctcctaaacctggggtcccggcagccccgaccccagcaaatctctaacattctttggggccgaggcttgggcagcccgcctctaacagcccacagcctctaaccacccccgggactgaggatcagcctccctaatctctaactgctcagggggccgggtgtacctccaaacctctaactgtcccaggccaagggtccccagggcccccacgccctctactgtccccaggagcctgtggccaagtagctgaaacctaacctacccgtgggtggggggatctgagcctcaaggtctcccaagtctcttcctccagctccccaagtgggtgctggaccccctcactcgggacccacttcccatgccaaggagggcagatggtgtcacttattagagatgaattaaaaacaaagaacaactaa

SEQ ID NO: 56 - DNA, 1849 bp, Bos taurus
  source 1..1849: mol_type = other DNA
gacctatctcggccctctcctgtttccggtgccgtcacgggatagagcagtcggtgacagtcccgagggcccccgccccgcgcccatggccgatccggatccctctgaccctgaggagacccaggcagggaaggtgcaggaggctcaggactcagattcagacactgaggcaggagccactggcggagaagcagagatggacttcctgcggaatttcttctcccagactctgggcctgggcacccagaaggagcgactcctggacgaactaaccctggaaggagtgagccgctacatgcagagcgagcgctgtcgcagggtcatctgtttggtgggagctggaatctccacttccgcgggtatccctgactttcggtccccaaacacgggcctctatgccaacctggagaaataccgtcttccctacccggaggccatctttgaaatcagctacttcaagaaacatccagagcccttctttgctctcgccaaggaactctatcctgggcagttcaagccgaccatctgccactacttcatccgcctgctgaaggaaaaaggactgctcctgcgctgctacacacagaacatagacaccctggagcgagtggctgggctggagcccgaagacctggtggaggcccacggcaccttctacacgtcccactgcatcagctcaggctgccggcaggagtactcactaagctggatgaaagagaagatcttctccgaggtgactcccaagtgtgagaaatgtcagagcgtggtgaagcctgatatcgtgttcttcggcgagaacctcccagcgcgtttcttctcctgcatgcagtcagacttcctaaaggtggacctccttatcatcatgggcacctccctgcaggtgcagcccttcgcgtccctcatcggcaaggcgcccctgtcgaccccgcgcctgctcatcaacaaggagaagactggccagactgaccctttcctcgggatgatgatggccctcggaggaggcatggactttgactccaagaaggcctacagggacgtggcctggctgggcgactgtgaccagggctgcctggccctcgccgacctccttggatggaagaaggagctggaggaccttgttcggaaggagcatgccagcatagatgcccagtcggggtcgggggcctccaaccccgctacttcagcttcccccaggaattctccaccacctcccaccaaggaggagcccaggaccactgagggagagaaaccccagtgaccactgcgtctcccaggcaggacacccagccttgggacagctgagccccaaccagccctggccctcctctaacttacagctcttttctggggagctcagaacatttcttcaatgtattaactattccctcccaaaactggtgtcccagcacccctgacctcaccaaatctctaacattcttgggggcagaggcttgggcagcctgtctctaacagcccacagcctctaaccacccctacggccaaggatcagcccctctaatctctaactgctcagggggccacaggtacctccaaactgctaactgtcccaggacaagagccccagggcccacacgctctctactgctcccaagggtctgtggccaagtagataaaacctaacctaccctgggtggggagttgtgccctccacgcacccagtggggagatccgagcctcaaggcctcccaggcctctgcctccaactctgaaagtgggtgctaggcccctctcttggatcccgcttcccaggcaggggtgggcagatggtgttgcttattagagatgaattaaaaacaaaaaacaactaacgattaaaaaaaaaaaaaaa

SEQ ID NO: 57 - DNA, 3646 bp, Bos taurus
  source 1..3646: mol_type = other DNA
ctcctcctccgaggaaacctgccgcttctagctccccaccctcccctttaaagggtgacttgctctgtgccagaccgctgctccagcctgtgccgccttgggttcagcgggtctactgttctgtccgcgctcgcgcctggtgccctgcatctcctacagagggacatcccccgagatggagagcaaggccctacttctgctggctctgagcgtgtgcctgcagagtctgaccgtctcccgcggagggctggtcgccgcagacaggattacaggaggaaaagattttagagacattgaaagtaaatttgctctcaggactcccgaagacacagctgaggacacttgccacctcattcctggagtgaccgaatctgtggctaactgtcacttcaaccacagcagcaaaacctttgtggtgatccatggctggacggtgacaggaatgtatgagagttgggtgccaaaactcgtggctgccttgtacaagagggaaccggactccaacgtcatcgtggtggactggctgtcacgggcccagcagcattatccagtgtctgcagggtacaccaagctggtgggacaggatgtggccaagtttatgaactggatggcggatgaatttaactatcccctgggcaatgtgcatctcttgggatacagccttggggcccatgctgctggtattgcaggaagtctgaccaataagaaggtcaacaggataaccggcttagatccagctggacctaacttcgagtatgcagaagctccaagtcgcctttctcctgatgatgcggattttgtagacgttttacacacattcaccagggggtcaccaggtcgaagtatcggaatccagaaaccagtagggcatgtggacatttaccctaacggaggcactttccaaccaggatgtaacattggggaagctctccgtgtgattgcagagagaggccttggagatgtggatcagctggtgaagtgctcccacgaacgttccgttcatctcttcattgactctctgttgaatgaagaaaatccaagtaaggcctaccggtgcaattccaaagaagcctttgagaaaggtctctgcctgagctgcagaaagaaccgttgcaacaacatgggctacgagatcaacaaggtcagagccaaaagaagcagcaagatgtacctgaagactcgttctcagatgccttacaaagtcttccattaccaagtaaagatacatttttctggaactgagagtaatacatacaccaaccaggccttcgagatctctctgtatggcactgtggctgagagcgagaacatcccttttaccctgcctgaagtttccacaaataagacatactcctttctactttacacggaggtggatattggagaattgctaatgttgaaactcaaatggattagtgattcctacttcagctggtccaactggtggagcagccccggctttgatattgggaagatcagagtaaaggcaggagagactcaaaaaaaggtgatcttctgttcccgggagaaaatgtcttatctgcagaaaggaaagtcacctgtgatatttgtgaaatgccatgacaagtccctgaatagaaagtctggctgaaacttggcaaagctacggaagaaagaacagcatatgaattctatgaagaatgaagtaacttttacaaaagatgcccagtgctttagatggtgaaatgtggattttccggagtattaaccccagctctagccttattagttattttaggagacagtctcaaatactaaaactaattcaatttgaggtgtatagtggccaaatagcacatcttccaacattaaaaaaataacagatatgaaaagcactgcattctgtcttttgaaaaaatatgagttatttaaaatgataaaataatcagatctcttcatgtagtaagtttagccatagcctaaaattgattaaaatctcatttttaattgggattctgcgttttctagactgataaccttctcagagttttctccgagtctaaatataggaagtaagtttttttgcagcgtgagtaggacccgtttacctatcaatcaaaatccctactttcttggaattactctcctcttgccattggaatgtagtccaagaatgaaccaggaactagtgacttggagatagaaatgaagaatagagttgataaagcactgaacctttaaaccccctctacggttggttgcatcataactaagttaccaattaaaggagatatataaagttgagatcaattaaatcttaataaacttaacagtttatggtttagtatttccccttccttttcctgctttgtctcaagattatattttaatagtttttccctagataggcttttcaactgagcctgttatcatcagctgatacagaattttaactacagaggaaaaaaaatgatactgtaattttattatcagactttcaaaattacattcattgaagtcctttaatgtatgatagttactgttcaattttaggcttatttcagccatgcttcagtcagactttgagcatgtctctttcttccctaactttgataagaaaacacgtattcaagatgaaactttgcatcatgcaaagtcctacaaattttagacaaagattgtttttactaagtaaagaaacagaaaagttacagtcagtcatatagtcactggcagtgcttaaaaccgttgtggacaatgaaaaggatcaattgctatatccacgtgaattgtcatgaaagttgtttttaacaactagtcaagagtgagtgaacatttatttataaactagatctcatattttcagaatgcttttctatatattattatcaaatgatagagaagtcaaatatttcagaggctattactgaaaatccaaactgtgaatgtgtgtgggtgtctgaacacatattcacgtatgaagccccacatacacgattattgctcagcatggctcggaaggtaaatgattaagaggtacattagaacacactgagtagaaaatgttccagtggtgccggaacactccttcatctgagagatacggttgtgcctgtacaaagtagaatgaatgctgtgattgacatcagccagtttggaatgaattctctctaaaaataaaatgttgtgtgacttgttgttggcatcccccttcattgttataattcattaattctcgggatttgggttttgagcaagggtacatcaacttaagaaagtataaattcagtcagtgtatggccctgggaactctgacttctacagacttcactatatatatatcagcaatgctttgactttcaatgttatttattagctgtaaatatatgtgtgtatgtgtaaagggagattgtacatactggaaaggtcattgtggctatctgcatttatagatgtttggtgctaattttgtacgtgtttttatcagtgacagtttcactgagccaacttactctgatgaaacaatacaataaaatgggctttaaaaaaacacaaaacttacttgtgtgaagaaatggaatctacttttaataaaattgacaacattttattacaaaaaaaaaaaaaaaaaaaaaaaaaaaaaaaaaaaaaaaaaaaaaaaaaaaaaaaaaaaaaaaaaaaaaaa

SEQ ID NO: 58 - DNA, 2946 bp, Sus scrofa
  source 1..2946: mol_type = other DNA
ggaaagctgccacttctggctgccccgcttcttctttaaagggtgacttgccctgcaacagatcgctgctccagcctgctcccgcctcgggctgtcggcgctcccgcccgtgccctgtaacttctaccccaggaacgcgtccccagatggagagcaaagccctgctcctagtggctctgagcgtgtggcttcagagtctgatcgtctcccgcgaagggttggccaccgccgacagaatttcaggaggaagagattttacagacatcgaaagtaaatttgctctcaggactcctgaagacacagttgaggacacttgccatctcattcctggagtgacagaatctgtggctaactgtcatttcaaccatagcagcaaaacctttgtggtgatccatggctggacggtgacaggaatgtatgaaagttgggtccccaaacttgtggctgccctatacaagagggaaccggattccaacgtcattgtggtggactggctgtctcgggcccagcagcattatccaatatctgcgggatacaccaagctggtgggacaggatgtggccacgtttatcgactggatggcggttgaatttagctatcctcccaacaatgtccacctcctgggatacagccttggagcccacgctgctggcattgcaggaagtctgaccaagaagaaggtcaatagaattactggcctagatccagctggacctaacttcgagtatgcagaagctccaagccgcctttctcccgacgacgcagattttgtagacgtcttacacacattcaccagagggtcacctggccgaagtattggcatccagaaaccagtagggcatgttgacatttatcccaatggaggcactttccaaccagggtgtaacattggagaagccatccgtgtgattgcagagagaggccttggagatgtggaccagctcgtgaagtgctcccatgagcgctccatccatctcttcatcgactctctgttgaatgaagaaaatcctagtaaggcctaccggtgcaattcaaaggaagcctttgagaaagggctctgcctgagttgcagaaagaaccgttgcaacaacttgggctatgagatcaataaggtcagagccaaaagaagcagcaaaatgtacctgaagactcgtgctcagatgccctacaaagtcttccattaccaagtaaagatgcgtttttctgggactgagagtgatacccacaccaaccaggcctttgagatctctctatatggcaccgtggccgagagtgaaaacatccctttcaccctgcctgaagtctccacaaataagacatactcctttctaatttacacagaggtggatattggagagttgctaatgttgaaactcaaatgggtcagtgattcatacttcagctggtccaactggtggagcagccctggctttgctattgagaagatcagagtgaaggcaggagagactcaaaaaaaggtgatcttctgttcccgggagaagaagtctcacctgcagaaaggaaagtcctctgtggtgtttgtgaaatgccacgacaagtctctgaatagaaagtctggctgaaactgagcaaatgtacagaagaaggaacagcacgtggagtctatgaagaacaaaacaaattttgacaaagatgcccagtgctttgggagattaaaagtgaattttcccaagtattaatcccagctctagccttattagttattttaggagacagtcttaaacattaaaaatgtggctacttcagtttgaggagagagtggccaaatgacacatcttccaacattagaaaacagcagatatgaaaagcactgcattctgtcctttgggaaagaaagaagaattgtttgggcttgtggtaaaataatcagcggtctttgtgtagtaagtttagccgtagcctaaaattgatgagaacctcatactttaattggaattctgcatttttcagactgataccttctcaaaattgtcttcaagtctaaatatagaaggtgatttttttatagcctgagttggacccatgtacttaattgtcaaaatactttcttggaatgaatcttctcttgccattgggatgtagcctaggagttaaccaggaactagtaacttggagagaggaagggaaaacagagttgataaagcactgaagctttaaacactcttgcagttggttgcatcataactaagttacaaattaaaggagatataaaagttagatcaattaagtcttaagaggctttgcagtttattggtcggtcttttcccttcggtttttttttttgctttgtctcaaattgtattttaacgatgttctttgggtaggcatttaaaatgagcctgttatcatcatctgacacgtaattttaagctatagagaaagaaattggtagccataattttattcatcagattgtcaaaattacattcgttgatttgaaatcctttaatatatgatggttactgttcaattttaggcttacttcagccatacttcagtcagtttttgagcatgtttttcttctctaattttgataagataacgtgttcaaggtcaaattttgcatcatgcaaactcctacaaattttgggcaaggattgtttttacaataggaaagttgggagctccaccatggcccaggggttaacgaacccgactagcatccatgaggatgtgggtttgatccctggccctgctcagtgggttacggatccagcgttgccatgagctgtggtctaggtcccacatgcagctcagatcccgagttgctgtggctgtggtgtaggccagtggctacagctctgattggacccctagcctgggaacttccatatgccgtgggtgcagccctaaaaagacaaaagaaagaaaagatactgggatacattcataagcagtgcttaaaaccattgtgggcaattaaaa

SEQ ID NO: 59 - DNA, 2710 bp, Sus scrofa
  source 1..2710: mol_type = other DNA
gattttgaaagggtcacgtaaacaaactacaactcccagcggtccccgcgcgcaacaggtcagggctaggctggggccgggttcgcggtgctagctgaggcggcggcggcggctacggctggaagagccgggccggggcccgcggcggaggccgtggcttgtattgggagggtggcggggagggactggggaggaagatggcgacgtcgggggcgaacgggcccggctcggccacggcctcagcttccaatccgcgcaagtttagtgagaagatcgcgctgcagaagcagcgtcaggccgaggagacggcggccttcgaggaggtgatgatggacatcggatccacccggttacaggcccaaaaactgcggctggcgtacacacggagctctcactacggcggctctctgcccaacgtgaaccagatcggctgtggcctggctgagttccagagccccctccactcgcctttggattcatctcggagcactcggcaccacgggctggtagaacgggtacagcgagatccccgaaggatggtgtccccgcttcgccgctacccccgccacatcgacagctctccctatagtcccgcctacttatcgccgcccccagagtccagttggcgcaggacaatgccctggggcaatttccctgcagagaaggggcagttgtttcgactaccatctgcgctaaacaggaccagctctgactccgcccttcacacaagcgtgatgaaccccagcccgcaggacacttatccaggccctccacctcccagcgtcctgcccagccgccgtggaggtttcctagacggcgaaatggactccaaagtacccgctattgaggagaacttgctagatgacaagcatttgctgaaaccctgggatgctaagaagctgtcctcctcctcctcccgacctcggtcctgtgaagtccctggaattaacatctttccatctcctgaccagcctgccactgtgcctgtcctcccacctgccatgaacacgggaggctccctacctgacctcaccaacctgcacttccccccaccgctgcccacccccctggacccagaggagactgcctaccccagcctgagcgggggcaacagtacctccaatttgacccacaccatgacccacctgggcatcagcgggggcctgggcctaggcccgggctacgaggcgccaggacttcattcacctctcagccacccatccttgcagtcctccctaagcaatcccaacctccaggcttccctgagcagcccgcagccccagctccagggctcccacagtcacccctcactgcctgcctcctccttagcccgccacacactgcccaccgcctccctgggtcacccctcgctcagcgccccggccctctcctcctcctcttcctcctcttccgcttcatctccggtgctgggtgcccctccttacccagcttctacccccggggcctccccccgccaccgccgtgtgcccctcagccccctgagtttgcccgcgggcccagccgacgccagaaggtcccaacagcaactgcccaaacagttttcgccaacaatgtcacccaccctgtcttccatcactcagggcgtccccctggacaccagtaagctgcccactgaccagcggctgcctccatacccatacagccccccgagtttggttctgcccacccagcctcccaccccaaagcctctacagcagccagggctgccatctcaagcctgctcagtgcagccctcgggtgggcagcccccgggcaggcagctgcactatggcatgctgtacccacccggctccagtgggcatgggcagcagtcttaccaccggccaatgggtgacttcagcctggggaacctggagccgttcagcatggacaacccatccaccagcctggcgctggatgcctctggcttttccgaagggcctggatttttagggggtgaggggccagtgagtggcccccaggagccccacaccctcaaccaccagaacttaacccactgttcccgccatggctcagggcccaacatcatcctcacgggagaatcctccccaggtttctctaaggagattgcggcagccttggctggagtgcctggcttcgaggtgtccacggctgggctggagctgggcctggggctggaggaggagctgcgcatggagccactgggcctggaggggctcaacatgctgagcgacccctgtgccctgctgcccgaccctgctgtggaggactcgttccgcagtgaccggctgcagtgagggcacctgatcgccatccctctcctcggccctgtcccccgccgtcactgtcccttttctcccttccccctggccagtagagactccactctctgcccccagatcctctttctaacgtgaacgagggatcctaagaatgagaagaagcaaggggtgtgtccaggtggcccctaaattctgcacaaagggtgggcctaggggagctcaagggagggcctgaagcacttgtaactttgaaccgtctgtctggaggtcagagcctgttggaaagcaggggggtagaggggagccccggaggcagggcttgtccggatgcctaggggtgggcagtgccagcccctcctcgccactcttccccttgcaatggaggaaagagccagagtggatattattttttattaaatatattatatgttaataaaaaaatcata

SEQ ID NO: 60 - DNA, 2609 bp, Bos taurus
  source 1..2609: mol_type = other DNA
ggaggccgtggcttgtattgggagggtggcggggagggacgggggaggaagatggcgacgtcgggagcgaatgggcccggttcggccacggcctcagcttccaatccgcgtaagtttagtgagaagatcgcgctgcagaagcagcgtcaggccgaggagacggcggccttcgaggaggtgatgatggacatcggctccacccggttacaggcccaaaaacttcggctggcatacacaaggagctcccactatggtggctctctgcccaatgtgaaccagattggctgtggcctagccgagttccagagccccctccactcccctctggattcatctcggagcactcgacatcacgggctggtggaacgggtgcagcgagatccccgaagaatggtgtccccgcttcgccgctacgcccgccacattgacagctctccctatagtcctgcctatttatctcctcccccggagtccagctggcggaggacaatgccctggggcagtttccctgcagagaagggacagttgtttcgactaccatctgcacttaacaggacaagctctgactctgcccttcacacaagcgtgatgaaccccagccctcaggacacgtatccaagccctgcagcccccagcgtcctgccgagccgccgtggaggttgtctggacggcgagacggactccaaagtccctgctattgaggagaacttgctagatgataagcacttgttgaaaccatgggatgctaagaagctgtcctcgtcctcctcccgacctcggtcctgtgaagtccctggaattaacatcttcccgtctcctgaccagcctgccactgtgcctgtcctcccacctgccatgaacacgggaggctccctacctgacctcaccaacctgcactttcccccaccgctacccacccccctggacccagaggagacagcctatcccagcctgagtgggggcagcagtacctccaatttgacccacaccatgacccacctgggcatcagcgggggcctggccctgggcccgggctatgatgcgccaggacttcattcacctctcagccacccatcctttcagtcctccctaagcaatcccaacctccaggcttccctgagcagtcctcagccccagctccagggctcccacagtcacccctcactgcctgcttcctccttggctcgccacgcactgcccaccacctccctgggccacccctcgctcagtgccccggccctctcctcctcctcttcctcctcttccgcttcctctccggtgctgggggcccccgcttacccagcttctgcccctggggcctccccccgccaccgccgtgtgcccctcagtcccctgagtttgcccgcgggcccagccgacgccagaaggtcccaacagcaactgcccaaacagttttcgccaacaatgtcacccaccttgtcttccatcactcagggcgtcgccctggataccagtaagctgcccaccgaccagcggctgcctccatacccatacagccccccaagtctggttctgcccacccagcaacccaccccaaagcctctgcagcagccagggctgccctctcaggcctgctcagtgcagccctcagggggccagcccccaggcaggcagctgcagtatgggacactgtacccgcccggacccagtgggcacgggcaacagtcttaccaccggtcgatgagtgacttcagcctggggaacctggagcagttcaacatggagaacccatcgaccagcctggcgctggatccccctggcttttccgaagggcctggcttcttagggggtgagggaccagtgagtggcccccaggaccctcacgccctcaaccaccagaacgtaacccactgttcccgccacggctcagggcctaacgtcatcctcacaggagacgcctccccaggcttctccaaggagattgcagcggccctggccggagtgccgggcttcgaggtgtcagcagctgggctggggctggggctggggctggaggaggagctgcgcatggagccactgggcctggaggggctcagcatgctgagtgacccctgcgccctgctgcctgatccggctgtggaggactcgttccgcagtgaccggctgcagtgaggccacctcagcgccatccctcttctcggccctgtcccttgtcactgtccctttcctcccttccccctggccagtagaggtgccactctctgcccccagatcctctttctgacatgaacaaaggatcccaagaatgagaaaaggcaaggggttttttccaggtggcccctgaattctgcacaagcggggggcacctgggggagctcaagggagggcctaaagcacttgtaactttgaaccgtctgtctggaggtcagagcctgttggaaagtggggggtagaggggagccccggaggcagggcttgtccggatgcctaggggtgggcagtgccagcccctcctcaccgctcttccccttgcagtggaggagagagccagagtggatattattttttattaaatatattatatgttaataaaaaaatcctattaaaaaaaaaaaaaaaaaaacaaaaaaaaaaaaaaaaaaaaaaaa

SEQ ID NO: 61 - DNA, 6582 bp, Bos taurus
  source 1..6582: mol_type = other DNA
gttgggttgccagccctccaggtgacttaaggttaaaaacattttttttaaggcttcttgtttttgtgttctcagtgggctatttacaccattttcaagcatccctcctcctttggaattttaattttctgtgaacgcttcccccatagggcattccctagtgccattttattcttgacccttttcaatacaaaatccttaataaagacaatggaaagtagggtgaaagcagggaagctgaaattttgtcatctagatactgagggtggcctctttgttaagaatctagcatccagaccttgaggtcacacgattagtattcccattggaatcgtagggactctctttcaaagagtcttctgtccagttttcaggccacgttaggcacgttcccgcccagaagcagggcctgaaaagaatgtcggtgttttccttcaggtataggtgcctgggggtcaagggagagggaactgcaaggggtggagtgggaggcgggtgatagacagggatagaaactgtgtctcctcctccctctgaatgcgaaatagccaatggggtggcgcggccgagccggcccggccgccagtgccatatagtatgcagcaaccggaggagtcacgttgggggaacggcagaaagcagctatccttttacactactttgctctagcagctatcttaatgattgcaagtgcggcggacatcaagcaggagaatgggatggaaagcgcctcggaggggcaggaggcgccccgagaagtggcggggggcgcggcggcggcggcggggctgagccccccggctccagcccctttccccctggagccgggggacgccgcggctgccgccgccagggtgagcggagaggaaggggcagtggctgcagtggcggcggcggccggggcggcggtggatcaggtacaactccactcggaacttctgggcaggcaccaccacgccgcggccgccgccgcgcagaccccactggccttctcgccagaccatgtcgcctgcgtgtgcgaggcgctgcagcaggggggcaacctggaccgcctggcccggttcctgtggtccctgccccagagcgacctgctacgtggcaacgagagcctgctgaaggcgcgagcgctggtggccttccaccagggcatctaccccgagctctacagcatcctcgagagccacagcttcgagtcggctaaccacccgctgctgcagcagctctggtacaaggcacgctacaccgaggccgagcgagcccgcggccggccgctgggcgcggtggacaagtaccggctacgcaggaaattccccctgccccgcaccatctgggacggcgaggagacggtgtattgtttcaaggagaagtcgcgcaacgcgctcaaggagctctacaagcagaatcgctacccttcgccggccgagaagcggcacctggccaagatcaccggcctctccctcacccaggtcagcaactggttcaagaaccgccggcagcgcgatcggaacccctccgagacccagtccaaaagtgagtcagatggcaatcctagcactgaagatgaatccagcaagggtcatgaagacttgtctcctcatcaactctccggttcatccgatggtgtcaccaacctcagcctttccagtcacctggagccagtatatatgcaacaaattggaaatgctaaaatatcgttaagctcctctggagttttattgaacggaagtttggtacctgcaagtacttcacctgtcttccttaatggtaattctttcattcagggacccaatggagttatccttaatggattaagtgtgggaaatacacagacagtatctttgaacccaccaaaaatggcatcaaacattatgagcaatggtatatccatgactgacatactggggtctacctcccaggacgtgaaggaattcaaagttctccagagctcttcagctaactcagcagccaccacctcctatagccccagtgctcccgtgtcgttcccagggctgatacccagtgccgaggtgaaaagagaaagtattcaaacagtggcttcccaggatggaggctctgtggtgacttttaccacaccagtgcaaattaaccagtatggcatcgtccagatccccaattctggagcaaacagccagttccttaatggaagcattggattctctccactgcagctgcctcctgtttcagtggcagcttcacaaggtaatatttcagtaaattcaagcacttcagatgggagcacatttacaagtgagtctgccacagtccaacaaggaaaggttttcttgagctctcttgctcccagtgcagtggtatacactgttcctaattcaggccagactataggatctgttaaacaggagggcgtggagaggagcctggtgttttctcagttgatgcctgtcaatcagaatgcacaagtaaatgcaaacctatcttctgaaagtatctccggaagtggcctccacccactgtcctcctcattagttaatgtatccccaactcacaacttctccctgacttccccaaccctactaaatcccactgagctaaaccctgacattgctgatagccagccaatgtctgcacctgtggcaagcaaatctactgtgacgtcagtcaacaacactaactatgcagctcttcagaactgctcccttatttctggtcaagatctactatcagtacccatgacccaggctgcccttggggaaatagttcccacaggtgaagaccaggtgggtcacccctccccaacagtacaccaggattttgtcagagaacatcatttagttatgcaaccagtagctaacataaaagaaaatttcctaacaaattctgagagcaaaacaacaagcaacttaatgatgctggactccaaatccaagtatgtcctagacagcatggtggagactgtctgtgaagacctggaaacagacaagaaagagcttgccaagctccagactgtccaattggatgaagatatgcaagacttgtaaacttaatttccttttttttccccctggcatcagcaggcaaatcttttcatgaagacttgaggacataaagcattttcccatttgaagggaaacattagttttgcaagtaaatgcattcaagagaccttggattgatctgcatgaagatcacagttaaatatgctgaagatcacagttaaatatgcaggagtagaactgcattactatagccttttggttctcttttcaatagatggagacagacatgagagatgaaatgtatcaagaagtcaaagtgtatcatttggttgacttaattctaaggtcaaatggaacttggtagttttttaaattaaaaatgtatacacttaaggtagaaaattaaatatcactacagaacattagaaacaatacagttctttttgtttacttttactccatgggcattggtaaggttaagaagcataagtggtacttcatacaattcgaaggatatatttttgaatatatgaattctgtttttcagcatttttgtgaaagtcttgctttgtctctatacagttccaaatgtgactgctaatagtttagttggctgagaaagttcacaactccactaagaaaactgaaacaagcttcagtatgagtgtaaatatattagtcctataagcaaggatttgggtaattacagtgaattttatttatgctgtatatgtttctcatacttcagacactatgaactgaaaaaaatgttttggtactgtttaatttatttttattgtttgaacaggaagtgaacagttatttccaacaaagttttactttttgtaggattttaaaagtaatgatttttatcagaagtctattaaggtcaatattaataacactgaaacaggaaatctaactcccattattggaattttggtactttttttcacattcttgttctttgtttagcaactctcttattcatttaaatatattttctatagaattctacgtgcctttttaccttgtggcctttttattcttattaaggtacagaagtgtcaacagaagcactgatgacttttttctactatccaacagggttactttcacagaattgctttaaatctgaagaacttaagtttcacttaatgttttatgatgcaaatggttgaacaactaattattgttgataacttttcattaaaagtaacaattacagactaccaacaaaaccaattctggaggctatgaaaacaaccattttaaagtaaggaaagagtttgtatcttctaatgagcagaattatgtcttgttgaaacttggttctattttgaatcggttgttgcattatgacgtatgactgttgtttgcactatgttatcgcatcttctttaaatcttttgggggtttctgtaagcagcttgtttgataatgctgtgagctgtggttatacagttgttatcaagactctgaagagaagatgtaataatacaaatacatttgtgtagtgagtaaagtctctggtctctgtaaagcaagaaaagtgaggaaattatctcttcagagatgaccgaaagttgtggaacatgaagatgtcatcacatcttgctttctttaagatcggaacaaagggtccactctagctctctgcatgctcgtcagtccagcatgttgagtaagggccatagaggatttcacttttgttgttattgcaaatggaacttgcaggcatacatgaagagccctttgaagaattcctcccccatcgtattttttctcctccatcattttgataacgaacttagatctcattttttaatggaattagtgaatgttcctaaactgctgaaattacatgttttttgttttgttttacaatgtcaggcattttagagttctcttccactccaccactacttatacagcttgtttgtttctttgggtgtgtgctgatcatgttactgagattaattacatggttctatagtgtagtttacataaaaatgatgttttccacttgattgctgcagatatcaagagtttgttgattttaaaatacccatccagtaagaaagatgggaaagtttaaaaagttatatatgttatacatacataactcgaaaagttttatattgtcttccaaaaatataataagcctctcttttattcacagttcttcctattttctaccttaagaaagaaaaaagatagcacaaaacttaagacttacctaggaaacctattcatactctaacattatgtaattatacccctacaattactccaggtagctacacaatataccaggtaagaatcaatgcacattataaagaacttactgttcttttctgttctttcagtaaaataagggccagttggtatgaagcccatatatatgtgaatgaaataagataaaccccatgatcttcctgcacagaattgcaaatattttttgcattgctaagggccagactcagaacccttggcagaatttctctcctacataaaccaatgcttaaagatgctgcaaatgaccctctaatgatcaccaaagaggaatcagaataggggtaaacaactctccttcaaaccccaacccctgctgctggctgcgggattttcaatgtgagcctctctatgcactcttatcagtcagaatatggcgtttagctcttagttacacccaataaaggacaatctattgtttaataaagagaacatgcgtttgtccctcagtcaagcaagagcacctgtgtcgtactgctttgtagctcacgtgtatccatagtaatgaaaagactgaatcgtacgcgtttcattgcctttcttgtgttatgaaaggcaagtagatgttatagccatagataaaattcatagttaaattgcacacagaccttcaatctccctgtatatgcccttgcgtcttgcatgttcaaagctagattatcgggtaggcgttgcagcctgctcgactacatgttagacaagtgtgcgttagtatataaccagaagttcttcattgtttaaaatgtttttgacagatcatctcatcataaaaatataaataattcaggaaggctgtggggaaaagaactacattttacagaagatgttttaaaagtcttcctagaacttagataatagagcgtaacagttattttgtatctaattgatacatttcatgttaatttcttattaccaccttacattttattaatcaaaattatatagcatgtgacttttggctttcagggttctcaaaaatttttattcttttgttgcatgtactgtatgtttagagacaaccagttatatactcaatatggttttactgtgccttacgcaaagagaaaatctataccgaatgtatatcgtggggtgggtaacggagtctctactgcactgtcaggtgatggcacacggagcgcgtcccagaacatttctgtgcttaattacccaaccaaagatctaaagtatgtatgttgtactgtaataggggtttatgcctggacaattaagtgttttatttgttttctgaaatgatgtgaacttatttttctaaacactatgctaaataaactttatatttatacataca

SEQ ID NO: 62 - DNA, 5924 bp, Sus scrofa
  source 1..5924: mol_type = other DNA
atgtcctcttcctcccccaccgggcagattgcaagtgcggcggatatcaagcaggagaatgggatggaaagctcctcggaagggcaggaggcaccccgagaagtggcggggggcgcgacggcggggctgagccccccggctccagccccctttcccctggagccgggggacgccgcggccgccgccgccagggtgagcggagagcaaggggcagtatcggcggcggcggctggagcagcggccgatcaggtacaactccactcggaacttctgggcaggcaccaccacgccgtggccgccgccgccgctgctgcgcagaccccactggccttctcgccagaccatgtagcctgcgtgtgcgaggcgctgcagcaggggggcaacctggaccgcctggcccggttcctgtggtccctgccccagagcgacctgctacgtggcaacgagagcctgctgaaggcgcgggcgctggtggccttccaccagggcatctaccctgagctctacagcatcctcgagagccacagcttcgaatcggccaaccacccgctgctgcagcagctctggtacaaggcacgctacaccgaggccgagcgagcccgcggccggccgctgggcgccgtggacaagtaccggttgcgcaggaaattccccctgccccgcaccatttgggacggcgaggagacggtgtattgtttcaaggagaagtcgcgcaacgcgctcaaggagctctacaagcagaatcgctacccttcgcccgccgagaagcggcacctggccaagatcactggcctctccctcacccaggtcagcaactggttcaagaaccgccggcagcgcgatcggaacccctctgagacccagtccaaaagtgagtcagatggcaatcctagcaccgaagatgaatccagcaagggtcatgaagatttgtctcctcatccactctctggttcatccgatggtgttaccaacctcagcctttccagtcacatggagccagtctatatgcaacaaattggaaatgctaaaatatctttaagctcttctggagttttgttgaacggaagtttggtacctgcaagtacttcacctgtcttccttaatggtaattctttcattcagggacccaatggagttatccttaatggattaagtgtgggaaatacacagacagtgtcattgaatccacccaaaatggcatcaaacattgtgagcaatggtatatccatgactgacatactggggtctacctcccaggatgtgaaggaattcaaagttctccagagttcttcggctaactcagcagccaccacctcctacagccccagtactcctatgtcatttccagggctgatacccagcactgaggtgaaaagagaaggtattcaaacagtggcttcccaggacggaggctctgtagtgacttttaccacaccagtgcaaattaaccagtatggcatcgtccagatccccaattccggagcaaacagccagttccttaatgggagcattggattctctccactgcagctgcctcctgtttcagtggcagcttcacaaggtaatatttcaataaattcaagcacttcagatgggagcacatttacaagtgagtccgccacagtccagcaaggaaaggttttcttgagctcgattgctcccagtgcagtggtatatactgttcctaattcaggccagactataggatctgttaaacaggagggcttggagaggagcctggtcttttctcagttgatgcctgtcagtcagaatgcacaagtgaatgcaaacctgtcttctgaaagcatctcggggagtggccttcacccactgtcttcctcattagttaatgtgtccccaactcacaatttttccctgactccccccaccctactaaatccaacagagctaaaccctgacattgctgatggccagccaatgtctgcacctgtggcgagcaaatctactgtgacatctgtcagcaacaccaactatgcaactcttcagaactgctcccttattactggtcaagatctattatcagtccccatgacccaggctgcccttggggaaatagttcccacagctgaagaccaggtgggtcacccctcccctacagtacaccaggattttggcagagaacatcatttggttatgcaatcagtagctagcataaaagagaatttcttaacaaattctgagagcaaagcaacaagcaacttaatgatgctggactccaaatccaagtatgtcctagagggcatggttgagactgtctgtgaagacctggaaacagacaaaaaagagcttgccaagctccagactgtccaattggatgaagatatgcaagacttgtgaacctaatttctttctcgcccctccccatcagcaggcaaatattttcatgaagacctaaggacaagcattttcccatttaaagggaaacactagttttgcaagtaaatgcattcgagaccctttggattgatctgcatgaagatcacagttaaatatgcagcagtagaactgcattactgcagccctttccacatatgttctcttttaaatagatggagacagaggagcaagataaaatacatcaagaagtaaaagtgtatcatttggttgacctaatataattctaaggtcaaatggaacttgacaaatagttttttaaattaaaaatgcatacacttaaggtagaagattaaatatcgctacagagcattagaaacaatgtaattctttttgtttacttttactccatgggcattggtaaggctaagaagtataaatggtactccacactattttaagggtatgtttttgaatatatacatttctgtttttcagctttttttgtaaaagtcctggttcgtctctacatttccaaatgtgattgttggtggttcagtgtggctgagcaaattcggaactccactaaaaactctgaaacttcagtatgagtgtaaatattttagtcttataagcaaggatttgagtaattacagtgaattttatttatgctgtatatgtttctcttatacttcagacactatgaactgaaaagaaaaatgttttcgtactgtttaatttatttttattgtttgaacaggaagtgaacagttgtttccaacaaagttttactttttgtagaattttaaaagtaatgatttttatcaggagtctattaaggtcaatattaataacactgaaacaggaaatctaactcccaatattggaattttggtactttttttttcacatacttgttctttatttagcaactctgattcgtttaaatatatcttctatagaattctatgtgcctttttaccttgtggcctttttattattcttactaaggtacagaagtgtcagcagaagcactgatgactcttttctactatccaacagggttaatttcaccacagaattgctttaaatctgaataacttaagtttcacttatattactgtagttaatgtttatgatgcaaatggttgaacaattattgttaatacttttcattacaatgaaagtaacagttttagactaccatgtaaaaccaattctagaggctatgaagacaaccattttaaagcaaagaaagaaaagtacattctatccagcagaattctgtcctcttgaaattatttttaaatattttgaatcagttattacattatgacatatgactgtttgcactatattatcatgtcttctttaaaagtaaatcttttggggtttctgtaagaagcttatttgataatgttgtgagctgtggttacagtttttatcaggactctgaccatagatgtaataatgcaaatacatttgtgtagtaaagtctctggtctctgaaaagcaggaaaagtgagaaaatggtcccttcagagatgaatgaaagttgaacatgcagatgtcacaacatcttgctttctttaagatgggaacaaagggtccactctaggtctttacatgcccatcagcccagcatgttgaacaagggctatagaggtaacttttgttgtcattacaaatggaacttgcaggcatagatgaagagtcctatgaagaattcctctcccataattttgatatgaacttagatccccccccttttttttaatggaactggtgaatgttcctgaactgctgaaactacgtggtttttgtttggttttgttttaaaacatcaggtttttagagttctcttacactccatcactccttatacagcttgtgtgtttctttgggtgtgtgctgatgatgttactgaggttatttacatggttctatagtgtaattgacatgaaaataatattatccactgattgctgcagatatcaagagtttgctaattatttcaaaatactacccatccaataagaaagatgggaatgtttggaaagttatatatgctatatataacaggaaaagttttatatgtcttccaaaatataagcttctcttattcatggttcttcctcttttcttccttaagaaaaaaaaaagatagcacaaaacttaagacttacctaggaaactattcatactccagtatgatgtaattatacccctacaattactccaggtagctacacaatataccaggtaagagtcaatgcacattataaagaacttattgttcttttctattctttcagtgaaatagggccagttggtatgtagcccacatatatgtgaatgaaataaaataaaccccatgatctccttgcacagaattgccttttcttgtttgtttgttttcctggaagtaccaagggccagactcagaacccttggagttcatgtagcagaatttctctcttacataaaccaatgcttgaagatgctgcaaatgatcctctaatgacccccaaggaggaatcagaataggggttaataactctccctacaaaccccaacccctgctgctggctgtggaattgttggtttgagcctacctatgcactctatcagccagaatttggcatttagcgcttagttacacccaggaaaggaccgtctatttaaggagaaggtgcatttgttccttaaacaagcaagaaaatctgtgttgtactgctttatagctcacgtgtattcatagtaatgtaaagactgaattgtacacaggtttcagtgcctttcttgtgttatgaaaggcaggtagatgttatagccatagataaaattcataggtgaattgcacactgaccttaaatctccctctatatgcccttgtgtcttgcatgttaaaagctggattattgggcagatgttgcagcctgccctactacatgttagacaattgtgcattagtacataaccagaagttcttcgctctttaaaatgtttttgacagatcatctcatcataaaaatataaataattcagaaaaactgtagggaaaagaattacgttttacgaaagatgtttcaaagtcttcctagaacttagatagagcctaatggttattttgtatctaactgatacatttcatgttaatttcttactaccaccatacattttattaagcaaaattatatagcatgtgacttggctttcagggttctcaaaacatttttactcttttgttgcatgtactgtatgtttagagacaaccagttatatactcagtatggttttactgtgccttacacaaagagaaaaactatacagaatgtatatcatggggtggggtaacagagtctttactgcactgttaggtgatggcacacggagcatgtcccagaacatttctgtgcttaaattacccaaccaaagagatctaaagtatgtatgttgtactgtaataggggtttatgcctggacatttaagtgttttatttgttttctgaaatgatgtgaacttatttttctaaacactatgctaaataaactttatatttatacat

SEQ ID NO: 63 - DNA, 1610 bp, Bos taurus
  source 1..1610: mol_type = other DNA
tatcgtctgcgctccgtggacgcgccctccgtccgccgaccgacagaactgcctatacccgcctgttctccttgcggcccggacacatagtatgaccattagatatttcgtctaccaaccattttccatggaaaaccaaagggaccaggccatgatagccactggcagctttgaagaacgggacatctttagagaagcttgaccttgaagacctcagcgtgggacctaacacagccggtctccggcagagttcctctgtctcgtcttgttgctgacagaaggtgcccccttctccagtttttgttcatctcctgggaggttgcaggaatcgtcattatggttgggttcaaggccacagatgtgccccctacagccactgtgaagttcctgggggcaggcacagctgcctgcattgctgacctcatcacctttcccctggatactgctaaagtccggctacagatccaaggagaaaggcaggggccaatgcaggctgcggccagtgcccagtaccgcggggtgctgggcaccatcctgaccatggtgcgcaccgagggcccccgcagcctctacagcgggctggtcgccggcctgcagcgccagatgagcttcgcctccgtccgcatcggcctctacgactccgtcaagcagttctacaccaagggctctgagcatgctggcatcgggagtcgcctcctggcaggcagcaccaccggtgccttggccgtggccgtggcccagccaacggatgtggtgaaggtccggttccaagcacaggcccgagctggagctggccggaggtaccagagcactgttgaggcctacaaaaccattgcccgagaggaggggtttaggggactctggaaagggacatctcccaatgtcgctcgcaatgccattgtcaactgtgctgagctggtgacctacgacctcatcaaggacactctcctgaaggcccacctaatgacagacgacctcccttgccactttacttctgccttcggggcgggcttctgcaccaccgtcatcgcctcccctgtcgacgtggtcaagacgagatacatgaactctgccctgggccagtacagcagcgctggccactgcgccctcaccatgctccagaaggagggaccccaagccttctacaaagggttcatgccctcctttctccgcttgggatcctggaacgtggtgatgttcgtcacctacgagcagctgaagagggccctcatggctgcccgcgcttcccgggaggctcccttctgagcagctgctgacctgatcagcgttggctctggctgcagcctggccctgcttccttttccttcctccccttctccccttccctctctccccatccctctcttctgctccctctcccaccacctcccttccccctccccacgttctcaccccttagctccctgtagcctctcacagtccaggtggacttgaccccagctgacactgtggagagcctggcatcagccaggatctcaagcccccagtcccctagaaagccctcagcttgactcttcttcctgcccccgagcccaactagcacgtcacccataaaacaagctcaaccttaaagaaaaaaaaaaaaaaaa

SEQ ID NO: 64 - DNA, 1366 bp, Sus scrofa
  source 1..1366: mol_type = other DNA
ggcacgaggaagcttgatcttgaaggcctcagtgtgagacctgacgaagccagattccggcagagttcctctatctcgtcttgctgctgagtggaggcgtctccttctgcggttcctctgtgtctcctgagagctagcaggaaatcagcattatggttggattcaaggccacagaggtccccccgactgccactgtgaagttcctgggggctggcacagctgcctgcatcgcagacctcatcacctttcccctggacacggctaaagtccggctgcagatccagggagaaaggcgggggccagtgcaggccgcggccagtgcccagtaccgcggggtgctgggcaccattctcaccatggtgcgcaacgagggcccccgcagcctctacaacgggctggtggccggcctgcagcgccagatgagcttcgcctccgtccgcatcggcctctacgactccgtcaagcatttctacaccaagggctcagagcatgctggcatcgggagccgcctcctggcaggcagcaccacgggggccttggctgtggccgtggcccagccaacagacgtggtaaaggtccggttccaagcgcaggcccgggccggcggaggccggcggtaccggagcactgtcgacgcctacaagaccatcgcccgagaggaggggctgcggggcctctggaaagggacctcacccaatgtcgctcgtaatgccattgtcaactgtgctgagctggtgacctatgacctcatcaaggacacgctcctgaaggccgacctcatgacagatgaccttccctgccacttcacgtccgccttcggggcgggcttctgcaccaccgtcatcgcctctcccgtggacgtggtcaagacgagatacatgaactctgccccgggccagtacagcagcgctggccactgtgccctcaccatgctccagaaggagggtccccgagccttctacaaagggttcacgccctcctttctccgattggggtcctggaacgtggtgatgtttgtcacctatgagcagctgaagagggccctcatggctgcccgcgcttcccgagaggctcccttttgagcctctcctgctgccgatccgactgcctcgggctttggctcctggccccgctgccctcctccctctatcacccgctgccccctttcccaccacgtcccttcccggtccccacattctcacccctcagttctccgttgcctctcatggtttgtccccatggagttgaccccaactgacactgtgggaagcctggcatcagccaggatctcaagtcctccgtcctttgaaaagtttagcccaactcctcatcctgccccacccagcccagttagcacatcacccat

SEQ ID NO: 65 - DNA, 1367 bp, Bos taurus
  source 1..1367: mol_type = other DNA
tcggcacgaggagcaatcagatccctgctgccgcctcctgggatggagccctgaggacccttaaagaggccccatgcttccgccaccctggctgcccctacagcctcctccatgcactgaagcccaaggctgggcagcagcctctctccttggacctccactgggccctggagggaccgggcagagcctttgggaccatggttggactgcaaccttcagagaggccccccacaacgtctgtgaagttcctggcggcgggcacagcggcctgctttgctgacctcctcacctttccactggacacagccaaggtccgcctgcagatccagggggagaaccaggcggccctggcggcccggagtgcccagtaccgcggagtgctgggcaccatcctgaccatggtgcgcaccgagggcccccgcagcctctacagcgggctggtcgccggcctgcagcgccagatgagcttcgcctccatccgcatcggcctctacgactccgtcaagcagttctacacccccaagggatcggaccactccagcatcattacccggattttggcgggctgtaccacgggggccatggcagtgacctgtgcccagcccacagacgtggtgaagatccgctttcaggccagcatgcacactgggctgggaggcaacagaaagtacagtgggacaatggatgcctacaggaccatcgccagagaggaaggggttcggggcctgtggaaaggaattctgcccaacatcacgaggaatgccattgtcaactgtggtgagatggtgacctatgacatcatcaaggaaaagctgctagactaccacctgctcaccgacaacttcccctgccacttcgtctctgcctttggagctggcttctgtgccaccctggtggcctccccggtggacgtggtgaagacgcggtatatgaactcgcccccaggccagtaccacagccccttcgactgtatgctgaagatggtgacccaggagggccccacagccttctacaagggatttacaccctcctttttgcgtttgggatcctggaatgtggtgatgttcgtcacctacgagcagatgaaacgggccttgatgaaagtccagatgctacgagactctccattttgactaagacaaggctactcagtatttaggacaaaaccagttaaggttggaataaagcagtgggtccgcgccacagggacacagacccacacatgtttgtttacagaactgtttacttgatgctgactcaagagacagaagaataggaaggattgtggtcttcagtgctttgtcttaagaacacctttgttttgcactgacaaggtggaaaataaattatattaatttgtgaaatcc

SEQ ID NO: 66 - DNA, 2245 bp, Sus scrofa
  source 1..2245: mol_type = other DNA
agggcactcgggtccctgctgccgcctcctggggtggagccctgaggagcctcaaggaggccctgcgcctcccccgccccggccggactgggcagggccagtcccctgctggtgctgcaggagccgggggaccgtcccagaagcccgggctccgtggctcggctcctgcagcctcccccgcacggaagcccaggccgggcagctctcctggaacctccctgctgggccctgggggacctggcagagcccctgggacatggtgggcctgaagcctcccgaggtgccgcccacgacggccgtgaagctcctgggggcgggcaccgcggcctgcttcgccgacctcctcaccttcccactggacacggccaaggtccgcctgcagatccagggggagaaccaggcggcccggagcgcccagtaccgcggggtgctgggcaccattctcaccatggtgcgcaacgagggcccccgcagcccctacaacgggctggtggccggcctgcagcgccagatgagcttcgcctccatccgcatcggcctctacgactccgtcaagcagctttacacgcccaagggatcggaccactccagcatcaccacccggattttggcgggctgcaccacgggggccatggcagtgacctgcgcccagcccacggacgtggtgaaggttcgattccaggccagcatacacgccgggcccaggagcaacaggaagtacagcgggacgatggatgcctacaggaccatcgccagggaggaaggggtccggggcctgtggaaaggaattctgcccaacatcacgaggaatgccatcgtgaactgtgccgagatggtgacctatgatgtcatcaaggagaaggtgctagactaccacctgctcacggacaacctgccctgccactttgtctctgccttcggagctggcttctgtgccaccgtggtggcctccccagtggatgtggtgaagacccggtacatgaactcgccccccggccagtaccagaaccctctggactgcatgctgaagatggtgacccaggagggccccacagccttctataaaggattcacaccttcctttttgcgcttgggaagctggaacgtggtgatgtttgtaagctacgagcagctgaaacgggccttgatgaaagtccagatgctgcgggaatctccgttttgagtaagacaaagccacctggcgtttcacgaaggcaaaaccagccaaggggtgacaggcgccgccgtgggtccgcgcacataaggccacaggcccacccgcatttacacaacagtctacttgccgccgactcggaaataggaggaggaaaactgtggtctccggtgcttcaccttcaggacacgttcgtgtggcactgacaagatgctcaataaattatattaatttgtgaaacccactaggatggatgcctaacatttaggcaaaagaagataaaccagaatggatccatatggataaaacagacgtttgcaaacttccgtcccctggaaaacgatcaggtgtcaacagataaggctgcaagcctgggacaggattcagactcgatacacttggaaatcagccgagtctagaggggcactgccaaagccacacaaggctttcaacggcagccttccgctacccacggatcaccagccacatttgccatgtgtctgcctggtagaacccaccaggattccgggattcgggaaaagaaaaagagcaaagccagctcctgtgacaaaggagccctgctgggccggctggggctgaggccgggatgctcgggagacccggagggtcaggagggcgggctcggcctcctctcccgtaaaatgggagcacaaagccctctctcctacctcccctggggtgtcacgaggatcaagcgagaagctgggcaaggaggcgctttataaaagctccatgaaatgtgtatataaggacgaggccttgggaatgttttcgtccgtcagtgaggcagtgccggggaagaagctttagaaagacacagatgtccagaggtcagaccaaaggaaccaggggctggaggggcagtggccggagccccgcgcccatgcggcctcccctccctgctggactgaggcaccttgaagggtgtggagtttggggacaaagaggtaaatgctgggcccagagcctcaagtaccaggactctgaatttggggaaaaggattatgactcaataaaggatgcgacaccttgggtgtgggaatgagaaga

SEQ ID NO: 67 - DNA, 7393 bp, Bos taurus
  source 1..7393: mol_type = other DNA
atgggggtccccaagaggaaggtctcgggcggccaggacggcgcggctgcccgggcgagcacagccaagcgggcccgcaccgaagagctcacgggcgtgcgcttcaaggctcagctgagggacccgcagggcgcggggccggccctggaggcatttgtttccgctgctaagaagctcccccgagaagatgtgtacgatgttgtggaggggtacatcaagatttctgtggagtgtgccgagattttccagctcctgagtggggagaagcgccctgaaagtgaaatgcttttagtatttcaagttttcgaggccatattattgcggacagccagtgacctttcccattttcacgtggtggggaccaacatcgtgaagaagctaatgaataaccacatgaagctcatctgtgagtccctgtacgcctcgggttacaggatggctcgagcctgcctgaacctgatggctgccatggtgacccagggtcccgaggccgccagggacgtctgcagccattttgatttgaataagaagaccttgtacaccctggtgaccaagagggatccaaagggagtacatgatgtccggctggcctacattcagttcgccctttcctttctgattgccggtgatgacagcacgatcacgcaagtgctggagttgaaagaatttattccttgcatttttagctcaggcatcaaggaagataggatctccaccatcagtatcctgttgtccacactgaaaacaaaggtagttcacaataaaaatatcacaaaaacccagaaggtgcgtttcttcactggccaagtgctgaaccacatagcatctctctacagctggaaggggattgtcgacgtgagcctggacagcgtcgagacttctgctaaagacgcagggaagaccatggtgcgggagcttgttcataacttcctgatggatctatgttgttcgctcaagcatgggattaatttttacgacgcatctctcggtaccttgggcagaggaggaaacctgactctcctgcacttcctgctgggtttgaaaaccgcagcggacgatgagctggtcgctgaactggtggtgaacattctgaaagtgtgcccggacctactgaacaagtacttcaaggaagtgacgttttcctttctcccaaggctgaagtccacctggtcaaataacatcagactgctaaacaagatctacgctgcccagccggacatctcccgggcgtttcagacccgagagttcatcccgctgcctcggctcctggccatggtgatggtgaccaccgcgcctctggtgtgcaacaagaccatgttcacgcaggcgctgaatttggacagcgtatccgtgaaacacacagctttatcccttgttgccgtcattttgaagagagccttaaagaccatcgagcactgcctgaataaagaggcctggcaggagtcaggcgtctacacggccgccatgatggaagacttcgtgcggctcttcagagaagctttgagcaagattttaccagacctgaatgccatcatctgggtctggcagtctctcaggaagcaagactcagaacacgatgacgagaaaggaaagaaaaaggccagcaagaccgctgctccctgcatggctcccgagagtgatgatgcagaaaccattcttctgaaagccgtgctgctccaggtcatctgcctctaccagaaggtggtcccgcacgtggtcatgcagtacaactttgacttcagcaagctcctgaaaggggtcatctcagaagagggcctgcgggaggaggtgcccccgcttctgcagcaccacattctcaaggtggccctggagctgcccgccagcaagtttctgtggttaaaggcccaggaaggcccagatgcagaaatcattggaggcaaacgatcggtgttttacttactgatgaaaatgtttgtgaccagtggccattcacagctcaagtcatcaaccaaacttctgattgtgaagatcctgcgggacacgggtgtgtttgagcacacctggccggagctggagctgtggctggagcacttggatgacatggcggaggagaacaaggaggctgtgattcagtttttggaacgcgtcctcctgacgttggtggcgaatccgtattcgtacacggacaaggcatctgacttcgtccaggaagccagcgccctgcaggcccccgggacgcggcaggacgctgatgacgccagcattcccatctcccacatcgacgatgtcctcgacatggtggatgtgctggtggaaggcagcgaaggcttggatgaggaggttggcttcgtcctgaatgaagacacgatcctcctcacgttcccgttcagcgccttggtccccgccgccctggaggccaggaataagttgctccttggaacagaaagtgaagcaggagagagcgttgtggcatatctgaccggggttctcacggacctgctccatacccagagagacccactggccctgtgtcttctgcttcagtcttacgaccagttggagcctccaagcctgccttgccgccaccggctgtccgggttcaacagatactacagcctctggatcccagagccagccctcgaggccctgccgctgcagacttcgggcagccctgccgcaccaggccccgcggccccgtccttctcctccctgttgcagacggcctacgccagtcaggaccaaggccagggcccaggcgggctcctactcctggacgacggggttcaggcgcagctgcgggcggccctcctgcgcctcccgatgcaccaggccctgcgcgccgccaaacacctgctgctgtacatcaggagcgccgtggagaacttcgcccagctgggccggagtgcggggccgcccctgctgcagctcctcctggatctcctccggcgcctggtcatccactgcctgcagctggacgcgcagaaccagcagaagtgcgaggccgcccaggccgagtccgacctctttgtggacatggagtccgtggcctcgctggagctggccagcgacaagacgctggaggaggtgctggtggctgttctcagacaccccacgctggagggctggttcctggccctggagcagcaggccctcccgccgcatgccctcagcccggtcctcgtgaagctcctggcagccagcctcagctcggggatccttccgctgctggtggccagtgctccagccctgcggagccttggtcagttgggcctgctggccaggtactcaaaggccatcacccagagtgtcctccgcgagctgcacagcctgagggccagctcggccacgtcaacgccaaaaaccctcctccctccgctcgaggccctgcgggggctgcacctctacatggaggacgccccactccgggaggtcaccctggccctgctgagcctgcccgaggcccgcctggcggcccagcgacccacccagcacccccggaaggagagaccgctgactgccctcgggaggaccctggtccagctgctgagtggccacgcccaggagcagccgcagagcggggagcccctctgggcctctgagtacgtgcggggcctggcagctctgctgcccgcgctggccgtggctgagctggacgccgtgctgcttggggctctgcaaagagagctggcgctggtccctgcggtcaggctggccctgcttgatgactgcctggcgcggcggacgccggcggccctggccacagcagccctgctcctgcagcacagctggtcgcaccggctgcgcttcgagcgctggtgccagcaggccggctccggactctgcctgcgggaggccccggacgacttcctccccatcgtccatgtgtacctgcagggcaggactcagggatgcttcacacgccccacagaagcttcctccgctgtcattcctgtcttgaggaaggctctgtggatgccgctgcaggccaggcttctcagcgccgacgggcccccagagtccgggctgccgcagcagatcctggcccagctggtcccgtttgccagagccaaggacctacgtgccctcatggaccgcttgcccagcttgctgcagactccagccagtcacacgagctgggtcgtggctgatgctgtctcagccgtcctggaagggtcagcagaagagctgcaagcctggaaaaagactctactgaaggcctgtgtgcagtggctaattgcatccttcagtggcggccagcaggaggacggtacccaggatcaggagaagcaaatgctgctgaggtttaacgaactgttgaattcacttaatgaagttgaccctggtgattggcagaaatttgtgaagaccggactcaagtttcggtatcaggaccgcgcgtttttggaagccctccgtgccgccacgcagctgttgtatctcccggaaagccccgtgtgctcgcagctcacccagctcccggtggtccacatgatgctcacccagcactccctgttcctgcccaccctgctgaggaccacagaggaggagcccgcagacacccccgtgagagaagcgctggtggacctgatggcggtggtggtgcggctgtgcccctctgtgtgtgcaagcagccacttcgcagtgctcctcggggcctatggtgcctcactcagcatcctggaccagaagatcttacttctcctccgggcctacgaacagaacaaccacagcctcgtcagcttcagggtactgctgtggggcccatcggccgtggagcatcagaagacgtgccggagcctgggcaagtcactatggcagcaaccgagcgttggggacatcctccgcctgctggatcgtgaccgcatgatgaagaccatcctccacttcccccagaaccggaggctgctgccccccgaggatgcacaggagccgatatttagagaccggagcacggtggatctcggggacctttatgacccctgtttcctccttcatctcttcagtgaactgaccaggccagaatttgtggtggattgtagaaaattcgtggattcaaatgctttgggcctgactgtcgcggccctcagcagctacgacccacagatgcgcgccgcagcctacgcggtgctggcggcctactactcacacctggagggggcgcgtttccgggagcagccccaggtactgtacctgttggacgtggtcaggaatgggatccgaaccaagaacatgagactcaccttcaccctgactctcttcattgccaaggcagccttgcagattttgaaaccagaggagcatatgtacatgaagatcaacaagttcctgctgtcccacgagtacctgaacatgagcaaagttccaggcttctaccagttcttctacagctcggacttcgagcagaagacggagcgggagtgggtgttggggctccttcgccagggaatgcgggacaagcactgcttcgagctgtgcgccaggcggggcgtcctccacatcgtcctgtccttcttcggcagcccgctgtgtgacgaggcggcccagaactggattctggagattctgcagaatgccgcccggaatgccaggtctgcctatgagatgctgcgggactacagcctcctgacgtgggtcctaaacatcctggaaagcaagtttctggagaccgagctgctttccaacctcatctccctgctgcacacactgtgggtgaccacgctgggcaacagagaagtggaggccggggcacagcctccctgcaagcctggctcccaggagcccccgaagctgctggccctgcacctggtggacgagttcctctacattctccttgtgctcacgaagcacctgaggcccaccttggcctccgcccagctgaccggcttcttcggggcactcgactcggtcctgaggtaccgggccaccgtgctgcaggccttcaaggacatgaaccgtctgaccatgaacgccacggtgctgtccacccgggatgtccttgtgctcctgcacaagtggagcctcatcgagagagacgtccggctccaggaggacctgagggccgccacggaaaagtgccaagtccgggagctgctgaagatgctcaaggagaagaacaagcccagcgcgccagcccgagccaaaggcatgcgggcccggaagcggagacctggcgaggcggaggaggcggccgaccccgagctgccggcgtccgccctggaggcgtgcagcggcctcctgaggtccatcctgacccactgggcacccgagttccccagcgccgacccggagcgcgaagacccgggccccgtgggtgccatcgcctccctggtagccggctgggtgctgcgctccgcagccaggagtacccttggccgggcggacgcggcaggcctcctgggctggctcaagagccacgtcctgcaacagccggcggtggtggctgagcttctccgcgatggcaccgtgacgagcagcctgttccgcctgtacagccggttctgcggtgctgtggagccggcgggatcagaggaccgcttggccggcctcttcaacgccgtcctgctgcgcctggtggcggcccggggcaccgcggggatccccctccagccggtgctggagaccgtccacctctcctccctggacgccgaggaggatgcggacacacgagccgccgccgcgttcctggtatctctgtacattaaggacatctggctgggggcccagcggccagacaccctcctgagccacgtccggatggtcctcgacactgcggaggacttgggagggggcaacgaggaggccatcgtccggctctgtagggacatcgcggccacggtccctgacatctgactcctgcggctggcacctccggctccaggtttcaggagtcgggttgagtcgggggtttccatggaaagcctgtgagacgcaggctgtgcgtgtcgacctgctgttctgacgggaaatgttggcttctctaggtgactgatacaaaagaagctcaagggaccgtgtatgcagagaggtcagggttacagtgggcctgggtggccctgggcccacgagccctggtcagggaagacccttcctctgaggtgcttgttgttcctcagagaaatacctcaaactgaggtaacgctaaagttgaaatgtctaaaagtatacgagacacagttgcatgaagagagatgtttcaagtttcctcttcataagaagtcacagcaaagctttgtgtaactttttgaataaataaacctttccagtattgtgttaaaggtattttactaaataaaaggatgttgtactaaattcttaaagtataaaactattttgtcttctgcttggaaaagactgctttatatttttaaaaattccatttgaaagcaaggactttgaattttataacttgttaataaatgtctatttttgttag

SEQ ID NO: 68 - DNA, 7576 bp, Capra hircus
  source 1..7576: mol_type = other DNA
tgtgatccctgagacgctggctgggtcaccgcgtgtttgcttccgccaggtctccggccgcgtggatcccggcgcgcccccgccctcttggccatgggggtccccaagaggaaggtctcgggcggccgggacggcgcggctgccccggccggcgcaaccaagcgggcccgcaccgaagagctcacgggcgtgagcttcaaggctcagctgagggagccgcagggcgcggggccggccctggaggcatttgtttccgctgctaagaagctcccccgagaagacgtgtatgatgttgtggaggggtacatcaagatttctgtggagtgtgccgagattttccagctcctgagtggggagaagcgccctgaaagtgaaatgcttttaatatttcaagttttcgaggccatattattgcggacagccagtgacctttcccattttcacgtggtgggaaccaacattgtgaagaagctgatgaataaccacatgaagctcatctgtgagtccctgtatgcctcgggttacaggatggctcgagcctgcctgaacctgatggccgccatggtgacccagggtcccgaggccgccagagacgtctgcagccatttcgatttgaataagaagaccttgtacaccctggtgaccaagagggatccaaagggagtacatgacgtccggctggcctacattcagtttgccctttcctttctgattgctggtgatgacagcaccatcacgcaagtgctggagttgaaagaatttattccttgcatttttagctcaggcatcaaggaagataggatctccaccatcaacattttgttgtccacactgaaaacaaaggtagttcacaataaaaatatcacaaaaacccagaaggtgcgtttcttcactggacaagtgctgaaccacatagcatctctctacagctggaaggggattgttgacgtgagcctggacagcgtcgagacttctgctgaagacgcagggaagaccatggtgcgggagcttgttcataacttcctgatggatctgtgctgttcgctcaagcatgggatcaatttttacgatgcatctctcggtaccttcggcagaggaggaaacctgactctcctgcacttcctgctgggtttgaaaaccgcagcggatgatgagctggtcgctgaactggtggtgaacattctgaaagtgtgcccggacctactgaacaaatacttcaaggaggtgacgttttcctttctcccgaggctgaagtccacttggtcaaataacatcagactgctaaacaagatctacgctgcccagccggacatctcccgggcgtttcagacccgagagttcattccgctgcctcggctcctggccatggtgatggtgaccaccgcgcctctggtgtgcaacaagatcatgttcacgcaggcgctgaacttggacagcgtatccgtgaaacacacagctttatcccttgtcgccatcattttgaagagagccttgaagaccatcgagcactgcctgaataaagaggcctggcaggagtcgggcgtctacacggctgccatgatggaagacttcgtgcagctcttcagagaagctctgagcaagattttaccagacctgaatgccatcatctgggtctggcagtctcttagaaagcaggatgcagaacaggaggacgagaaaggaaagaaaaaggccagcagggctgctgctccctgcgtggctcccgagagcgatgatgcagaaaccattcttctgaaagccgtgctgctccaggtcatctgcctctaccagaaggtggtcccacacgtggtcatgcagtacaactttgacttcagcaagctcctgaaaggggtcatctcagaggagggcctgcgagaggaggtacccccgcttctgcagcaccacattctcaaggtagccctggagctgcctgccagcaagtttctatggttaaaggcccaggaaggcccagatgcagaaatcattggaggcaagcgatcagtgttttacttactgatgaaaatgtttgtgaccagtggccattcacagctcaagtcatcaaccaaacttctgattgtgaagatcctgcgggacacgggcgtgtttgagcatacctggccagagctggagctgtggctggagcacttggatgacacggtggaggagaacaaggaggccgtgattcagtttctggaacgcgtcctcctgacgttggtggcgaatccgtattcgtacacggacaaggcgtctgacttcgtccaggaagccagtgccctgcaggctcccgggacgcggcaggacgccgatgactccagcatccccatctcccacatcgacgatgtcctcgacatggtggatgtgctggtggagggcagcgaaggcttggatgaggaggttggcttcgtcctgaatgaagacatgatcctcctcacgttcccgttcagcgccgtggtccccgccgccctggaggccaggaataagttgctccttggaacagaaagcaaagcaggagacagcatcgtggcctatctgactggggttctcacggacctgctccacacccagagagacccgctggccctgtgtcttctgcttcagtcttacgaccagttggagcctccaagcctgccttgccgctgcctgctgtcccggttcaacaaatactacagcctctggatcccggagccagcccgcgaggccctggtgagccacggcgaccctgcggaaaccaggccttcctttccctgggaagccccggaggcccctgcccgtgggccacggcagcttgtagcccagccgctgcagacttcaggcagccctgccccgccaggccccgcggccccgtccttctcctccttgctgcagacggcctacgccagtcaggaccaaggccagggcccaggcgggctcccgctcctggacgagggggttcaggcgcagctgcgggccgccctcctgcgcctcccgatgcaccaggttctgcgtaccgccaaacacctgctgctgtacatcagaagcgccgtggagaacttcgcccagctgggccggagtgccgggccgcccctgctgcagttcctcctggatctcctccggcgcctcgtcatccactgccagcagctggacgcccagaaccagcagaagtgccatgccgcccaggcggagtccgacctctttgtggacatggagtccatggcctctctggagctggctagtgacaagacgctggaggaggtgctggtggctgtcctcagacaccccacgctggagggctggttcctggccctggagcggcaggccctcccgccgcatgccctcagcccggtcctcgtgaagctcctggcggctagcctcagctcggggatccttccgctgctggtggccagtgccccggccctgcggagcctcggtcagctgggcctgctggccaggtactcggaggccgtcacccagagtgtcctccgtgagctgcgcagcccgagggccggctcggccacatcgacgccgaagaccctcctccctccgctcgaggccctgcgggagctgcacccctacatggaggacgccccgctccgggaggtcatcctggccctgctgagcctgcccgaggcccgcctggccacccagcgacccacccagcccccccggaaggagaaaccgctgaccgccctcgggaggaccctggtccagctgctgagcagccacgcccaggagaagccgcagagtggggagctcctctgggcctccgagtacgtgcggggcctggcggctctgctgcccatgctggccgtggctgagctggacaccgtgctgcttggggctctgcaaagggagccagcgctggtccccgcggtcgggctaaccctgctcgacgactgcctggcacggcggacgccagcagccctggccacggcggccctgctcctgcggcacagctggccgcaccggctgcgcttcgagtgctggtgccagcgggctggctccagactccgcctgcgggaggccccagacgacttcctccccgtcgtccacgtgtacctgcagggcaggacgcaggggcgcttcacacgccccacaggagcttcctccgctgtcattcctgtcttgaggaaggctctgtggatgccgctgcagaccaggcttctcagtgctgacgggcccccagagtccgggctgccgcagcagatcctggcccagctggtcccgtttgccagagccaaggacctgcgtgccctcatggaccgcttgcccagcttgctgcagactccagccagtcacacgagctgggttgtggctgacgccgtctcagccatcctggaagggtcggcagaagagctgcaagcctggaagaagactctactgaaggcctgtgtgcagtggctaattgcatccttcagtgacggccagcaggaggatggtccccaggatcaggagaagcaaatgctgctgaggttaaacgaactgttgaattcacttaaggaagttgaccctggtgattggcagaaatttgtgaagaccggactcaagtttcggtatcaggactgcgcgtttttggaagccctccgtgccgccacgcagctgttgtatctctcagaaagccccgcgcgctcgcagctcatccagctctcggtggtccacatgatgctcacccagcactccctgttcctgcccaccctgctgagggccgcagaggaggagcccgcagacagccccgtgagagaagcgctggtggaactgatggcggtggtggtgcagctgtgcccctctgtctgtgccagcagccacttcgcggtgctccttggggcctacggtgcctcactcagtatcctggaccagaagatcttacttctcctccgggcctacgaacagaacaaccacagcctcgtcaacttcagggtactgctgtggggcccagcggctgtggagcatcagaagacatgccggagtctgggcaagtcgctatggcagcagccgagcgtcggggacatcctccgcctgctggatcgtgaccgcatgatgaagaccatcctccacttcccccagaaccggaggctgctgccccccgaggacgcacaggagccgatatttagagaccagagcgcggtggatctcggggacctttatgacccctgtttcctccttcatctcttcagtgaactgaccaggccagaatttgtggtggattgtagaaaattcgtggattcaaatgctctgggcctgactgtcgcggccctcagcagctacgacccacacatgcgcgccgcagcctactcggtgctggcggcctactactcgcagctggagggggcgcgcttccgggagcagccccaggtactgtacctgttggacgtggtccggaacgggatccgaaccaagaacatgagactcaccttcaccctggctctcttcattgccaaggcagccttgcagattttgaaaccagaggagcgtatgtacatgaagatcaacaagttcctgctgtcccacgagtacctgaacatgagcaaagttccaggcttctaccagttcttctacagctcggacttcaagcagaagacggagcgggagtgggtgttggggctcctccgccagggaatgcgggacacgcactgcttcaagctgtgtgccaggcggggcgtcctccgcatcgtcctgtccttcttcagcagcccgctgtgcgacgaggcggcccagaactggattctggagattctgcagaatgccgcccgggatgccaggtctgcctatgagatgctgcgggactacagcctcctgacgtgggccctaaacgtcctggaaagcaagtgtctggagactgagctgctttccaacctcatctccctgctgcacacactgtgggtgaccacgctgggcaacagagaagtggagcccggggcacagcctccctgcaaacctgggtctcaggagcccccgaagctgctggcgctgcacctggtggacgagttcctctatgttctccttatgctcacgaagcacctgaggcccaccttggcctccgcccagctgaccggcttcttcggggcgctcgactcggtcctgaggtaccgggccaccgtgctgcaggccttcaaggacatgaaccgtctgaccgtgaacgccacggtgctgtccacccgggacgtccttgtgctcctgcacaagtggagcctccttgaaagagacgtgcggctccaggaggacctgagggccgccgcggagaagtgccaagtccgggagctgctgaagatgctcaaggagaagaacaagccggatgcacccgcccgagccaaaggcctgcgggcccggaagtggagacccagcgaggcggaggaggcggccgaccccaaactgctggcgtccaccctggaggcgtgcaggggcctcctgaggtccatcctaacccactgggggcccgagttccccagcgccaacccagagtgtgaagacccgggccccgtgggtgtcgtcacctccctggtggccggctgggtgctgcgctccgcagccgggagcacacttggccgggcggacacggcaggcctcctgggctggctcgagagccacgtcctgccacggccggcagtggtggctgagcttctccgcgacagcaccgtgacgagcggcctgttccgcctgtacagccggttctgtggcgccacagagccggcggagccggaggaccgcttgcccggcctcttcaacgccgtcctgctgcggctggtggcggcccggggcaccgcggagagccccctccagccggtgctcgaggccatccacctctcctccctggacaccgaggagaatgcagacacacaagccgctgccgcattcctggtgtctctgtacattaaggacatctggctgggggcccagcagccagacaccctcctgagccatgtccagatggtcctcgacgccgtggagaactcaggagggggtgacgaggaggccatcgtccggctctgcagggacatcgcggccacggtccccgacacctgacccttgtggctggcacctccggctccaggttccaggagtcaagttgagtcggggatttccatggaaggcctgtgagacgcaggttgtgcgtgtcgacctgctgttctgacgggaagccttggtttcctttaggtgactgatacagaagaagctcaaagaaccgtgtatgcagagaggtcagggttacaggtggcctgggcggccctaggcccacgagtcccggtcagggagacccttcctctacggcacttgctgttcctcagagaaatacctcaaactgaggtaaccctgaagttgaaatgtctaaaagtatatgagacacaggagcctgaagacagatgggtcacgtttcctcttcataagaagtcgcagcaaagttctatgtaactttttgaataaacaagcctttccaggattgtgttaaagttattttactaaataaaaggatgctgtactaaattcttaaagcataaaactattttgtcttctgcttggaaaagacagctttatatttttaaaaattccttttgaaagcaaggactttgaattttataacattaataaatgtctatttttgtta

SEQ ID NO: 69 - DNA, 7316 bp, Ovis aries
  source 1..7316: mol_type = other DNA
gtttgcttccgccaggtctccggccgcgtggatcccggtgcgcccccgccctcttggccatgggggtccccaagaggaaggtctcgggcggccgggacggcgcggctgccccggccggcgcagccaagcgggcccgcaccgaagagctcacgggcgtgagcttcaaggctcagctgagggacccgcagggcgcggggccggccctggaggcatttgttaccgctgctaagaagctcccccgagaagacgtgtatgatgttgtggaggggtacatcaagatttctgtggagtgtgccgagattttccagctcctgagtggggagaagcgccctgaaagtgaaatgcttttaatatttcaagttttcgaggccatattattgcggacagccagtgacctttcccattttcacgtggtggggaccaacattgtgaagaagctgatgaataaccacatgaagctcatctgtgagtccctgtatgcctcgggttacaggatggctcgagcctgcctgaacctgatggccgccatggtgacccagggttccgaggccgccagagacgtctgcagccatttcgatttgaataagaagaccttgtacaccctggtgaccaagagggatccaaagggagtacatgacgtccggctggcctacattcagtttgccctttcctttctgattgccggtgatgacagcaccatcacacaagtgctggagttgaaagaatttattccttgcatttttagctcaggcatcaaggaagataggatctccaccatcaacattttgttgtccacactgaaaacaaaggtagttcacaataaaaatatcacaaaaacccagaaggtgcgtttcttcactggacaagtgctgaaccacatagcatctctctacagctggaaggggattgttgatgtgagcctggacagcgtcgagacttctgctgaagatgcagggaagaccatggtgcgggagcttgttcataacttcctgatggatctgtgctgttcgctcaagcatgggatcaatttttacgatgcatctctcggtaccttcggcagaggaggaaacctgactctcctgcacttcctgttgggtttgaaaactgcagcagacgatgagctggtcgctgaactggtggtgaacattctgaaagtgtgcccggacctactgaacaaatacttcaaggaagtgacgttttcctttctcccgaggctgaagtccacttggtcaaataacatcagactgctaaacaagatctacgctgcccagccggacatctcccgggcgtttcagacccgagagttcatcccgctgcctcggctcctggccatggtgatggtgaccaccgcgcctctggtgtgcaacaagatcatgttcacgcaggcgctgaacttggacagcatatccgtgaaacacacagctttatcccttgtcgccgtcattttgaagagagccttgaagaccatcgagcactgcctgagtaaagaggcctggcaggagtcgggcgtctacacggccgccatgatggaagacttcgtgcggctcttcagagaagctctgagcaagattttaccagacctgaatgccatcatctgggtctggcagtctcttagaaagcaggatgcagaacaggaggacgagaaaggaaagaaaaaggccagcagggctgctgctccctgcgtggctcccgagagcgatgatgcagaaaccattcttctgaaagccgtgctgctccaggtcatctgcctctaccagaaggtggtcccacacgtggtcatgcagtacaactttgacttcagcaagctcctaaaaggggtcatctcagaggagggcctgcgggaggaggtgcccccgcttctgcagcaccacattctcaaggtagccctggagctgcccgccagcaagtttctatggttaaaggcccaggaaggcccagatgcagaaatcattggaggcaagcgatcggtgttttacttactgatgaaaatgtttgtgaccagtggccattcacagctcaagtcatcaaccaaacttctgattgtgaagatcctgcgggacacgggcgtgtttgagcatacctggccagagctggagctgtggctggagcacttggatggcacggcggaggagaacaaggaggccgtgattcagtttctggaacgtgtcctcctgacgttggtggcgaacccgtattcgtacacagacaaggcatctgacttcgtccaggaagccagcgccctgcaggctcccgggacgcggcaggacgccgatgactccagcatccccatctcccacatcgatgatgtcctcgacatggtggatgtgctggtggagggcagcgaaggcttggatgaggaggttggcttcgtcctgaatgaagacatgatcctcctcacgttcccattcagtgccgtggtccccgctgccctggaggccaggaataagttgctccttggaacagaaagcaaagcaggagagagcatcgtggcatatctgactggggttctcacggacctgctccacacccagagagacccgctggccctgtgtcttctgcttcagtcttatgaccagttggagcctccaagcctgccttgccgctgcctgctgtcctggttcaacaaatactacagcctctggatcccggagccagcccgcgaggccctgctgctgcagacttcaggcagccctgccccgccaggccccgcggccccgtctttctcctccttgctgcagatggcctacgccagtcaggaccaaggccagggcccaggcgggctcccgctcctggacgagggggttcaggcgcagctgcgggccgccctcctgcgcctcccgatgcaccaggctctgcgcgccgccaaacacctgctgctgtacatcaggagtgctgtggagaacttcgcccagctgggccggagtgccgggccgcccctgctgcggttcctcctggatctcctccggcgcctggtcatccactgccagcagctggacgcccagaaccagcagaagtgccaggccgcccaggcggagtccgacctctttgtggacatggagtccatggcctcactggagctggccagcgacaagacgctggaggaggtgctggtggctgtcctcagacaccccacgttggagggctggttcctggccctggagcggcaggccctcccgccgcatgccctcagcccagtcctcgtgaagctcctggcggctagcctcagctcagggatccttccactgctggtggccagtgccccggccctgcggagcctcggtcagctgggcctgctggccaggtactcggaggccgtcacccagagtgtcctttgtgagctgcgcagcccaagggccggctcggccacatcgacgccgaagaccctcctccctccgctcgaggccctgcaggagctgcacccctacatggaggacgccccgctccgggaggtcaccctggccctgctgagcctgcccgaggcccgcctggccacccagcgacccacccagcccccccggaaggagaaaccgctgaccgccctcgggaggaccctggtccagctgctgagcagccacgcccaggagaagccgcagagtggggagctcctctgggcctccgagtacgtgcggggcctggcggctctgctgcccacgctggccgtggctgagctggacgccgtgctgcttggggctctgcaaagggagccggcgctggtccccgcggtcgggctaaccctgctcgacgactgcctggcacggcggacgccagcagccctggccacggcggccctgctcctgcggcacagctggccgcaccggctgcgcttcgagtgctggtgccagcgggctggctccggactccgcctgcgggaggccccagacgacttcctccccgtcgtccacgtgtacctgcagggcaggacgcaggggcgcttcacacgccccacaggagcttcctccactgtcattcctgtcttgaggaaggctctgtggacgccactgcagaccaggcttctcagtgctgacgggcccccagagtctgggctgccacagcagatcctggcccagctggtcccgtttgccagagccaaggacctgcgtgccctcatggaccacttgcccagcttgctgcagactccagccagtcacacgagctgggtcgtggctgacgccgtctcagccatcctggaagggtcggcagaagagctgcaagcctggaagaagactctactgaaggcctgtgtgcagtggctaattgcatccttcagtgacggccagcaggaggatggtatccaggatcaggaaaagcaaatgctgctgaggttaaatgaactgttgaattcacttaaggaagttgaccctggtgattggcagaaatttgtgaagaccggactcaagtttcggtatcaggaccgcgcgtttttggaagccctccgtgccgccatgcagctgttgtatctctcagaaagccccgcatgctcgcagctcatccagctcccggtggtccacatgatgctcacccagcactccctgttcctgcccaccctgctgagggccgcagaggaggagccctcagacagccccgtgagagaagcgctggtggaagtgatggcggtggtggtgcagctgtgcccctctgtctgtgctagcagccacttcgcggtgctccttggggcctacggtgcctcactcagtatcctggaccagaagatcttacttctcctccgggcctacgaacagaacaaccacagcctcgtcaacttcagggtactgctgtggggcccagcggctgtggagcatcagaagacgtgccggagcctgggcaagtcgctatggcagcagccgagcgtcggggacatcctccgcctgctggatcgtgaccgcatgatgaggaccatcctccacttcccccagaaccggaggctgctgccccccgaggatgcacaggagccgatatttagagaccagagtgcggtggatctcggggacctttatgacccctgtttccttcttcatctcttcagtgaactgacccggccagaatttgtggtggattgtagaaaattcgtggattcaaatgctctgggcctgactgtcgcggccctcagcagctacgacccacacatgcgcgccgcagcctacgcggtgctggcggcctattactcgcagctggagggggcgcgcttccgggagcagccccaggtactgtacctgttggacgtggtccggaacgggatccgaaccaagaacatgagactcaccttcaccctggctctcttcattgccaaggcagccttgcagattttgaaaccagaggagcgtatgtacatgaagatcaacaggttcctgctgtcccacgagtacctgaacatgagcaaagttccaggcttctaccagttcttctacagctcggacttcgagcagaagacggagcgggagtgggtgttggggctcctccgcgagggaatgcgggacacgcactgcttcaagctgtgcgccaggcggggcgtcctccacatcgtcctgtccttcttcagcagcccactgtgcgacgaggcggcccagaactggattctggagattctgcagaatgccgcccgggatgccaggtctgcctatgagatgctgcgggactacagcctcctgacgtgggtcctaaacgtcctggaaagcaagtttctggagactgagctgctttccaacctcatctccctgctgcacacactgtgggtgaccacgctgggcaacagagaagtggagcccggggcgcagcctccctgcaagcctgggtctcagaagcccccgaagctgctggcgctgcacctggtggacgagttcctctacgttctccttgtgctcacgaagcacctgaggcccaccttggcctccgcccagctgaccggcttcttcggggcgctcgactcggtcctgaggtaccgggccaccgtgctgcaggccttcaaggacatgaaccgtctgaccgtgaacgccacggtgctgtccacccgggacgtccttgtgctcctgcacaagtggagcctcctcgagagagacgtgcggctccaggaggacctgagggctgccgcggagaagtgccaagtccgggagctgctgaagatgctcaaggagaaaaacaagcccgactcccaagccaaaggcctgcgggcccggaagcggagacacggcgaggtggaggaggccgccgaccccaaactgctggcatccaccctggaagcgtgcaggggcctcctgaggtccatcctgacccactgggggcccgagttccccagcgctgacccagagtgtgaagaccctggccccgtgggtgttgtcacctccctggtggccggctgggtgctgcgctccgcagccgggagcacgcttggccgggcggacatggcgggcctcctgggctggctccagagccacgtcctgccgcggccggcagtggtggctgagcttctccacgacagcaccgtgacgagcggcctgttccgcctgtacagccggttctgtggagccgcggagccggaggaccgcttgcctggcctcttcaacgctgtcctgctgcggctggtggcagcccggggcgctgcggggagccccctccagccggtgctcgaggccgtccacctctcctccctggacaccgaggaggatgcagacacacaagctgctgccgcattcctggtgtctttgtacattaaggacatctggctgggggcccagcggccagacaccctcctgagccatgtccggatggtccttgacgccgtggagaactcaggagggggcgatgaggaggccatcgtccggctctgcagggacatcgcggccactgtccccaacacctgacccttgcggctggcacctccagctccaggttccaggagtcaagttgagttgaggatttccatggagggcctgtgagacgcaggctgtacgtgtcgacctgctgttctgacaggaagccttggtttcctttagatgactgatacagaagaagctcaaaggaccgtgtatgcagagaggtcagggttacaggtggcctgggtggccctaggcccacgagccccgctcagggagacccttcctctacggcgcttgctgttcctcagagaaatacctcaaactgaggtaaccctgaagttgaaatgtctaaaagtatatgagacacaggagcctgaagacagatgggtcatgtttccttttcataagaagtcgcagcaaagttctatgtaattttttgaataaacaagcctttccaggattgtgttaaagttattttactaaataaaaggatgctgtactaaattc

SEQ ID NO: 70 - DNA, 1655 bp, Bos taurus
  source 1..1655: mol_type = other DNA
cccgcggcggggaggaggcgctggcagcctgggccgggaggtggcagggggccgcggagacgtcggctaccgactcgtcctgccatgtgacgccgccctccgtcccgcgatcccccgtgcgccccgggcccgcgcccgcgctccgtcgcgcggcacatcatgctgctgccgggccacgcgcgcccaccgtccgccccgcaacccgcgcagcatcccggcctccgcaggcaggtagagcctccggggcagctcctgcgtctgttctactgcaccgtcctggtttgctccagagagatcgcggcgctcacggacttctccggttacctaaccaaactcctgcaaaaccacaccgcctatgcctgtgacggggaccatttgaatctgcagtgtcctcggcattccacgataagtgtccagtcggcattttatgggcaagattaccaaaagtgtagtccgcagcagcctgcctcccagagggaagacagcctaacctgtgtggtgcccaccacactgcagaaggtgctggacgagtgtcagaaccagcgggcctgccacctcctggtcaatagccgtgtttttggacctgacctttgtccaggaagcagtaaatacctcctggtctcctttaaatgccaacctaatgagttaaaaaacaaaactgtgtgtgaagaccacgagctgaaactgcactgccacgagtccaagtttctcaacatctactctgcgacctacggcaggaggacccaggagaggcatgtctgctcctcggaagccgagcggctccccccctttgattgtctgtcttactcggctctgcacgtgttatccaggagatgttacgggaagcagaggtgcaaagtcctcgtcaacaaccaccatttcggaagcccctgtctgccaggagtgaaaaaatatctctcggtcttctacgcgtgtgttcccaagaatatactcaccgcaattgacccatcagttgctaatctaaaaccttctgtgaagcagaaagatggcgactatggcgtagaccttgacccaagggaaccgagggttctgaggaaagatggtgtgattgtcagccactcgttggcagcatttgcttacattagaggctttcgatgcgatgcaaggaaggagacagcctccctggtggctcacatggtgaagcgtcggcctgcagtgcgggagacctgggatcgagccctgggtggggaagatcccctggagaaggaaatggcaacccactccggtactcttgcctggaaaattccatgggcggaggagcctggtgggctacggtccatggagtcgcaaagagccggacacaactgagaacttcactttcacacagaaggagacactgaaggggaggctgagtgtacgagggattctgtgactgttccacaggctctcgagagcctaatggaaggatgtcgggtcagcgtgagagacggctacatactctgtggctccttgggaccatcaaaaatgggaacaaaggacacgatgggaccacccaggatggtccccaagcagattacggggccctggacactctacattctagtgaagttcctcattcaacagctgctatttttttttttttttaattggaggataattaccttacaatattgtgttggtttctgccatacagc

SEQ ID NO: 71 - DNA, 1941 bp, Capra hircus
  source 1..1941: mol_type = other DNA
tgcaaggcgctccagtcgcgtgctgttgggggcgcccttcgcggcgcgcagcgcgccagggccccggcctctggcagcctcgggggcgcgaaggggctacactttcccttcgccccccgccctcgtttcctcctgcgcggcccgcagcagagatttcctctcggggaaactacgcggatccttcccggggctcctcgccccgccccaggtctccgccccctcctctgcgctcgggagccggtgctcgcccgcggcaaggaggaggcgctggcagcctgggccgggaggtggcagggggccgcggagacgtcggctaccgactcgtcctgccatgtgacgccgccctccgccccgcgatcccccgtgcgccccgggcccgcgcccgcgctccgtcgcgcggcacatcatgctgctgcccggccacgcgcgcccaccgtccgccccgcaacccgcgcagcatcccggcctccgcaggcaggtagagcctccggggcagctcctgcgtctcttctactgcaccgtcctggtttgctccaaagagatagcggcgctcaccgacttctccggttacctaaccaaactcctgcaaaaccacaccgcctatgcctgtgacggggaccatttgaatctgcagtgtcctcggcattccacgataagtgtccagtcggcattttatgggcaagattaccaaaagtgtagtccgcagcagcctgcctcccagagggaagacagcctaacctgtgcggtgcccaccacactgcagaaggtgctggacgagtgtcagaaccagcgggcctgccacctcctggtcaatagccgtgtttttggacctgacctttgtccaggaagcagtaaatacctcctggtctcctttaaatgccaacctaatgagttaaagaacaaaaccgtgtgtgaagaccacgagctgaaactgcactgtcacaagtccaagtttctcaacatctactctgcgacctacggcaggaggacccaggagagggatgtctgctcctcggaagccgagtggctccccccctttgattgtctgtcttactcggctctgcacgtgttatccaggagatgttacgggaagcagaggtgcaaagtcctcgtcaacaaccgccacttcggaagcccctgtctgccaggagtgaaaaaatacctctcggtcttctacgcgtgtgttcccaagaatatactcacggcaattgatccatcagttgctaatctaaaaccttctgtgaagcagaaagatggagactatggtgtagaccttgacccaagggaaccaagggttctgaggaaagatggtgtgattgtcagccactcgttggcagcatttgcttacattagagcccatccggagagagccgccctgctgttcgtgtccagcgtctgcgtcggtctggcactcacgttgtgcgctttggtcaccagagtgtcctgcaccaaggacttccaggagctgcagctggcaagggagcacctcgtgccaggaagcgacaaggctgaggaggacagccaggatgaggagggggaggaggactcctctgactccgacttgccaggggatctgtcagggtttggtaggactacatatcctgtctacagttccatagaggctgcagagctggcagagaggattgaacgcagagagcagatcatccaggaaatatggatgaacagtggcttggacacttcactcccaagaaacatgggccagttctactgagagccagatccatcctgatgtgagcacactttctgaagagggaaggttccaaaagcccctccagctctgttccacttgtaccttctgtgaaggagaatttgtcatgccatccaatactggggaagccagaaagttcttaaagggacatttcaaactgtttacagcacatcccaaaataaatgtggagggaagaaatctttgtcttctgt

SEQ ID NO: 72 - DNA, 2881 bp, Bos taurus
  source 1..2881: mol_type = other DNA
gcccccgcctcccgcgtcgctcgggaagaacggctgctcgagtctccgcttccctgggtgagccattgaggagctcccgccgctgctccagggtgagggcgagccgagccgcctctgcttttcgctctagctttgagttattttcagaagctaagcccagcacgcttcatgagtggacagaggatacccattttcacatgtagatcttcaacgaagttgagagttaacaactgactcatttttggtattaagaaatgactctgcaccatatttttatgtagaccaaaacatttaacctctcagctgcaaagagaatcacgttggattatttttcccttccagtggctaagttgaagacctgccatgacaatgaagaaagagaccgcgtttgctgtttccacctaaaaaaaaatgaccaacaagaaccaaagttttggtgtgggacaggactccatgtcctccatgacttgtctaatccacgtccttgaagcgtggtttggtgtggagcacttggaagactactggaattttgcaaactacctcttgtgggttttcacaccactcctccttttaatactcccttacttcaccatctttctgctctaccttacaataattttcttacacatttataagaggaagaatgtgctaaaagaagcctactcccataatctgtgggatggtgcgaggaagacggtggcgaccctgtgggacggacatgcggctgtctggcatggttatgaagttcatgggatggagaaaataccagaagaaggaccagcactgatcattttttatcacggagctattcccatagatttttactacttcatggctaaaatttttattcataaaggcagaacttgccgagtagtagcggatcactttgtctttaaaattccagggtttagcttattacttgatgtattttgtgctattcatggaccgagagaaaaatgtgttgaaatcctgcagagtggtcacttgctagctatctcaccaggtggtgttcgggaagcactgatgagtgacgagacctacaacattgtgtggggtaatcggaaaggcttcgctcaggttgcaatcgatgcaaaagtgcccattattcctatgtttacacaaaatattcgagaaggatttagatcacttggaggaacaaggctgtttaggtggctttatgaaaagttccgctatccatttgcccccatgtacggaggttttcctgtgaagttgcgtacgtacttaggtgatcccattccatacgacccaaagatcacagcagaagaactagccgaaaagacgaaggatgctgttcaagctctgattgacaagcaccaaagaatacccggaaacataatgagtgctttgctagaacgttttcgtaacaaacaaaagatcaaccagaagactctttaatacatttatattaatctgtttgtatctataggactgtattctgaattttgtaggttctataattagtatttttttttaaaaatcatgtcaagaagcatctttcattgaacttgtttgcttaaaattatgttgtcaattatgtttctgcaatcagttgtgtcaaatttaaacagtaactcactacatgcatcataattcagaaaatgatcatggttctttttataattttctaatcatgtatcataaacatagatccttcaatctatgatcttccctggtagctcatctggtaaagagtctgcctgtaatgcaggagacccaggtttgatccctgggtcgggaagatcccctggagaggggaatggctacccactccatgcctcataatttggaaaatgatcatggttctttttatagttgcctaatcatgtatcctacacatagatccttaaagtttagtttatcaataatagcagtggaggttaaacaaacattcctaaataatgcatctagtttctgcatctcttgcactgagctagttttgcaagatggcttaacctagggttttagtacagatgcttaagttagagaaaaacaagctctttcctctgcttctcattcctttcatgaccttcagcaaatcacatatttttgtgcctcaacaattcactttttaaaaacatgttaatcctttggtgaggaatattgttttaactctttatatttggtgtgattgatatccatagtatggaaattatgactgtataaactaaacaataccaagatcttttagaagtttgtgtatttattttgtccataggatgtttaaattattccttcagatacgatgtcaagttgccaagcaacaatgaaaaagatgtactgtttttcaaactgagtaaaacagaaattagggaaagtaaaatttctaattattgtggttattctttttagtattatatttttaatgagaatctgtagacaaaccatagaaggtatgctctgcgtcgtctgccctgtaatgtggcatttaatagtgtcaggttagaaatcacagtcatgccctgcatttcgtactttagaagctgactctaggctgctgacagccccgggagaaagctctgctcttctcagcattctggctcattcagcttgactctgcatccagacggatggtttattgtttcaataaaaatatacttgaatgataaatttatgtgaacatagggctctctgaaaaattaatgggtttcgatttgttgtctctttgaaatctgaatttaaaagtatttaaacagaatactgccaggaatcaaatgcagtatgtccctctccctttttaagatattaaattacttggatttttgtatacacttttgtataacaagtatgtatgtatgatatgtgtaatgtatgtacaattaagaaaaataaaaaaaagtaaagctgaaaaaaaaaaaaaaaa

SEQ ID NO: 73 - DNA, 3859 bp, Bos taurus
  source 1..3859: mol_type = other DNA
ccgccctcccgaaccacttccggcggcagctactcgcctgcgaccagcttgagccgtcccgggccggttcgtgccctgcagcaaggggtttttcctccgtccgcccctcggccccgggcagccgaaccgcgacggggtggccggcccgcgtcgtttgcagcgtggccgcctcctctgaggcgctgagatgtactgcaagaagtggaggcctgtggcggagatgtgtctcttcctcgaagacctgcgggagggctcgcgcatcctctgcctctgctcgcgggcgtttgtggaagatcggaaattgtacaatttgggactaaaaggctactacgttaaagatgatgacaacagtgcaggcgaacaagccacggaagaggaggaaggcggccactcccagggccctgcagagcctcatgacaacagatgcccagacccagatgagagtgaactggattccgaggctgaactcatgaggagtatgggattgccccttcagttcggtaggatgtccgcacataagaattttgaggaatctatgaatactagaaacaaagttaaaataatgaaaaagaaaaagaaaaaacatcaaaagaagtacttagatgagattaagagagaatcttggagacaatgtgaagaagatgacgtttcggcttcggacgatccttcttcatctgagaatgagaacactagaaagtacaaacttcaaagccaaaaaggcactgaggctgaaagttttcccgttgaaaacacagtaggtccaaagctggaaattacagagatgtgggaaaagtactggaatgaatacggtggaggactgctgtggcaaagctggcaagagaaacatgcagatcagaccctgtgtccagaaccatggaatgttccagacacaaaggaggagtgggagcagcactacagtcagatgtattggtactatctggaacagtttcagtactgggaagctcagggctggacttttgatgcctcacaaagccaggatacagaccctggtgggtctgaaatagaagttgacaacaagaaagatgaaaatcacatgaaagcagatgaacttttttctccatctttatctgttggcagcgaaagctctagttcaagtgataaagatcacagtgaaattctcaatggaattagtaacctaagtctgaattcagagaaagtagaacagagacaactagatccctccctaagttgtgatggacgtcagtgtttgagtgaagttcccagtagaagagaatgccctgcttctggccacagtgaaccgagtcatggaggaaacacggaaagcagcttgtctgggagcagaagcacagaccagccagctcaggattcacaggagtcctcagaagcaaacacagtcagagacggacgccaccgcagtgggactgatggggacgagagtggagacgatccgcctgaagagaagcgaagcaaactcaagaggagtcatgagctggacctcgatgaaaacccggactcagactttgacaacgatggttctcttctaggattcaagcacggctcagggcaaaaatatggaggaatttcaaacttcagtcaccggcgggtcaggtatttgcagaagaacatgaagcagaagtcaaagtccttggacatgagacgacaaatcaatatgaaaaacaagcacatcttcttcactgaagagtcagagaaaactggttttaagaaaagcagaactttgagtaaggtagaaaaatttctaaaatgggttaaggaaccagtggatgaagaagcatcgccagagtcggtttctcataacaacatgcaagacacttgcacaagcagtggctcagaggaaccagaagtgtctgttgagaagggggatgacccggtggaaatgagtgaggcagaacctggacagcatcctgctgtgtgtccagccggggaagtgggagcagagacgcatgacgggaccagcccagaagccgccgggacagatacagagggctgcgtgccccaggctgcttcggaccctcgccaggaagaggctgaagctgaaagtcagaaaaagaagaaaaagaaaaagaagaacaaaaaggagctgaatctgccccctgacatcgctgccgtgcctgagcttgtgaagtactgggctcagaggtacaggctcttctcccgtttcgatgatgggattaaattagacagagagggctggttttcagttactcccgagaagattgccgagcatatcgcgggcagggtcagccagtccttggagagtggcaccattgtggacgccttctgtggagttggaggaaataccattcagtttgccctaacagggaagagagtgattgccatcgatattgatcctgttaagatcgatcttgctcgcaataatgcagaaatatatggcgtagtggacaagatcgagttcatctgtggagatttcctgcagctggcttctagtctaaaggccgatgttgtgtttctcagcccgccttggggaggaccggactacgccactgccgagatcttcgacatcagcaccatgatgtctcctgatggctttgaaattttcagacttgcccagaagatcactaataatatcgttttctttcttccaagaaatgctgataccgaccagtagaacattcatgacaaaattcaccctaaatcactctgaagcatacggtccaggagcattaaatacattcgtgttgtgggacagccgtcagcaccatgtgcctcgggacatttctgttttctgacactgcagctctgtacccatcacagactgattccacacccgtccaccactgcctccagcgcccgccttctacccgctgtgtctgtaaatctgactgctcgtatatgcctcagtgagtggagccatacgatatttttccttctgtgtctggctattttcacttagtatactgtccttaaggttcatctgtgttgtagcgtgtgtcagaatcttcctttatttttaaggctgaatgacagtctgttgtatgcacacgcgttttgttcatcctgttacccgtctgcggacattcagggttgtttccaccttttgactgctgtggataatgccgctatgaacacgggtgtacacacatctggtcaagtccttgctttcggctttcttggttatatatccacaaatggaacagctggatcagatggtgattctatttttaggttttggactgttctccacagcagcagaatgattttacattcccaacagtagtcacaagagttccaacttgcccacagtctcagtaacatttgtttattttgtttggtaataactatcctagtgggtatggaacggtacctcactgtgcttttagcttgtgctttcctaagtgatgctgagcgtctttcatgtgcgtgtcttctgggtattaaccctttattggatatatgctttcccagttttttctctcatcccatgggttgcctcagaagtgttttttgttactcttgttctggccacaccgcacagcctgggggatcttcgttccccaaccagggactgaacctgagctccctgcagtgagggtgcagggtcctagggactggctcaccagggaagtccccctttactctctcagtaatgccttctaatgcacaaaatgttttcattttgatgaagttcagcttagctaatttttttgtttgttgcctgtgcttttgatgtcatctccagaaacgatggctaaattcagtgtcttgaagatgttctcctgttttcttcttggtacagttgtcactgagtagcttttttaaactactgatactcaagtctactggggaagattctctccagtttgttttctaggatgaggcccacacatggatttttaaa

SEQ ID NO: 74 - DNA, 2851 bp, Bos taurus
  source 1..2851: mol_type = other DNA
cgtcctgccccgccgggccgccccgtcgcggccccagctcggacccccggccaccgtggagcgccgccgccgccccgcgactcacacacggagctggaactatgggatgtataaagtcaaaaaggaaagacaatctgaatgacgatggagtagatttgaagacgcaaccagtacgtaatactgaccgaactatttatgtgagagatccaacgtccaataaacaccaaaggccagttgcagactctcagcttttaccaggacagaggtttcaaactaaagatccagaggagcaaggggacatcgtgatcgcattgtatccgtacgatggcatccacccagacgacttgtccttcaagaaaggggagaagatgaaagtcctggagcagcacggagagtggtggaaagccaagtccatttcgacaaagaaagaaggcttcattcccagcaactatgtggccaaagtcaacacgttggaaactgaagagtggtttttcaaggatataaccaggaaggatgcagaaaggcagcttctggccccagggaacagcgctggagctttccttatcagagaaagcgagacattaaaaggaagttactctctgtctgtcagagactatgaccccgtgcacggtgatgtgattaagcactacaaaatcagaagtctggacaatgggggttattacatctccccacgaatcacttttccttgtatcagcgacatgattaagcattatcaaaagcagtcagatggcttgtgccggagattggagaaggcgtgcattagccccaaaccacagaagccatgggacaaggatgcctgggagatcccccgagagtccatcaagctggtgaaaaggctgggcgccgggcagttcggggaagtctggatgggttactacaacaacagcaccaaggtggccgtaaaaacactgaagccaggcacgatgtctgtgcaggcgttcctggaggaggccaacctcatgaagacgctgcagcacgacaagctggtgcggctgtatgccgtggtcaccagagaggagcccatctacatcatcactgagtacatggccaagggcagtttgctggatttcctgaagagcgatgaggggggcaaggtgctgcttccgaagctgattgacttctctgctcagatcgccgagggaatggcgtacatcgagaggaaaaactacatccacagagacctacgagccgctaatgtgttagtctctgagtctctcatgtgcaaaatcgcagactttggtcttgctcgagtgattgaagataacgagtacacagccagagaaggtgctaagttccccattaagtggacagctccagaagcaatcaacttcggatgtttcaccatcaagtctgacgtgtggtcctttggaattctcctgtacgaaattgtcacctatgggaaaattccctacccagggagaactaacgcggacgtgatgactgccctgtcgcagggctacaggatgccccgcatggagaactgcccagatgagctctacgacatcatgaagatgtgctggaaggaaaaggcagaggagaggccaacatttgactacctacaaagcgtgctggacgacttctacacggccaccgagggccagtaccagcagcagccttagtgctggggactcaagccacgcacgggaccgccgcctccctccaaagggaaaaacaacaatccccggtcactagttatttcctgggctggcttccctggctatccttgcttccttcacacaaggctaaaactcaggaaggacttcttccatgggaaaggattctcctctcaagggcaatatgcccaccagcctgcacccagtgggcacactggctctgcgagtccaacacctggacatggatttctgagaagaaaacacctatcccaccaaagtgcacccacatcggcctcttgtttacaagggggcatgtgactcagaggttcggaggccatttcagtgagtcgcagacaggagcacaacagaaataaccagatgtctccattgcacttccttgtatgttctctatgcttgagcttatttgttaaaaaaaaaaaaaatctactaatccaacctgttagatttgcaggtgaagtcagatgcttccatgtccttcccagatttctgtgacctcccaccccatgaaatctggactggagatcacttccttccacggagatcactcagaaccccacctgggcagaggaatctctttccttgaaagtgtcatcagcaagcaggcagggccaccgtgttaagccatgcggattccccaaccacccaggtttgcccagctcccctggaagggagcttcaggaactgctctgccaatagaaggaatgttgttttaaagtccagcttttcagttccccaatttcctctctccagacttctttggaccttgtagtttggacgaacaagatgcttctaatctctgcggaaccttctagggccttacaaaacatcagagctttctttattgcttcaaatgattctgaacattattttattgatcaagtattcattttagttgtactctagaaagtaaaaatgccgtattcgggggctatttttgtgattcagcaatcttttctaaattgtgtgtggtctgtgtgagtctatttatatgaggctatgcgggaaactaaatgagtgaggaggcttttctgagtcactggagcaattagatagaggttttgtttgttaaatttttctactgtagcttgaatggaaaacgctatgtcatatcacatttcacctatgctgtttgtgtatatacctaatatttctatttttgatcttattttaatacacctgggcccgtaacatctcaa

SEQ ID NO: 75 - DNA, 5442 bp, Bos taurus
  source 1..5442: mol_type = other DNA
ccagcccggcgggagagccggcgcaggggcaggaggaggggagccgcacgccgggagggaagccggggcgcggcgaggaggtggcgggaggaggagacagcgcggaaaggtgccacataaaggagggcgctcctccgcgctggaggcatcatggccgctaagtcagacgggaggctgaagatgaagaagggcagcgacgtggcgttcaccccgctgcagaactcggaccactcgggctcggtgcagggactggcgcccggcctgccgtcggggtcgggggccgaggacgaggaggcggccgggggcgcctgctgcccggacggctcgcgctgctgctgctgctgctgctgcgccgggagcggcggctcgggcggcggctccgggggctcgggcggcccgggcggcggctccgggccgggctcggcggcgctgtgcctgcgcctgggcccggagcagcggcgctactcgctgtgggactgcctctggatcctggcggccgtggccgtgtacttcgcggacgtgggcactgacatctggctcgccgtggactactacttgcgcggccagcgctggtggttcgggctcacgctcttcttcgtggtgctgggctcgctctcggtgcaggtgttcagcttccgctggttcgtgcacgatttcagcaccgaggacagcgccgcggccgccgccggctgcgcgcagcccggggccgagggcaagaccgcggtgggcggcggggccgcggccgcggaaggcgaggctcgcccctccacgccgcagaggcaggcgtccagcgccggcaagggccacgtcgccgccaccaacagcggcagcaacagcagcggggccgcccgggccggcggcaaacccaggtctgcgtcctgttccttctgcatctggctcctgcagtcactcacccacatcttgcagcttgggcagttctggaggtatttccacacgatatacctaggtattcgaagtcggcagagtggggagaacgaccggtggaggttctactggaagatggtgtatgagtacgcggacgtgagcatgctgcacctactggccaccttcttggagagcgccccacagctggtgctgcagctctgcattatcgtacagacacacagcctgcaggccctgcaaggtttcacagcggcagcctccctggtatccctggcttgggccttggcctcctaccagaaggcccttcgggactctcgcgatgacaagaagcccatcagctacatggccgtggtcatccagttctgctggcacttcttcaccatcgcggccagggtcatcacctttgccctcttcgcctcggttttccagctctactttgggatcttcattgtccttcactggtgcatcatgaccttctggatcgtccactgtgagacggagttctgcatcaccaagtgggaggagatcgtgttcgacatggtggtggggattatctacatcttcagctggttcaatgtcaaggaaggcaggacacgctgcaggctcttcatttactatttcgtgatccttctggaaaacacagctttgagtgctctctggtacctctacaaggctccccagatcgcggacgcgtttgccatcccggctctgtgcgtggtcttcagcagcttcttaaccggcgtggtttttatgctgatgtattatgccttctttcaccccaacggaccccgatttgggcagtcaccaagctgtgcttgcgaggacccttctgctgccttcgctctgcctccagacgtggccacgagcaccttgcggtccatctccaacaaccgcagtgtcgccggcgaccgcgaccagaaattcgcagagcgagatgggtgtgtccctgtctttcaggtgaggcccacggccccatccaccccatcctctcgcccaccacggattgaagaatccgtcattaagattgacctgttcaggaataggtacccagcgtgggagagacacgttttggaccgaagcctccgaaaggccatcttagcctttgaatgttcgccgtctcctcctaggctgcagtacaaagacgatgctctcattcaggagcggctggagtatgagaccactttataaaacgagaggacttggaggggccacgacctccagacagaggggtgacagcagggctgtggcaataaggaaactctcccctagagcagggcgttgggctgtgagactcccagtttgaccctcatcggatacgtgtctgaccctcggacagcagagtgcccgctgctggcctcttttcaaagagcagccccaagaccctcctactctgtctgaagggaacagcccagctcagtgggactccttgttgccgccaacccctccagctcaatcttcagcacatccatccaagggtactgttgtgatgggaaatactgcctccaatcagtagggtgtttggatgagttaactgatctttgcataaaaatatactgaatcacacatacacacgagtacacacacactaacacacatgccaatcctctgtcaaaacagtttaggtgatttctcttgatgcaaagctgtgattcctgcaggaaaactaagagagaaaacatatccctcaagactccagaaaaacaaaaaacaaacaaataaaaaaaaatatagtgaagcttagaaagaattcataaaccatcctcatgtgaaaaccttacccaatggacagaaaacccaagtgtgtttacacatgtattagaattgatgaaatggttcattttcaacacacatcaggatttttttttcattttaaattttcagttgagaacatctcctattacagaaatcctatgcagcccatggatggcttctgacaagaccaaggagcttgataccttagtgatgtaagttcagtgataatcaggatgcagtattcagttgcaaaaacatgtaataaaaccacggagaggaagtgggagcaaaaggcagaatgagtgtctgatttcctggcctgtgcagctcacacgggaaggtcagggtggagcggcgggagagacagccagtgttcaggtgcaggaatggaggttctatgtgatggcgggcaggcaggatttcagaatacgccatgccacacccttctagtgccaaagtccatccaacacgagactgacgggaaagccctgaacggctgagcatgggtggcgtgagccccaccaaagcagaagactgctcaaacagtcaagtcctctttgtgtaaaggtgaccctctccccccacccccaaggtcgaagtgatgtgtcttttagaggctttgggacacatttttgtaagtatgagcccacaggtgcagtgaatatgctgtgagaaaacccttgtgaactgattgagggcttatcactagatccagctaagatctgtatttcagtcattttgtaaagtcccactcttacaacaagcttctgggttttaaatacctctgtccagcaagtaaacattccccactttctgttcccagtgtccccccccacccccagtcatggtgctttttgttgcattaaaagtgccggtcaaactttgatagtatttttttatagttggtgcagagtggaataactcatggattatttcaatatttttgtaataaaaaatatagggtatacatataggcatcatcactttttttataaacctgggatcgtttaaaatactttaagcatcataattacttgggatgtagaaactggtccacagatcccatctgcctgcctcagcagatcgagaacacatgtttcttgcaagatcatcctactttgccatgtcggtgcccccaggaacctggccaggggtgctatcagaatatcaggggacgagagaatcagcttaactagaaagggcttgtcaagactggccaatgtttcccaggaaaccaatgatggaagtgatgactcttccatctcttatgacaaacctgaccacagcgggcaatgtcttggatgtcctttcccagttttatatctacccaactgatgcactaaatattagtcaagtcactttttaaaaaaaaaaaagtcagtttaacttaattattcagttatctggactgtccaattcacttaaacagtcaagttctagagccactacctgtcatgactgtgtcgtacagaggcagccgtccctatgacgacacccacagcaagtgacagagggctcgaactaggactttttttggtactaagtccaaaggcagtgtttaaaaattagatgatttttatcagtagacatattttcctggtactccatgatggatcctccaagcagcagtccactaaaaaataatctttggtttggatgtgaaaacgttacctggtcaccaggaaaggcccagaaagatgctcttcctgccagcaataggcaaaaacattccactccccactctagaagggaaaaatgattatattggcaaattcttcactttggtgccagcccgtgggtcattagcttcataattaatgatcttttggaaaccaaattgctcagttgaactatccctttgattccatgagtaaatcgcagatgataatgtcagtcaacaaacacaacaggtcccatcactcttcgatcatgtaaatcctaagttatttctcaacttgatccttcgtttaacaggtgaagaatcaaaatgagcgtggtgtgactaaaaaaacgctgtactgtgctttgtcttcaaagtgagaatgcagtcaggagagtgcaagaagtttgatattttcagtgattttaccaaatactgtaaatactctgttatccaacatacttaaaaggtgagggtttaatcaaaaatacttgttaaggaaatgacatctccaccatgtcataaataacaagacttctcaagattaccgtcaagaacaacacgacaacaaatgttacttccaaggagaatcatggcttaatcttgcttccatgattcagaaagcactgtgttgccatcgagcatgtatttgtttcagatacactgataacagagcatttccagtattaggatttaaagagacattgtcaatgtacaaaggaacaaagtttgattttcatatttatactctagtataaaccctaagattttattttcagttgaccactaaattctccacaagagtagctcaagaatccaaaaggaacaggccttacaaattggaacctacaaatttgttaggatacaaatcggaaacactggattctttatctgtgagctccaacatcatgtgctgggtctgttgggtcacttgggggaggggtgcggtgaagggtctgtccttagtaattaattttgcataggaccatgagttttctaaaaggaaatattataaatcttcttcacttttaaatgaacatttctcaatgaaacttaagttgaggactgtatgtatcctaaccacactgagactttaagtatttgtgtggaagaaaaaaaaaaaaaaa

SEQ ID NO: 76 - DNA, 2090 bp, Bos taurus
  source 1..2090: mol_type = other DNA
atgcactcggcttccagtatgctgggagcggtgaagatggaagggcacgagccgtccgactggagcagctactacaccgagcccgagggctactcctcggtgagcaacatgaacgccggcctggggatgaacggcatgaacacgtacatgagcatgtcggcggccgccatgggcagcggctcgggcaacatgagcgccggctccatgaacatgtcgtcgtacgtgggcgcgggcatgagcccgtccctggccggcatgtcccccggggcgggcgccatggccggcatgagcggctcggccggggcggccggcgtggggggcatggggccgcacctgagcccgagcctgagcccgctaggggggcaggcggccggggccatgggcggcctggcgccctacgcgaacatgaactccatgagccccatgtacgggcaggcgggcctgagccgcgcgcgcgaccccaagacgtaccggcgcagctacacgcacgccaagccgccctactcatacatctcgctcatcaccatggccatccagcagagccccaacaagatgctgacgctgagcgagatctaccagtggatcatggacctcttccccttctaccggcagaaccagcagcgctggcagaactccatccgccactcgctgtccttcaacgactgcttcctcaaggtgccccgctcgcccgacaagcccggcaagggctccttctggaccctgcaccccgactccggcaacatgttcgagaacggctgctacctgcgccgccaaaagcgcttcaagtgcgagaagcagctggccttgaaggaggccgcgggcgccgcgggcggcggcaagaaggcggccgcctccgccggggcccaggcctcgcagggtccgctcggagaggcggccgccgggccggctcccgagactccggcgggcaccgagtcaccccactcaagcgcctccccgtgccaggagcacaagcgagggggccttggggagctgaaggggacgccggcctcggcgctgagccccccggagccggcgccctcgcccgggcagcagcagcagcaggccgcggcccacctgctgggccctccgcatcacccgggcctgccgcccgaggcccacctgaagccggagcaccactacgccttcaaccaccccttctccatcaacaacctcatgtcgtcagagcagcagcaccatcacagccatcaccaccaccagccccacaaaatggacctcaaggcctacgaacaggtgatgcactaccccggctacgggtcccccatgccgggaagcctggccatgggcccggtcacgaacaaagcgggcctggatacctcgcccctggccgcagacacctcctactaccagggggtgtactcccggcccattatgaactcctcgtaagaagacagcggcgacctggctagcccgggacccagatgggagagacaggaagtggggtggagactctgaggaagggctgctggaaaggtgcaagggagaaggacccttcacaccctgccctgcccccccacagaggtctcccctcaccctctgcagtccttccctccctcacgtgggccacactgatgtctaccatcctatataaggagggaaaaagaaaaagctaaacagtttttaaaaaagaagaaaagcctcagtttccactactgtgtagacgcctgcttctctaaaaacctgccaattcttgactttttgttgttgtggttcctcttcattgctgtcgttgcagggaagtcttactttattaaaaaaaagaaaaagaaaaaaacacaaacttttgtgagtgactcagtgtaaaactatgtagttttaacagaaaacaagagttgtactattgttttaaaaagagaaaaaaaaataataatgtaagggactgttgtaaatgcccaagaaaagaaaagaaaaaagcattcgcattcttgatacggtgaaatccaggtctcaagtctgactaatttatggtttctgcgtgctttatttatggcttataaatgtgtgttctgactgcaaggaccagagttccacaaatctatattaaagtgttattgccagttttttgctttgaa

SEQ ID NO: 77 - DNA, 5339 bp, Bos taurus
  source 1..5339: mol_type = other DNA
gaaggcctcgcactggcgaagccgcggtgagcactgctcgtgcgcgcgcgtgtacgtccgccgggagctccgctgctcggtgctcgtgcccgcctgcccgtcgccggcgctcgcgccggggagcgccgccgggtccttagagccggcaggcagctccctttgtgcggcgctcgcgccgcggtccctccggacggaatgtgggctctgtgggagtttttctcttcttcccaaattcttgaaacaatggactgtaaggggcgcggggattaaggggggcgagctgggaagcgacctgcactggggaatcatgacttctgctgcagagattaagaagccaccagtggcccccaaaccaaagtttgttctggcagctaataagccagctccacctcctgtcgcacctaaacctgagatcttggtttctagtgttccacagtcaacaaagaaaaccaaaccatccatagctccaaaaccaaaagttctgaaggcctcacctgtccaggacatcacacagtcaccatcaaggaaaatcgtcgtgaacctggaagagcatacacaggaattccctgaaagtattgacaactcgaactgcagaaacagagggcatccgagcagtgattatattttaccagtgtgttcctgccatcctggatgtaaccataagcttgagagcagaaagaatttgtatgtcaagcagcttgtcttagggcccctggaaatgcatgagaatttagaaaacaacaaaattggtgagtccccttcagctgtgaaaaatcagcataaatgtgattctgttggtgaaagggtcaagagccagagtaaagtagttttaaaggcaagcatcctcgaagagaagctcaaagatgttttaacacaaccaatgtcaccctttcttttcccccagaagcacagatccacagacagtccagaaatgagtgatggctgtaattcccacagacaattcagcattgaatttgcagatttgtcaccttccctgtccagctttgaaaacgtttctaatcatcacccttgccacctacaacttcctagcactgaatttcaaaattttgaaactaatcagaatgacagtgaaaaaagcaacacttgctatcattcatctgaacaagaagctctagaaaatggaaaaaggagtcctttcttatcttcagatggagatagtaagaaatcagaagccagagaccttggtcccttggaaatccacttagtgccatgtactcccaaatttccaactcccaagcccagaaaaacacatgttgcacgcctcttacgccagagacatatagatacttctagtgaaagtactgaagaaccagaggatgtagacagcttgcctcatgttttcgaagatagtttgaaaaacaataaaatcagtgtctttcatcagactgctttgtgtaaccaggaacaggtggacaaagtgaagccagaaaataaaggtgaattgaatgtggacctcactggtgacagacaaaactcggtctattcacagaaagccttgcgtcctgaaacattttcctctgaaaaaacggcacctcctttagaaacagactctaacttgagttctgacgacaagtcaggagatagttctggtatgtcacttgccgaggacaaaggaatcagttttataagatgcagtactctatctatgagcctgcccaagcagctcaaattaacctgcagcgaacatttgcctactgcctctaacccaggagtgtctgcccctcaaatgcaaaaggaatctatgataaaaggtgaaagttcctcaaaagttgtccccaaaaaacctcaaagacacagtttgcctgctgccggagtgcttaaaaaggctgcctctgaggagcttttggagaaaagttcttatccctcaaatgaagaaaaagatgcagagaagggtctagaaagagatcacgtgcagcatttatgtgccccaaacaatggtatgtcatcctcctttgatatgcctaaacgggcttcagagaagccagtgtggaaattacctcatcccattatccccttttcagggaacccagaatccttaaagtctgtcaccatatcctcaaacagtgagactcccacacccctgaccaagcctagagcaaaatcattatctgctatggatgtggaaaggtgcactaagccatgcaaagactctcacaagaagaactctttaaagaagttgctcaatatgaagctgtccatctgtttcatgaagagtgacttccagaaattttggtccaagcatagccatcttggagacacaacctcaggcagcctggcaggtggggagcaaagagggagtgagagtgattggcatggcctgttggtaggagaagagaagagaagtaaacccatcaaggcatattcggcagataactatagcctggagtcccacaagaagaggaagaagttgcggggccacaccaatgcagctaatggaccaagagccgagtctctggatgaccaaatgctgtccagggagtcgtccttgcaggcaccttgcaaatctgttaccagcgactgtgtaccagagtatgaaaatatacggcattatgaggaaataccagagtatgagaacttgccctttgttatggctatggggaaaactcccgagttggaacgacggaactccagcagcgtggaggacactgatgcaaatgtgtatgaggtacaggagccgtatgaagctccagacggccagctccaactcggacccagacaccagcattccagcagttctggagcatcccaggagggacaaagtgatctggaccttggtcttggtgaccttccttcagatgaagaggaagtcatcaacagttcagatgaagatgatgtcagctctgcttctagtaaaggggagcctgatctgctggacgataaacagggtgaagatactggaatgaaatgtaaagtccatcatattgccaaagagatcatgagctcagaaaaagtgtttgtggatgtgttaaaacttttgcatattgatttccgggatgcagtagcccatgcttccaggcaacttgggaaaccagtgattgaggaccggatcctaaaccagatcctgtactacttgcctcagctgtatgagctcaatcgggatctcctgaaggagctggaggaaagaatgtcaaactggacggaacaacaaagaattgctgatatctttgtaaagaagggaccatatctaaaaatgtattccacatacatcaaagaatttgataagaatatagctttgttggatgagcagtgcaagaaaaacccaggttttgctgctgttgttagagaatttgagatgagcccacgctgtgctaatctggccctcaagcactacctgctcaagccggttcagaggattccccagtacaggctgctgctaacagattatttgaaaaatctcttagaagactctggagattacagagacactcaagatgcccttgctgttgttatagaggtagccaaccacgccaatgacaccatgaagcaaggagacaacttccagaaacttatgcaaattcagtacagcttaaatggacaccatgaaatagttcagcctggacgggtttttctcaaagaaggaattctgatgaagctatctcggaaagtcatgcagccccgaatgtttttcctgtttaatgatgccctgttgtatacaacaccagtgcagtctgggatgtacaaactgaacaacatgctatctttggctggaatgaaggtcagaaaacccacccaagaagcatatcaaaatgaacttaagatcgaaagtgtagaacgttccttcatcctctcagccagttctgccccagaaagggatgaatggctcgaagccatttccaggtcaatagaagagtatgccaagaaaagaatcacattctgtcctagtcggagtcttgatgaggctgactcagaagataaagacgaggttagtcctcttggatctaaggctcccatctggattcctgataccagggccaccatgtgtatgatctgcaccagtgaatttactctcacctggagacgacatcactgccgggcctgcggaaagattgtatgccaagcttgttcatcaaataagtatggcttagattacctgaaaaatcaaccagcaagagtatgtgaacactgtttccaagaactgcagaaattagatcaccagcactctcctaagtttggatcccctggaaatcacaaatctccttccagcgccttatcatcagtcttacatagtattccatcagggaggaaacagaaaaaaatcccagctgctctgaaagaagtatcagcaaacacagaagattcctctatgagtggctacttgtacagatcaaagggcaataaaaaaccttggaaacacttgtggtttgtcataaaaaataaagtactgtatacatatgctgcaagtgaggatgtggcagctttggagagtcaacctttactaggattcacagttactcaagtcaaagatgagaattcagagtctagagtgtttcagttactgcacaaaaacatgctattttatgtattcaaagcagatgacgctcactcagctcagaagtggatagaagcatttcaagaaggcacaatactgtagttgcgttggtttcgtctcctgtggttccaaaagggtggaatttcatcagaataaatgcagtacaaacattttatataaatgaacaatgccaagatgaatccaactaaaatcttcatcagagagattattttattaggtataaggtaatttttaaaaaatgtttgtttttcatgtatgttatttattaaaattttaatgtttacttaatggtcagagttatttctgagactcacactgtattctgaagtaccttttttttagaaaccagaacacttaccttaataccgtattaactgtttgtgacacggtccctcctgttttctcagtgtgttttacaatgttactttgtaacattctcaccagtggtatgaatttctatacagtctttgggttggaaattatgattaaacagctgatgagaaaaatgtatgtctttgaatccatacatttagatgaattttttttatcttttaatggaaaaacatatggccaatttctacctcagtaactgaaacaaaattccagtaaattgtctaaagtatttctactgttacgtacctcttgttccatgagataatgttatatatcatcaaggaattactgcaaagggatagaatctgaattttcacttaactcaattgtgagtaggaaaaagaatgtttccattttcagggttacacaaataattaactctatggcctttacattaagtctaatctttaaaaaa

SEQ ID NO: 78 - DNA, 7737 bp, Bos taurus
  source 1..7737: mol_type = other DNA
atgctgtccctggtagggaagctgccctgtagtccgtccccagtttcagtatatgtacggctgaagtgggctctggcattgtcaggagccggcccttggctgtccccaaggcctggcctcacttaccctctgacccctgcccagtgccaggatggtggagactggcctgaagggctggttgctctgggccctgctcctgcactcggcccagagtgaggtctacacacccatacaccagcctggctactgtgccttctacgatgagtgcgggaagaacccggagctgtccggaagcctggcttcgctgtccaatgtgtcctgcctggataactcacctgctcgccacatcacaggtgaccacctggccctcctacaaagcatctgtccccgtctctacactggcgccagcaccacctacgcctgctgctcctccaagcagctggtggccctggacatgagcctgcggatcaccaaggccctcctcactcgctgtcccgcctgctctgacaacttcgtgagcctgcactgccacaacacctgcagccccaaccaaagcctcttcatcaatgtgacccgagtggtcacacagggggacagccagtcccaggccgtggtggcctacgaggccttctaccagcgcagctttgccgagcagacctacaactcctgcagccgtgtgcgcatccccgcagctgccacactggccgtgggctccatgtgtggcgtctatggctctgccctctgcaacgcccagcgctggctcaatttccagggggacacgagcaatggcctggcacccctggacatcaccttccacctgtgggagcctagccaggccgaggggagcacgatacagcctctaaatgatgaggtcgtgccctgcaaccagtcccagggggacggtgcagcggcctgttcctgccaggactgtgccgcctcctgccccgtgattgcccagccccgggccctggaccccaccttccgcctgggccggatggaagggagcctggtcctcattatcatcctctgttctctctttgtcttgctcacggccttcctcctgcggtcccgcctggccgagtggtgcaggggcaagcgcaagacgcccaagcccaaggcaagcatcaacctggcccacaggcttagcctctccacccacaccctcctcagccgctgcttccagtgctggggcacttgggtggcctcatggccactgaccatcctggcggtatctgtgattgtggtggtggccctggcagggggcctggcttttatagaactgaccacggaccccgtggagttgtggtcagcccccaacagcctagcccgacgtgagaaggcgttccacgacaagtatttcggccccttcttccgaaccagccaggtgttcatgacagcacctcaccggcccagctataggtatgactccctgctgctggggcccaagaacttcagcgggatcttgtcctcggatctgctgctggaggtgctggaattgcaggagaggctgcggcatctgcaggtgtggtcgccagaggagcagcggaacgtctccctgcgggacacctgctatgcccccctcaacccacataacgccagtctgtccgactgctgcgtcaacagcctcctgcagtacttccagaacaaccgcactcagctgctgctcacagccaaccagacgctgtcagggcagaccgcccaggtggactggagggaccacttcctctactgcgccaatgcccccctcacctacaaagatggcacggccctggccctgagctgcatggctgactatggggcacccatcttccccttcctcgccgtggggggctacaaaggaaaggactattctgaggcagaggccctgatcatgaccttctccctcaacaattaccctcctggggaccccaagctggcccaggctaagctctgggaggcagccttcttggaggagatgcgagcctttcagcgtcggacggctggtgtgttccaggtcacattcatggcggagcgttccctggaggatgagatcaacagcaccacggccgaggacctgcccgtctttgctgttagctaccttgtcatcttcctgtacatctccctggccctgggcagctactccagctggcgtcgggtaccggtggactccaaggccacgctgggcctgggcggggtggccgtggtgctgggagcagtcgtggcctctatgggcttcttctcctacctgggtgtcccttcctcactggtgatccttcaagtggtgcccttcctggtgcttgccgtaggggccgacaacatcttcatcttcgttcttgagtaccagaggctgcctcggaggcctggggaagaacgggaggcccacatcggccgagccctgggcagagtggcccccagcatgcttctctgcagcctctctgaggccatctgcttcttcttaggagccctgacccccatgccagctgtacggacctttgccctgacctccggctttgcagtgcttcttgacttcctgctacagatgtcagcctttgtggccctgctctccctcgacagcaggaggcaagaggcctcccggatggacatctgctgttgcaagacagcccagaagctgcccccgccgagccaggatgaggggctcctgctccgattcttccgcaagttctacgtcccattcctgttgcactggctcacccgcgtggtcgtggtgctgctcttcctggccctgtttgcagcgagcctctatttcatgtgctacattaatgtggggctggaccagcagctggctctgcccaaggactcatacctgatagactatttcctctttatgaaccgctactttgaggtgggggccccggtctattttgttaccactgggggctacaacttctccagtgaggaaggaatgaatgccatctgctccagtgcaggctgcaacaacttctccttaacccagaagatccagtacgccaccgacttccctgatatgtcttacctggccatccctgcctcctcctgggtggacgacttcattgactggctgacttcatcttcctgctgccgcctttatatctctggtcccaataaagatgagttctgcccctcgactgtcaactccctggcctgcttgaagacctgtgtgagccccacagcgggctctgcccggccatcagtggagcagttccacaaatatcttccctggtttctgagtgacgagcccaacatcaaatgtcccaaaggtggcctggcagcatacagcacctcagtgaatatgagctcagatggccagattttagcctcgcggttcatggcctacaacaagccgctgaagaactcgcgggatttcacggaggctctgcgggcaactcgggcactggcagccaacatcaccgctgacctgcggaaggtgccaggcactgacccagattttgaggtcttcccttactcggtcaccaacgtgttctatgagcagtacctgaccatagtccccgaggggctctttatgctcaccatctgcctggtgcccacctttgtcgtctgctgcttcctgctgggcatggacgtccgctccggcctcctcaacctcttctctatcatcatgatcctggtggacactgtgggcttcatgatgctctgggacatcagctacaatgctgtgtccctcatcaacctggtcacggcggtgggcatctctgtggagtttgtgtcccatatcacccgctcctttgccatcagcaccaagcccacccggctggagagggctaaggaggccaccatctccatgggcagtgcggtgtttgcaggagtagccatgaccaacctgcctggcatccttgtcctgggcctggccaaggcgcagctcatccagatctttttcttccgtctcaacctcctcatcactgtgctgggcctgctgcatggcctggtcttcctgcccgtcatcctcagctacctggggcctgatgtcaacccagctctggtgcagcagcagaagcagcaggaggaggcagctgcggccaaggagacctcttgctcaaagcaccccgctcagatgagcactgattatggtgtctacgtcaactgcagctttgaacatcctgccaagagtgtaggaggcctcgagagctctccatcagagaacaggcagaagttctgatggggccagagcccctgtccaagctcatcagccatgacctgaggggccatgaaggctcctcctgggggtcctttgacttatccttcccccaggccctgagagaggctgtctccacaggaccgtccaccatcggtcatcctctagccaccagatctcacccataaggacactgattgtgatagaggtgccacgtgatggcttgggtgtgacctggtccaaatccatctcttgaatttgacatgtgtcctcctcatgcaagttttaaaactttagttccatatatgtgaacaacatatttgttcacatttaaaattttccttaattgataccatatgtgacctatccttttgcaatgaggtttttttttttttttatgcagcatacattttttcgatttattcgtgacgatacatggagatctagtccaatcattttaactgtgacacagctttccattgtgtgaatgaacaggatgtttttttatgcattcccctgctagtgggcaatcacattgtcctgaattattttttctgctataagtccacagtcacttatgtggaatttcagaacccccagaactctgaaaacctagtgtgtttgtttttttcttttgtaagtttgggacaaactcatttcatagcaaagactgatcagacacgatgtatctatttatactctttataaataggtttccctggtggctcagatggtaaagaatttgcctgcaatgcaggagattcaggttctatccttgggatgggaagatcccctggagagggaaatggcaacccactccagtattcctgcctggagaattccatggacagaggagcctggcgggctatagtccacggggttgcaaagagtcagacacaactgagtgactaacacttctctttttttttttttctcccataaatagcatacttacactcttcagccaacttgatgtgaatagtcatatatttcagcacattaacaagtttgatttcatgttgcttccccagatcctgatgaggtctaatatctaatgtactgtaagcatcaaattacctttctaaaaaaacgttctaaattctgaaattcagcttgctatgggatttcaggtatggaaccatggacctgtgtgatgagcaggtgtttacacgtctcctgtctatgtgtggggagctaccatcgctgtagcactgaatggatacagactagtgtcttccagggcgtctgccctattcacacctccaccagcaggggataagggtgcctctttccctgcaccaataccaacacaacgctccacttgtttctgggagggaggggagtggggttatagattgttatccctttctaaaaaatacttttttaaaagaaaactttttttattttagaatagttttacagaaaaattacaaagataatacagagagttcctgtatgccccccgcccgccccccaaaatttacttgagtaacacattagtgtgataacacttgtcagtgtaccatgaatgaaccagtactgataattactactaacccaaatccatattttgtttagatttccacaattttacccagggtccttttcctgagccaggatcctgtccaggacactccacaacactgttgttatgtctacttaggctccttttgtttgtgacagtgtttcaggctctccttttctttgtttttggtgacccggacagttgaagattggtcaggtattttaaagaatgtccttcaaatggggtttgtctagtgtttctcccgattagacaaaggtgatgggtttttcggagggagatcacgaggcagagagccggtctcagcctattgtgtcaagggtgcctgcagtcaccgtaacttgtcgctgtcacactggccctgaccacctggctgaggttgtgttggccaggtgtctcctctgtacacttatcttgcacctgtttgcgtggtgcactctttgcagcgaagttgctatgcacagcctacacttaagggtgagtggttatgctctacttcctggagggtggagtgtttacataaattatttggaactcttctgaacaggaaaattgttctccatttattatttattcagtatgctatctttatcagtatggaaccaaggatgctcattttttactttgggttttaatatagtgactatttgctccacatttggccattgggaactcttccagttggctcctaggtccctttgatgtattcctttcactgtggggttccccccactttttgagcacttttgtacttcctggcattataagatagtataggctcaacttgtatatttctatcccagtcctagagtcagccatttctcccaagagttccagtccttcatctggagataagattagaaatcaggatctgggtgttcagtgtgttttgctgctggggctttgttgcttctgatcctctcagctgagagagcaagggcatacatatgtgaatactgacctgtatatgtacacatttctagaagtatttctatgaaaagccatctgtatctacattaagctaaacaggattttatgctgatacctccagttataacccataatcacatgaatcattctagactcctttcttgcttctctgtagcctctcatgccactaatgagaagccgggctccacaaccctcaccatttacttagttgttgattacctttatacatatgtaggagtctgagaaatgataacctacactcctgtggaaaacaactttagaatttagtgtttacatgcaattccttttgcctttagtcttacagacatcattcctttccaaagttacttcggttagcatctttcagatggggtcagtatccctttcagagaagctgcttcatacattgaaaaaatatttatttatttatttttggctgagctaggtctttgtttctgccttcaggccttctctagttgcggagatcaggggctactctttagttgtggtgcgtgggcttctcattgcagtggcttctgttgttgcagagcacaggctctaggtgcatgggttcagtagtagtggggcatgggtttagttgttctgcagcatgtggaatattcccagaccagggattgaactcgtttctcttgcattggcaggattcttaacccctggaccaccagggaagcccttatacatttttaatatagttaagattcttgcatcacattctttattccattctgattttacaaaatcatgtgtgtaaaagtttgttgtgggtattcagcagaacacgatccctggttgaccctctggctagacccaggcaatgggagactcctcacgccttcctctgtccttgtaatgtatgctctgctgctctgcgtaccattctcacagcgagagttgttttcaacaacatcttcctctcaacaccttgagagaggaaggtgtttttgagaccatctggacagtgtatgtgattgaacccagttaaggtgtctgtataaacatttgcgattctggcaaagtggacgtgaaaatacactggtcttgtggctgcccaggacaagtaactttccttgcttattaaacctcctacctatca

SEQ ID NO: 79 - DNA, 4552 bp, Capra hircus
  source 1..4552: mol_type = other DNA
atggtggagactagcctgaagggctggttgctctgggccctgctcctgcactcggcccagagtgaggtgtacacacccatacaccagcctggctactgcgccttctacgatgagtgcgggaagaacccggagctgtccggaagcctggcttcgctgtccaatgtgtcctgcctggataactcgcctgctcgccacatcacaggtgaccacctggctctcctacaaagcatctgtccccgtctctacactggtgccaacaccacctacgcctgctgctcctccaagcagctggtggccctggacatgagcctgcgggtcaccaaggccctcctcacccgctgtcccgcctgctctgacaacttcgtgagcctgcattgccacaacacctgcagccccaaccagagcctcttcataaatgtgagccgagtggtcacacagggggacagccaggcccaggccgtggtggcctacgaggccttctaccagcgcagcttcgccgagaagacctacgactcctgcagccgtgtgcgcatccccgcagctgccacactggccgtgggctccatgtgtggcgtctatggctctgccctctgcaacgcccagcgctggctcaatttccagggggacacgagcaatggcctggcacccctggacatcaccttccacctgtgggagcctagccaggctgaggggagcgtgatacagcctctaaatgatgaggtcgtgccctgcaaccagtcccagggggatggtgcagcggcctgttcctgccaggactgtgccgcctcctgccccgtgattgcccagccccgggccctggaccccaccttccgcctgggccggatggaagggagcctggtcctcattatcatcctctgctctctctttgtcttgctcacggccttcctcctgcgatcccgcctggccgagtggtgcaggggcaagcgcaagacgcccaaacccaaggaaagcatcagcctggcccacaggcttagcctctccacccacaccctcctcagccgctgcttccagtgctggggcacttgggtggcctcgtggccactgaccatcctggcggtatctgtggttgtggtcgtggccatggcagggggcctggcttttatagaactgaccacggaccccgtggagctgtggtcagcccccaacagcctagcccgacgtgagaaggcatttcacgacaagtatttcggccccttcttccgaactagccaggtgttcatgacggcacctcaacggcccagctataggtatgactccctgttgctggggcccaagaacttcagcgggatcctgtccttggatctgctgttggaggtgctggaattgcaggagaggctgcggcatctgcaggtgtggtcgccagaggagcagcggaacgtctccctgcgggacacctgctacgcccccctcaacccacataacgccagtctgtctgactgctgcgtcaacagccttctgcagtacttccagaacaaccgcactcagctgctgctcacagccaaccagacgctgtcagggcagaccgcccaggtggactggagggaccacttcctctactgcgccaatgcccccctcacctacaaagatggcacagccctggccctgagctgcatggctgactatggggcacccgtcttccccttcctcgccgtggggggctacaaaggaaaggactattctgaggcagaggccctgatcatgaccttctccctcaacaattaccctcctggggaccccaagctggcccaggctaagctctgggagggagccttcttggaggagatgcgagcctttcagcgtcggacggctggtgtgttccaggtcacattcatggcggagcgttccctggaggatgagatcaacagcaccacggccgaggacctgcccatctttgctgttagctaccttgtcatcttcctgtacatctccctggccctgggcagctactccagctggcgtcgggtaccggtggactccaaggccactctgggcctgggcggggtggccgtggtgctgggagcagtcttggcctccatgggcttcttctcctacctgggtgtcccttcctcactggtgatacttcaagtggtgcccttcctggtgcttgccgtgggggccgacaacatcttcatctttgttcttgagtaccagaggctgcctcggaggcctggggaagaacgggaggcccacatcggccgagccctgggcagagtggcccccagcatgcttctctgcagcctctctgaggccatctgcttcttcttaggagccctgacccccatgccagctgtacggacctttgccctgacctccggctttgcagtgcttcttgacttcctgctacagatgtcagccttcgtggccttgctctcccttgacagcaggaggcaagaggcctcccggatggacatctgctgttgcaagacagcccggaagctgcccccgccgagccaggatgaggggctcctgctccgattcttccgcacgttctacgtcccattcctgctgcactggctcacccgcgtggtcgtggtgggggccccggtctattttgtcaccactgggggctacaacttctccagtgaggaaggaatgaacgctatctgctccagtgcaggctgcaacaacttctccttaacccagaagatccagtacgccaccgacttccctgatgtgtcttacctggccatccctgcctcctcctgggtggacgacttcattgactggctgacttcatcttcctgctgccgcctttatatctatggtcccaataaagatgagttctgcccctcgactgtcaactccctggcctgcttgaagacctgtgtgagccccacagcgggctctgcccggccatcagtggagcagttccacaaataccttccctggtttctgagtgacgagcccaacatcaaatgtcccaaaggtggcctggcagcatacgccacctcagtgaatatgagctcagatggccagattttagcctcgcggttcatggcctacaacaagctgctgaagaactcgcaggatttcacggaggctctgcgggcaactcgggcactggcagccaacatcaccgccgacctgcggaaggtgccaggcaccgatccagattttgaggtcttcccttactcggtcaccaacgtgttctatgagcagtacctgaccatagtccccgaggggctctttatgctcaccatctgcctggtgcccacctttgtcgtctgctgcttcctgctgggcatggacgtccgctccggcctcctcaacctcttctccatcatcatgatcctggtggacactgtgggcttcatgacgctctgggacatcagctacaatgccgtgtccctcatcaacctggtcacggcggtgggcatctctgtggagtttgtgtcccacatcacccgctcctttgccatcagcaccaaacctacccggctggagagggctaaggaggccaccatctccatgggcagtgcggtgtttgcaggagtggccatgaccaacctgcctggcatccttgtcctgggcctggccaaggcgcagctcatccagatcttcttcttccgtctcaacctcctcatcaccctgctgggcctgctgcacggcctggtcttcctgcccgtcatcctcagctacctggggcctgatgtcaacccagctctggtgcagcagcagaagcaggaggaggaggcagctgcgaccaaggagacctcttgctcaaagcaccctgccccgacaagcactcatgacggtgtctacatcaactacagctttgaacatcctgccaagagtgcaggaggcctcgatagctctccatcagagaacaggcagaagttctgatggggccagagcccctgtccaagctcatcggccatgacctgaggggccatgaaggctcctcctgggggttctttgacctatccttcccccaggtcttgagagaggctgtctccacaagactgcccaccatcagtcaccctctggccaccagatctcatccataaggacaccgattgtgatgtaggtggcacgtgatggcttgggtgtgacctggtccatctcttgaatttaacatgtgtcctcctcatgcaagttttaaaactttagttccatatatgtgaacaacttactcgttcacatttataattttcctaattgataccatacgtgacttatccttttggaatgaggcttttttttttttttatgcggcatacattttttcaatttattcatgtcgatacatggagatgtagtccaatcattttaactgagacagctttccattgtatgaatgaacaggatgttttttatccattcccttgctaatgggcaatcagattgccctcaattatttttctgctataagtccacagtcacttatgtggaatttcagaacccccagaactctgaaaacctagtgcgtttgtctttttcttttgtaagttttggacaaactcatttcgtagcaaagactgatcagacaccatgtatctatttatactctttataaacaggcttccctggt

SEQ ID NO: 80 - DNA, 4807 bp, Ovis aries
  source 1..4807: mol_type = other DNA
agtttcagtatatgtacggctgaagtgggctctggcattgtcaggagctggcccttggctgtccccaagccctggcctcacttaccctctgacccctgcccagtgccaggatggtggagactagcctgaagggctggttgctctgggccctgctcctgcactcggcccagagtgaggtgtacacacccatacaccagcctggctactgcgccttctacgatgagtgcgggaagaacccggagctgtctggaagcctggcttcgctgtccaatgtgtcctgcctggataactcgcctgctcgccacatcacaggtgaccacctggctctcctacaaagcatctgtccccgtctctacactggtgccaacaccacctacgcctgctgctcctccaagcagctggtggccctggacatgagcctgcgggtcaccaaggccctcctcacccgctgtcctgcctgctctgacaacttcgtgagcctgcattgccacaacacctgcagccccaaccagagcctcttcataaatgtgacccgagtggtcacacagggggacagccaggcccaggccgtggtggcctacgaggccttttaccagcgcagcttcgctgagcagacttacgactcctgcagccgtgtgcgcatccccgcagctgccacactggccgtgggctccatgtgtggcgtctatggctctgccctctgcaacgcccagcgctggctcaatttccagggggacacgagcaatggcctggcacccctggacatcaccttccacctgtgggagcctagccaggccgaggggagcgtgatacagcctctaaatgatgaggttgtgccctgcaaccagtcccagggggatggtgcagcggcctgttcctgccaggactgtgccgcctcctgccccgtgattgcccagccccgggccctggaccccaccttccgcctgggccggatggaagggagcctggtcctcattatcatcctctgctctctctttgttttgctcacggccttcctcctgcgatcccgcctggccgagtggtgcaggggcaagcgcaagacgcccaaacccaaggaaagcatcagcctggcccacaggcttagcctctccacccacaccctcctcagccgctgcttccagtgctggggcacttgggtggcctcgtggccactgaccatcctggcggtatctgtggttgtggtcgtggccatggcagggggcctggcgtttatagaactgaccacggaccccgtggagctgtggtcagcccccaacagcctagcccgacgcgagaaggcattccacgacaagtatttcggccccttcttccgaactagccaggtgttcatgacggcacctcaccggcccagctataggtatgactccctgttgctggggcccaagaacttcagcgggatcctgtcctcggatctgctgttggaggtgctggaattgcaggagaggctgcggcatctgcaggtgtggtcgccagaggagcagcggaacgtctccctgcgggacacctgctacgcccccctcaacccacataacgccagtctgtctgactgctgcgtcaacagcctcctgcagtacttccagaacaaccgcactcagctgctgctcacagccaaccagacgctgtcagggcagaccgcccaggtggactggagggaccacttcctctactgcgccaatgcccccctcacctacaaagatggcacagccctggccctgagctgcatggctgactatggggcgcccgtcttccccttcctcgccgtggggggctacaaaggaaaggactattctgaggcagaggccctgatcatgaccttctccctcaacaattaccctcctggggaccccaagctggcccaggctaagctctgggagggagccttcttggaggagatgcgagcctttcagcgtcggacggctgatgtgttccaggtcacattcatggcggagcgttccctggaggatgagatcaacagcaccacggccgaggacctgcccatctttgctgttagctaccttgtcatcttcctgtacatctccctggccctgggcagctactccagctggcgtcgggtaccggtggactccaaggccactctgggcctgggcggggtggccgtggtgctgggagcagtcttggcctccatgggcttcttctcctacctgggtgtcccttcctcactggtgatacttcaagtggtgcccttcctggtgcttgccgtgggggccgacaacatcttcatcttcgttcttgagtaccagaggctgcctcggaggcctggggaagaacgggaggcccacatcggccgagccctgggcagagtggcccccagcatgcttctctgcagcctctctgaggccatctgcttcttcttaggagccctgacccccatgccagctgtacggacctttgccctgacctccggctttgcagtgcttcttgacttcctgctacagatgtcagcctttgtggccttgctctcccttgacagcaggaggcaagaggcctcccggatggacatctgctgttgcaagacagcccggaagctgcccccgcctagccaggatgaggggctcctgctccgattcttccgcaagttctacgtcccattcctgctgcactggctcacccgcgtggtcgtggtgctgctgttcctggccctgttcggagcaagtctgtatttcatgtgctacgttaatgtggggctggaccagcagctggctctgcccaaggactcatacctgatcgactatttcctctttatgaaccgctactttgaggtgggggccccggtctattttgtcaccactgggggctacaacttctccagtgaggaaggaatgaatgctatctgctccagtgcaggctgcaacaacttctccttaacccagaagatccagtacgccaccgacttccctgatgtgtcttacctggccatccctgcctcctcctgggtggacgacttcattgactggctgacttcgtcttcctgctgccgcctttatatctatggtcccaataaagatgagttctgcccctcgactgtcaactccctggcctgcttgaagacctgtgtgagccccacagcgggctctgcccggccatcagtggagcagttccacaaataccttccctggtttctgagtgatgagcccaacatcaaatgtcccaaaggtggcctggcagcatacgccacctcagtgaatatgagctcagatggccagatcttagcctcgcggttcatggcttacaacaagctgctgaagaactcgcaggatttcacagaggctctgcgggcaactcgggcactggcagccaacatcaccgccgacctgcggaaggtgccaggcaccgatccagattttgaggtcttcccttactcggtcaccaacgtgttctatgagcagtacctgaccatagtccccgaggggctgtttatgctcaccgtctgcctggtgcccacctttgtcgtctgctgcttcctgctgggcatggacgtccgctccggcctcctcaacctcttctccatcatcatgatcctggtggacactgtgggcttcatgacgctctgggacatcagctacaatgccgtgtccctcatcaacctggtcacggcggtgggcatctctgtggagtttgtgtcccacatcacccgctcctttgccatcagcaccaaacctacccggctggagagggctaaggaggccaccatctccatgggcagtgcggtgtttgcaggagtggccatgaccaacctgcctggcatccttgtcctgggcctggccaaggcgcagctcatccagatcttcttcttccgtctcaacctcctcatcaccctgctgggcctgctgcacggcctggtcttcctgcccgtcatcctcagctacctggggcctgatgtcaacccagctctggtgcagcagcagaagcaggaggaggaggcagctgcgaccaaggagacctcttgctcaaagcaccctgccccgatgagcactcatgacggtgtctacatcaactacagctttgaacatcctgccaagagtgcaggaggcctcgatagctctccatcagagaacaggcagaagttctgatggggccagagcccctgtccaagctcatcagccatgacctgaggggccacgaaggctcctcctgggggttctttgacctatccttcccccaggtcttgagagaggctgtctccacaagactgcccaccatcggtcaccctctggccaccagatctcatccataaggacactgattgtgatgcaggtggcacgtgatggcttgggtgtgacctggtccaaatccatctcttgaatttaacatgtgtcctcctcatgcaagttttaaaactttagttccatatatgtgaacaacttactcgttcacatttataattttcctaattgataccatacgtgacttatccttttggaatgaggcttttttttttttatgcagcatacattttttcaatttattcatgtcgatacatggagatgtagtccaatcattttaactgagacagctttccattgtgtgaatggacaggatgttttttatccattcccttgctaatgggcaatcagattgccctcaattatttttctgctataagtccacagtcacttatgtggaatttcagaacccccagaattctgaaaacctagtgcgtttgtctttttcttttgtaagttttggacaaactcatttcgtagcaaagactgatcagacaccatgtatctatttatactctttataaacaggcttccctggtggc

SEQ ID NO: 81 - DNA, 1600 bp, Bos taurus
  source 1..1600: mol_type = other DNA
catcaccttgcatttcacccacgactacgttagtaacgggaaacacaccaacagaaaggtctcagtttcctcttgggactcgcaagcaaagggttctcgctgtcccgcctagtttctggatatccagtctggaacacagcccacaaaccacgtgacaacagcgtggctcacgtaacagttgttgcccgcgctacaacttttccgccagtttccaacatggccactaggcgccctcaatgccgcagccccgggccctcttgcgcttcccggcccacttctgctcctcagcgagacctcccgagaagcccgggggcaataggggcggtgcctgggtccagtcggctccagcgcagtccagcccatctccaccgcgggggcgaacaagagcggaccggaagtttcccgggcgcgctagcggccgaggccgttactgcagagcaccggaagtggccataggccggcgtccgggccgcgcccgcagtgagtgagctttcctccggccggcgacgccatggagcccggggcggccgagctgtacgatcaggccctgctgggcatcttgcagcacgtgggcaacgttcaggactttctgcgtgtgcttttcggcttcctctaccgcaaaacggacttctaccgcctgctgcgccacccgtcggaccgcatgggctttcccccgggggcagcgcaggcgctggtgctgcaggtattcaaaacctttgaccacatggcccgtcaggatgatgaaaagaggagaaaagagctggaggagaaaatcaggaggaaggaagaagaggaggccacggcggtggcagctgccacagcggagcaggagccagttccagccccggttcaggaggtggaggtagactccaccacagactcaggtgggccccaggagcccccaggcccacggggcaccctgcaggaagtggctgtcagctctggggaggcagaggcaccgggagcagatgctggcgctgctgaagcccccagggaaccgctagctcttcccaagagacaggagcagttccagagaaaccccgacagctacaacggtgctgtgcgtgagaactacacctggtcccaggactacaccgacctagagctcaaggtgccggtgcccaagcacgtggtgaagggcaagcaggtctcggtgaccctcagcagcagctccatccgggtggctgtgctggagggaaatggggagcgtgtcctcatggaagggaagttcacccacaagatcaacaccgagagctccctctggagcctcgaacccgggaagtgcgtcttggtgagcctgaacaaggtgggtgagtactggtggagcgccatcctggagggcgaggagcagatcgacatcgacaagatcaacaaggagcgctccatggccacggtggacgaggaggagcacgccgtcctggacaggctcaccttcgactaccaccagaagctgcagggcaagccgcagagccacgagctgacacggcaggagcagtttggccactgagctccatcagctgaggtccaaggaaaatggggtgcctgcctgcgaaatcaccctgtctccggtgttggtttgcaataaatctgtatttaagca

SEQ ID NO: 82 - DNA, 2785 bp, Capra hircus
  source 1..2785: mol_type = other DNA
agcaccggaagtggccataggccggcgtccgggccgcgcccgcagtgagtgagctttcctccggccggcgacgccatggagcctggggcggccgagctgtacgatcaggccctgctgggcatcttgcagcacgtgggcaacgttcaggactttctgcgtgtgcttttcggcttcctctaccgcaaaacggacttctaccgcctgctgcgccacccgtcggaccgcatgggctttcccccgggggccgcgcaggcgctggtgctgcaggtattcaaaacctttgaccacatggcccgtcaggatgatgaaaagaggagaaaagagctggaggagaaaatcaggaggaaggaagaagaggaggccacggcggtggcagctgccacagtggagcaggagccagtcccagccccggttcaggaggtggaggtagactccaccacagactcaggtgggcctcaggagcccccaggcccacagggtgccctgcaggaagtggctgtcagctctggggaggcagaggcaccaggagcagatgctggcgctgctgaagtccccagggagtcaccagctcttcccaagagacaggagcagttccagagaaaccctgacagctacaacggtgctgtgcgcgagaactacacctggtcccaggactacaccgacctagagctcaaggtgccggtacccaagcacgtggtgaagggcaagcaggtctcggtgaccctcagcagcagctccatccgggtggctgtgctggaggaaaatggggagcgtgtcctcatggaagggaagttcacccacaagatcaacactgagagctccctctggagccttgaacccgggaagtgcgtcttggtgagcctgaacaaggtgggcgaatactggtggagtgccatcctggagggcgaggagcagatcgacattgacaagatcaacaaggagcgctccatggccacggtggacgaggaggagcacgccgtcctggaccggctcaccttcgactaccaccagaagctgcagggcaagccgcagagccacgagctgaaagtccatgagatgctgaagaaggggtgggatgctgaaggctctcccttccgaggccagcgattcgacccggccatgttcaacatctccccgggggctgtgcagttttaatgaccggaggagaggacgccctcgccggcagggaagcggggccgcagccgtgccccccagctccctcctgccggggactcgctgtctagtttcaagggtgaacccggcctccaccctgccctgcgtctccagactgtttcagagcggggcggggctgtgcgccttgcgagggggtgtcgggagcaggggctgtctcagcgggcgcagctctgggccctgccggcaccgctcgccactccctccccgcgctcgtggctccgctcctgtgctctcttgcttgctgtcctccgtgcgctcggggcgggcgtgggtgcccctcggctgtggaagcgcaggcagggatgtgcaggctgagggctgggctcagggccagtttggggaagctgctgccttgccctcttgctgggtgtgttcttgtgtcgtcttgcctatctgcttgtgcgcgtccaccgcctccctcagcctcatcgaggagccatggaaaccggggtgctggtctctggttctgggtgctggaagcatctctggtggcctcaggccctgcacagtagggactctgtcttctgattggcctgtcctgctgccacccggcctagcactttcccctgggggacctggttcttgtcagagcgagctgggccaggcgccctcaggagaactcatggtgtgtcactgctcacggcagccccaggtcctttcttggctgaagccagactgtgccacccagagcttgggcccctgctcctgcggctctcccaggacccagagcccttgcccttcgccaggaccgcgcggctgccctattggtctgttaccttgcaggcctctgcaccctggaacctgccctcgggctctgtgtgctggactgcgggaggaggagccctgggggtggggacgttccttctagaaacttccaccttcagttgcgtgggtgggagctggttgattgctgtgtgtgggggcaggtttctggctcacagggccctgtagacacactcctgggaagcacttggcttgctccaatgccatcacgggaggtgtgtacacctggtgactggtgggagcaccaccaagggggcacggaacactgttgttggctgtcatcgccaaggctggtcacggagctgctgaggctccccttcagcgtcctccggatctcacctgtcccacgtagtgagaggtctcactggtctgtgtgagaaccattctggcaccgtgcctgtctcctcccctccgccacccccagggggccctctgccgttaccgggtggggtatgagccctgcaccttggctgttgggagctgtggactccatggtcagttgtcttccttttgcccaattttattttcaaatgtttgcatttctcctcaccccactggggataatagcagcttgccggcatctgggagtggcaggctggggggcttgccatcttgtgggctgcctgcacccctcgctgctggcctgggccctggacctgtgcactctgcctgttttctctcttcagacacggcaggagcagtttggccactgagctccatcagctgaggtccaaggaaaatggggtgcccgcctgcgaaatcaccctgtctccggtgttggtttgcaataaatctgtatttaagcact

SEQ ID NO: 83 - DNA, 2741 bp, Ovis aries
  source 1..2741: mol_type = other DNA
agtgagtgagctttcctccggccggcgacgccatggagcctggggcggccgagctgtacgatcaggccctgctgggcatcttgcagcacgtgggcaacgttcaggactttctgcgtgtgcttttcggcttcctctaccgcaaaacggacttctaccgcctgctgcgccacccgtcggaccgcatgggctttcccccgggggccgcgcaggcgctggtgctgcaggtattcaaaacctttgaccacatggcccgtcaggatgatgaaaagaggagaaaagagctggaggagaaaatcaggaggaaggaagaagaggaggccacggtggtggcagctgccacagtggagcaggagccagtcccagccccggttcaggaggtggaggtagactccaccacagactcaggtgggcctcaggagcccccaggcccacagggtgccctgcaggaagtggctgtcagctctggggaggcagaggcaccaggagcagatgctggcgctgctgaagtccccagggagtcaccagctcttcccaagagacaggagcagttccagagaaaccccgacagctacaacggtgctgtgcgcgagaactacacctggtcccaggactacaccgacctagagctcaaggtgccggtacccaagcacgtggtgaagggcaagcaggtctcggtgaccctcagcagcagctccatccgggtggctgtgctggaggaaaatggggagcgtgtcctcatggaagggaagttcacccacaagatcaacactgagagctccctctggagcctcgaacccgggaagtgcgtcttggtgagcctgaacaaggtgggcgaatactggtggagtgccatcctggagggcgaggagcagatcgacattgacaagatcaacaaggagcgctccatggccacagtggacgaggaggagcacgccgtcctggaccggctcaccttcgactaccaccagaagctgcagggcaagccgcagagccacgagctgaaagtccatgagatgctgaagaaggggtgggatgctgaaggctctcccttccgaggccagcgattcgacccggccatgttcaacatctccccaggggctgtgcagttttaatgaccggaggagaggacgccctcgccggcagggaagcggggccgcggccgtgccccccagctccctcctgccggggactcgctgtctagtttcaagggtgaacccggcctccaccctgccctgcgtctccagactgtttcagagcagggcggggctgtgcgccttgcgagggggtgtcggggagcaggggctgtctcagcgggcacagctctgggccctgccggcaccgcttgccactccctcctccctgcgcccgtggctccgctcctgtgctctctgcttgctgtcctccgtgcgctcggggcggtcgtgggtgcccctcggctgtggaagcgcaggcagggatgtgcaggctgagggctgggctcagggccagtttggggaagctgccgccttgccctcttgctgggtgtgttcttgtgtcgtcttgcctgtctgcttgtgcgcgtccaccgccttcctcagcctcgtcgaagagccatggaaaccagcgtgctggtctctggttctgggtgctggaagagtctctggtggcctcaggccctgcacagtagggtctctgtcttctgattggcctgtcctgctgccacccggcctagcactttcccctgggggacctggttcttgtcagaggagctgggccaggtgccctcaggagacctcatggtgtgtcactgctcacggcagccccaggtcctttcttggctgaagccagactgtgccacccagagcttggggccctgctcccgcggctctcccaggacccagagcccttgcccttcgccaggaccgcgcggctgccctattggtctgttaccttgcaggcctctgcaccctggaacctgccctcgggctttgtgtgctggactgcaggaggaggagccatgggggtggggacgttccttctagaaacttccaccttcagttgtgtgggtgggagctggttgattgctgtgtgtgggggcaggtttctggctcacagggccctgtagacacactcctgggaagcacttgcttgctccagtgccatcacgggaggtgtgtacacctggtgactggtgggagcaccaccaagggggcatggagcactgttgttggctgtcgtcgccaaggctggtcacagagctgctgaggccccccttcagcgtcctccggatctcacctgttccacgtagtgagaggtctcactggtctgtgtgagaaccattctggcaccgtgcctgtctcctcccctccgccacccccagcgggtcctctgccgtcaacgggtggggtatgaggcctgcaccttggctgttgggagctgtggactccatggtcagttgtcttccttttgcccaattttattttcagatgtttgcatttctcctcaccccactggggatactagcagcttgccggcatctgggagtggcaggctggggggcttggcatcttgtgggctgcctgcacccctcgctgctggcctgggccctggacctgtgcactctgcctgttttctctcttcagacacggcaggagcagtttggccactgagctccatcagctgaggtccaaggaaaatggggtgcccgcctgcgaaatcaccctgtctccggtgttggtttgcaataaatctgtatttaagca

SEQ ID NO: 84 - DNA, 3426 bp, Bos taurus
  source 1..3426: mol_type = other DNA
gggctctgaggcgcgggactctgaggcgcgatggcggcgcgctgcttgggccgcggcgtcgggcggctactgggcggcctccgcggggttggggtgcgggcgctggcggccccccggctctggagcagcgcctccgcctccgtagcgccacccagctcctaccaagagcgaatcgcgcttgcagctcgcgagcccgccgccttctgggggccactggcgcgggacgctctcgtgtgggacaccccttaccacacggtgagcgactgcgacttccgcagcggcaggatcagctggttcctgggaggccagttaaacgtgtctgtcaactgcttggatcagcacgttcagaagtccccagagagcatcgctttgatctgggagcgagatgagcctggaactgaagtgaagatcacctacagggagctactggagaccacgtgccgcctggccaacacactcaagaggtatggagtccgccgaggggaccgagtggccatctacatgcctgtctccccactggctgtggctgccatgctggcctgtgccaggatcggagccatccacaatgtcatctttgctggcttcagtgtggggtccttggctgggaggatcaacgatgcccaatgcaaggtggttattaccttcaaccaaggactccggggagggcgcgtggtgcagctgaagaagatcgtggacgaagccataaaggtctgcccatccgtccagcatgtcctggtggctcacaggacagacaacaaggtccacatggggcatctggatgtttcccttgagcaggagatggccaaggaggagccggtatgtgccccggagagcatgggcagtgaggatatactcttcctcctgtacacctcggggagcaccgggaagcccaaggggctcgtccacacccaggcgggttacctgctctatgctgccctgacgcaccggcttgtgtttgactaccggccaggtgacgtcttcggctgcgtggctgatatcggctggatcacgggacacagctatgtggtgtacgggcccctctgcaatggagcgaccagtgtcctttttgagagcactccggtttaccctgatgcgggtcggtactgggagacggtgcagaggctgaagatcaatcagttctatggcgccccgacagcctaccggctgctgctgaagttcgaggactcctgggtgaagaagtatgaccgctcttctctgcggaccctgggctcagtgggagagccgatcaaccacgaggcctgggaatggctgcacagggtggtgggggacagccggtgcacgctggtagacacctggtggcagacagaaacggggggcatctgcatctctccacggccctcagaggagggggcagagatcctgccctgcatggcgatgaggcccctgtttggcattgttcccgttctcatggatgagaagggaaatgtcctggagggcggagacgtgtctggggctctgtgcctgtcccaggcctggccgggcatggcccggaccatctacggagaccaccagcgattcctggacgcctacttcgagacctaccctggctactacttcacaggagacggggcttacagaacagaggagggctactacgagatcactgggcgcattgacgacgttatcaacatcagcggccaccggctggggaccgcggagatcgaggacgccatggtgagcacggccgccgctggtctgggggctgagtctgagtcagtgcctcacctctgcgtgttttgtcctgcccaggctgaccacccctcggtgcccgagaccgctgtcattggctacccgcatgacatcaagggagaagctgccttcgccttcgttgtgttgaaagatgatgtgggtgatgtggacgtggtggtgaaggagctccggtcagtggtggccgacaagatcgccaagtacgcagtgcccgatcaggtcctggtagtgaagcgtcttccaaaaactcggtctggaaaagtcatgcggaggctcctgaggaagattgtcatgggccgagctcaggacctgggggacaccaccaccctggaggaccctggtgtcatcacggagatcctgagtgcctaccaggagtacaaagacaagcggggaggtgccaagtgatggctctgctgagaccaggcccctaatgcgtgctcctagagcacagctcccgagtcggcttgttcctagacaccccagcagaacccaggctgccctcctcgcgaagcctgaagtccccgcctcccctcctggggacccacgacaggtcctcgcccagggcgagcttcctcccgcttgtcccgaatctggtttcctggtgaaaagtccatccccctgtccccttgacagcacaggaaggtggagcccctgtgtctgctcaggtggactgggagcaaacagcagtcatgtttcctccagatgtttctagaaacaaaaggtgaagattttatttttatacttttatattttagaagaaacatggtgcccacacacgtgcagtgtatgctgtacttgtacacactcttcttaacagttatgaaaagctccttttttgcctccgctcgtggggaagtgtggcgggctggagggtgcactttgggttactgtttggcttgttggaaatttaagttggaaatgtttgtgcctctgtcttaacacagctatcttgaaacctctcaagcccacagcatctatttttggtgtgtttccacttctcttggtttgtgtgtgatgaaacctagctgctagaagcacctgtggggatgagttctggtcgtgtgtgtgtgtgcaggctgtggtctgggatgattaccctcccctggcctgggaccctcttagctgccggttggtgtgctccactcttggggctcttcagggaagctggggactcactaatgcaactgaaactgggtccctgcaaaccgccctacctccctgctctgagccagtctttccctttgccaatgtgccttgagtcctctggacttaacaaaatgatgtggaccgctaggctgcaaagggtctatttatagccaagtacatgagggcccacttgtggatcaaggggaggatggcctgctgagctgggtcccggagaacagggagggctgcccccacccggccctgtgccccacagtgggcggtgccctgcaggtgtacaggtgttcacagggcattgggttgcttaacacctgctgtggccatgctgggtggccaggtgaggatgggtggcctcggctatagcacatggctctaacaaaaccacacacctgcatctgtggtgtttcactggacttggttcattatagtcagggcactacttaggaaatgacttcgctataaactgggacagtgaccagggcctcaaaatgatctgcagggccgttcaaataaagctttattttgttcattctc

SEQ ID NO: 85 - DNA, 3345 bp, Capra hircus
  source 1..3345: mol_type = other DNA
ggcgcgggactctgaggcgcgatggcggcgcgctgcttgggccgcggcgtcggacggctactggggggcctccgcggggtgggggtgcgggcgctggcggccccgcggctctggagcagcgcctccgcccccgcagcgccgcccggctcctaccaagagcgaatcaagctggcagctcgcgagcccgccgccttctgggggccgctggcgcgggacgctctcgtgtgggacaccccctaccacacggtgagcgactgcgacttccgcagcggcaggatcagctggttcctgggaggccagttaaacgtgtctgtcaactgcttggatcagcacgttcagaagtccccggagagcatcgctttgatctgggagcgagatgagcctggaactgaagtgaagatcacctacagggagctactggagaccacgtgccgcctggccaacacactcaagaggtatggagtccgccgaggggaccgagtggccatctacatgcctgtctccccactggctgtggctgccatgctggcctgtgccaggatcggagccatccacaatgtcatctttgctggcttcagtgtggggtccttggctgggaggatcaacgatgcccagtgcaaggtggttattacctttaaccaaggactccggggagggcgcgtggtgcagctgaagaagatcgtggacgaagccataaagatctgcccatccgtccagcatgtcctggtggctcacaggacagacaacaaggtccacatgggggatctggatgtttctcttgagcaggagatggccaaggaggagccggtatgtgccccagagagcatgggcagtgaggatacactcttcctgttatatacctcggggagcactgggaagcccaaggggctcgttcacacccaggcgggctacctgctgtatgctgccctgacgcaccggcttgtgtttgactaccggccaggtgacgtcttcggctgcgtggctgatatcggctggatcacgggacacagctatgtggtgtacgggcccctctgcaacggagcaaccagcgtcctttttgagagcactccagtttaccccaatgcgggtcggtactgggagatggtgcagaggctgaagatcaatcagttctatggcgccccaacggcctaccggctgctgctgaagttcgaggactcctgggtgaagaagtatgaccgctcttctctgcggaccctgggctcagtgggagagccgatcaaccacgaggcctgggagtggctgcacagggtggtgggggacggccggtgcacgctggtagacacctggtggcagacagaaacggggggcatctgcatctctccacggccctcagaggagggggcagagatcctgccctgcatggctatgaggcccctgttcggcattgttcctgttctcatggatgagaagggaaatgtcctggagggcggagatgtgtctggggctctgtgcctgtcccaggcctggccgggcatggcccggaccatctacggagaccaccagcgattcctggacgtctacttcgagacctaccctggctactacttcacgggagacggggcttaccgaacagaggagggctactatgagatcactgggcgcatggacgacgttatcaacatcagcggccaccgagtggggaccgcggagatcgaggacgccttggctgaccaccctgcggtgcccgagaccgctgtcattggctacccgcacgacatcaagggagaagctgcctttgccttcattgtgttgaaagatgacgtgggtgatgtggacgtggtggtaaaggagctccggttggtggtggccgacaagattgccaagtacgcagtgcctgatcaggtcctggtagtgaagcgtcttccaaaaactcggtctggaaaagtcatgcggaggctcctgaggaagattgtcatgggccgagctcaggacctgggggacaccaccaccctcgaggaccctggcgtagtcacggagatcctgagcgcctaccaggagtacaaagacaagcgggggtgtgccaaatgatggctctgctgagaccaggcccctaatgcgtgttcctagaacacagctcccgagttggcttgttcctagacgccccagcagaacccaggctgccctccttgtgaagcctgaagtccccgcctcccctccccggggacccatgacaggtcctcgcccagggagagcttcctccctcttgtcccgaatctggtttcctggtgaaaagtccatccctctgtccccctgacagcacaggaaggtggagcccctgtgtctgctcaggtggactgggagcaaacaaacagcagtcatgtttcctccagatgtttctagaaacaaaaggtgaagattttatttttatacttttatattttagaagaaacatggtgcccacacacatgcagtgtgtgctctgcttgcacacactcttcttaacagttacgaaaagctccttttttgcctcggctcgtggggaagcgtggcgggctggagggtgcactttgggttactgtttggcttgttggaaatgtaagttggaaatgtttgtgcctctgtcttaacacagctatgttgaaacctctcaagcccacagcatctatttttggtgtgtttccacttctcttgatttgtgtgtgatgaaacctagctgctagaagcacctgtggggatgggttctggtcctgtatgtttgtgcaggctgtggtctgggatgattactctcccctggcctaggaccctcttagccgccggttggtgtgctccactcttggggctcttcagggaagcggggactcactaatgcagctgaaactgggtccccacaaaccgtcctacctccctgctctgagccagtcttttccctttgccagtgtgccttgagtcctctggacttaacaaaatgatggggaccggtaggctgcaaagggtctatttatagccaagcacatgagggcccacttgtggatcaaggggaggatggcctgctgagctgggtcccggagaacagggagggctgcccccacctggctctgtgccccacagtgggcggtgccctgcaggtgtacaggtgttcacagggcatcgggttgcttaacacctgctgtggccatgctgggtggccaggcgaggatgggtggcctcggctgtagcacacggctctaacagaaccacatacctgcacctgtagtgtttcactggacttggttcattatagtcagggcactacttaggaaatgacttcactataaactgggacagtgaccagggcctcaaaatgatctgcagggccgttcaaataaagctttattttgttcattc

SEQ ID NO: 86 - DNA, 3352 bp, Ovis aries
  source 1..3352: mol_type = other DNA
acagggctctgaggcgcgggactctgaggcgcgatggcggcgcgctgcttgggccgcggcgtcgggcggctactggggggcctccgcggggtgggggtgcgggcgctggcggccccgcggctctggagcagcgcctccgcccccgcagcgccgcccggctcctaccaagagcgaatcaagctggcagctcgcgagcccgccgccttctgggggccgctggcgcgggacgctctcgtgtgggacaccccctaccacacggtgagcgactgcgacttccgcagcggcaggatcagctggttcctgggaggccagttaaacgtgtctgtcaactgcttggatcagcacgttcagaagtccccggagagcattgctttgatctgggagcgagatgagcctggaactgaagtgaagatcacctacagggagctactggagaccacgtgccgcctggccaacacactcaagaggtatggagtccgccgaggggaccgagtggccatctacatgcctgtctccccactggctgtggctgccatgctggcctgtgccaggatcggagccatccacaatgtcatctttgctggcttcagtgtggggtccttggctgggaggatcaacgatgcccagtgcaaggtggttattaccttcaaccaaggactccggggagggcgcgtggtgcagctgaagaagatcgtggacgaagccataaagatctgtccatccgtccagcatgtcctggtggctcacaggacagacaacaaggtccacatgggggatctggatgtttctcttgagcaggagatggccaaggaggagccggtatgtgccccagagagcatgggcagtgaggatacgctcttcctgttatatacctcggggagcactgggaagcccaaggggctcgttcacacccaggcgggctacctgctgtatgctgccctgacgcaccggcttgtgtttgactaccggccaggtgacgtcttcggctgcgtggctgatatcggctggatcacgggacacagctatgtggtgtacgggcccctctgcaacggagcgaccagcgtcctttttgagagcactccagtttaccccaatgcgggtcggtactgggagatggtgcagaggctgaagatcaatcagttctatggcgccccgacggcctaccggctgctgctgaagttcgaggactcctgggtgaagaagtatgaccgctcttctctgcggaccctgggctcagtgggagagccgatcaaccacgaggcctgggagtggctgcacagggtggtgggggacggccggtgcacgctggtagacacctggtggcagacagaaacggggggcatctgcatctctccacggccctcagaggagggggcagagatcctgccctgcatggcgatgaggcccctgttcggcattgttcccgttctcatggatgagaagggaaatgtcctggagggcggagatgtgtctggggctctgtgcctgtcccaggcctggccgggcatggcccggaccatctacagagaccaccagcgattcttggacgtctacttcgagacctaccccggctactacttcacgggagacggggcttaccgaacagaggagggctactacgagatcactgggcgcatggacgacgttatcaacatcagcggccaccgggtggggaccgcggagatcgaggacgccttggctgaccaccctgcggtgcccgagaccgctgtcattggctacccgcacgacatcaagggagaagccgccttcgccttcattgtgttgaaagatgacgtgggtgatgtggacgtggtggtgaaggagctccggttgggggtggccgacaagatcgccaagtacgcagtgcctgatcaggtcctggtagtgaagcgtcttccaaaaactcggtctggaaaagtcatgcggaggctcctgaggaagatcgtcatgggccgagctcaggacctgggggacaccaccaccctcgaggaccctggcgtagtcacggagatcctgagcgcctaccaggagtacaaagacaagcgggggtgtgccaaatgatggctctgctgagaccaggcccctaatgcgtgttcctagaacacagctcccgagttggcttgttcctagacgccccagcagaacccaggctgccctccttgtgaagcctgaagtccccgcctcccctccccggggacccatgacaggtcctcgcccagggagagcttcctccctcttgtcccgaatctggtttcctggtgaaaagtccatccccctgtccccctgacagcacaggaaggtggagcccctgtgtctgctcaggtggactgggagcaaacaaacagcagtcatgtttcctccagatgtttctagaaacaaaaggtgaagactttatttttatacttttatatttgagaagaaacatggtacccacacacatgcagtgtgctctacttgcacacactcttcttaacagttacgaaaagctccttttttgcctccgctcgtggggaagcgtggcgggctggagggtgcactttgggttactgtttggcttgttggaaatgtaagttggaaatgtttgtgcctctgtcttaacacagctatgttgaaacctctcaagcccacagcatctatttttggtgtgtttccacttttcttgatttgtgtgtgatgaaacctagctgctagaagcacctgtggggatgggttctggtcatgtatgtttgtgcaggctgtagtccgggatgattactctcccctggcctaggaccctcttagccgccggttggtgtgctccactcttggggctctttagggaagctggggactcactaatgcagctgaaactgggtccctgcaaaccgtcctacctccctgctctgagccagtctttccctttgccagtgtgccttgagtcctctggacttaacaaaatgatggggactggtaggctgcaaagggtctatttatagccaggcacatgagggcccacttgtggatcaaggggaggatggcctgctgagctgggtcccggagaacagggagggctgcccccacctggctctgtgccccacagtgggtggtgccctgcaggtgtgcaggtgttcacagggcatcgggttgcttaccacctgctgtggccatgctgggtggccaggcgaggatgggcagcctcggctgtagcacacggctctaacagaaccacacacctgcacctgtagcgtttcactggacttggttcattatagtcagggcactacttaggaaatgacttcactataaactgggacagtgaccagagcctcaaaatgatctgcagggccgttcaaataaagctttattttgttca

SEQ ID NO: 87 - DNA, 4289 bp, Bos taurus
  source 1..4289: mol_type = other DNA
ccttgcagcttccaccctgaaagcccatgggacagcaacaggtgaaagttacacaggaactgaaaaacattcaagttgaacagatgacaaaacttcaagccaaacatcaagcagaatgtgatttgcttgaagatatgaggacattcagtcagaagaaggctgctattgaaagagagtatgcacagggtatccagaagttggctagtcagtatctgaagagagattggcccggaataaaagctgatgatcggaatgattacaggagcatgtatcctgtttggaaatcttttctcgagggaacaatgcaggtagcccagtctcggatcaatatatgtgaaaactataaaaacttcatttctgagcctgcaaggacagtgagaagcctaaaagaacagcaactaaaacggtgtgtggaccagttgacaaagatccaaactgaactgcaagagacagtaaaagatttagctaaaggcaagaagaagtactttgagactgaacagatggctcatgcagtacgagagaaagctgacatcgagtcaaaatctaaacttagtctttttcaatcaagaatcagtttacagaaggcaagcgtaaagttaaaagcccggcgatctgagtgtaattccaaagctacccacgcaaggaatgattatcttcttacattagcggcagcaaatgcacaccaggatcgctactatcaaacagatttagttaatatcatgaaggctcttgatggaaatgtgtatgaccatcttaaggattatttaattgccttcagccggactgagctagagacatgccaagctgtgcagaacacattccagtttttattagaaacctccagcaaggtggtacgagactataatcttcagctgtttttacaagagaatgctgtgtttcacaaaccccagcccttccagttccagccttgtgacagtgatactagccgacagctagaatcagaaacagggaccacagaagagcacagtttaaacaaagaggctcggaaatgggccacacgtgtggcacgggagcacaaaactattgttcaccaacaacgggttctagatgatctggaatgccatggagtttcagtatcagaacaaaaccgagcagagttggaacagaaaatagatgaagctaaagaaaatattcgtaaagcagagataattaagttgaaagctgaagcccgactggacctactaaagcagattggcgtttctgtcgacacatggctaaagagtgcaatgaaccaagtgatggaagaactggaaaatgagcgatgggcccgccttcctgcagtgactaataatggaaccttacactcgcttaatgcagatactgaaagagaagaaggagaagagtttgaggacaacatggatgtttttgatgacagcagttccagcccttctggtaccttaagaaattacccactcacctgcaaagttgtttattcctataaggcttctcaaccagatgagttgaccattgaggaacatgaggtgttagaagtgattgaagatggagatatggaagactgggtaaaggctcgaaataaagtcggccaagtgggttatgtgccagaaaagtacctacagtttcccacctcgaacagtctgctgagcatgctgcagtccttggctgccttggacagtcggtcacacacatccagcaattccacggaggccgaactcgtttcaggcagcctcaacggagatgccagtgtctgtttcgtgaaagcactttatgactacgagggccagacagatgatgaattatcttttcctgagggagcaatcatccgaatcttgaacaaagaaaaccaggatgacgatggcttctgggaaggagaattcaatgggcggattggagttttcccatcggtgctagtggaagaactttcaacctcagaaaatggtgacactccatggatgcgagagattcaggtctctccttcccccaaaccgcccggctccctgcctccactgccgctgtacgaccagcctcccagcagcccttaccccagcccagataagaggggctcccagtacttctcccgttctccttcagcaaacgaaaacagcttccatgccgaatcgccaggattctcgcaggcatccaggcatactcctgagacctcatatgggaaactgcgacctgtccgggcagctccccctcctcctacgcagaaccaccgaaggcccgcagagaagatcgaggatgtggaaatcacactggtgtgatgatgggtttgcctgtccgttactgctacaatcaaggccaggcttggagtttggccagtcttgttttttaggcacctttgcttgcatgatgatgactcgaacagagcaaaaacaaggaggactatatgtgaccgggtggcctggttgacacctcccacgtttttaatatttcgtgcctttttttgttgtcattttctatgtcatctctcctagcacagcacaaatcctagtgggccctagaagcagagaagggggacagtcctcttgcctaccagggacccatttttgtatgagactcaagaccaggcctcgttcagggcacagtcacagagatgctgatcatatgcactcgtacagtatattactgtggccacgaggacgtggcaaagatgctcatctttcttcaagtggctttggcttatctgattgagtttaatcagatcatgttggctacataaggaaagcagcaggagggatttcaggagaggctggctcctccccgcagttggtccccaaactgagaaactgaaaccttattggaaaaaagtagactgccctgagtttagcaccgattgcaataatgcacatctcttccacagctaacagatttaaaacaattaacagtgtcctttgtattaatatttatgccattcatttagtattagcagagctaacatgaaggggctgaactagtagccgaatctcatccatcacatagactaatagaaagggggctgcggctaaagcagaaatggagcttccagatctgcaatgagccgacttagtgttctttttatatttgggtatttttcatcttaatttttgcagcatatactatcatgaccagtatccttagaatttgaatgtcttgataaaactggggacacatacttgccttgtcaagcttctctactggagaggcacctagcattattttattcccaagctagtccactggcccaggcagccaagttgtggctggcccagaccagcagccacccaccacatgaaatgcagcttcccaggtcccactgacctgacatttccttgggaggaagaatctgtggctcaaggagtaaaccagcttcctatgaagaaggacaaaaggattgcacggtgcaaattgacaaggaaaggacaagttctccatgtcacagggagagccaggaggagccttccctgcagactaacaaacccacagcagtggctcccagaggcctctggtggccctccacctccaatggaagatagggagtgcattacttaggaactaagggatgtttgccagttgctttttttttccttttttttaaaaaatcaaaattctcagaatttaatccaaaagttgtcttttgctttgaaaatgccatccctcctaagaatctctaaaaacttgaaatgctcgccaaatgtccccatgggatttttgaccagaaataaaactgaagaagttttcagatagtcatttgatcactttaatgacagtatccattgatgaatcttctctgatttgaggatttttattttttttttgcatttcttaaaaccttttgatctatttgaggtctttttgtgtttatcaaacttattcttaagtttacaaaagaaattaaaagggtgggttttttttttttaggattttaaggtttgtggacaagttaaaaatcttcaaagcactttaaaaatcttcaaagtgctgaaattgggcagttcttgccacccagtcttgtattctctctttagctagcaaaattgtccttgaatctgggttttccacattctcagagagttatttgaaatcttttgttatattttaaatattttttggaagagctgctaattttattttaaaaaaacgaagttgtaaaaatatgcctcctttttaataagaacccgtccctccaggacatgtaaccctgcgccacagtgcacagggctgggctcagaggaggcacctctgtgcagaagtgctttctttacagtggctgtaaaaactttgtatttattatgtataaaatgttgaataataaaaaaaaatggaattac

SEQ ID NO: 88 - DNA, 5092 bp, Capra hircus
  source 1..5092: mol_type = other DNA
ctgccttagtgggtgtgggagggccgcgtgtgaggaaccgcggagccggcggcgggtggggaggcgcggagggcaggggacgggacggcagggttgctgctccggaggcgctgggagaccctgcgcccccgcagccgggcccccgccccctcgcggtctccgcctccttgcctcacacaccccccgggccgccagagcccaggccctcccggggctcctcctccttctcttcacactcaccctccgacccgggttctcctgccacccgccggccttgcagcttcccgggcgagtcttcgttctcaggctctccttacacacacacaagcacacgcccgctcccggggcggccctctcctctcgaccctccttttcctctcctcacttacgccgccgtgggaggaggattgatgtcccttcagcatcatgcagccgccgccgaggaaggtgaaagttacacaggaactgaaaaacattcaagttgaacagatgacaaaacttcaagccaaacatcaagcagaatgtgatttgcttgaagatatgaggacattcagtcagaagaaggctgctattgaaagagagtatgcacagggtatccagaagttggctagtcagtatctgaagagagattggcctggaataaaagctgatgatcggaatgattacaggagcatgtatcctgtttggaaatcttttctcgagggaacaatgcaggtagcccagtctcggatcaatatatgtgaaaactataaaaacttcatttccgagcctgcaaggacagtgagaagcctaaaagaacagcaactaaaaaggtgtgtggaccagttgacaaagatccaaactgaattgcaagagacagtaaaagatttagctaaaggcaagaagaagtactttgagactgaacagatggctcatgcagtacgagagaaggccgacatcgaggcaaaatctaaacttagtctttttcaatcaagaatcagtttacagaaggcaagcgtaaagttaaaagcccggcgatctgagtgtaattccaaagctacccatgcaaggaatgattatcttcttacattagcggctgcaaatgcacaccaggatcgctactatcaaacagatttagttaatattatgaaggctcttgatggaaatgtgtatgaccaccttaaggattatttaatcgccttcagccggactgagctagagacatgccaagctgtgcagaacacattccagtttttattagaaacctccagcaaggtggtacgagactataatcttcagctgtttttacaagagaatgctgtgtttcacaaaccccagcccttccagttccagccttgtgacagtgatactagccgacaactagaatcagaaacagggaccacagaagagcacagtttaaacaaagaggctcggaaatgggccacacgtgtggcacgggagcacaaaactattgttcaccaacaacgggttctagatgatctggaatgccatggagtttcagtatcagaacaaaaccgaacagagttggaacagaaaatagatgaagctaaagaaaatattcgtaaagcagagataattaagttgaaagctgaagcccgactggacctactaaagcagattggtgtttctgtcgacacatggctaaagagtgcaatgaaccaagtgatggaagaactggaaaatgagcgatgggcccgccttcctgcagtgactaataatggagccttacactcgcttaatgcagatactgaaagagaagaaggagaagagtttgaggacaacatggatgtttttgatgacagcagttccagcccttctggtaccttaagaaattacccactcacctgcaaagttgtttattcctataaggcttctcaaccagatgagttgaccattgaggagcatgaggtgttagaagtgattgaagatggagatatggaagactgggtaaaggctcgaaataaagtcggccaagtgggttatgtgccagaaaagtacctacagtttcccacctcgaacagtctgctgagcatgctgcagtccttggctgccttggacagtcggtcacacacatccagcaattccacggaggccgagctcgtttcaggcagcctcaacggagatgccagtgtctgtttcgtgaaagcactttatgactacgagggccagacagatgatgaattatcttttcctgagggagcaatcatccgaatcttgaacaaagaaaaccaggatgacgatggcttctgggaaggagaattcaatgggcggattggagttttcccatcggtgctagtggaagaactttcagcctcagaaaatggtgacactccatggatgcaagagattcaggtctctccttcccccaaaccgcccagctccctgcctccactgccgctgtatgaccagcctcccagcagcccttaccccagcccagataagaggggctcccagtacttctcccgctctccttcagcaaacgaaaacagcttccatgccgaatcgccaggattctcgcaggcatccaggcatactcctgagacctcatatgggaaactgcgacctgttgcgatggacttgggaagttaaacagagctctggaaaaggaaggctataaaaataaacacatcttggaaggtctagcttattacgctggcactttcctcactaagccatggtgagactgccaggtgacaactatggaaagtgtatttaaattataacaagttctgtgagcattaaagcagtctaattgtgaaacagaattttttttttttttccagcaggcgtttattgagaactttttttccaggcctctactttcctcttcgacagacagtgagatgatataagatacatgcccttttgaggtttgagagcttctattctcaaagaaattatttgttctttacaaataatcacctctacccaccccttactcggctgtcatcaaatctatcccactacctcactgtgaaatagttcttccagggtcataacttccgcatggtaatctagttacatttgcagttttattttcctagaccctcttatcagcatttgacacaattctttgagccacttacaacctttgtctttgttcattcagcagatactgagtgccttgttaaggacttggttcgtgatcctaaatgtgataggaagatgttgtaagattgtaaaccagatagtgatatgactggttgttcatttttaaaaattatttttacttaataatttttataattttatgttggctgcacttggtcttacttgtagcatgtgggctctttgtcactgagcatggacttctctttagttgcagtgagtgggcttagttgccttgaagcatgtgggatcttagttccccaaccaaggatcaagcctatatcccttgaattgggagaccaccagggacatcccagctgtgcattttaaaaacactactgtggcagctaggtggaaaatgagtagaaagtgtcagaatgaacatgaggagttgttaggaggctggagcaatggtccaagcaagagaggacagtaatgtgaactagtggggaagagcagtagtggagagaagtggacccatttgttccaccacacttttcctggtctgctttctacttcctggccttccttttcattgacctttgagggcttctctgcctttatccagctctgttgatgcttctcagggttccatccactaaccacttccctctttttttttttaatttgtttggctgcgtcaggttttagttgcagcaggtggagtctttgttgcatcgtgtgggatctttttgatgcgctgcacggagtctgtagtgtgggctccagcgcacacaggctttagtagctgctgtgtgcagcctcggtatttgggcactcaggtcagcctctcagctgttccatggcatgtgagatcttagttccctgactaagggttgaacccatgtcccctgcattgcaaggcaaattcttcaccattgagcaaccagaggagtcccagggctactgctcttaaagtcgttaaacttactcctgagttatctcatttactcccaagactttgataaccatttatttcctgacagctcccagatttataactacctatccaatctcttctctgagcttcagatctatggaaatcttagtgtatatatatagcaaacccccaaaactagcaaaaaaactagttaattatcctccttctcagacatcatccttctcccttatttcccatcctggtgagtggtacccctattctgctagtgctttgggatcactcgtgacacctccctgacctgcctttcatgaagagtcatcgcaaggcccgccagggttacctagagatgactgtcaagttgtccccctccattcccccagtgccagccaacatcagctctctttcagacgatcacaggaggctccaaaactgtcaacctgtattcctgccctccaccactcttttcttcaaacttttacctgagtcatcttttcacacatctaatattactgcttaaatccctcaaagacgcttctttgcctacagaataaggctcagatttttaaatatgagttaataaggcccttcatgacttaccccagcctgtctctctagtctcatctcttgcatctctccttccaagacacatttaaaggtttttcagttctccaagcattgctctctctcgcttccaactctaggcctttgcttgggccaaggttgtcaggactacaaagactcttgtggccatgaggctttcagcccagggaagaacctgagtcttgagtttcacatttcatccagagcagtcggtccttctcaatgtaagaagctataaaaattaagttgaaagcacatgcctagaacatttctgagagccctcgggtgcccatgtccttgaataagtgattggtcggatttcgatgcttcagccactgaatcaggaagctgactgaccatgtgatggtttcctgctcaggtccgggcagctccccctcctcctacgcagaaccaccgaaggcccgcagagaagatcgaggatgtggaaatcacactggtgtgatgatgggtttgcctgtccgt

SEQ ID NO: 89 - DNA, 4474 bp, Ovis aries
  source 1..4474: mol_type = other DNA
ctcttcacactcaccctccgacccgggttctcctgccacccgccggccttgcagcttcccgggcgagtcttcgttctcaggctctccttacacacacacaagcacacgcccgctcccggggcggccctctcctctcgaccctccttttcctctcctcacttacgccgccgtgggaggaggattgatgtcccttcagcatcatgcagccgccgccgaggaaggtgaaagttacacaggaactgaaaaacattcaagttgaacagatgacaaaacttcaagccaaacatcaagcagaatgtgatttgcttgaagatatgaggacattcagtcagaagaaggctgctattgaaagagagtatgcacagggtatccagaagttggctagtcagtatctgaagagagattggcctggaataaaagctgatgatcggaatgattacaggagcatgtatcctgtttggaaatcttttctcgagggaacaatgcaggtagcccagtctcggatcaatatatgtgaaaactataaaaacttcatttccgagcctgcaaggacagtgagaagcctaaaagaacagcaactaaaaaggtgtgtggaccagttgacaaagatccaaactgaactgcaagagacagtaaaagatttagctaaaggcaagaagaagtactttgagactgaacagatggctcatgcagtacgagagaaagccgacatcgaggcaaaatctaaacttagtctttttcaatcaagaatcagtttacagaaggcaagcgtaaagttaaaagcccggcgatctgagtgtaattccaaagctacccacgcaaggaatgattatcttcttacattagcggctgcaaatgcacaccaggatcgctactatcaaacagatttagttaatattatgaaggctcttgatggaaatgtgtatgaccaccttaaggattatttaatcgccttcagccggactgagctagagacatgccaagctgtgcagaacacattccagtttttattagaaacctccagcaaggtggtacgagactataatcttcagctgtttttacaagagaatgctgtgtttcacaaaccccagcccttccagttccagccttgtgacagtgatactagccgacaactagaatcagaaacagggaccacagaagagcacagtttaaacaaagaggctcggaaatgggccacacgtgtggcacgggagcacaaaactattgttcaccaacaacgggttctagatgatctggaatgccatggagtttcagtatcagaacaaaaccgaacagagttggaacagaaaatagatgaagctaaagaaaatattcgtaaagcagagataattaagttgaaagctgaagcccgactggacctactaaagcagattggtgtttctgtcgacacatggctaaagagtgcaatgaaccaagtgatggaagaactggaaaatgagcgatgggcccgccttcctgcagtgactaataatggagccttacactcgcttaatgcagatactgaaagagaagaaggagaagagtttgaggacaacatggatgtttttgatgacagcagttccagcccttctggtaccttaagaaattacccactcacctgcaaagttgtttattcctataaggcttctcaaccagatgagttgaccattgaggaacatgaggtgttagaagtgattgaagatggagatatggaagactgggtaaaggctcgaaataaagtcggccaagtgggttatgtgccagaaaagtacctacagtttcccacctcgaacagtctgctgagcatgctgcagtccttggctgccttggacagtcggtcacacacatccagcaattccacggaggccgagctcgtttcaggcagcctcaacggagatgccagtgtctgtttcgtgaaagcactttatgactacgagggccagacagatgatgaattatcttttcctgagggagcaatcatccgaatcttgaacaaagaaaaccaggatgacgatggcttctgggaaggagaattcaatgggcggattggagttttcccatcggtgctagtggaagaactttcagcctcagaaaatggtgacactccatggatgcaagagattcaggtctctccttcccccaaaccgcccagctccctgcctccactgccgctgtatgaccagcctcccagcagcccttaccccagcccagataagaggggctcccagtacttctcccgctctccttcagcaaacgaaaacagcttccatgccgaatcgccaggattctcgcaggcatccaggcatactcctgagacctcatatgggaaactgcgacctgtccgggcagctccccctcctcctacgcagaaccaccgaaggcccgcagagaagatcgaggatgtggaaatcacactggtgtgatgatgggtttgcctgtccgttactgctacaatcaaggccaggcttggagtttggccagtcttgttttttaggcacctttgcttgcatgatgatgactcgaacagagcaaaaacaaggaggactatatgtgaccgggtggcctggttgacacctcccatgtttttaatatttcgtgcctttttttgttgtcattttctatgtcatctctcctagcacagcacaaatcctagtgggccttagaagcagagaagagggacagtcctcttgcctcctagggacccatttttgtatgagactcaagaccaggcctcgttcagggcacagtcacagagatcctgatcatgtgcactcgtacagtatattactgtggccacgaggacgtggcaaagatgctcatctttcttcaagtggctttggcttatctgattgagtttaatcagatcatgccggctacataaggaagcagcaggagggatttcaggagaggctggctcctccccgcaattggtccccaaactgagaaactgaaaccttattggaaaaaagtagactgccctgagtttagcaccgattgcaataatgcacatctcttccacagctaacagatttaaaacaattaacagtgtcctttgtattaatatttatgccattcatttagtattagcagagctaacatggagggggctgaactagtagccgaatctcatccatcacatagactaatagaaaggaggctgtggctaaagcagaaatggagcttccagatctgcaatgagctgacttagtgttctttttatatttgggtattttttcatcttaatttttgcatatactatcatgaccagtatccttagaatttgaatgtcttgataaaactggggacacatacttgccttgtcaagcttctctactggggaggcacctagcattattttattcccaacctagtccactggccgaggcagccaagttgtggctggcccagaccagcagccacccaccacataaaatgcagcttcccaggtccctctgacctgacatttccttgggaggaagaatctgtggctcaaggagtaaaccagcttcctatgaagaaggacaaaaggattgtatggtgcaaattgacaaggaaaggacaagttctccatgtcacagggagagccatgaagagccttccctgcagactaacaaacccacagcagtggctcccagaggcctctggtggccctccacctccaatggaagatagggaatgcattacttaggaactaagggatgtttgccagttgcttttttttcctttttttaaaaaaatcaaaattctcagaatttaatccaaaagttgtcttttgctttgaaaatgccatccctcctaagaatctctaaaaacttgaaatgcttgccaaatgtccccatgggatttttgactagaaataaggtcatagctgaagaagttttcagatagtcatttgatcacttcaatgacagtatccattgatgaatcttctctgtgatttgaggattttttttttttttgcatttcttaaaacctgttgatctatttgaggtctttttgtgtttatcaaacttattcttaagtttacaaaagaaattaaaagggtgggctttttctttttaagattttaaagtttgtggacaagttaaaaatcttcaaagcactttaaaaatcttcaaagtgctgaatttgggcagttcttgccacccagtcttgtattctctctttagctagcaaaattgtccttgaatctgggttttccacattctcagagagttatttgaaatcttttgttatattttaaaatattttttggaagagctgctaattttattttaaaaaaacacgaagttgtaagaatatgcctcctttttaataagaacccgtccctctaggacatgtaaccccgcaccacagtgcacagggctgggctcagaggaggcgcctctgtgcagaagtgctttctttacagtggctgtaaaaactttgtatttattatgtataaaatgttgaataataaaaaacaatggaatta

SEQ ID NO: 90 - DNA, 5748 bp, Bos taurus
  source 1..5748: mol_type = other DNA
gagcgcatgcgcactggccgaagggcacctccctgcccggatctcgcgaagttacgttagattggccgccggtgacgggtggccgggccgggggcgggagggcttctgaggtaggaagggaggtccgttcggccgggcgcgccgccccagtgctctgtgggatactggaagtctcgagcgtctcctcctggttcccagccctcctctggccgcactcatagaaacagtcacacaccccgtgcctcccctctcttccctctccgcctcccccttccccccctcgcgataagaagacccggcggcaggagaggggatgaagatggcggacgcgaagcagaagcggaacgagcagctgaagcgctggatcggctccgagacggacctcgagcctcctgtggtgaagcgccagaagaccaaggtgaagttcgatgatggcgccgtcttcctggctgcttgctccagcggcgacacggacgaggttctcaagctgctgcaccgcggcgccgacatcaattacgccaatgtggacggactcactgccctgcaccaggcttgcattgatgacaatgttgatatggtgaagtttctggtagaaaatggagcaaatattaatcaacctgataatgaaggctggatacctctgcatgcagctgcttcctgtggatatcttgatattgcagagtttttgattggtcagggagcacatgtaggagctgtcaacagtgaaggagacacacctttagatatcgctgaggaggaggcaatggaagagctactgcagaatgaagttaatcggcaaggggttgatatagaagcagctcgaaaagaagaggaacgaataatgcttagagatgccagacagtggttaaatagtggtcatataaatgatgtccggcatgcaaaatctggaggtacagcgcttcacgttgcagccgccaagggctatacagaagttttaaaacttttaatacaggcaggctatgatgttaatattaaagattatgatggctggacacctcttcatgctgcagctcattggggtaaagaagaagcatgtcggattttagtggacaatctgtgtgatatggagatggtcaacaaagtgggccagacagcctttgatgtagcagatgaggacattttaggatatttagaagagttgcaaaagaagcaaaatctgctccatagtgaaaaacgggaaaagaaatctccactaattgaatcaacagctaatatggataataatcagtcacagaagacctttaaaaataaagagacattgattattgagcctgagaaaaatgcatcccgtattgaatctctggaacaagaaaaggttgatgatgaagaagaaggaaagaaagatgaatctagttgctctagtgaagaagatgaggaagatgactcagaatcagaagctgaaacagataagacaaaacccatggcttctgtaactaatgccaatacttctagtacacaagcagctcctgtagctgttacaacacctactgtgtcatctggtcagccaacacctacatcacctattaaaaagtttccagcttcagctacaaaaatttctcccaaagaagaagagagaaaagatgagtcccctgcatcttggaggttaggacttagaaagacgggcagttacggtgcacttgcagaaatcacagcatctaaagaagctcagaaggaaaaagacactgcaggtgtcatgcgctcagcttccagtcccagactttcctcctctttggataataaagaaaaggagaaagatagtaaaggaactaagcttgcatatgttacgcctacaataccaaggcggcttgccagtacatctgacattgaagagaaagaaaacagagattcttcaagtttgcgaacaagtagttcatacacaagaagaaaatgggaagatgatcttaaaaagaatagctcaattaatgaaggatcaacttatcacaaaagttgctcctttggtagaagacaagatgatttgattagttctagtgttccaagcaccacatcaacaccaacagttacctctgcagctgggcttcagaaaagcctgctttccagcacaagcactactacaaagattacaacgggttcttcctcagcaggcacacaaagcagtacctcaaatcgtttgtgggctgaggatagtactgaaaaggaaaaggacagtgttcctacggcagtgaccattcctgttgctccaactgttgttaatgctgcagcttctaccacaaccctgactacaactactgctggcactgtctcctccactgcagaggtcagggagagacgcagatcatacctcactcctgttagagatgaagagtctgaatcccaaagaaaagcaagatctagacaagcaagacagtctagaagatcaacacagggggtaacattgactgatcttcaggaagctgaaaaaacaatagggaggagtcgttccacccgaaccagagaacaagaaaatgaagaaaaagaggaaaaagagaaacaggataaagaaaagcaggaagaaaagaaggaatcagaaacatctagagaagatgaatataaacaaaagtactccagaacatatgatgaggcttatcaacgctacagaccagtatcaacttcaagttcaaccactccctcctctttactttctacaatgagcagttcactttatgcttcaagtcaattaaacaggccaaatagtcttgtaggtataacttctgcttactccagaggattaactaaggaaaatgaaagagagggagaaaagaaagaagaagaaaaagagggggaagataaatcacaacctaaatcaatcagagaacgaaggcgaccaagagagaaaagaagatctacgggagtttcgttttggacacaagatagtgatgaaaatgaacaagaacaacaatctgatacagaggagggatccaataagaaagaagttcagacggattccatttctagatatgaaatcagttctacatcagctagtgatcgatacgattccttgctgggtcgctctggatcatacagttactcagaagaaagaaaaccttacagtagcaggctagaaaaggatgattcaactgactttaaaaagctttatgaacaaattctagctgaaaatgaaaagctgaaagcgcagctccatgatacaaatatggaactgacagatcttaaattacaattggaaaaagccacccagagacaagaaagattcgctgataggtcactattagaaatggaaaaaagggaacgaagagctctagaaagaagaatatctgaaatggaagaagagctgaaaatgttaccagacctgaaagcagacaaccagaggctaaaagatgaaaatggggccttgatcagagttataagcaaactttccaaataaaaaaaagcagcaagtaatggaattgcacatactagtaacccagtggaccataattgaccgtcactggaagcctgggaagaatccttggagactgtcatttttggatgtcctgccaaatgccctcttatctaggagttttgtttaattttctgttttttttttttttttttttctcttgtatcaagaccattgtttcatgttaatgcagctgctgagaagaattttttttatgactgagaaaacttgtttacagctccagcatataaggaaagtgttcaaggccagatatgcctcagatatttaaccagtaagccttagttgtacataaatacttttgtgtcaacaaaaactcagctctcacagaagacagttattcagcattttttgatgtgccatgccacagtttgcagtttttcaatatttaattttattttattttgttttgttattgctttacatctaagtttgggtttgtatcttttccttctaactcttcatgtggcagactttctgtcgtctcacagctttctcatttacagaaaagaacacttgctcctctgaaatcattgtcatatattaggctaatgttgttgtccccacctggaactgaattgcttggtggaacatttgctttcactgtttgtgcaatatgcatttattccttttatgaatgctttaaagtcatttgagattagattcttttaagtcctattttctgccttcattggtcacttttttgtttttgtttgttttttattattgtagtgtaagatgttaggttctgtaatcttcacattcattttagcaggtactgagtgatgctgtatatataaataagtgtattgtttgatttttagaccaccacatggcatgcttgactattatttcaaatgtctgttaatgcaaagtaggctactccataatagtgttaagaacaaaacttactaacaaagtgatataaagacttaaattaacacattatgtggagccctatctttacaaaagttttctactgtaaaatgcttttacttttatttgcagttttcatttgatagtattcaaccataattaaagttgcataagataattgcttcacatttcacatacctatatttatctgagtgctgtctaaaactgttgtgctagccaaagtaatgctatgaaatcatttgcagacttaacccgtgagtttgtgttaaatgcactgttattgccatgtgaagaggcattgactttgataccaccatcatgttcagaccattttatacatttcagtggccttttttaaaaaaaagaaaaagaaaagcaaaaccaagtaaatagtgaagatggcatttatttggacaaatagcttttatattttcattaaaccatgcaaaaagtactatatctttctggcacataactgtctccttaaccactgaacagttcagccatttgaataaattgtacattgtaaagcttatagtagctaattgtattattgattgtattgtatactattcaatgtgaatttgtaccccttcattttaaaaggtcattgtgttctactgcctattttagattacgttggggttgggaaagggtgttttggtaagcaggattttaagtcctctcttgattggttttatgaagatataaggttatgctcttactaccaagctcaggattcttgatttttaaaggtacaaggatagttgtgggaactttatttttggttacatttgaactgtatgaatggtgggtctgaatatagagaatttccatggtcttacgtggacgtaaaacatacagataattgatcagtttgagtgactgcaacatattctggttgcacaacagttaggcatgctaaggattatatttgtgaagtttggatgcctgtgaagaatgattaaatacgtgtttctaatgttttagtgttttgtatttaggttatttattctttgtatctaaaactgaacagctactgtgctatattgattttattggtagtattgagcagaccttgtttctgcatgaaactagttgcaaaacccactattttggaaataaaaatgtattttgacatatacaaataaaatgaataaaactattttaaatgtgtgaacttttactgaagacgtttaaaactttgttctgatatttaaattctgcatgggtatcttcactaactgctttacttggattccattggtataatttttgaaattgtaatattgtgataacttgcacaacgttgtacatgcacatctgtaaatttaagcttaattgccatatttatgcaatctgtgtatcaatttgaacaaatgtttatatattttacataaagctatctgtgtgcagaa

SEQ ID NO: 91 - DNA, 5472 bp, Sus scrofa
  source 1..5472: mol_type = other DNA
cgaagttacgttagacgggccgccgggacggacgggcggccgggccgggggcgggcgggcgtctgcggtcgaagggaggtccgcgcggccgggcgcgccgccccagtgctctgtgggatactggcagtctcgagcgtctccgcccggtgcccagcccgccgccgggccgcgctcgccggggcagccccacgccccctgcccgccgcccctccgctcgccgcctcccccgccctccgcgcgctcggcagacccggcggcaggagaggggatgaagatggcggacgcgaagcagaagcggaacgagcagctgaagcgctggatcggctccgagacggacctcgagccccccgtggtgaagcgccagaagaccaaggtgaagttcgacgatggcgccgtcttcctggccgcctgctccagcggcgacacggacgaggtgctcaagctgctgcaccgcggcgccgacatcaactacgccaatgtggacgggctcaccgccctgcaccaggcttgcattgatgacaatgttgatatggtgaagtttctagtagaaaatggagcaaatattaatcaacccgataatgaaggctggataccactacatgcagctgcttcctgtggatatcttgatattgcagagtttttgattggtcagggagcacatgtgggagctgtgaacagtgaaggagatacacctttagacattgctgaggaggaggccatggaggagctgcttcagaatgaagtcaatcggcaaggggttgatatagaagcagctcgaaaggaagaagagcgaataatgcttagagacgcaagacagtggctgaatagcggccacataaacgacgtcaggcacgcgaaatctggaggcacagcacttcacgtcgcagctgctaaaggctatactgaggttttaaaacttttaatacaggcaggctatgatgttaatattaaagattatgatggctggacacctcttcatgctgcagctcactggggtaaagaagaagcctgtcgaattttagtggacaatctgtgtgacatggagatggtcaacaaagtgggccagacagcctttgatgtcgcagatgaagacattttaggatatttggaagagttgcaaaagaagcaaaatctgctccatagtgagaaacgggagaagaaatctccactaattgaatcaacagccaatatggacaataatcagtcacagaaaacctttaaaaacaaagagacattgattattgaacctgagaaaaatgcatcccgtattgaatctctggaacaagaaaaagttgatgatgaagaagaaggaaagaaagatgaatctagttgctctagtgaagaagatgaggaagatgattcagaatcagaagctgaaacagataagacaaaacccatggctcctgtaactaatgccaatacttctagtacacaagcagctcctgtagctgttacaacacctactctgtcgtctggtcaggcaacacctacatcacctattaaaaagtttccaactccagctacaaaaatttctcccaaagaagaagagaggaaagatgagtctcctgcatcctggaggttaggacttagaaagactggcagttacggcgcccttgcggagataaccgcatctaaagaggcacagaaagaaaaagacactgcaggtgttatacgttcagcttcaagtcctagactttcctcctctttggataataaagaaaaggagaaagacaataaaggaacaaggcttgcatacgttgcacctacaatcccaagacgactcgccagtacatctgacatggaggagaaggaaaacagagattctttaggtttgcgaacaagtagttcttatacaagaagaaagtgggaagatgatctcaaaaagaatagctcaattaatgaaggatcaacttatcaaaaaagttgctcctttggtagaagacaagacgatttgattagttctagtgttccaagcaccacatcaacaccaacagttacctctgcagctgggcttcagaagggcctgctttccagcacaagcactactacaaagattacaacgggttcttcctcagcaggcacacaaagcagtacctcaaatcgtgtatgggctgaggatagtactgagaaagacaaggacagtgttcccaccgccttgaccgtccctgtcgctccagctgttgtcaatgctgcagccgctaccacaaccctgaccaccactactgctggcactgtctcctccacctcggaggtcagggagagacgcagatcatacctcactcctgttagagatgaagaatctgaatcccaaagaaaagcaagatctagacaagcaagacagtctagaagatctgttcagggggtgacattgactgatcttcaggaagccgaaaaaacaataggaagaagtcgttctacccgaaccagagaacaagaaaatgaagaaaaagaaaaagaagaaaaagaaaagcaggataaggagaagcaagaagaaaagaaagaatcagaaacatctagagaagatgaatataagcagaagtactccagaacattcgatgagacttaccagcgctacagaccagcctccacttccggttcagccactccatcctcttcgctgtccaccatgagcagctcactctatgcttcaagtcaactcaaccggccgaacagtcttgtaggtataacttctgcttactccagaggatcaacaaaagaaaatgaaagagaaggagaaaagagagaagaggagaaagagggagaagataaatcacaacctaaatctatcagagagcgacggcgaccacgagaaaagagaagatctacaggcgtttcattttggacacaagatagcgatgaaaatgaacaagaacaacaatcagatacggaggagggatccagtaagaaggaaactcagacagattccatttccagatacgagaccagttctacgtcagctagtgatcggtatgattccttgctgggtcgctctggatcatacagttacttagaagaaagaaaaccttacagtagcaggctagaaaaggatgattcaaccgactttaaaaagctttatgaacaaattctagccgaaaatgaaaagctgaaagcccagctccatgatacgaatatggaactgacagatcttaaactancattggagaaggccacccagagacaggaaagatttgctgataggtcgctattggaaatggaaaaaagggaacgaagagctctagaaagaagaatatctgaaatggaagaagagctcaaaatgttaccagacctaaaagcagacaaccagaggctaaaggatgaaaatggggccttgatcagagttataagcaaactttccaaataaaaaaaaagcagcgaatgatggaattgcacatattattccccaatggaccataattgacagtcactggaagcctgggaagaatccttggagactgtcattttcggatatcctgccaaatgccctcttatctaggagttttgtttaattttctggttttttttttttttttttttttttttttttcttccttgtatcaagaccattgtttcatgttaatgcagctgctgagaagattttttttttttatgactgagaaaacttgtttacagctccagcatataaggaaagtgttcaaggccagatatgcctcagatatttaaccagtaagccttagttgtacataaatacttttgtgtcaacagaaactttcagctctcacagaagacagttattcagcattttttgatgtgccacagtttccagtttttccatatttaattttttttgctttacatctaagtttgggtttgtattttttccttctaactcttcatgtggccgactcctctgtgttctcacagtttccttacttacgggaagactcctgctcttccgagatcgttgtcgtgtgttaggctcatgctgtgtttccccccacctggaactgagttgcttggtggaacatttgctttcactgtttgtgcaatatgcatttatcccttatatgaatgctttaaagtcagttgagattagattcctttaagtcctattttctgccttcattggtcacttttttgtttttgtttgttttttattactgtagcataagatgtaggttctataatcttcacgtccattttagcaggtactgagtgatgctgtatatataattaagtgtatcgttttgatttttagaccaccacatggcatgcttgactcttttcttatttcgaatgtctgttaatgcaaaataggttactccatgatagtgttaaaagacaaagtttgctaacaatgtgatataaagacttttaaagtaacacattatgtggagccctatctttacaaaagttttctactgtaaaatgcttttacgtttattcgcagttttcgtttgatattatccaaccataattaaagttgcataagataactgcttcacatttcacatacctgtatttatctgagtgctgtctaaaatactgttgtgctagccaaagtaatgctatgaaatcatttgcagacttaacccaagagttaatgttaaatgcactgttatcgccatgtgaagaggcgttgactttgatgccaccatcgtgttcagaccattttatacatttcagtggccttttttttaaataaaaaaaaaaaaagcaaaaccaagtacatagtgaagatggcttttatttggacaaatagcttttatattttcattaaaccatgcaaaaaatactatatctttctggcacataactgtctccttaaccactgaacagttcagccgtttgaataaattgtacattgtaaagcttatagtagctaattgtattattgattgtattgtatactatattaaatgtgaatttgtaccccttcattttaaaaggtcattgtgttctactgcctattttagattacttttgggttgggaaagggtgttttggtaagcaggattttaagtcctttcttgattggttttatgaagatataaggttatgctcttactaccaagctcaggattcttgatttttaaaggtacaaggatagttgtgggatttttattttttggttatatttgaactgtatgaatggtgggtctgaatatagagaatttccatggtcttatgtggacgtaaaatgagaagataattgaccagtttgagtgactgcaacatgctctggttgcacagcagttaggcatgctcaggcttatatttgtgacgttcggatgcctgtgaagaatgattaaatacatgtttctaatattgtagtgttttgtatttaggttatttattctttgtatctaaaactgaacagctactgtgctatattattggtagtattgagcagaccttgtttctgcatgaaactagttgcaaaacccactattttggaaagaaaaatgtattttgacatatacaaataaaaagaataaaactattttaaatgtg

SEQ ID NO: 92 - DNA, 3762 bp, Bos taurus
  source 1..3762: mol_type = other DNA
gctccgggggctctatatagggcgcgcgctcgcgggccgctgagttccaccagtccgcgagctgccgtcggctccgcgcggggggggcgggccgggcaccccggggcgcggaggtgcgctctagcttaattcttctttccccccaactccgcactcccgggcaccctcaaacccagtccacgctgctgcccccgctccctcttttctcggcccccagcaaactttcggtcccttcctcgcccctcgcccgttactcgtcgtggctcgggcccggccgcgccgccccgagggctcctccggatctcccagtttccagctccgaccattagaggatccagaaacatgtcgaccacacttctgtccgccttctacgatatcgacttcttgtgcaagacggagaaatccctggccaacctcaatctgaacaacatgctggacaagaaggcggtggggacacccgtggccaccgcccccagctcgggcttcacgccgggcttcctccgacggcactcggccagcaacctgcacgcgctcgcccaccccgcgcctagccccggcagctgctcgcccaagttcccgggcgcggctaacggtagcgcggcggccggcggctcggcctcctacggcaccctcaaggagccgtcggggggcggcggcaccgccctgctgaacaaggagaacaaattccgggaccgctcgttcagcgagaacggggagcgcagccagcacctcctgcacctgcagcaacagcagaaggggggcggcggctcccagatcaattcgacgcgctacaaaactgagctgtgccggcccttcgaggagagtggcacctgcaagtacggcgagaagtgccagttcgcacacggcttccacgagctgcgcagcctgacgcggcaccccaagtacaagaccgagctgtgccgcaccttccacaccatcggcttctgcccctacgggccgcgctgccacttcatccacaacgctgatgagcggcgacccgcgccgtccgggggcgcctccggggacttgcgcaccttcagcgcccgcgatgcgctgcacctaggcttcccgcgggaaccgcggcccaagctgcaccatagcctcagcttctcgggcttcccgtcgggccaccaccagccccctgggggcctagagtcgcccctgcttctcgacagccccacgtcgcgcacgccgccgccaccctcctgctcctcggcctcgtcttgctcgtcgtcgcttcgtcctgctcctcggcctcggcagcctccacgccctccggcgcccctacgtgctgcgcggccgcggcggcggccgcgctgcctgtacggcaccgggggcgccgaggacttgctcgcacccggcgcaccctgcgccacctgctcgtcggcctcgtgcgccaacaacgccttcgccttcggcccggagctgagcagcctcatcacgccgctggccatccagacccacaacttcgccgccgtggctgctgccgcctactatcgcagccaacagcagcagcagggcctggtgccccccgcgcagcccccggcgccgcccagcgcgcccctccctgccagcgccgccgcgccgccctcgccgcccttcagcttccagttgccacgccgcctgtccgagtcgccggtgttcgacgcgccccctagccccccagactcgctgtcggaccgcgacagctacttgagcggctccctgagctcgggcagcctcagcggctctgagtcccccggccttgacccaggtcgccgcctacccatcttcagccgcctctccatctccgacgactgaggcaagagggcgccagtgaggaggaggaagggaaggccgttctgggggtgttggagggtgcccctgcttaggggcagggggccacgggagtgtggggggtgacatcggccacaagcagactgcaggctgtgccgagcacttggactcgaacttgagtgccgggaggggcccccacccctcccctttcggttccctcttcgttttttgtttttttattattattattatttttattattattacgaagtttcattcttgttgagcgggaaaaaagccaacgaactttttgtattgatgaacaaaagactcacaacagagcagttgtgatgcgaatagaacaaaaaccaaacaaacaacaaacttttttaggtctccgatgagtttgggattttaggaaaaatcaacccagcaccagcagctatcaaccaccattccatatcttcacttgaaaagcattagttacagtccagatgtcaggactcttatcttgagagaggttccttcctaattgtttgtcccagaccagtgtaaacaaaccagccaaccaccgccttgctaataaacctataagcttttagaatccaatattctgccaagaatatgccttgatagtagacctcagctccataggtgtttgttttttaacaaagttatatatgtctggaaaaaaaaaaaccttgcattccaaagtttcatactggttactggtttcattgccaccacttagtggatgtttaatttagaaccattttgtctgctctttctggaagccttgggcagagcttagttcataattgatggggaataactgctgaatttttagctgttttgagttgattcgcaccactgcaccacaactcaatatgaaaaaaaactatttaacttatttattatcttgtgaaaagtatacattgaaaattttgttcatactgtatttatcaagtatgatgaaaagcaatagatatatattcttttattatgttaagttatgattgccattattaatcggcaaaatgtggagtgtatgttcttttcacagtaatatatgccttttgtaacttcacatggttattttattgtaaatgagtaaaaaaatcttaatttaagagattgtatgtaatatttatttcattaatttctttccttgtttacgtaaattttgaaagattgcatgatttctttacagaaatctatcttgatgctgtggaagtagtttgaggaatatcttacgagtttttcttagaatgtataatggttgtagcccatccaactttaaaagaaaaaatgaccacattgcaatcaggctcacatgtggtatgcttttctcattccaactttataaattagcaaaagatgtttttgtttgcttgttccaccagttctactgtgacatactctgcatataaagacatgtagcaataatggggcgagggtgatctcacagtgcctttggaagggccagaacttgccttaaattttcctcaaccaaataagtattttgtctattagtgcttgagagaatctgaatgtaggatgggttcaactgcacaaaaggagaagatttttaccactttttttttatatagatataaagtgaagaggccacctcttagtgctaaaatggtatgtagtacatgaatatgccttgtttaattacagaaaattccaaaacttgtactattttttttcccccgtgtggaaaggcaggaatgcttttctggacagcagctgaatgagcagtagctttagttcgtttgtacgtaggtacagtcggagcactatgtatgtgtgtattgtggactactttgacagaagtaggtattttgaatgtaacaaggttaagtcaacttgagttgtaatatatttggggaatcagttcactacaaattgtgactgtaaacattgtactgtaaatgttttgtagttttcccccaataaaacttttgggaaaaaaaaaaggta

SEQ ID NO: 93 - DNA, 3888 bp, Bos taurus
  source 1..3888: mol_type = other DNA
gtggcagcagcagcagcagtatcttcaaaatggctgataatttggatgaatttattgaagagcgaaaagtcagattggccagagacaaagcagaattggaaagtgatccaccgtacatggaaatgaagggaaaagcatcagagaagctttctgaaaacagtaaaatattaatttccatggctaaggaaaacataccacctaacagtcaacagacaaggcgttctttaggaattgattatggattaagtttaccacttggggaagagtatgaacagaagaaacataaactgaaagaagaattgcgacaagattacagacgctatcttactcagggtattacacaagcaaaaagaaagaaaaattttctatccacgagtgaaacagatccatctactctgggagtttcccttcctattggtgagaggttatctgctaaggaaaggttgaaacttgaacgtaacaaagaatacaatcaatttctcagggataaggaagaatccactgaaaagttcagacaggtagaaaagagtactgagcagaagaatcaaagaagtaaaaaacctatcagtcaggttaaacctgatttaccttcacaaatacaatcatctttcgaaaattcagagggtcctagaaaagatgtcttaactccttcagaggcatatgaagaacttctaaaccaaagacgactagaagaggacaggtaccgaaaattagatgatgaaattgaattaaggaacaatagaagagtcattaaaaagactgatgaagaagtgggcgtttccagtaaaaaacatcaaaggtttgacagcacgactgacattccagatagaagatttcacaggtttaatgaggatcttgcttttgataaacagtcttacagaccaggctatgatccagaagtaagtgaagacatggatgagaggtttagatatgaaagtgattttgatagaagacttttgagagtttatacaaaaaacaggatgtacgggaacagacgaggcaatgagcctcctatggagtgtgatggtgatgtcacagaacagtcaaacatacgaatttcatctgctggaaatcaaagtgctcgagacaatgaaccatccaaacctacttatcgagagacatgcagtccttttgcgggaatgctctttgggggtgaagatcgagaacttattcagagaaggaaagagaaatatagacaagaactattggaacaaatggctgaacaacagaggaacaagagacgagaaaaagatttggatctcagggttgcagcttctggagcacaagaccctgagaaatcgcctgatagactaaagcagtttagtgtggcaccaagacactttgaagagatgataccacctgagagacccagaatagctttccagacacctctccctcctctgtccgccccatctgtcccacccatcccatcagtttactccatcccatctcagagtgaagatgtgcacagtggactcagcagcactcttggtgacatggtgcctcccagagttgcacctctgcctccacctcccatgctgccccctttggctgcgaactaccgaactccttacgatgacgcgtactatttctatggggccaggaatactttggatcctagtcttgcttattatggttcagggatgatgggagtacagcctgctcctgctccttatgttacagctcctgtcacccaccagccagtacagcctattgtgagtacagttggacagaatgagctgaaaattacaagtaatcgagcaataaattcaggattgttttttgaagataaaccaaagccttcaaaacagtcatttcagtcttaccaggaagctttgcagcagcagattcaagaaagacaagaaagaagaaagaaagaacgtgaagaaaaagaagtatatgaagccaaattagaagctgaaatgagaagttacaacccctgggggaaaggtggaggtggtgctcctctgagagatgacaaaggaaatctgataactgatttgaataggatgcacagacaaaatatagatgcctaccataacccagatgcaagagcatatgaagataaaagggctgttgtatctctagaccaaaatttagccacttcaaatgcggagaaccaagaagatgctgcaaataaaaactcaggtcatatgcaatcacagagctctccttttgctcggggaaatatttttagtgagcctccaactgaacttcaaattaaacagcaggaattatacaagaattttcttcgttttcagattgaggaaaagaagcaaagagaagaagcagagcgagagagattaagaattgctgaagaaaaagaagaaaaacggcttgcagaacagagagctcgaatccagcaagagtatgaagaggagcaggaaaagaaaagggagaaagaggaggagcaaagactgaaaaacgaagagcttattcggttagctgaagaaagacgaaaagaagcagaaagaaagaagaaagaagaagaagaaaaacataatctacaacttcagcactactatgaaagagaaaacatgattggggaagatacaaagcacttgagacagccttctcctgtggttcctgctcttcagaataagattgcaagcagactccaaagacctccttcagttgacagcattataacttcctttactcaggacagttccatgtccatggcacaatctcctccggtacctgccaggaaaaaccagcttcgtgcagaagaggagaaaaaaaatgtaattatggaattatcagaaatgagaaaacagcttcgtagtgaagagcgacgtctgcaggggcggttactacacattgatagtgacgacgacattcatataaggaaaagagaaagttccatggatgtatttgatatggccagacatcgattacaagctcctgtcagaagaccgtcacctaagagcttagactctgcttcttctcagaatattcatgactttaaggagctgaaagatagagattcagaaacgcgagctgatctgaaatctatgtacccagatgatccaagagataatgacaccttggagatagaacaacaagccttgctaagagagcagcagaagagactgaataaactaaaaatgcaggaagttgacttggatgctgtcccaagcactaaagtacgggaccacagaatgcccagagaggacactgatgatttcttgaaaaactcattattagaatctgatagtgcttttattggtgcgcatggcgagacgtacccagccattgaggatgggggcttcctgctgctatctccgctgccctccgccagagagcgcagaaggaacaaactgaaaggattggactttgataacagtcaccctgatgtgccaccagatggtgtctctttaaaatctgtatccagtataaatattgatcagcttagaatgagaaatgaagaacgaatgcgaagactgaatgaacttcagaataaacctattactacagatgatgagagttcactggtggaccctgatgacatcatgaggcaccggggtgacgacggatcaaactctgtagccactgagccctggctccgcccaggcacctcagaaacactgaagcgattcatggtggaacagctgaggcaggagcagcagcagggtcctggcaaaccaggcgctttcacctggcagggactgtctactgcacacggttaaaatcagtctgtactggacccagtagtgcctttgaaggtgaaaggcatgtgaaacctgaacctgaataatatgtgctacttttggcctgtacatggcagttactcatattccatctgtatagaaggtatattttttccatgatgatgtatatgatgtatttatattatgtacataaataaaaagccatggctatttatat

SEQ ID NO: 94 - DNA, 2343 bp, Bos taurus
  source 1..2343: mol_type = other DNA
acttcttgcttccttcccataaaaactcagaggcataaaaggcccagagaatgagcatcccagagaaactgaccagggatatgggagtgatcgccatggatcagaagtctttctgggaaggtttagtggtcttgctgcttcttcaggaaggatgtgcctacaaactggtttgctactttaccaactggtcacaagacaggcaggagccagggaagttcacccttgagaacactgatcccttcctatgttctcacctcatttactcattcgccagcatcagtaacaacaaggtcatcatcaaggacaagaatgaggcgaagctttaccagaccatcaacagtctcaaaaccaagtcagtaggagaggagtggggagcacccagggaatcccaaactgaaaattctgttgtctattggagggtatctgtttggttccaaaggaagttagcagaagcttttcagcaggactttgtaaaatccaccaaagaaaggcttctcttgactgcaggagtttctgcagggaggcagatgattgacaacagctatcagatcaaggaattggcaaaagacctggatttcatcaacctcctgtcttttgacttccatgggtcttgggaaaagccccttgtcactggccacaacagccccctgagaaagggacagctggacagagggacaagctcctactacaatgtggaatatgctgtgggatactggataaacaaggggatgcctgcagagaaggtggtcatgggcatccccacgtatggacgttcctttacactggcctctgcagaaactgccgtgggggcccctgcctctggtcctggagccgctggccccatcaccaagtcttcaggcttcctcgcttattatgagatctgtcagttcctacaaggagccaagatcacaagacttcaggatcagcaagttccctatgcggtcaaggggaaccagtgggtgggctatgatgatgtggagagcgtggagaccaaggttcagtttctaaagaatctaaacctgggaggcgccatgatctggtctattgacatggatgacttcactggaaaactctgcagccagggcccctaccctcttgttcaagctgtcaagaggagccttggctctctagaaaggtgagggtgctgctgaccaaccaggtgtgctgacatcatctcagtggaggagcaggacactggaaatcactgcagacagacaactggagaggactggccatattgtcctggagaagggcccagagcagtgcagaatctcctaaactatatgattgggcagcagcctttcaactctgaacatctatgtcttagtaacttgactgaggccttatgttactgattggatttgaaatactttctttttctttccaggcttaattcatagctaagtaggaaaggtgaccttgctgcgtactcctgctacaggaattcatgtgtgggattcttggaggttagatgaggccctcaggatgtctcttctccaagtctccccttgactttctctcagacctcagactgggtctggctttgttctcctgcagcgctgactgcttcccttgaggcacaataaagaaaaagcatgttgttccagccagcgtggcctcctctgtgcaatcttggaccttgtggtataaggctgtcctctcccaccatttatgggtacaagtgtaggcctgcgtatcatatgtctaaatatttaagagccaagggttaaaatatgatttatactcttaacgaacatgattttgcaatgacccaaaagaccaggtttgaagttagaattcccagactcctgggaattctgtgccagaatgcagcagcttgtgcccttggttcccagcccactggtagcccctttcccttcacatctaaagccctgactcagagagcaaggacctcacatacacactggcagatgttggaaaggggaagttgttaggggaaacaatgactgaaactgcccaccctgtcaggcactacagtaactatttgcatgagttattttacaacaggaggtcctggtaaggaacatagaactaacaagccatcaccaactggaagaattcaggagaactcaaaaggagactccatgtgtcctaccacctcccagaatcctcactgaaatccatctcggctgagcaatgcatgtgccaccaagaagcaccctgattcagagtgagtggctggaaacaacccagaaactaaccccatcgccataaaatccgagactgagggccatgtggtagagcagccctgctgggttccccttaccttcctgctctctgcctgggtgcctttttccaataaaatcgcatgctttgtcagaa

SEQ ID NO: 95 - DNA, 1053 bp, Bos taurus
  source 1..1053: mol_type = other DNA
tagaaagagcgataagtcccccagcacagtgcccacactttgggaggggaggcaggagagtatggagggagtgaaggacaagcagcctgcagggagctttcctcctgccgctcggtatctcggtttggacctgaggcagctggcaggtaacagcctgtgcaccatgtcctggatggatgctcctcctccagcatccatccaggagtgcagaaaggagcccaccacggaatacagaggtttctgtctgggctccacagtgcagtctgaaaccaagggaatctggatgtggtatgtgccccacccctccaaggagaaccacaccctggtccttctggacactgagggtctgggtgatatggaaaagggtgactccaaaaatgactcatggatctttgccctggctgtgctcctgagcagtacctgtatctacaacagcatgggcaccatcaaccaccaggccctggagcagctgcactatgtgactgaattaacagagctgatcagaatcaaatcatctcccatctctgatgatgtagaggactcagtcgagtttgtgagattctttccagactttgtctggactgtgcgggatttcatgttggagctggagtttgatggaaaccccatcactgaagatgagtacctggagaatgccttgaagctgattccaggcatcagagcaggggcaaggtggtagaaagctcagtaggaagtgggatcaagagctctgatattcagcacttgatgtcactcttaatttggggttggaatgaggtcatgctaatggtgcctggtgaatgactggtgggtggattcagtcattgtttttactggaagaacttcagagaccagagcataagttcgctgaggaaatttagaatctagtattaaacgaacaattttagaaaaatttatatcatgaagaaaaagtctcacatattgtattaaatattaagtgcctcttttttcctgtttgagaaaaataacccatcaaattgaaattggtagatgtagaatcataaggtaaataaaaggattagtttgtgaaatatt

SEQ ID NO: 96 - DNA, 393 bp, Bos taurus
  source 1..393: mol_type = other DNA
atgaggctggtgtacctttttcttggccccatgaccctcctgctcctggctggctgtggctgtgtcatcagcacctccagtgagaacctgcgcacatttgtgggctgtgcagtgagggagtttacttttctggccaagaagccgggctgcaggggccttcggatcaccacggatgcctgctggggccgctgtgaaacctgggagaagcccattttggaacccccctacatcgaagcccatcatcgggtctgtacctacaatgagaccaaacaggtgacggtcaagctgcccaactgtgcccctggagtcgaccccttctacagctaccctgtggcggtccgctgtgactgcggggcctgctctaccgccaccacggagtgtgagaccatctga

SEQ ID NO: 97 - DNA, 5228 bp, Bos taurus
  source 1..5228: mol_type = other DNA
ggaggggaggtacctgggactgggagtgatgtcagctcccagctcggtgcctgcccggattcctgacatggtgtagggccgagagggtggggaaggcgggagccgggcgagagaggactcggggcagccagcctgcggcggggccggagtgccggcgctttgaactccgggcgaaggtgaagcacaacttttgcaactcgtgctggcggctctcctccccccagtataaaaagaacttcatggatcagactgctaacaagtacatggaagatttgaagagctgaagaaaaaaaaactcactgttaaacttgttttttaaaatatctaggttggaatttgcccttgacaaataacaagatgatgagtgatgcaagtgacatgttggctgcagcgctggagcagatggatgggattatagcaggttctaaggccctggaatattccaatgggattttcgattgccagtcccccacctccccattcatgggaagcttgcgagccctgcaccttgtggaagaccttcgtgggttgctggagatgatggaagctgatgagaaggaaggcttgaggtgccagatcccggactctacagcggaaacgctcatcgagtggctccagagtcaaatgacgaatggacacctccctgggaatggagatgtgtatcaagaaagactggcccgtttagaaaatgataaagaatccctcgttcttcaggtgagtgtgttgacagaccaggtagaggctcagggagagaagattcgcgatttggagttttgccttgaagagcacagagagaagttgaatgccacagaagaaatgctgcagcaggagcttctgagtaggacatccttagaaactcagaagttggacctgatggctgaaatatctaacttaaagttaaaactgacagctgtagagaaggacagactggattatgaagatagattcagagacacggaggggctaatgcaggagatcaatgatttgaggttaagagtcagtgaaatggaaaatgaaagacttcagtatgagaaaaagctgaaatctactaagtctttaatggccaaactttctagcatgaaaatcaaggtgggtcagatgcagtatgaaaagcagcggatggaacaaaagtgggaatcactgaaggaggagctgacatctctgaaagaacaactggaagagaaggaatctgaagtgagaagactacaagaaaaattggtttgccagatgaaaggagaagggattgaaattcttgatcgagatgaaaattttaaaaagaagctcaaagacaaaaatattgaagtacaaaaaatgaaaaaggctgtggagtctttgatggcagcaaatgaagaaaaggatcggaaaatagaagatcttcgacagtgcctgaacaggtataagaaaatgcaagaaacagtggttttggctccaagtaaaaaaggccaagatggcgactttgaagacctgcttccatccagctccttttccaccctgctggatgctcagggctttagtgatctggagaagaacctgtcgccgacgccagtaaggggatctcccagtcatgacccatttaacacaagtgctccagaagagttccacaccagcatcttgcaagtttcaatcccttcattattgccagcatctaaaggcttggaaacttctgaaaaagcaaaattgccttctaagccagagactccatttgaggagagtgatgaaattggttctgctgctgaaactcaactgtgtgatagtcttgcaacttcaagtttgcagaagtctagcagcctgggcaatctgaagaaagagtcatcagacggggaaaaggagtctattccaaagccttcagaagataaagctccggtagagagcagtccctttgggaccctccctcccaaagctccaggacatgatgcctctggggatgacaatccctttggcactcggaaagccagatcttcctttggtcgtggtttttttaaaatcaaaagtaacaagaggacagcaagtgcgccaaacttggatcgtaaacgaagtgccagtgcacccaccttagctgaaacggagaaggagacaccggagcacctagatctggctggtgtatcttctcggccgaaaaattcacagggaagtagtcccttccagatgtctccaccatctccagattccaaaaagaaatcccggggtattatgagactctttggaaaacttaggagaagtcagtccactacattcaacccagatgacatgtctgagtctgaattcaaaagaggagggacaagggcaactgcggggccccgattgggttggtctcgtgatttgggacagtctaacagtgacttggatatgccatttgccaagtggaccaaggatcaggtttgcagttggctggtggaacagggtttggggtcctacctgaattctggcaagcactggattgcatctggccagacacttttgcaggcttctcaacaagacctagagaaggagcttggaatcaagcattcccttcatcgaaagaaactccaactggcactgcaagccctgggatctgaagaagaaaccaatcatggaaagctggatttcaactgggtcactagatggttggatgatattggcctcccccagtacaagacccagtttgatgaaggacgggtagatggtcgaatgcttcattacatgactgttgatgacttactgtcgttgaaggtcgtgagtgtgctccaccatctcagtatcaaaagggccatccaggtcctaaggatcaataactttgagccaaactgtctacggaggcgaccctctgacgagaacagcatcaccccatcagaagttcagcagtggaccaatcaccgagtgatggagtggctgcgctccgtggacctggcggaatatgcccccaaccttagaggcagtggtgtccatggtgggctcatggttctagaacctcgttttaacgtagaaacaatggctcagttactgaacatcccaccaagtaagaccctgttgcgaagacatctggccactcacttcaaccttctgatcggtgcagaagcccagcaccagaagcgggatgccatggagttaccagattatgtacttctaaccgcaaccgccaaagtgaagccaaagaagcttaccttcagcaattttgggaacctgagaaagaagaaacaggaagatggggaggaatatgtttgtccgatggagttgggacaggcatcaggagggacatctaagaaaggatttaagcctggtttggatgtacgcctgtatgatgaggatgacctggaccgattagagcagatggaagactcagaagggacagtgagacagataggtgcattctctgagggcatcaacaacctaactcacatgttaaaggaagatgacatgtttaaagattttgctgcccgttcccccagtgccagcatcaccgatgaagactcgaacgtctgaccatagcacctggatgagagcagggagcccttcgtctttgttccactctattcctcggacactcacagtatctacagcaagccacaggctgtgtgttgtttatcgttcccgcttctcatgtggaagtggaggtggaaagcagggattaggtgctaattctaaagttgccatgacaattgagacactggtgaatgagagtataattgtttctcctctatttaatgtaaaaaaaaatctgtgatatattatatttaaagtgttgcatttaatatgagtattttaccatagtctttccatccacattcacagcatggaggatttgggaagcagtgaccagtatgtgaatgattttttgtcacgttgatgtgatagcactgcggccctcgaagcaggatgttacatgcttttgaaatcaaattgtggaatttcttaggcattcagtgtgtccactatatgccagccacacctccacttgcttctttttgtcctatttttatatatttttctaaatatatgtatatacttagtacatagaaaatagaacttttattttgtaacataaggaagatggtaaaaagatcacgttaaaaaaacacagtgagcaaaatttttgtataccagctgtttcttgaagaggtccaaatgatatttctgtcccccaaactgaactctaatgatgttgatcattcagactaaattaacatatgtgaaataccccttctttgatgacacttcacaaaattattgaatagctttttaaaaatttcagccttagtcttggatcctcttaactcataaggaagaatctgagggacattttaagtgctattaacctgagtgtattcaagaaactccccccaaatttgtagaaataattcctgaagaaaccaaaacaaaaccctttacagtattaagcttcttatcttcctctttgctaaacatatcattgcatcaaaatactaacagtctgcctaatgttattaattgcacatttctcagactattagcaaatggattttttttaaagctgattacttgtctgaacattttgtctcagacttcagtaactcaaagactgcaaaattagtttcataatggctatagtgtgatgataaaatgcagttcatgtttttctgtagcacagggcccaaatattctttatcaagaaaattgaaagctgcaataaaaactgttattgtgtttattattttctagaggtatcagaaaaatatccaattttttgttcactactatctcattacttatgatagtcagtggtggtaaagacaggttttcaaaaattagcgtagggtgggcagttcaatatatgtctgaagacttgcctaataaactatatttgaatgtcagtggcatgacctttcaagttagcaagtttccaagtttcttatcaaggaaccctgtgatactaaattactatttattcataattaaaatgactcaatgctaataatatttttggtccctatcatgtactatttggtaactgtattggactctttgtgtatttgtgttttattttaatgtgaacatatgtcaccatttgaaaaaccatttgttattcaatcctggttaaaaatactaggagactgttacagatgccaaatattccttattcaggacagtgtaacatcactgatgatatgtgataggttaaatttttttgatgatatatatcctttttacaagatatttacactgctggtattaccatatgaaaagaaataaagtcagttgataactgcctcata

SEQ ID NO: 98 - DNA, 1609 bp, Bos taurus
  source 1..1609: mol_type = other DNA
ctctcagaaatttgtttgaggggggtctataacctgtatcagggtttccccagtggctcagcagtaaagaactacttgcaaagcaggagaggtaagagacatgggttcaatccctgagtcaggaagatcccctggaggagggcacagcaactcactccagtactgttgcctggagaatcctatggacagaggagcctggaaggctacagtccatagggttgaaaagagtcagacactactgaagccactaagcatgcatagcctgtatcaacagatgaaaatttaaggcctttacttcagaaactttctcatgaaaggaaacacaggcagctgagcttgggggatgaggaatcatggcaaaggggtatcggaaaagcaaaccaaaatgacttgcctacaagtttgaagaagttagttgtagaaagaagttagttttatacatatgtttctttctttaaagaattaaatagtattttttaataaaaactaaaacttgaaaaaaggaaacttgtctcagaagaggttaccaaatatttgaagttaggtgaatataactagaagacagatgattttttgtgaaagtatctctgacttaataaaccagcaggactccatatgagtacaacacacccagttggcaaacaggaagacctaacatgtttgtttagctgtgtgtatatatgcttaattgtagagttcgaaaactgggaccacattcacagatgtgacttcactgcagaatttttactctgaaaaaaaacagttctcgtgctcaatatctagaagtagtgttcagcgtgtgcagggaaatggggcagaaaccatcacaacagctggctccaaaggacaaagaggttctgaacgtctgtgaggtggtcagtggagctatcatccacgcagctcagaaactgaaggcctatcttggatttgaagacacactgagcaatctgtgcccagattcaaacactctcaatgagatcttcttaatccactttgtcactttctgccaagagaaaggagctgacaagtggctcaccaccaccaagatgaccaagcaccaagccttcctgtttggggcagattgggtttggaccttctggggatctgataagcaaatacagcttcagctggccgtgcagacactgcagatggcttctcttcctcctgtggaatctgacccctccaacccagattccaaggcagagaagtctttcagcaagaaaagtaggtttgacaagctggaagaattctgtaacctgataggagaggactgtcttggcctgtgtatcatctttggtgtgccaggaaaccctaaagacatccgaggagtcgtcctggacagtgtcaagagagagacgacgaagggtcatctgccaggagggacagctgtggcacgattcatcctggaaacggaagattgtatctccatcagggagctgcttggaaactgtctgagtaagaaagatgggctgagagaggtgggcaaggtttatattagcatcctctgaactggccgtgtgggatgagactgacctgtaagataaaacaaaaatatgttcttctaataagtttatcctctcatgggatttttttctcccctcgctccacc

SEQ ID NO: 99 - DNA, 2111 bp, Bos taurus
  source 1..2111: mol_type = other DNA
tggctggcttagtgtggatcctggcagttttttcttgggtcagggtcagaccacgccgtcccaggaggaggaggggagttgtgcagtacaagcagctcttgacagaaggatgctggagctgagcctatctcggctgggactcgggccgctggcagcctccccgtggctgctgccgctcctggctggggtctcctggatcctggcccgcgtcctggcctggacctacaccttctacaacaactctcgccgcctccgatgtttcctgcagcccccaaaaccgaactggttcttgggtcacatgaacctggtgccctccacggagcagggcctgatctacttcactcagatggcggccaactacccccgtggatatttgatctggtttggccccattatcccaatggtcatcttctgccaccctgacatgctccggagcatcaccaacgcctcagctgccattgcacccaaagacatgcagttctatggcactctaaaaccctggcttggggatgggctcctgctgagtgccggtgacaagtggagcagccaccgtcgcatgctgacacccgccttccacttcaacatcttgaagccctatatcaagattttcaccaagagtgcagagatcatgcacgccaagtgggagcacctgatcacagagggccacacccatctggacatgtttgaacacatcagcctcctgaccctggacaatctgcagaaatgtgtcttcagttttgacagcaattgccaagagaagcccagtgaatatatcgccgccatcttggagcttagtgcgcttgtgtcaaaaagaaaccaacaactcttcctgtatatggacttcctgtactatctcacctcagacgggcagcgcttccgcaatgcctgccgcctagtgcacgacttcacagatgccgtcattcaggagcggcgccgcactctccccaaggagaatattgatgacttcctcaaggccaaggcgaagaccaagactttggacttcattgatgtgcttctgctgaccaaggatgaggatgggaagggattgtcagatgaagatatccgcgctgaagctgacaccttcatgtttgagggccatgacaccacagccagtggtctctcctggatcctgtacaaccttgcaaagcacccagaataccaggagcgctgccggcaagaagtgcaagatcttttgagggaccgtgagtctaaagagattgaatgggacgacctggcccagttgcccttcctgaccatgtgcatcaaggagagtctgcggttgcaccccccagtcaccagcatctcccgccgctgtacccaggacattgtgctcccagatggccgggtcatccccaaaggtgttgtctgcctcattgatattttcgggacccaccacaacccatctgtgtggcaagaccctgaggtctatgaccccttccgcttcgacccagaaaacatcaaagggaggtcacctctggcttttattcccttctcagcaggtcccaggaactgcattgggcagacattcgccatgactgagatgaaagtgatcttggcgctcaccttgctgcgcttccgcgttctgccggacaaggaaccgtgccggaagccagagctgatcctgcgcaccgagggaggactctggctgcgggtggagccgctgagcgcaggccagcagtgacccagcaccttctctcctgagatcctctctgaccctaaacacctcacgtgcagattggaaatcaaacttcgtggtcacctctgccctagtctcgtctagaagacagcaaggagcacatgggatctgctaggttccaggcaggtgggggttgctgtaaacctggtggcctttagcggggcggctcagaagggaacttggatcctaagtgtaagactactcattaggaaagtccaactgcttgatgcagagagggtcagatgaccttggatttgctccctgacatggtaatataaaggtcaccgtcccagtctgccacttactagttgtttggcttaagaaaattgtttacctctcagttcctttattacataattgtagccactttactgggctgttggggagattgaataaacaagcatatgcaaaatga

SEQ ID NO: 100 - DNA, 3174 bp, Bos taurus
  source 1..3174: mol_type = other DNA
cccgccttccggctccggcggcgacgctcgcttcttgccgccgccgcgctgccggtgtcagcctccctcgggagactccatttgctctcagattggtctggtcgccgcgaagtcaggcagagagcgatgtccccggcccgtggactggtgtcccccgggccaagcgccctctccctcgctggctggcttcggtggtgacatcctggttgctggcgaccacggaggaatccgggctccctctccatgagcaaagacgtcccgcgtttctgcggggaaaggccctcgctaacacggctgctctctgacctcttaatttcaggaggttttcggaggcttggagtgcctatacagcccccgacaggaactgcttctgttggcccatcgaggatcatctttcaccaggattatcgtaacatcttcactgagccttccaccccatagtctctaccatgagtcctgatttctgccaatgaagttttgtttttttttttttttttacgctttctttaatcatgtgacaccacttctggtcagccttttcccttttcagttgccaaacacattaggtggaagtttgtgtcgtgttaaagtacggagtttcttgcttgtccactctcagccttgtttgcactgctcacttctgttcagcttgccttctcctgcttgcctatgatttccattatcactctcttgcgtaacctgtagcagtccttgcccggggaggcagcattccatacagtgcactgcctgacaaggacagcagtcagggggtgagttttcccattataatttcttattaagtttgcaagtcctgtcttaaaaactttgttccctagcacatttctctaaattcgtccaccagtgacttagttcctttaacatagagcctcacccataggatggcaaaaacaattaagtacacaataggactgttttagagaccttgctaatacagtatgggttatcatcaccattgagattacagttcatgaaaagatcttaaacttaggagaagcaagatagaagataggattaattggagctagtctttgttgttttgagtttgtaaattttaaaagagcaaagtgaagagagtcacataggcttggcctggaaattggccaacacactgtccatctaggtcctagtggggcaggaaagggatcttaaaacaccattttttaaatctcattgtgtccaaatctaacttattaaaggtcttgttgtagtaatcctctaaaatattaggtacaatgttggggaaacgaaagcgtgtggtgttgacaattaaggacaagcttgacattattaagaaattggaggagggcatctctttcaaaaagctttccgtggtatatggaattggtgaatccacagttcgtgatattaaaaagaacaaagaaaggataataaactatgccaacagttcagatcctacaagtggggtatccaaacgtaaatctatgaagtcgtccacatatgaagaacttgacagagttatgatagagtggtttaaccaacagaaaacagatggaattccagtgtcgggaacgatttgtgcaaaacaagccaagttcttttttgatgctctggggatggaaggtgattttaatgcatcctctggctggctgactcgattcaagcagcgccatggtattccaaaggctgctggtaaaggaacaaaattaaaaggagacgaaactgctgccagtgagttttgtggtaactttcaggaattcgttgagagagagaatctacaaccagaacaaatttatggtgctgatcaaactggattgttctggaaatgtctaccatcaaggacattagctcttgaaactgagcaaagttcttctggttatagatcaagcagagagagaatcattattatgtgttgtgcaaatgccactggtttacacaaacttaatctttgtgttgtgggaaaagcaaaaaaacctcgtgcattcaaaggagctgatctttcaaaccttcctgtcacttatttcagtcaaaaaagtgcatggatagaacattctgttttcagacagtggtttgaaaaatactttgtgccacaggtacagaagcatttgaaatccaaggggcttctagaaaaagcagtgctccttttggactttcctccagcacatccaaatgaagaattgttgagttcagatgatggcagaataattgtgaaatatttaccaccaaatgtcacaagtctcattcaacctatgagccaaggagttctagccacagtaaaaagatactaccgagcaggacttctccagaaatacatggatgaaggaactgaccccagaatgttctggaagaacttgacagtgttagatgcaatttatgaagtgtcaagagcttggaacatgataaagtcaagtaccataaccaaagcatggaaaaaacttttccctggcactgaagagaattcaggcatgaacattgatgaaggagccattttagcagctaacttagcaacagttttacagaacacagaagactgtgaacatgttgaccttgagaatattgatcagtggtttgactctcggagtaatgattcaagctgtcaggtcctaactgacagtgaaggtgtggaggaccaggccaggcctgccgagaccaagccttccagtaagactagaaaaacagaactgaatccagagaagcatatcagccataaagctgcacttgaatggaccgaaaatttactagattatctagaacaacaagatgacatgcttctgtctgataaactggtattgcgaaggcttcgaaccataataagaagaaaacagaagatccagaataacaaaagtcattaataaggctcttaagagtttcagtgtatttgcatctgtgtgactttatctgcagtggaacttaatttcttatttgaattgctgtggatttcaaagccaaatgcattttataaatgactttagaattagatgccattttggattacgtaaattactgcacttgaatgttgattctaggaagttgacatgtaccttgtttgtctactgttcctaatctgtattgtactaatttagatcataaggtacctgaggccagggcgcctatgtctgttttgtacctgaatgtcattgtctaatagaggaccacgcacactataggcaatcaataaatctttcttaaattgaa

SEQ ID NO: 101 - DNA, 1354 bp, Bos taurus
  source 1..1354: mol_type = other DNA
agagcaccggggggcagggccaggcccgtggggctggattcactggtatcgattagagctgcccgggtggccgaggacgtgctcggtcgcagctgccagaccatgctgagtggagtgtgcgcccgactcgcgtcagcgctgcgagggacacgcgcgccgccgtccgcggtcgccctgaggtgtctgcacgcgtccagctcgcggccttccgcagacaagagcgataggacggagaaaccgccccgcgccttcgaccaggccttgctggagttcctggtgtgtccgctctccaagaagccgctcagatatgaagcatcgacaaatgaattgattaatgaagagttaggaatagcctatccaattattgatgggattcctaatatgataccacaggcagctaggatgacacatcaaaataagaagcaagaagaagtggagcagcattagatcatacttaaaaacaacacacacaactaaatattctaaataccacctaccttttaaaaagtcagagtggcgggtattaagtggagaagaatgtttctgtctcttcctatgctgactgtcctgattccactggtatctttagcaggactgttttactcagcctctgtggaagacgacttccctcagggctgcactagcaccaccagtctgtgcttttacagtctgctcttgccgattaccatacctgtttatgtgttcttccacctttggacttggatgggaattaaactcttcaggcataattaacgcagccagagccaagatggtatattctaaagatatgcttgagtgcctgtctctgaaggctcgtctacatgggaatactggaaacccgcttaatttgcaggagagatgctttgcctagctttctgtcaggggtctagggtctatgagaggcttaagctgcagaggcgtctttattgtactttggttctgcccttgttttttgaaggttctcatttatacctggaatatcaaaaggaacattacaaactgtctttttggaaattaacagccccaaccatcacttgatggcttatcccattcataattaaaaattactattttgaaattttatgtttattttagattaaaggctttggtaatgtcacatgttaacgatgactaaaataattccgaaggagctatgttggagtagttgtagagaaatgcatttgaactaatgtgatataaaactatagtaaaatggaaatttcatggacttgccaaaattctgagtttttaaaatcaccatcatttctttggcatcaaatgcttacattaattttaataaaataataaataaaaagatattgacacttgccataaagcaaaaaaaaaaaaaaaa

SEQ ID NO: 102 - DNA, 1386 bp, Bos taurus
  source 1..1386: mol_type = other DNA
cctctctgaggtggctttggaaggagacaccaggtgacacgtgtttgaaaagggtttaaaagttggatgcagttggagtggacggtgtttatctagtaggagcagtgaactggaactgctgtggacttcagtgagcgtttgagcagaacagccagcaatggataactcaatggtctgtttggccaatgcaacagtttgcaatggcacgtcctgtctggtaccagagagcaacttcaacgccattctaagtacaatcctgagcgccgtcctgactatcctgttggctctggtgatgttctccatgggatgcaatgtggaactcaagaaattccttgggcacataaagcggccatggggcatctgtgttggcttcctctgtcagttcgggatcatgcccctcacaggattcatcctctccgtggcctttgacatcatgcccatccaagctgtagtggtcctcatcatgggatgctgtccaggagggactgcctccaacatcctggcctattgggtggatggcgacatggacctgagcgtcagcatgaccacctgctccacgctgctcgccctagggatgatgccgctgtgcctgttaatctacaccaaaatgtgggttgattcggggatgatcgtgattccctacgataacataggtatatctctggtggctcttgttgttcccgtctcccttgggatgtatgttaaccacaagtggccccaaaaagccaagatcatccttaagattggatccatcacaggcgccctgctcatagtgcttatagccgtggttggaggcgtgctgtaccggagcgcctggatcatacagcccagactctggatcataggaaccatatttcccatagctggctactccttagggttttttctggctaggatagccggtcagtcctggcacaggtgccgaaccgttgctttggaaacagggatgcagaacacacagctgtgctccaccatcgtccagctctccttcacccccgaggagctcaacctcatattcaccttcccgctcatctacagcatcttccagatcatcgcagcggctctgttcctggcagtgtacgtggtatacaagaaatattataggaaaaacaatgtggaatctccggagggcagagacaacgaaccagtacctaagtcatcgttctaacaggtcaatgaaggatttcaaccagacgaaaagcaaacaccagtggaccaaaaagcatcctaaagaatatgctttagctctaacaaatacttaattctttgtttggataggatatttggcagaaaaaaagttacagtgaaaatttaaatttgaattgaattcgtgtattggttttggtaccaagcgactggcgatccaaatatttaatgtaacaaata

SEQ ID NO: 103 - DNA, 7511 bp, Bos taurus
  source 1..7511: mol_type = other DNA
atggcggacggggccccccggccccagctatatcgcagcgtgtcgttcaagctcctggagcgctggagcggcgggcccgggcagagggaagaggccacggacgctccgggactgcggcgccgctcctcgtgccgaccggtcgcggccgtcccggcccagccctcccggcgcgtgtccaagctggcgtcggggcccccggccgcccccgcgcagccgcgcccgctccgcagcctctcgccgtctgtgcgccagctctcccggcgcttcgacgcacagcgtctggacgacaactccaccgggccccgagacgggggcgtctccgcggcggcagcagaagaagcggcagagggagccgcgcgcggagcctggcccagcgtcatggagatgcgcaagctcttcgggggccctggctccaggcggcccagtgccgactcagaggcctcagggacgcccagccctgacggggccgcgtgggagcccgcgacccgggaaccccggcagcccccgacgccacctcctcggacgtgcttccccttggcgggcctgcgttcggcgcggccgcttcccggcccggggatcgaggggaggaggcggcggcagcaacaggagcgagcgcagcgtccggcggatggtttacattcttggcatagcttctcccaaccgcaggcctgggcccgggcctcctcctccttctcctcctccatcgtttcctcctatcctgtcagccgcagccgggctgccagctccagcgaggaggaagaggagggcccgccgccgccgctgcccggggcgcagagtccggcctaccacggcggccactcctcaggcagtgacgaggaccaagacggtgagggtggccactgccggggagggaggacgggacccaggcctggaagctcctctttggataaggactgtaggccggacagttatgggttaaatccgagcagcatggactcggcagggggatctgggagccccgatcccccaactcctccaagagctcctagcgaggaaggaccccaagggtggggtgctggctctcccccctgtgtaccaggcccccaggaaggacttcggcccttgtcagacactgtggggggaacgttccgcgtggccaaggtgagctttcccgcgtacctggccagccctgcaggctcccgcggtagcagccgttattccagcacagagactctcaaggatgaggagctgtgggctagccggggttctgggaactggggtgtgtatcgctcccccagctttggagccggggaagggctcgtcaggccccaggtgcgaagccgcaccaagggacctgtgggcaccacgagggtatcgagggatggaggattggatctggacaagagccggcaacggaagtccctgtcgaatccagatattgcctcggagaccctgacgctactcagttttctgcgttcagacctttcagagctgaggatccgaaagcctggtgggcgacctggagaccttggaagtggcccggtagatggcagagattcaccatccgcagatagctcggtggggcaacttgggcccatgcctgccccagccccagcatcatctggcacccgccccacactgaaggacttgacggccaccctccgaagggcaaagtcctttacctgctctgagaagcccatggcccgccgcctgtcccgcactggtgtcctgaagcccagctcctctgaactcctgctggcaggcccaggtgccgaggaggatccactgcccctcgtcgtccaggatcagtatgttcaagaggcccgtcaggtttttgagaagatccagcggatgggtgcccagcaggatgatggcaatgatgtcccccctgggagtcctgactgggcaggagacgtgaccggagggcagcggtctcaagaagaactctccggccctgagtcgagtctgacagatgagggcattggagcggaccctgagccttctgtttcagcattttgcagcctgggtcccacaggtgtttggcgacccctctcctcatcctcagcccagaccaaccaccatggccctggggctgaagagagtctgggagggtgggccctggtgtcccctgatacccctcccacacctggtgccctccgccgcagacgcaaaatcccaccttcaggccctggtgggactgaactgagcaatggggaggcaggtgaggcctaccggtctctgagtgacccaattccacagcgccaccgggccgccacctctgaggagccctctgggttctctgtggatagcaacctcctgggttcactgagccccaagacagggcttccgacagccccagctatggatgagggcttgaccagtggccacagtgactggtctgtaggcagtgaagagagcaagggataccccgaggttattcagagcattgttcagggcccgggtgccttagggtgtgtggtggatgacagggttgctggcaaagcccccaagaagaaatctctgagtgaccccagccgccgtggggaactggctgggcctggattcgagggtcctggaggggagcccatcagagaggtcgagcccatgttgcctccatccagcagcgagccaatcctggctgagcggcgggcagaaccagaagagcccagtcctgccaggggccgggcacagtctgagagggccctacctgaggccccacctgcctcttccactgcccaccatgatttccaccttgacccgaaacttgctaacattctgtccccacggctcattcgccgtggttccaagaagcgcccagcccggagcagtcatcaggagctctcaagagaggagggcaatcaggaccaggctggcagcctgtctctggcccgaccctcctccaaacacgttcgccatgctagtgtgcctgccaccttcacacccatcgtggtgcctgagacaccgacttccattggcccccctgtggctgtgccagagcccatgggcttccctgccaaagcccaccccacgatgcaggcaccatctcttgaggacgtcactaagcagtacatgctgaccctccactccagtggggtccctgccccagtgcccgtggatatgccctgcctgactccgactgcaccaccatcttcggaggtcaagccccctgaggcagcccgggctccagatgagcctgccccaaccagcaagtgctgcagcaagccacaagtggatatgcggaagcacgtgaccatgaccctgctggacacagagcagtcgtatgtggagtcactgcgcactctgatgcagggctatatgcagccactgaagcagccggagaactccttgctctgtgacccgtcactggtggatgagattttcgaccagatcccggagctcttggagcaccatgagcagttcctggagcaggtgcggcactgtgtgcagaactggcacgcgcagcagaaggtgggagacctgcttgtccagtcgttctccaaggacatcctggtcaacatctactctgcctacatcgataactttctcaatgcgaaggatgccgtgcgtgtggctaaggaggcgaggcctgcctttctcaagttcctggagcaaagcatgcgtgagaacaaggagaagcaggcactgtccgacctcatgatcaagcctgtgcagcggatcccacgctacgagcttctggtgaaggacctcctgaagcatacgcctgaggaccacccggactaccctctcctgctggacgcgcagcggaacatcaagcaggtggccgagcgcatcaacaagggtgtgcgaagtgctgaggaggcagagcggcacgcccgcgtgctgcaggagattgaggcccacatcgagggcatggaggatctccaggcccctctgcggcggttcctgaggcaggagatggtcattgaagtgaaggcagtcggtggcaagaaggaccggtccctcttcctgttcacggacctcatcgtctgcaccaccctgaagcggaagtcgggctccctgcggcgcagctccatgagcctgtacacggcagccagcgtcatcgacacggccagcaagtacaagctgctgtggaagctgccgctggacgacgcggacatcatcaaaggggcatctcaagctgccaatcgagagaacatccagaaggcgatcagccgcttggacgaggacctgaccaccctgggccagatgagcaagctctcggagagcctcggtttcccccaccagagcctggatgatgcgctgcgggacctctcagctgccatgcaccgggacttgtcggagaagcaggccctgtgctacgcactctccttccctcctaccaagctggagctgtgcaccacgaggcccgagggcaccgactccttcatttttgacttcccccaccctgatgcccgcctcggcttcgagcaggcctttgatgaggccaagaggaaactggcctccagcaaaagctgtctagaccctgaattcctgaaggccatccccatcatgaaaacccgcagcggcatgcagttctcgtgcgcagccccgaccctgagcagctgccccgagcccacccccgaggtgtgggtgtgcaacagcgacggctacgtgggccaggtgtgtctgctgagcctgcgcgctgagcccgatgtggaggcctgcatcgcagtctgctccgctcgcatcctctgcatcggggcggtgccaggtctgcagcgctgctgccaccgggaacagactgcgtcgctgaggaaccccccagagacggcacccgagcctaccgggccagacctggacgtcgaggcggcggacgaggaagcagcgacgctggccgatccagggccccagccatgcctgcacatctccatagcgggctcaggcctggagatgacgccaggccccgccgagggtgatccccgcccagagctagtgccctttgacagcgactcagatgatgagtcttcgcccagcccctcggggaccctgcagagccaggccagccggtccaccatctcctctagctttggcaatgaggagaccccgagctccaaggaggccacagcagagaccaccagttcagaggaggaacaggagcctggcttcctgccactgtctggctccttcgggcctggcggtccctgcggcaccagtcccatggatgggagagcccttcgtcggtccagtcgtggctccttcacccgcggcagcctggaggacctgctgagcgttgaccctgaggcctaccagagttccgtgtggctgggcaccgaggatggctgtgtccacgtgtaccagtcctctgacagcatccgcgatcgcaggaacagcttgaagctccagcactcggcctctgttacctgcatcctgtatctgaataaccaggtgtttgtgtctttggccaatggagagctcgtagtctaccaaagagaagcaggccatttctgggacccccataacttcaaatcggtgaccctgggtgctcaggggagccccatcaccaagatggtgtctgtgggtgggcggctgtggtgtggctgccagaaccgagtccttgtcctgagccctgacacgctgcagctagagcacacgttctccgtggggcaggattccagtcgcagcgtggcttgcatggtggactccagcctgggcgtgtgggtgacattgaagggtagtgcccatgtgtgtctctaccatccagacacgtttgagcagctggcggaggtagatgtcacacctcctgtgcacaggatgctggcaggctcagacgccatcatccggcagcacaaggctgcctgcctgaggatcacagcgctgctggtgtgtgaagagctgctgtgggtgggcaccagtgccggcgtggtactcaccatgcccacctcacccagtaccatcagctgtccacgggcgcctctcagccctgctggcctcggccagggacacaccggccatgtccgcttcttggccgcagtccaacttccagacggcttcaacctgctctgcccaactccaccacctcccccagacgcaggcccggagaagctgccctcattggagcatcgggactcccctcggcaccgaggccctgcctcggccaggcctaaaatgctggtgatcagtggaggcgacggctacgaggacttccgactcagcagtgggggcggcggcagcagtgagaccgtgggccgagacgacagcacaaaccacctcctcctatggagggtgtgaccctgtccacggtggcccaggactcgcccacccacccgccctccgcctgcttgcctcttccaagcccactggcagaccctcacacgagtgtctgactggggcacacctgtgcctgcacagactgcctcccatccttgcggaagcattgccaatggagcacctgctggccagcggggtctaggcctttcccagccctctggctgctccggtgcacccagtcatgatgggagggtggagagggggcacatgggctgcccaggacctctgtccctggagcttctaccaaagacacggccgggcctggaccccacagggctggggagggccatgtgcaatatttggagggttttcctggggacagtgggagggctgggggatggaatcctttcccgtgtacagtatttatgtttctttttagatgtgtaccttcccaagcacttatttatgcaatgacctggggcagggagtgattggagaaaataacagaagagggaaaaaaaaccctgttcctgccctgggagtcaccctgggaccccaaccagccttcctggagggaccggtcccccctcattatgtacgcacacacacactgcatgtctggggcactgaaacactgccccttaacataaactttccacggccttgtcctgactgggttgccctccagtatccagagcaaaggaccaagtcccctcagtcatccagaacaaggcgatgaccggcggggtgggcagtgactcccatccggggggtttccccaggctcggcgtttccttgcttgttggcagtgcctttagcggtttccgaatgtgggaaccctgtcggggcctcgtccagggccctctcctggcctgagcagggaaggggccctcgctgtccgcacgttgccccccagcccctgtgtctcatgcactggcacacatggtcaccgcagagggtgggcctaggggcccctcactctgaccactgactccctctctccacatcctcctcttgcagcggcagcccttccaaggacgcccaaagctgaggggccagacatggcctccctggctgggaaggccactctggagactagcttcccctgcccccacccccaaaccgcagaagccagtctgaccccagccccacgcttcttcagctgcagctccaaaggtctggtgtcagatggggtttgtttggttctgtttttgtttggggtgggtgggtcattgcggtcttagattatgtttttcttgctaccaaacagtcatgtattaactttccttggatgatgaagtttaaagggtcaataaatagaaacaccaaacaactggtcccca

SEQ ID NO: 104 - DNA, 2567 bp, Bos taurus
  source 1..2567: mol_type = other DNA
gccgtgcgtcttccaggccggggcggcgattcattcaaaaggcgcgcagcttgcgcggccgcccgggcgctcaccgacacgggcccgggcaccgcgtagacccaggagctgggctgctggagggggcggccagccccgtccgggaacacacgctgggaacacagtggccgcctcttccgtctccacccgctggggcctgaggatgaagctgagcccgccaccctggtccctgtcctgtctcctcgtgctgctgccctggcctctggccactgccacaccgacagcccctcggcagtgcccacctgggcaggagcccaacctggaaccagggtggggcacattatgtaggtcctgccccccaggcaccttctcggcttcctggggctctcgcccgtgccagccacactcacgctgcagccctcgagggaggctagaggcccaggtgggcacggcaacacaagacgcactatgtggagactgccagcctgggtggttctcccctgcagaggcccccggtgttccctgctggccatgctcccggacccctctgggtggccgcagctgtttcaaacgggggcgacgggcccgacgtggcgtggaggtggccgcaggggccggtggtgcagcggagacgcggcagcctgggaacagcacgcgggcaggcagccccgaggagacggctgcccagtacgcagtgatcgccatcgtgcccatcttctgtctcatggggctgctgggcatcctggtgtgcaacctgctcaagcggaagggctaccactgcacggcccagaaggaggtcgggcctggccccggaggcggaggcagcgggacgaaccctgcctgccggcctgaggacgccagtgaggacaccattggggtcttggtgcgcctgatcacagagaagaaagagaatgccgcggcgctggaggagctgctgaaggagtaccacagccgacagctggtgcagaccagccaccggcctctgcccaggctgccgccgggcccgcccagcatgccgcacgtttgcccgcatcgccagcacctccacaccgtgcagggcctggcctcgctctcgggcccctgctgctcccgttgtagccagaagaagtggcccgaggtgctgctgtcccctgaggctgcagctgccaccactcctgcccccagactcctgcccaacccagccaggacccccaaggctggggccaaggccggacgccagggcgagatcaccatcttgtctgtgggcaggttccgcgtggcccgaattcccgagcagcgcgtgagttcagtggcctccgaggtgaagaccatcacggaggctgggccctcagggggtgagctccccaactccccacgacctggcctccccagtgaacagcgggcacttctaggaagtggtggaagccatgctaagtggccaaagcccccagcagagaacaaggctgaggagaaccgctatgtggtccggctaagtgagagcaacctggtcatctgaggagctgtcccgtctgaggaccctgcagccctgccctgggcggttccgagggcttcctggaggagggggagctgcagctgggcccatgaggatccagaaacgccagcagacaaaaccgccaaggcctggaggtgggagtgtccaccccggtgaggagacaggaggccggccatcgggtgctgtgtgcaggagcaggtggaggccccctcccgtccccgccacccatttccctcccgcaggtccttgagcctgtgccctgccctgacctctgctccactgggcggctaggccttgtggtggggaagggccctctgcctggcacccctggccccacccttgcccctgtaggggggacttctccctccttttctgacaggtcctataaaggacctgggctgggagctgagttttgggttctcgtccaggtcaagcccctggccattactgggtcccagttctttcgactgtgaagtgggggtagcggcaccgcggcgcacagggccttcccgcctcgcaggctctgggctgggtcgtgggggtggagagctcgactccacactgcccctcccactagtagctttatctgaccccgtttttgctgcttcaggccctccgccttcaagtccccccagggctgcctgaatgtggctctgcatacataaggggcctaatgcatgtgcctgcctctgcccctgctgtttttattgaaactgaagaaacagacttgacctgggcaggactgtggagcagagccccagccacccccacgatctcaggcgcttcggagggaagtggcgtgtgcgacggggctgggaaggggcctctggcctcggtggtgtgtgtccctgcggtgttggtgtttgtcccctggcctgggcagtgcaggcgactgctccacctccgtttccacactggctcagcagtctcgttatttatgtggggctgtgcaggcacgggcccacggctgtccccgtttctcttatttattgaaacttggctgttgtccatccttgtgtctccatactcatccattgttgaataataaaaatgtgggaaagtagtgtcaggagga

SEQ ID NO: 105 - DNA, 7158 bp, Bos taurus
  source 1..7158: mol_type = other DNA
gcggagcggtggcggcggttcgggcggcggctctccgcgctctgaggcgcggggcgcggcgggtgcagccagggctcatgtaatcaaagaagtttctttgttgtgtgtatcttttcagaacacaacaggaattgaaaatgaatcagaacgcagctgagcctgtggtagccacggagaccctggccggggtacctgaacatgtgtttcgaggacttccggaggaagtgaggcttttcccatctgctgttgacaaaactcggattggtgtctgggccactaaaccaattttaaaaggcaaaaagtttgggccatttgttggtgacaagaaaaaaagatctcaggttaagaataatgtatacatgtgggaggtctattacccaaatttgggatggatgtgcattgatgccaccgatccagagaaggggaactggctgcgctatgtgaattgggcttgctcaggagaagagcaaaatttatttccactggaaatcaacagggccatttactataaaactttaaagccaatcgcgccgggcgaggagctcctggtctggtacaatggggaagacaaccccgagatagcagctgcgattgaggaagagcgagccagcgcccggagcaaacggagctccccgaaaagcaggaaaggccccaaagacgaagacgagaatccttccgcttccaccaccgagcaaccagccatccctcaggaggcggtgagccaggatgtgcctccggatctcgccctccctccccctgcccacgagccccagacaggaccagactcggagctggaagcagcgctctgtgaggcgaacgaggtggtggaggaggaggaggaagaggaagaggaggagctggaagaagagggggacgaagcagctgatgtgccaaatgaaagttccctgagagagctggagatccggtgtgacgagaagcctgaggatttgttagaagaaccaaaaacggtttcaaaggaaactctggcagactctgcggaggcagcccctgtcgtgaaaactcccagagccagagaagaggccaacggggacgtgtttgaaacatttctgttcccatgtcagcactgcgagaggaagttcacgaccaagcaggggctggagcggcacatgcacatccacatgtccacggtcaaccacgccttcaagtgcaagtactgcgggaaggccttcggcacacagatcaaccggcggcgccacgagcggcgccatgaggccgggctgaagcgaaagctcgaggatccggccggcggcaaggcgcccagcgacggtgctcctccccaggacgacgcacagccccccggtcttgggcaggacggtctcaccttgagctcagagaaggcgtcccaggactcagtgactccttccgttgtagaagagaacggcgaagcaaaagagcttcacccgtgcaagtactgtaagaaggttttcggcactcacacgaatatgagacgacatcagcgcagagttcacgagcggcacctgattcccaagggggtgcggcggaaagggggcctcctggaagagccacagcccccagcagagcaggcccctcccaccccgagtgtctatgtccccagcacagagccagaggaggaaggggaggcggacgacgtgtacatcatggatatttccagcaatatctctgagaatttaaattactacattgatggtaaaattcagaccagcagcagcaccagtaactgtgacgttattgagatggaatccaattcggcagacttgtacggaataaactgtctgctcactccagtcacagtggaaataactcaaaacataaagaccacgcaggtccctataactgacgatctccctaaagagccttccagcagcacaaacagtgagtccaagaaacggaggactgctagtccgcccgtgttacccaaaattaaagcggagactgagtctgatcctgccacaccttcctgttccttgagtctgcctctcagcatatcaaccacagaggcggtgtctttccacaaagagaaaagcgtgtatttgtcatcaaagctcaaacagcttcttcagacccaggataaattaactcctcctgtggggatttcagccgctgagatacccaaattagggcccctctgcgtgtctgctcctgcgtcgatgctacccgtgacctcaagcaggtttaagaggcgaaccagctctccccccagctctccccagcacagccccgccctgcgagactttggaaaaccaggagatgggaaagccgcgtggactgaggcagttctaagctccaaaaaacccaaattagagagtcacagcaactcaccagcatggagtttgtctgggagagatgaaagagaaactgggagcccaccaggctttgatgaatataaggtgtctaaagagtgggcagctagctctactttcagcaatgtgtgcaaccagcagccactggatttgtccagtggtatgaaacagaaggctgaggggccaggcaaggctccggtccagtgggaagctgtgttagatctcagtgtgcataaaaagccctgtggtgactctgaaggcaaggaattcaaagaaaaccatgttctgcagccatcctgcagtgctataaagaaaaggaagcccaccacctgcatgctgcagaaggtccttctcaacgaatacaacggcgtcgacctccctgtagaaaacactccagatgtgtccggaagtgccagtccctgtaaacctctagatcctcagccagactctgaccgtggtctcaactccagcttgtctgccgctgtcgttgagtctcttcctgacgtttcctcgtcgtcacctgccctgcaaccatcttccctctcttgcgggcggctgccgcctctcctgacccccaccaccccctcctcccctccaccctgccctcccgtgttaactgtggccaccccgccccctccactgctccctactgtccctcttccagccccctcttccggggcatcccctcacccgtgtccctctccactctcgaatgccaccgcccagtccccacttccaattctctcccccacagtgtctccctcgccgtcccccattccttctgtggagcccgttatgtccgctgcttcaccggggcctccaacactctcctcatcttcctcctcctcttcctcctcttctttctcatcttcctcctcttcctcttccccttcgccacctcctctctcagcagtgtcatccgtggtttcctctggggataatctggaaacctctctccccatgatatcttttaagcaggaggaactagagagtgaagacctgaaagccagggaggaagccccatccaccactgagcaggagatcgtccaggagacattcaacagaaactttgtctgcaacgtctgtgaatcaccttttctttccattaaggatctaaccaaacatttatccattcacgctgaggaatggcccttcaaatgtgaattttgtgtgcagctcttcaaggtgaaaactgatttgtcagaacatcgctttatgctccacggcgttgggaatatctttgtgtgttcagtgtgtaaaaaggagtttgcattcttgtgcaatctgcagcagcaccagcgagatctccacccggataaggtgtgcactcaccatgagtttgaaagtggcaccctgaggccccagaactttacggatcccagcaaggcccatgtcgagcacatgcagagtttgccggaagaccctctagaagcgtctaaagaagaagagttgaacgattcttccgaagagctttacacaaccatcaaaataatggcttccggaataaagacaaaggatccagatgttcgattgggtctcaaccaacattacccaagctttaaaccacccccatttcagtaccatcaccgcagccccctgggcattggtgtgacggccacaaacttcactacgcacaacatcccacagacgtttaccaccgccatccgctgcaccaagtgcgggaaaggcgtggacaacatgcctgaactgcacaaacacatcctggcatgtgcctctgcgagtgacaagaagaggtatacccccaagaaaaatccggtcccgctgaagcagactgtgcaacccaaaaatggcgtagtggttctggacaactctgggaaaaacgccttcagacgaatgggccagcccaaaagactgaactttagtgtggagctcagcaaaatgtcctcaaataagctcaagttaaacgcattgaagaaaaaaaaccagctcgtccagaaagcaatcctacagaaaaacaagtccgcaaagcagaaggctgacctaaaaaccactcccgagtcgtcctcgcacgtctgcccatactgccacagagagttcacgtacattgggagtctgaacaagcatgccgctttcagctgtcccaaaaaacctgtttctccttccaaaaaaaaactttcccactcttccaagaaaggggggcacccgtcacctgccggaagtgacagaaacaacagcagcagccaccgcagacggacagcagacgccgagatcaaaatgcagagcatgcaggcacctctgggcaagaccagagctcgaagctcaggcccctcgcaggtcccgccacccccctcgtccttcaggtccaagcagaacgttaaatttgccgcttcggtcaaatccaaaaaaccaagctcctctttaaggaactcgagccccatacgcatggccaaaatgactcacagcgagaccaagaagcccaaagctgcagccaagaaccatgcagctcccctctcaggcaagatgtcgaggagcctgcatgtgcgggcgcagaagagcagagccgtcttacagagcaagtcggccttggccagtaagaaacggacagacaggttcagtgtaaaatccagagaacggagtggggggccgatcacccgaagcctgcagctggcggcttctgccgacccgagtgagaacaggaaggaggacgggagtggcaagcaggagctgaaggacctcagctacagtctccgcctggcatcccgatgtcctccgccagctgccccgtacatcaccagacagtgtaggaatgtcaaaggccacagctgcagcccagctccagggatccctcttcaaagagtaggtgcttcgcctgctccctgacagcacctgaagtgacctggagtcagtgaagccaaagggactggccttctgccctgcaggaagtaccgacctattccagatccatgagcctgcgagagaggagtgcgtgtgtatatgtgtgtgagcgtatgcgtgtgcctgtggtcttcagacgagggtcctgaaccccagggaggctggaaagtagctgactcggcaccgaggatgacacttgggtgctgtcccgggccgtgacctgcagctcagccttcctgttgggagggtcccctcctactatgcaatttttcaagagctccttgatcctgctttttgcttctccggttgtcttttgccacggcggggatttgggtctggccccgggggtcagctccagccctgtccaagcaaggccttcaggaggaggccaagttccctttctggccagctccgctcccgccccctcctccacggcccccctcatctccccccacccctgccctccctcacccctaatgtctctgggaatctgtagcctgtagacacttcggagcctcgctgatcatcccccttggcccttgcacttggaagacttttctctctgcctgagcgagcaatctgagctgctggctcttgagttgtttccagatttgccattctcatccgtcaggccctgggcgtccgggaaaaagtcctggtctccatttatagatcagactcctcctgtgggagaggcagaggacaggaagggatctggtcagggcagggggtgagctcctcgtgtacttctttgcctcacttggtgggaccatcaacacctttagtgtctgagcttctcaaattggctgaaaagggaaacaaacaaacagaaactgggaaatgaaaaataagttggacgattaaaaagcctgggggttggggggctggggaggggggaagagatttccggagaccgtccatcgcctgccagggaacaatggagctggcttcgtctctgatggagcagtccatgtctcggagaaacgggtgagctgagctaggagtttggacccagttcagtgagggtcttgggttttgtgcctttggggcagaccccagacaaggatgtctgagaccactttggcgctgtcttctcagccccatttttgatggtgatgcgccagacccaaagtggaaagaggggaccctttagagtatgtaggaatggtcatgcaaaaaaaaaaaaagggacttttttcagggctattatggttgatcttgcaactctgtaaatatgtatgtagacacttttaaaagcacgtatttatgtccctgactgtaaatgccccatttttaaagttttataacttgtgttatttaatgaatccgtcgactggctgcagcatggggtctgataacggtccaggaggaaagttttgtgagagcctcacgtgggcagaaaagtgctgatcgttggcttgttcccatcctttttcccacccacccagctacagaccgtgatttgttttaaattctcacaaactggaatcctttagcccgctcctaaaattaaccctcatgagcatgaaaatctgtggccgttttttatcctaacaatacaattttgtcccctccatgttgacactcgtgaactgtaccccagcctctcagttcttgagggtttgtttttcctttcagcagtgaggaaggcccacaaacccaaagcttggaaagttgccttcagccagtggaaaattctggaaaatgtggtgggagggctggggggcactcatttttttttgcatgtgcaaacgtgctggtcatcctccgaccctcagactatcaggagaactttcacgaatgttccaatcatgaaaatgggggtgggtgggttggtctttttgttttttaaaaagcggtgtttcctggtcagtgatggtcttcgagcgtgggtttggattgcagaagaggacatgtgaagaaaattaacaatgaaagttaatttatctgccatgtgaagaaaattaacaataaaagtgtgaggcgagattgtgaggaa

SEQ ID NO: 106 - DNA, 5661 bp, Bos taurus
  source 1..5661: mol_type = other DNA
ttcccgggggccgccccacccggcccaggggcgggcgctcgacgcgggctgggtggggatgggggtggggtgggactccttttcctcgccgtggcggcggaggcgcacggcgtggctggggaggcgcggcgtcgtcggtcggtgtcggagtcagagtcggagtcggagtctgagcagccggatctgaggaggaggaggaggaggaggaaggccgaggcgtctgaggaggccgaggccgggcacccagccgcgtccgcggcgccgcgagaaatggaggcccccggcccgcgccccgtgccggccctcggcggtcccggcccactgggggagttcctgccgccgcccgagtgcccggtcttcgagcccagctgggaggagttcgccgatcccttcgccttcatccacaagatacggcccatagccgagcagaccgggatctgcaaggtgcggccgccgccggattggcagccaccatttgcctgtgatgttgacaaacttcattttaccccgcgtatccagaggctgaatgaactggaggctcaaacacgtgtaaaattaaatttcctggaccagattgcaaagtactgggaattacagggaagtactctgaaaattccacacgtggagaggaagatcttggacttgtttcagcttaataagttagttgcagaggaaggtggctttgcagttgtttgcaaggatagaaaatggaccaaaattgccaccaagatgggctttgctcctggcaaagctgtgggctctcatataagaggacactacgagcggattctgaacccctacaacttatttctgtctggagacagcttgaggtgtctgcagaagccaaacctgagcacagacacaaaggacaaggagtacaaaccccatggtattccccagaggcagtctgtgcagcccgcggaaacctgccccccagcccgacgagcaaaacgcatgagagcggaggccatgaatattaaaatagaacctgaagagacaacagaagccagaactcataacctgagacgtcgaatgggatgtccagctccaaagtgtgagaatgacaaagacgtgaaaagcagcgtcaagcaagaacctgttgaaaagaaagagtgtgttacagaaagtgagaaggaaaaacccaagagtcgagctaaaaaaactaccaatgctgttgacctgtacgtctgcctcttgtgtggcagtggcagcgacgaggaccggcttctcctgtgcgatgggtgtgatgacagctaccacaccttctgcctgatcccgccgctccatgacgttcccaagggagactggaggtgtcctaagtgtttggctcaggaatgcagtaagccacaagaagcattcggctttgagcaggcggccagggattatactctccgtacttttggtgaaatggcagatgcattcaaatctgattacttcaacatgccagtccatatggttcccacggaactggtagagaaggaattttggcgactagtgagcactattgaagaggatgtcaccgtggaatatggagccgatattgcctcgaaggagttcggcagtggcttccctgtccgagatgggaaagtcaagctctcacctgaggaggaggagtatcttgatagtggctggaatctgaacaacatgccagtgatggagcagtctgttcttgctcacatcactgccgacatatgcggcatgaagcttccttggctgtacgtgggaatgtgcttttcgtccttctgttggcacattgaagaccactggagctattcaattaactacctgcactggggtgagccaaagacctggtacggcgttcccgggtacgctgctgagcagctggagaccgtgatgaagaagctggccccagagctcttcatctcccagcccgaccttctccaccagcttgtcaccatcatgaaccccaacaccctgatgactcacgaagtgcctgtttaccgaaccaatcagtgtgctggggagtttgtgatcacgtttccaagagcctaccacagcggtttcaatcaaggtttcaattttgctgaggccgttaacttctgcactgttgattggctgcccttaggccggcagtgcgtggagcattaccgtctgctccaccgctactgcgtgttctcccacgacgagatgatctgcaagatggcttccaaggcggatgtcctagacgttgtagtggcctcaactgttcagaaagacatggccatcatgattgaggacgagaaggcactcagggaaactgttcgcaaattgggagtaattgattcggaaagaatggattttgaactgttgccggatgatgagcgtcagtgtgtgaaatgcaaaaccacgtgcttcatgtctgccctctcctgctgctgcaaacctggcctgcttgtttgcctgcatcacgtacaggagttatgttcctgtcctccttataaatacaaactgaggtacagatacactctggatgacctctaccccatgatgaatgcactgaagcttcgagcagagtcttataacgagtgggccttgaatgtgaatgaagctttggaggctaagattaataagaagaaaagccttgtcagctttaaggctttgattgaagaatctgagatgaagaaatttccagacaacgatcttctacgtcacctccgcttagtcacacaggacgcggagaaatgcgcctctgttgcccagcagctacttaatggaaagaggcagacgagataccggtctggtggagggaaatcccacaatcagctgaccgtgaatgagctccggcagtttgtgacccagctgcacgccctcccctgtgtcctcagtcagacaccgctgctgaaggatctcttgaatcgtgtagaagatttccaacagcatagccagaaactgctctctgaggagatgcccagtgcttctgagctgcaggacttgctggatgtcagctttgaatttgatgttgagctcccccagcttgctgagatgcgtgtccggctggagcaggcccattggctggaggaggtgcagcaggcttgcctggacccgagctccctcaccttagatgatatgagacgtctcatagacctgggagtgggcctggccccatattcagccgtggagaaggctatggcgcggcttcaggaactgctcacagtatcagagcactgggatgacaaagccaagagtctcctcaaggccaggcctcggcattcactgaacagcctcgccgccgtggtgaaggagattgaggagatccccgcacacctgcccaacggcgcggcgctgaaagactctgtgcagagagccagagactggcttcaggaagtggaggccctgcaggctggaggacgtgtgccagtgttggacacgctggtggagcttgtcactcgaggccggtgtatcccggtgcatctaccctcgctgccgagactggagtccctagtagctgaagttcacgtttggaaagaatgtgctgctaatacattcctgactaagaactcgtcctattctctcctagaggtgctgtgtcctcgatgtgatattggccttttgggattaaagaggaagcagagaaagttaaaggatcccttgccaagtggcaagaagaagagtccccgtgtagagagtctgagcgacctggagagaacgctgatggaaagcagagagactgcttcagctatggcagcactcggggaagctcgcgcacgagaaatggaagccttgcagtctctccgacaagccaatgaggagaaactgctgtcgcctgtgcacgatgtggagatgagagtctgcgtgtgtcagaaggctcccgcggcccccatggtccagtgtgaactctgcagggatgctttccacaccagctgtgtggcagctcccagcattccacagggcccccgagtctggctgtgtcccaactgtcggaggtcggagaagcctccactcgagaagattctgcccctgctggcctccctgcagcgcatccgcgtccgcctgcccgagggagacgcactgcggtatatgatcgagaggaccgtgagctggcagcacagagcccggcagctgctctcgtcagggcacctcaagtccttgcaggacccagtgggctcaggactgttgtgtggcaggtggcaggccacagcaggacaggtgccagagaccagcaagatatctcagcctcctggccccacgtccttctccttgcccgacgactgggacaacagaacctcctatttacactctcctttctccactggtcggagctgtatccccctccatggtatcagcccagaagtgaatgagctgctgatggaggcccagctgctccaggtgtcccttcctgaaattcaggaactctaccagacgttacttgcaaagccgagccctgcgcagcaggctgaccacagctctccagtgagacccggcagtgagaagagtgacggctgccgagggaagcgagatggagccagcagcctcgagagcaagctgaagagacgcctggaaagagaaggcctctccagtgagcggtgggatcgaatcaccaaaatgcgggcccccaaaaagaagagaatcaaactgagccaccccaaggacacggacagtctcaagttagagagagagtgtagctatgagttggccccctctgccgacacgcagtccctgccctcagactcgtcctcctcggagcaggaggactctgaggacgaggacgccatctgcccggccgtgagctgcctgcagccagaaggagatgaggtggactgggtccagtgtgatggcagctgcagtcggtggttccatcaggtctgtgttggtgtctctcccgagacggccgaggaggaggactacgtgtgcgtgcgctgtgcggagacggacgtgccgggccggagataaaacccgcagagccctcctcagtcagggctcggcccggggactcccagtttctcagcaaagctgcaggactggcgttcaaaccagcctgtgaacggtgctatttctattccttacgggatcatttttccagaactctttgaaaaaaagaaaaaaacgacaaaaattttttgacactttttgtatttttttccttaagagctgtttgtggttgttgaggtttgaaacgctgactgtttttgcaggggttcccaccagtttggaaggattggcgcttgcaccttttcatgtacagcattaaaatcttacctcttcctggggtttaccggctgagtccaactcggtttcagtgcccagaagggcccagtaaatcctcatttgaggaatttctctctcgtttactctgttaccatattggggagcttaagtttttcacttttgaggtttttgtttgttttctttatccacttttgttttccctggtagactaggtttatttatctaagcaataacttccatgttggtttcagtggctggaattaaaaccaaaccaacttccaaatggtgtgtggtgctttagcatctggcggatgtcagaacacggacgtctggcttctggggtcactggtgaactggtgacagaagaggctgctccctaagcacctgtcacagctgtgctggctccccaccccccaaccccccgcctgctcatttggaagcttgtatcaagtgttgcggtttataatgtggaataaatccttggtcctaacataa

SEQ ID NO: 107 - DNA, 8384 bp, Bos taurus
  source 1..8384: mol_type = other DNA
ggcggcggcggcggcagctgcaagttgggctgcaggggcagcgcatacactacaatggctgctggaaagaggcgtaaggaaacaatttccaggcccgccgcgtccagcccgaaatatgagaaaaaaattattagaaattccgcgggcggtgtaaaggcggcggacgggccggagggaggatgttaaagccccgcggttggccaaaatgggaaggactggattccgctctcttccacgaagagtcaatgggactggctaagatcaaggtctgaggcttcgtccatccatcatcaggcacatggctacgcactctcccgagaaaacccacaagtgtaactattgcgagaaaatgtttcaccggaaggaccacctgaagaaccacctgcacacacacgaccccaacaaagagacgttcaagtgcgaagagtgcggcaagagctacaacaccaagctgggcttcaagcggcacctggccctgcacgcggccacgagcggcgacctcacctgcaaagtgtgcctgcagacgttcgagagcaccggcgtgctgctagagcacctgaagtcgcacgcgggcaagtcggcaggcggcgtgaaggagaagaagcacccatgtgagcactgcgaccgccgcttctacacgcgcaaggacgtccgccgacacatggtggtgcacacgggaaggaaggacttcctctgccagtactgcgctcagaggtttgggcgcaaggaccacctgacgcggcacatgaagaagagccacaaccaggagctcctgaaggtcaagacggagcccgtggacttcctggatcccttcacctgcaacgtgtcggtgcccatcaaggacgaactcctcccggtgatgtcgctgccctccagcgagctgctgtccaagccgttccccagcacgctgcagctgaacctctacaacactccgttccagtctgtgcagagctcgggctctgcccaccagatgatcaccaccctgcctttgggcatgacgtgccccatagacatggatgctgtccacccctcccaccacctttccttcaagtacccgttcagttctacctcatacgccatctctattcccgaaaaagaacagcccttaaagggggagattgagagctacctgatggagctgcagggcggcgtgccctcctcctcccaggactctccagcatcctcgtccaaactggggttggagccgcagctcgggtccctggaggacagcacgggggacctctccctgtccaaaagctccatctccatcagtgaccccctgaacacaccagcattggatttctctcagctgttcaatttcataccattaaacggccctccctataacccgatctcggtggggagcctggggatgagctactcccaggaggaggcacactcttcagtgtctcagctccccccgcagagccaggacctgccggatcctgccagcaccatagggctgggctctctgcactcgctgtccgcagccttcaccagtagtctgagcacgagcacaaccctgccccgtttccaccaggctttccagtagagtcccacggtgcgcatcgatggccgaaacgtacgtgcagccctgccttagctgaccgttctggttgtagtgcctactttaagacagtatcacagtttctgcttctgtatagtaccaattttcattaagccagtataaaatagaaactagctttttaaatgagctacagaaccatttgtgttcagctatgtttacctgggtattttgtcctgattcactgccaactgtcatattttaagacgatttctcacataggaaagccattattagcagtcaagtcttacaaatcccatgttctgacttactgttagatcttaaactttgcatttttgtccgagatcaatggctctaccttttgacatttggctccttaaaaatttttgctacagaatgtaagttttctaaaacgtacatgaacgacctgagagtttaaaaaaattttactagcctctaaatggattaataaggaaatgcttcaaatgaatttagaatccagtctggggacatagcatatgtttcttagatataaccataatttcaggtcagtatattttgaagacttgctattgtctggctcaaatgtttgctgtcttttttatgcacatataagaaaaacaagtctagacacaaagcgcaccagtatttgtagcaaacacaccccctaggaaggtgacacggcagtccatgttacttaagagttggccttaaattagtacacaggatgtttgccacgttttatccttgttgctgtctttcttgcattcatgctaatagtttatggcgtcaagagtattcagcgtcagtctttaggtgcatctctttcatcttctccatctttcatggccagagggtaggtcgaggacagccagccacttcccttggcttctcactgcacaattggtccagagtccacagctgttggggaacacaggtgtgcttatcccaggggtggggctgtcccatcttgtagggtgagagaccccaagtagagagaccagtgcgagacctgccactctgctagggacttgtctggggaccacgtcccctgttgccagggtggaaatgaggggtcaagtcacagcatatcacttacatcactcttgctccctttgattcagttttctctccctgagcaccaaaagcagaaaacagaacaacccggctcaccaaccaccaccaccccccgcactgtggtatctttctcattcagtgtttttttttttttttccattcagtgttaattcatttttccctgctgtttttggacagagactttaaagttaaatgcattcaaaaactatttgaggaaagatcacaaaatttaagtgactaaaagtatatacagtaaaccccacttttacaagtccctgggaaagtgggagggacatcacaaagaagctgaatttgcagacggagggagaagagcatagtaaaacaagcactgtcttaggtgatgtgaaaaataacaagagcgtggtgttcagtgaatacagtagttattacaggctttcctcactttagacaaccagcacattttctcaaaggtcaaactattattctatttttctaataatcggttggggcgtcaacctaagaaacatatcttattaagcacttgttaacaccttatttggggaccctgtctgtcggggtgggtttgaggagggagggtttatagaagagtatatatctctttaaaaaaaaaaaaaatatttctgagcactcatgagccctgtgtggaaggttctcttccctttgacagggccagctatcactgcggatttccatgtttaccaagaagttctaaagatgagtgcagattactgaatataatacattatttaaactattggggagtagtataatttgtggagagatgtaaattgtaataatgtaaatggggtttcactatatattatatatatgtacacacacgtgtatcgcacttcatagaatcagagttgctctcgtacggagctttggctccagtatttttatcatgctcctatttgtttttgtttttttaaatcccaggagcagtagtttttatacgtatgtatttaaattttattattaaaagttacattttattaaaaaagtgtgttccaaaggcattaaaattatatatgttaataaggaaatacatttaaaaatttttcaaactgctcctgagcgtttgcctaggagactgtttgctctcaaagtaggcttttagctctgctgtgttgctgcccgctttagtttctatgaaacagattgcttacttaaatcattagttgaatgattagtgttcaatattgctttaatcaccgtcgagaggaaaagaaaaaagaaaaaaaattttggtgacagagcacaaataaatagaaaacctgtttttaaatagaaatcacaaatcacaagtgtggaagcactactttatttttgtctaaaatttgcttaaagagaagtcagatgaacgaactgagatgttggccttttcgtgggctgtatttcactgctaataaaaaagtaaaaagggagtaccaggatttattaaataaagcattttggaaatggggaatggcgccatgtaggtgtatatatatatttttagcctaattctcgcccagcataccttccagggcaccagggctgaaattgtggactctattttcacgtgtacacacccctttgcctgttgctttcccacagacttgaaatcacacttccaagtaagagggaattctgctcattcattttttaaactgactatagtggtcactaaaccccctttgagagaatctctattaaacgtgaggtagactcattttattttaattgcacaatggtctgacaaggctgtaacaccgaactggctctttttttttttttttccttaaaaagaaaattgacacttttgatgtcaactggatgtgattaaaaataatgattgccaattctgttttcattctccagtggccagaatcatcatcctcgaaatttctagtagtgcctttgcttggggttttttttttttttttttcataaagggattgttactaagttaaaaatatttcagtttagaatttggacctcagacaagatactgaacctcattcggtttgaccttccatgtgtttgtacaaattaaggtcaccagataccgaagcacgagcacggaaggatactgacactataagcaatattctccgcggatggacccaaacatcaccttgatagctactggtcactgctacaacgtgcatcttaaacccctgttaccgagttccgttgttcatgtcctgactgatacgcgctactccttgtgcagcttgttaatttcatgtaggtgtttcttccaaactatgtatttttgccgatgtgttgcttgtgtttcattcttcttagtcacttgtgcaacatagtgatatattgtgttgatttggacagtagcggtttttaaaaaccatatactgactgaaaacatgagccagagctgactgctttattaagctaataatgaatgttaaagagtacatattttcaggattattcatctagtaagcaatacacatatataggccaatatttttttaaaaaatagagcttggtcaacctctatactacacatattacaagatatagcactttcaaaacgaatctaaacctttacagaaaaactttcttataggttatgccttttattttaagacttattataatttgtttcaagtgccattagaggatagatatataggcctttgatgtataatgctttgtgttcaagaatggtagacggtattttaaacaggtacagttttacagtgttttcttatcaatttgctatattgcacagaatcagtgtgtcttttcataaggttttacaatggtttattttttttacaaggtttacgtgtctcaaaacacactgtcttcccagtatgtaagttaaaatacaaaaaaaaatattgacccaagttgcttccagcctactgagatccaggcgacattggaggagatcttttagacgtcatgtattcttcattagcaatggcagactgttacctctgagaagtgagtgcctacgttgagcccttgtcttatttttctcacgctgtgtcagcagccacatcaatcacacagatggtctgtggtgatcccatagatccacgttaggctgtgttgtgtagctctgtgctgagaacccatgcagagctcccttctgatccatccaaatggatcctttcttacacatccaaatgatacgacccacaacgggcaaggcacgttcctatcaccccccggcccttctgcaatattgtcaacctctgaggttggaaggcagtgtcttttgataagttacctagcttagaaaggacagaactattgctaatgtgtaaaacacttcattgtataagcttcagtggtacagatgacccagaatgaatgtgtatcttctcagaaacactccttcaatattctgtcggatcatgctgctaatgtaacttgggatacaactcttcatggtgctacagacttctctgtctcgttcagtcgtattttttttttatccataggaaaaaaaaggactacattaggtgtaaaggtgtacaatatatttttatactgtgacttcatttgtctttaacaagacttttacaccacccaccacaatgtattcatgtgcacttgcaaaaggaaaccttggacgtgcaaatgttataccagaacaaacccagctttgtccacaaaggtgaccgtgactcggaaagaaacatgggctttataatatggtgtggagtgaagaacatgctgtacgttactaacagccctttgaatttaacaaaaactgggcatccattcggaaatggattgcatcatgcctgaacataagctggactgctgaaattgtatttttagctgacgaaaaagtgtttggactagtactctaaaaatgttctgatgataaagttttgagtcaaaatagaaaagagaaactgcattccaggctgcattttgtatatttttattgcatttaaaattcgctcttctgtgataccgggaaatcaagtggcttatcctgtatatcatgtacttaaaatgtattcacaaactactgttgtgtttgtataaaatatagacaaagatcatattttctgtgtgtgtataagctctgtaaaatagcaatcacattatgaagctgcagcaagactacattttaaacatccacatccaaagaagcaggctatttattgtccagttacccagttataaaatactcatttgctgctaattaaacaatagtactgcagcttctcatggcctacagtgttatgtttgctgtaagaaaaagatatgtgaactccacaaaaatatatgaataaaaattatagaatggctttagataatgtagatcttcctgaaattataacagatgataaatttctatttggtataacatttttttccctttgaatttgaaatattttctcatttgataaatataagattctaatagctgcctctaggacaatacttggttgttgaaatgtaaagttctagggtttcttttgttttgtcttaacttttgtaccttgaaaggaaggatagaaattgtaaataaaaaagtcaaaatgttgtgcatcaaaactttaatccaggtgaaaattcaaacaaagaatctcccaaaggctgttagaatgtatagagcaatcatgtttttattcatatgagataaaatgggcattaagtatttctttggcacttactgtccatcagtcctgtaaattatctttttagacgttctctttgggctatttgtctaaaacttggctgaaaatcctcccttgaccatttacctggaccagagtaatgagctcacagatgtcattttgtctcaaattcatcagttaaacagtgatgcatctttgaaagaaagagattaattctgaaaatccagttaataatctggccaggaaccagtggcaggaaaaaaattttttttggatttctcatagcacctctttcattatgataggaaaaattgcttcttaacctttgactttcctgtcctaaggttctctaccttcagaaaggtaaagaagatgcacagtgactccccatttggcagttcctcttttcacatacctaattccagcaaatctaaaagcatttctttgttgattcttagcattaacactacatacatttttatttaaattcacgcatatgaaatattttaaggactatttatagaaaggaaaatgctattgctttttatagtcagaatactctgaaaaaggatgtttggggttaattcacttaaggtgtattctcccttatggaaaagcaactagaagtgcagtttaagcaaagcattaagcataagcattgcaccatagatccagtggggctaaaagacttcttcatgcaggtttttgttagatgaaaagttagtactgatgatttatcattaaggggaggaaaacttcagtctgactaaagacagcctgagtttttgctgccacttttccttaaatgagcatttcttaagcagaaggcaccttttcatgcgaagtcctgggctgttcccaaggagtcagctttacagaaagaggctggtttcctgatgaaccgttgcaatgaagatgacaccatcattacaacagtgggaaggtgtaaatgctgtattgagttgtacccagttacccttttttgtagatcaaaaatgatcaaatattggcggtttcagactaaaactaaaaacaaccaagcccagaatgtctcaacttcacctgctttctattcaacacagaggtcaataataaatctgtctactgaagatgtttga

SEQ ID NO: 108 - DNA, 5129 bp, Bos taurus
  source 1..5129: mol_type = other DNA
atgcgatcggagaaatccctgacgctggcggcgccgggggaggtccgcgggccggagggcgagcaacaggatgcgggagacttcccggaggccggcggcggtggcggcggctgctgtagtagcgagcggctggtcatcaacatctcggggctgcgcttcgagacgcagctgcgcaccctgtcgctgttcccggacacgctcctgggggaccccggccgccgcgtccgcttcttcgacccgctgaggaacgagtacttcttcgaccgcaaccggcccagcttcgacgccatcctctactactaccagtccgggggccggctgcgccggccggtcaacgtgccgctggacatcttcctggaggagatccgcttctaccagctgggggacgaggccctggcggccttccgcgaggatgagggctgcctgcccgagggtggcgtggacgagaagccgctgccctcccagcccttccagcgccaggtgtggctgctcttcgagtacccggagagctcggggcccgcccggggcatcgccatcgtttccgtcctggtcatcctcatctccatcgtcatcttctgcctggagacgctgccccagttccgtgcagacggtcgaggtggaagcaacggtggcggcgtctccccagggtccaggggcagtcaggaggaagaggaggatgaaggtgactcctataccttccatcctggcatcgcccccgggggaatggtggcagggggctcatcctcactaagtactctcgggggctccttctttaccgaccccttctttctggtggagacgctgtgcatcgtctggttcaccttcgagctgctggtgcgcttctccgcctgccctagcaagccagccttcttccggaacatcatgaacatcatcgacctggtggccatcttcccgtacttcatcaccctgggcaccgagctggtgcagcagcaggagcagcagtcggccactggtgggggcggccagaacgggcagcaggccatgtccctggccatcctcagagtgatccgcctggtccgcgtgttccgcatcttcaagctgtcacgccactccaaggggctgcagatcctgggcaagacgctgcaggcctccatgcgggagctgggcctgctcatcttcttcctcttcatcggggtcatcctcttctccagcgccgtctatttcgcggaggctgacgacgacgattcgctcttccccagtatcccggatgccttctggtgggcggtggtcaccatgaccaccgtaggttatggggacatgtaccccatgacggtggggggcaagatcgtgggctcgctatgtgccatcgctggggtcctcaccatcgccctgcccgtgcccgtcatcgtctctaacttcaactacttctaccaccgagagacagagcaggaggagcaagggcagtatacccacgtcacttgtgggcagcctgccccggacctgaaggcagctgacagcggacttggcaagcctgaattctcagaggccacccgggagcggagacccagctaccttcccacaccacatcgagggtatgcagagaaaaggatgctcactgaggtttgatggatgcgggcagtgcctgcatggaaggaagacaccaaacacacccttctgtagggacccttcttatcctccactactcgcaccccagctccaggtgacctcctgactccctcaccccacagtggttggtgccaggaccatacacctggactgtcagccttgttacttgacccctgcagcatcccaggtttctccatctaagtgacatttttgaaattctaatggtgccacccattcgtgcccatcttatgtcacgtggatgagatttccatctgaccttccctcgagactctgatttttcacgtttgggacttgtgatgtctctaggaagagtgacgtcgacaggctggaaaccaggcatacctgggtcttgtttctccttagcatccttggctttggggcatgcaccctcccatcgtagcttctggccacatgctaactagccgaagccggtggacagaaacagtactcatcctgttgttttcctccagtgccctgtaaggtcagctggtcattctgcagagaagccttaggagagccttcagttgcacagcctgaacatatgttatctgtctctaatcttggatgcaattagagaaactacgatgacacttggccatttggaggacgtgagaggtcagtcctggaccaaaagggccagtggattcttctagcgtgacctggcacgacagacgttagtctctggtctccacttagctttctcgactctgattcccctgactctctggcttccaggaaggctcctccttctcagagccaaatactctgtctgttcaggtgccttcctgtgcagaggaactgacaaaggggaaccacggagctgcaagaaatggccgaacggaaccaagtacctggcctgaccacttcttagagaagcagtgtcccttagacagtgccatctcagagcggtggaggagtctttttgctgacagcaaattcctttccctcacttcccccagccctctctagccttccatccttcttcctgggaatctgacttaggtttgcagctgaaggcttgctaaagcagactccagcttaaatctgccgccaggtaaaagcacacattggatgctagcgtgggtttctgtttgcattttgcgggcaatgagacaggcggtttagagtaaggaagaagcattgttcttttgctgacagcctcaccggtagagggttgttttttttttttttgctgagtcaggtaaacattgtcctgctccagccctggagatgcagctgttggctgtcaccggggagatgcccggtggtgcccccacctggtctgagccatcttcttccatggtgaactgaactgctcttcctgtaccacctgcccacgtcccctttttctcctctttgcactgccattcatttgctgccttgatcaactgtgatgtactgttctgagctctcctgggtgcagttcagaaaccgaaaggactgttaacatgtttttagttttataatctaggctttaagactccttgatgagatttttttttttctaaagcttttctcgaagagccaacttacgaggtacagcctcttgagggttttcttgagagacactgtgtctggatgttgccgcactttacagggtaacttgggggccacagttccactttgtgcctcggtgtccccacccatttgatgggaacattgatgtctgtccccccacccccacttcttggggaatggcccaggaactcagcttggatcctgggggtgatgctatgcaaatgcgtgaacttctgtaatgatttctagctcgttaccctgcttccccctgtcctataggccaagcctcctcccagggttcccgctttgggtccaaagcccacatccaggccattctgggtccttagctttttaaatccctccctcgcctcttgaactgggccttattctttgcttcttccccttcagcaaggaagatgctctttggagcgattcagtggcatccttttagggccagcaccatcttgattgggagtttggcttctccaaatctgaaatgcagtagaaaatgctcatgattcacacacctgctctgaggctatgaagtgccagttttccacatctgttctggaatattactcctgacagttcagggggaggggggaagagaaggcccacccagaggcaaaagggatgctcatgttgagagtgaatgttgaaacatggatgtgtcattctcagaatgggagtgtgtgtgttttcatcgtcctcaccatttcaagtcagagcatgcccgagtggaaagggtttatgaagagctgggctctccggggattctggttgccgtagcaaccttccagactgttctatcttctgatttgggcagccagggagaacgagagacgatgtagtcccagcttctgaaatggggacgcctctcacataacgttgtttaggggctggcagggctgggacagggatgtgtggcctttggaaaatgagggtaagcagatccccaaagcttcctatctggacctttcttcctggtttccccagcccatcactggagctatctcatctcactcgagtcttcagaaacttgaatgctaggcaactgccagttagaccatattctctctttcgaaatggccttggaggggactagcttctcaagaggcagctgctactagttttagtccataatagcaccatggagcactcacgtcggcccctcccagctctaagacagagagatgatgcttcttcccaaggaaatgtctagctcctgggagggacctcatagatggttcttcttaaagacatccaagacaaactgggaggctgtgacctgacctacttcctctgatggatttcgccaagtgaccccctttgactctgagcctcttgatgtggattttgttccctgagcaccgagggaatctgtgccccatgtcgccgtgacagctggggtggcgttttcacagctgtcagcaaaggggttcaggtctttgttacacctgacttgggccggcaggagctcccagaggtcttcctggatctgtcaggctcagctgagacgtggggggattccttctgcctgagaagccccaggagagaaggggacagctggccgtctttgcacccgagggaactaaggaagaggctggcccagagttccaaaccaggagaactacacctgtatttctgatagcggctggacaaagaggaccgggaccctcaacttctccagaagacgagacaccccccgcccctgctcctagggtggacaggcctgacgctcctccagtggcctcccatgagccatgaccaggctggaaaaaactgggatgggacccatgggcatttaaagatgggacttgggatactgggctgaggctggggcagggaggtgagacagaagaactgagggtgcagacagaagaaaccaggtgtggaggggtcaggggtcgagtgacgccggcaagcgcatggctgcgctgcaggaccgtccaggaaaagggtggccatggaggactcacaataaatacagccggagtctcgga

SEQ ID NO: 109 - DNA, 1270 bp, Bos taurus
  source 1..1270: mol_type = other DNA
gattctgggagaatatggcggccgccgttcacccccgagttgtccgggtcctgccaatgtcacgttcttctgttcctgccctagccgcctctgtgtttcacagcccaccccagcgccagcttcatcatgcagtcataccacatgggaaaggtgggcgttcctccgtcagtggaattgtggccactgtgtttggagcaacgggattcctgggccgctatgtcgtcaaccaccttggtcgcatggggtcacaggtgattgtcccccaccgctgcgagccatatgacaccatgcaccttcgtcccatgggtgacctgggccagatcatctttatggactggaacgggagagacaaggactcaatccgaagagctgtggagcacagcagcgtggtcatcaatctggtcgggcgagagtgggagacccaaaactttgactttgaggatgtttttgtcaagattccccaagccattgctcaagtgtccaaagaagctggagttgaaaaatttattcacatttcacatctgaatgctgatattaagagctcttctaagtatctgagaagcaaggctgttggagagaaggaagtgagagagacgtttccagaagccaccatcataaagccggctgaaatctttggcagagaggacagattcctcaattattttgcaaatatccgctggtttggtggcgtccctcttatttccctgggcaagaagactgtgaaacaaccagtatatattgtggatgtcaccaaaggaattattaatgcaattaaagacccggatgccagagggaaaacttttgcctttgttgggcccagtcggtacctgctgttcgacctcgtgcagtacgtctttgctgtggctcacaggcccttcctgccgtaccctctgccacactttgcctatcgatggatcggtcggctctttgaaattagtccatttgaaccctggaccacgagggataaagtggagcggattcacaccacagacaagatcctgcctcacctgccaggcttggaagacctgggcgttgaggccacgcccctggaactcaaggccatcgaggtgctgcggcgtcaccgcacctaccgctggctgtcttctgagatcgaggacgtgcagccggccaagacgattcccactagtggcccctgagctgcccgcggcttttggtggtgtttgggttgctaccaaccaggccacctttggcggagaatcctgtatatagtgaatgagagcctctattaaaacctgagatgacaaaaaaa

SEQ ID NO: 110 - DNA, 3089 bp, Bos taurus
  source 1..3089: mol_type = other DNA
gcgagtcggagggtggggtggacggtggctcagaagagctcataggaaactgcggggaggggatgagggatgtgagggaggccgtgaggaaagcgggagaaagagaggagtcttgagatcggagttccatgtcaggagctgaccgtaaagatgacgtcggagtatcgaggaacctgaagttcaggattccttatatttcatggaaggagtgggtaaacttaggaatagtgaagacaacaagaaattaatccagagctttcctcatatctcagaacctgtcctcagtaagaatgtcggatagggttgactggttacaaagccaaaatggagtatgcaaagttgacgtctattcccccggagacagccaaccccaagactggaaaatgtcggatgaatccttgtctatttttaaggaagcctcacacgatcccatcagagtgctcagctggctccgcagagacctggaaaaaagcacagcaggattccaagacattaggttcaagcccggagaatcatcgctcggcagggaaatggtcagctcaggagacccacgcaaaggtttctgcgtggactattacaacaccacctccaggggaagtccagggaggctgcattttgagatgagtcacagagagaacccacaccagggccccacctgcaatgggagctccgtagatgaagtctccttctacgccaaccgcctcacaaatcttgtcatcgccatggcccgcaaggagatcaacgagaagattgacggctctgaaaacaggtgtgtccaccaatcagtgtacatgggggatgagcctccacccaacaagagcctgagcaaggtggcgtcggagctggtgaatgagaccgtcactgcctgctccaaaaacaccccatcagataaggctccgggctctggtgacagagcctcggggacattacagagccccccaaatttaaaatacaaaagcactttgaagatcaaggaaagcaacaagggaggcaggggtccagacgacaggcctagctctaagaagtctttcttctacaaggaagtattcgaatctcgtaacgcgggtgatgccaaagagggaggaaggaccttaccggcggagagaaagatgttcagagggtatgaaaggcccgatgacttcacagcttccatcagtcaagggatcatgacctatgccaacagtgtggtgtccgacatgatggtctccatcatgaagaccctgaagatccaggtgaaggacacgaccattgccaccatcgtgctgaagaaggtcctgatcaagcacgccaaagaggtggtctccgacctcatcgactccttcatgaagaacctccacaacgtcacggggaccctcatgacggacacggactttgtctcggccgtgaaaagaagcttcttctctcatggaagccagaaggccacggacatcatggacgccatgctggggaaactctattcggtgatatttgccaagaaaccccctgagacagtcaggaaaaccaaggataagtctgaaagttactccctcgtctctatgaaagggatgggtgaccctaaacaccgaaacgtcaacttcgcctccatgaaatcagaaggtaaagtgagggagagagtatgcacccccacgcccaaacccgagaagtcctgtgtggaaactctgggggaacacattatcaaagaggggctgaccctgtggcacaacagccagcagaaggaaggcatatctgcatgcctccagggctcacccttcgtcactcccaaaagacagtataaacctgtgccggactttcccctggggttcccttttgacccctgcaacttcagcctccctatgcagtgcccagaaaaacctgagaattttatgtgcgactcagactcctgggccaaagacctgcttgtgtctgctttacttttgatccagtaccacctggcccagggaggaaacatggatgcacagagctttctcgaagctgctggcacctccaacctgtatcccactaagtctccagccgtttcccatgagtccagcctccggtctccccaagtgggagctgacccagaagaagtggagaagaaggacctaatgagcgttttcttcaactttatccggaacttgctcagtgagaccattttcaagagcgaccatagctgtgatcccaaggcaaccaaggaagacaacagcccccagtgtgaaagacccgtcaccccttctcccgccaaattaaatgaatgtgatgagactgggggtgcttttgcggggctcaccaagatggtggctaacaaacttgatggccacatgaacgggcagatggtagaccatctgatggactcagtgatgaagttgtgtctcatcattgccaagtcctgcgactctccattggcagaactgggagacgacaagtctggggatgccagcaggccaacatcagccttcccagagagtttatatgagtgtttgtccaccaagggcacagggactgcagagacattcctacagaatgcctatcaggctatccacaacgaactgaggagtctatcagcacagcccccagaagggtgcacagcacccaaagtgatcgtcagcaaccacaacctgactgacaccgtgcagaacaagcagctccaagccgttcttcagtgggtggccgcctctgagctcaacgtccccattctgtactttgcgggtgacgacgaaggaatccaggagaagctacttcagctctcggcagctgccgtggaaaaggggcgcagcgtaggtgaggttctacagtcggtgctgcggtacgagaaggagcgacagctggacgaggcggtgggcaacgtcacacggctgcagctgctggactggctgatggtgaacctgtgatgggggcccgccgccgcccctctttcccgcagtgggccccgccctcgcccctcccttcgtcctcactgccgcggcccccttctcccgcctcctcacagagccccaacattatcttcataccactcacatctaagacacgtcatcgtgcgctagtccctggattttgcagatattcttgtccatgcgagcaaggacgtaaaataaaaattacagataaatggc

SEQ ID NO: 111 - DNA, 2263 bp, Bos taurus
  source 1..2263: mol_type = other DNA
tcgccgcttcactttcgtcatgagagtttactctgcgctgtctccaaaatgcacaataatagtataaatacatcttccgacagtttttaaatgattaaaaataggtaggatggaacaagggaacatttagaccgaggaatttttcacgaggtgccaggaaagaggaaaataattggaagaatcagccgtagcggatcttccggctgtaaaaacaaggcagcagtcagctgatgggcccagcgccaaggccgatggtcaggaccggtactgccgacagctgggaactcggaacaagaggagaatcctgctaaagatgaaaaaaaacagagaaagattctgcaaccgagagagggaatttgtgtataaattcaaagttggaagtcagtgctttgaactgagggtacccctcaaatttcctgttcaagagaatgccagtcatttacatggacgtctaatgctgctgcacaacttaccgtgctttatagaaaaagacttaaaagaagctctgagccagtttattgaagaagaatccctcagagattatgacagagaggccgaagctgccctggaagctgtgaagtcaggtgaagttgacctccatcagctggcaagcacatgggccaaagcctatgcagagaccacactagagcatgcgaggcccgaggagcccagctgggatgaagacttcgcggatgtgtaccatgacctcatccattctccagcctctgaaactctcctaaacttggaacacaactacttcgtcagtatctctgaactcattggcgaaagagacgtggagctgaaaaagttacgggagagacaaggtattgaaatggaaaaagtgatgcaggagttgggaaagtcactgacggaccaagatgtgaacaccctagctgctcagcatttcgaatcccagcaggacttagaaaataagtggtccaatgaattaaaacaatcgactgccattcaaaaacaagagtatcaggaatgggtaattaaacttcatcaagacctaaaaaatcccaacaacagctccctcagcgaagaaattaaagttcagccgaatcagttcagagaatccgctgaagcaaatggaagggtttatgaggaacagagaaagttagaagaaagttttaccatccacttaggggcccagctgaagaccatgcataacctgaggttgctgagagcagacatgctggacttctgcaagcacaagaggaaccaccgaagcggtgtgaagctccaccgactacagacggcgttgtcgctttactccacgtccctttgtggcctggtgttgctggtcgataatcgaattaattcctacagtggtattaaaagagatttcgccacggtttgccaagaatgcactgactttcatttcccccggattgaagagcagctggaggttgtccagcaggttgtactttatgcaagaacccagcgcaggagcaagttgaaagaatctcatgattctggaagccgaaatggaggaagtgacgataagactaaaactgcagatagaaactatttaaatattttacctggagaattttacatcacacggcactctaacctctcagaaatacatgtcgctttccacctttgtgtggatgacaacgtgaaatcgggaaacatcactgctcgggaccccgccatcatggggctccgaaacatcctcaaggtttgctgtacccacgacatcacgaccgtcagtattcccctcctgctggtgcacgatatgtcagaggaaatgactataccatggtgcttaaggagagcggaacttgtgtttaagtgtgtcaaaggtttcatgatggaaatggcctcgtgggacggagggatttctaggacagtgcagtttctggtgccacagagtatttctgaagagatgttttaccaacttagtaacatgcttccccagatcttccgggtgtcatcgacactgaccttgacctccaagcactgagcccccacagaccaacacactgaagaagatgctgggatcctgtcaagcaaaagatgcccagaaagagaacttgccacccaaggcacaaatccgatcatgataaatgtctgctaaacattccaaaaacccatatgttgtacagactggtgatcagccccttacttatatattcattcttaagctctgtttatgggggaaaaaaaagagtcttggtttttttttccctactgtttccctactgtcctatctgtgaatattagtgagttaaaactttgtctgtttcacacatgg

SEQ ID NO: 112 - DNA, 2176 bp, Bos taurus
  source 1..2176: mol_type = other DNA
gggcgggaattgaaattgccgcggcggctgcctgctagagcctgagagctgcaggtttccagcctcggaggggaccatggtgcggcctgcgaggcataagaagccagtcaattactcccagtttgaggactctgatagtgatgatgattttgttcctgcaaccgcaccgttaaataagaaatccagaacaacacaaaaggagttgaaactagaaaaaccagaacctaagctgaagaatctccaggaaaaagacatctcactacaagagaaaacccctaaaaaaaggatggctttggacgataaactctacaagagagacctagaggtcgcactggctttgtcagtgaaggaggttccagctgtcaccattgatgtggagcagtctcaaggctttgacaaacatggcaagaaggaaacaggagcaacgagtaagtctcctcatgtctccaactgcagtgtagccggtgattatttagatctggataagatcactgaggaggatgagtctcaggacactccacggagaaggaaggctgcatcccaagccgtggtccagcagaggaagattctcttggaaaacggttatgacgacggagccaacgactctgagccagactttgcaactggtgaagactctgaggatgactctgattttagtgggagtgaagatgatgaaagcttcactgtgagaagaagtaaagttaaagaaaccaaacggaaagaaatgaaggcgaaacctccagctgaaaagaaagagaagcctaaacccaagcctggtgctagggtgacttcagtagactgcagtccagctgccgtcaagtcagaatctcggtcctcaccaagaaaggaggccagtaggacgccttcacagatatgcagtcctgcagcagaaagcaagagacctaagtgggtcccaccagccatgtctggaagcagcagccgcagccccctggcaggagtggccgtgaagtctccgagtcagagcctccgcctcggcctgtcccgcttagcacgggttaaacctctgcatccaaatgccaccagtagctgcgtgtagccacaggaagatgtttgattaagaagctctcactcgtataagcgggtttagatttgaggagggaaatgtaatataaaggaatcctttaacgtcatataaattacctctacctgttttgtgactgttctctgcaaagctgcccaagacttcagcaagagttctatgtggattctcttggaatgtccttccttctgaagagcatattctcctccctcagtgtatgtaagcagtcactgtgatcaccttgctgtcagcatgacacggaagaggtccacgttacacgctgctcgttgccagtaacaggatgctgcttcctgcctttgtcaagttgatggaccctgaacttcgtgtgcagagtttatctcgcaagaggaagattcccgcttctcccagggacgtttctgcactgacaacctcactgtcagttttattgccatgatgtttcatattttagccaaatgtttttataatgagaattcagaattattttccacattgaaacagttgatgtactatagtgtatgtctcctacctagaagtattaatagatggttatagcaaataaaatttcatgacatttcatgaagccttcattgtcaaaacctttgatatgcactttcccttgataccattgcatccgtctgtctctaactgtctgcatttcatgataaaataagtgctctgcagaaaaaaaaaagttatccgttttgttgtaaatagaaagaggaagagctgtttcttaaagtcatcattgtacgtgaagcccatgtttaacaggtggctgtcaagttgtcatccctgttgtattagcatagctcttcaggaagcttagatacgagttcgctgtgaataaaagtagtaatagatgttttctatttacagtgaaaatttcaaccagctcctgatcactttttaataaaaattctctgaattttaaccattaaaaatgtatataatacttaattcttggtttatgaaatatgttaatgtgaaagtaagtaattcagacttatgattaaagtgagcatttgcccaaaaaaaaaaaaaaaaaaaaaaaaaaaaaaaaaaaaaaaaaaaaaaaaaaaaaaaaaaaaaaaaaaaagaaaaaaaaaaaaaaaa

SEQ ID NO: 113 - DNA, 627 bp, Bos taurus
  source 1..627: mol_type = other DNA
atggcccggggacagacgccgctcatcactatgtcccggggagcaggacgtccgcagggcacgctgcgggctctcgtcttcctcggcgtcctagtgggcatggtggtgccctctcctgcgggcacgagagccaacggcacactgctggcttcgaggggctggggcaccctgctatccaggtctcgagccgggctggccggagagatcgctggcgtgaactgggagagtggctacttggtggggatcaagcggcagcggaggctgtactgcaacgtgggcatcggcttccaccttcaggtgccccccgacggccggatcagcgggacccacgaggagaacccctacagcctgctggagatttccacggtggagcgaggcgtggtgagcctctttggggtgaagagcgccctcttcgtcgccatgaacagtaaaggaaaattatacgccacgcccagcttccaagaagagtgcaagttcagagagaccctcctgcccaacaactacaatgcctacgagtcggacctgtaccgaggcgcctacatcgcgctgagcaaatacggaagggtgaagcggggcagcaaggtgtcccccaccatgactgtcacgcacttcctccctaggatatga

SEQ ID NO: 114 - DNA, 5362 bp, Sus scrofa
  source 1..5362: mol_type = other DNA
actataaggataggtcaaatcaaatataatcaactcgggtcggagcaagcgggcgagctagacagcgtccccaagccatggtccctaccggccgcggctcagcctgggtccctctgctccagacccgagtgcccaaagcaggctttggtttcatgtcagcagccttcaactgccttccaaaaataagcccctgccgccatgccgaggggagaaaaacaagaagggcggtatttttagggccattaattctgaccacgtgcctgagaggcaaggtggatggccctgggacagaggctcttcatcactatgtcccggggagcaggacgtctgcagggcacgccgcaggctctcgtcttcctcagcgtcctagtgggcatggtggcgccctccccagcgggcacccgcgccaacggcacgctgctggcctcgaggggctggggcgccttgctgtccaggtcccgcgctgggctggctggagagatcgccggcgtgaactgggaaagtggctacttggtggggctcaagcggcagcggagactctattgcaacgtgggcattggcttccacctccaggtgccaccagacggtcggatcagcgggacccacgaggagaacccctacagcctgctggagatttccacggtggagcgaggcgtggtgagtctctttggggtgaaaagcgccctcttcattgccatgaacagtaaaggaagattatacacgacgcccagtttccaagaggaatgcaagttcagagagaccctcctgcccaacaactacaacgcctacgagtcagacctgtaccgagggacctacatcgctttgagcaaatatggacgggtgaagcggggcagcaaagtgtcccccatcatgaccgtcacccacttcctccccaggatataaggaccctggaggaaggctcacaggtggcaagcaacgtcttttctgttggaattttgcacaagttgaacagtttcccctccccccatgactcctgagtcgggctcctcttttgcgtggggagggctgagccagtttcttcggccaccagaccccatggcatgtgagccaatcaagtggggggtggcagccggcatcctagaacagtgggtccacgctctggcctcacctgaggaaagaaggtacaggtgacactcgtgcaaggggaccttgggcccaggacaggcagggggaggaagcgtgtgtgcgtcgatgtatagccatagtctcagggtgaatcctcagcgccaaggcatttggaacatcaggtttcttcctcccggcgtcattgctgcaggaatctgcctgcaagaatgtcacctgtcaaaccagcaggattgacacgtacccccattctgcctctttccaatcccctttccattcggtggccactgactgtgtgggttgcatgggggagggggggtggtggggagcagctgtcctctaagtgcaaagcgggcaggtgacagggagtaaggtttagctgcaaggcagaaagtgaggggcactccaggcatcctgcctgtgagggctctggggaccctggaatgcatggtcagggaagctgggggaggagggaccgcctcctggaggcgaggagtcgagcagcttccagggcagtggagggaggcgctgggggttccatggagagctgctctgcccggacgattcaggactgcgcatggctccaggaacaagctggggccgggctgcctgcccatgacttttcctcacgttgaaccgggtcttattttaacctcagatgacaaggatcctaataatggcaacctctttggcgaagggtgagggggtacaaaatagggcacgaaggaagtagagaggctttggacggcagatgctaattcctctccagccacgtccaggaaactcacccaagttctcatctgataccctcacttccttctcggcgtcctcttcttgcacacacgctgggggctggatgcagcagaatgagcacggtgatctccagagtttgccccctctgaccccctacagtaggagaagagtgataatcccgtgaaatccagcagccaagcccaagcggctcctagccgtacgatgacttcctgcctcctgtcctcggagagtacttaaagcagaggttccccaagtgactataaatgttgctgcctggagccttcagattcctgaaccattggaacgttcctaaatctgaaaccagagagagctgtatcagagggcacatattttcagcgtacgtgaaccgacgagttaccggatgagcggacgccctctgcaactcccaggggcccccgcctgcaagacaggccctcatctgctgcacataccttcgtctctgacatacctctcgagctgcagatgcgtccatctatatatagatttgagatgagctgaagacagaggttggccatccccgggagagccaccctgctggccacctcctctgcccggccaaaatcaggtgaggcacatgctgggggatgagggaaaggccccatcaaggcgagcaaaagtggaagagacggtcagaggtcaggatgagagcggatgtggtcaagccttggctagaaactggatggagctggagccttggtcaagtgggaggggagaaaatcggcccatcttgactgttcagaatgtttcaggtgtgggtctgttggaagcagggaacagctcatagcttttgaggaggtcttgtcctgagtaagtgatctcttttaaaacagggtacatcagcagcagatgcacccagaacctgagctacgtgtcagttcctgatgggacatagtgggcgggtaggagggctgggtggatgcacggacagggacccagagagaagaaggaagttgcaggttccagagcatccatccattcccttcttcccaggtcttgggccaccttggtatgaggcggctctaagagagtcacgaggccacggacggctaggtgtttcctgcaactccctcaaggctcatcagaatgcccagatcctgaccagaagttcccccaaatgtccactcccatcttttcttgctgagagtgagaatgagaagctctgaaatgcccaggaccaggcaggcttggcacccagtcactcaagcacattggttacccacggctggcagcgctctagcacctgggcgtctgttcttctaggcacgagggcccatctgaaccgatggttatctgggatgtctcactggtgctttaaaatcctccgcattcaaaggaattctccgaagggctttcactttgacctgtgagtgacaggcttaggaaaagtcagtccccatctgcagaagtgggtcaccttaccgggcaggagcagagcgggatcagcaaggcgccccccaagcccctctctcccaaggccgcgtctgtgtgcaactgcaaggagtcctctccggtctgaccaccatctgagtcctgagctcagctctcagcctcgtgggacccacagggctgcagaagagcttgcagaagcaggggaacaagagtgaaccccaccttctcgctgagaatggactgagaatgacttgagtcctcccttcctcacccacaatctgagctttgatcctggtttatagagttttgtagctaactggcctctcttccaacccgaaccttttcgagcaaaggtggggctccagaagcccagaggctgccctattccctcttctagagcaaacgatgaagaggagagcagcacatgtgcacccctggaaggtggcttctcagctgggatgaggttggcccagaacagagaggtccccaggcccacggagagtctataccctctggggccacagagagacctcagaggaagcccgggaactccctgcttgctcctgccgttgacagtgttcatcattcaccgccttctaacccccatcccaccccacagcccagacgggattgaactttcctggaagcagcacatgattttgtaattaataccctgtccttatcctcataacctcccctctgccaccatgtccaccctccacctccaacaccgtactccaagccccaggtcagggctgttctttggcttaagaaagagggttctctctctctctttccttttttttttttttttttttttgtctttttagggccacacccatggcatttcggaattcctgggctaggggtcaaatcagagctgtagctgccggcctacgccatagacactgcaattccagatccgagccacatccgcaacctacaccacagctcatggcaacgccggatccttaacccatggagcgaggacaaggattgaaccctcatcgttatggatactagacgggtgcattacctctgagccacaacaggaactccaagctgggattttctcctggcctctgatccaggctacaaacctctctcctttagacacagctccttctccccaggccatttgccccagcctaaaattccctcccagcccggtcctatctggggccgctgggaccctccttccctggctggctccggccctttcttgttctggacctcagctttttcacattcttttcctatgcgagtccccagtgagaagggagacacaaaaggccatgagctgtcggtccccttcctcccagggttgggccactttgggccacagtcactggtgcaaatcacctgcagtcgctggcccccaagacaccaggcgtcctctctgccctcctgccccttttgcctcttctcgtgtggacacagccagaaaactcctgtatctttctagcccagtgctttcccccagaggtccatctgggtccgtcaccacccaggaggtgttctctgtcacttgccactcctgtgcctgcgacgtgggtgtctagtatttgcacccctcacccccagcaagaacccggccatggattctgctgcctcctaggaaatcgccaaaggcagggatattggggtcaaggccgaacgagtggatagaaaccctgtagcccatgcctgcccctgagccagagcttcctcttcccagaacagatcctgcaatctggtccagactgcagcgagccgacccggaaccgtgtttgtatacctcactgaactcgtgaaattccaacatccagacagctgtggcccccatggtttccctgtaaatcaagaagtacctcaggctgggtcccagcgcagagctgtcctcaagcggggggcctagcgggtaggaaaactcagaattcgttcagtcatgtgcttgtaaaaatgtgatgcttctattttttttaaatatgtatgtgctatgcacaaaccatatcctgttccagcaaaaaggaaaaggtctttttatacgttcctctaaactgactccacccctcctattaaaaccgacaacgaa

SEQ ID NO: 115 - DNA, 1179 bp, Bos taurus
  source 1..1179: mol_type = other DNA
taactgcccagccagtgtgcgtcactgcctcggcgagcagagagccgagcgagctggggagagcagacaagtttgaaggggagggcagagagagtcagccgccccaggggcgctcctctcccaccgcccatcctatccctttccactcttcttccccggcttctctctctctgcccttaactcccccccccaaaaaaaacccttatttagccaaaggaaggaggtacggggaatcctctcccctcccccctcccaaaaaaaccccccaacttttccagtccggagatcgcaggggcacgagcgagtagccagctggccatggagctgctgtgcggcgaggtagagccggtccgcagggccgtgccggacgccaacctgctccacgacgaccgcgtgttgcagaacttgctgaccatcgaggagcgctacctgccccagtgctcctacttcaagtgcgtgcagaaggacattcagccctacatgcgcaggatggtggccacctggatgctggaggtctgtgaggagcagaagtgcgaagaggaggtctttcctctcgccatcaattacctggaccgcttcttggccggagtcccaactccgaagacccatctgcagctgctgggggccgtgtgcatgttcctggcgtccaagctcaaagagaccatcccgctgaccgctgagaagttatgcatttacaccgacaactccatcaagcctcaggagctgctggagtgggagctggtggtgctgggcaagttgaaatggaacctggcggctgtcaccccccacgacttcatcgaacacatccttcgcaagctgcctcagcccagcgagaagctgtcgctgattcgcaagcacgcccagaccttcatcgctctgtgcgccactgacttcaagtttgccatgtacccgccgtcgatgattgcaactggaagcgtgggggccgccatctgtgggcttcagcaggatgaggatgtgagctcgctcactggagacgccctggtggatctcctggcaaagatcaccaacaccgatgtgcctgcttctcctccaacatggagccttctcgcctgctcacacacgctgaggctggcaggcagggccggagcggggggcccccctgcaccacagggcgtgggagaggagggccccccgctcctgtgaggaggtgtccgcctc

SEQ ID NO: 116 - DNA, 1227 bp, Bos taurus
  source 1..1227: mol_type = other DNA
acttcgagctgtgaccgcgcaccaacaacagcaacaactccactgcgccgggctgaggagcaggaattaggagctcgcgaataatatgaaagggatccgcaaaggggaaagccgagcgaaggaatccaaaccccgggagcctggcacgagaaaatgcgctaaatgtggccgcctagacttcatcctgatgaagaaaatggggattaaaagtggatttacgttctggaacctcgtctttttattgacggtgtcttgtgtgaaaggtaggcttgctttcggggtcccctctgctttggatggaggtgagctgacttgtcgccagagacgggtagcttactttgcaatttgcacggataaaataatcatgattatactgaaggagtcgattcacagctctttttctccctcagataggaaatgcattttgcttgccagatttggcgttcaatgcagtgattgagcttgtgctcaataaatattcttgcagtatgctcaaacgaatacgtgagttggttcctatagcctctaactactggataccaggcagaattcctctgcaagacttttcttggtgatgagggttaccttgatttctagtcaaatcagtagggtttgatgctttggttggaatatttatattaaaggcgctcctggaaatgctttttaaccctttcaggtaggaaagttatgttaggaatgtgctgcttttaaatgcattagggcacaggggtctttcaagaatccccccactccccacccccaaccaacatgaatgtgtgcatgcatttgtaaattatttctacgtgggctcttttgcactgataaataatagaggctgtttcctaggtcctgtggtagaggtttggcttaaaatggggcttttgaacggtctgagacgccaggaagtgtaaccaggtacctttggggatgaatgaaggtaggaggaaagtcatcagctgaagaccaaaaagaaaatccccagcaagtctcggcgttcagacaggtagagtgagtgtcacatgcacagacaaggggaggaggtgggtgggatgtaaaattcaatcctaagcaggaaggcaagggacagtcaggatcactggaaggagagctgtcctctgggagaaaatagattcccagctctgttctacaacttctcctgcgccctgctgcccattaattgctgcaaaaaaagaaaagaaagaaagaaaaccaaaaaaaaaaaaaaaa

SEQ ID NO: 117 - DNA, 1777 bp, Bos taurus
  source 1..1777: mol_type = other DNA
cgcctgcccgcccgaagcgccgtcgcctgccaccgccatgggtcgacagaaggagctggtgaaccgctgcggggagatgctgcacatccgctaccggctgctccgccaggcgctggctgagtgcctggggaccctcatcctcgtgatgtttggctgtggctctgtggcccaggtcgtgctcagccggggcacccacggtggtttcctcaccatcaacctggcctttggcttcgccgtgaccctaggcatccttattgctggccaggtctctggggcccacctgaaccctgccgtgacatttgctatgtgcttcctggcgcgcgagccctggatcaagctgcctgtgtacaccttggctcagactctgggagccttcctgggcgctggaattatcttcgggttgtattacgatgcgatctgggccttcgccaacaaccagcttattgtttcgggccccaatggcacagctggcatctttgccacctacccctctggacacttggacatggtcaatggcttcttcgaccagttcattggcacagcctccctcatcgtgtgtgtgctggccattgtggacccctacaacaaccccgtcccccgaggcctagaggccttcaccgtgggcctggtggtcttggtcatcggtacctcaatgggcttcaactctggctacgccgtcaaccccgcccgggacttcggcccccgccttttcaccgccatcgccggctggggctcggaagtcttcacgacgggccgccactggtggtgggtgcccattgtctctccgctcctgggttccatcgccggtgtcttcgtgtaccagctcatgatcggctgccacctggagccgcccccaccctccaccgacgaggagaatgtgaagttgtcccacgtgaagcacaaggagcagatgtgaatgggcgggggcaccccccacgccctcccatgagcgtccattgactgtgcaggggctgctccccagaagccctcttcatgactcccctcctggactaggaagcttcttgtccaccacctcccactagacagccccctccaggcttttccatcctgaaggaaataggacccagcaggaccttcagcctgtaagcggtggtggcacagggagcctgaccgatctagcccctcatctcccagagggagacagtaggcagcaggtgtgtgagtgtgtgtgcaggtacgtgtgcgtggttttccagatattcagggcaaaagaccgagtggaaaagattctgtcggtggagttgggtgggggtccagaggaagagaggggaagtgtggtgtctatctgtgaatgagaaaagaacgtttcaggggctccatgtggaggagggtccaggtacaggcatttctcagtatgtgtgtgtctgccctttcttctattcagggggctcttataggcttcagtagggtgggaatataagtagggctaggaggagggggccgtagcccagttgtggagctctgtctattcataaataaggagcagcagagtgatgtgttcatgtcacagtggaggcatgaggggagtgggggtgaagtccaggtcacaatttcatgtttgctcttaaaaatatatgttaatatatgtgtattaatacatatatgtagtagatgttacagtttcagggatggggattgggagactccatggtctgccctcctttaatcctccactcaacacatgtatttttgccttttataaaaaaacaataaaatgtatgccaattgtgtaaaaaaaaaaaaaaaaaaaaaaaaa

SEQ ID NO: 118 - DNA, 3088 bp, Bos taurus
  source 1..3088: mol_type = other DNA
tgtctgcacttggctcgagtgacaagggccaaacccctgctccactgacctcttccctggaggagcagagcagagcagcactcgggtcgccctgaggagggctgagaggcgggctctggctggggacatagggaccacagagccctggcaggggctgcagccagagcctgagacggtcatggaggaagctgtccaggggctgctgtgactgagaggccccaaggcaggcaactgggagcataaggcctttcagcagagtccacccgcgtgtccaaggtggtctcgacacctacagcaacaaagatgcaagtcatatggcagaacgagaatgttcgagagttcctggcggagttcatcagcacgtgttcggtctcggcgccgtggcccacatggttctaggaggtaaaatggggagctacctcgctgtcaatttgggttttggcttcggagtcaccataggtgtgcacatggcggggaacatctctggggcccatatgaatgcggccttgaccttcaccagctgtgcgctaggccgcatgtcctggaagaagtttcccgtgtacgttctgggtcagttcctgggctccttcgtggcggcggccaccatctacggcctcttctacaacgctattatcgacttctcggggggaaagctgatgacgaccggccccacagccactgctaacatttttgccacctaccttcctgaccacatgacactgtggaggggcttcctggatgaggtggtggtgactgggatgcttcagctgtgtctcttagccatcgcagacaaggggaacaacccaacgctgttggggacgcaggccattgtgactggcatccttgttgtcatcatcggaatatccctgggcatgaactcaggatatgccatcaacccatcccgggacctgcctccccgcttcttcaccttcctcgctggctggggctcacaagtcttcagaaccaaggactggtggtgggtgccggtggtggcaccacccctgggtgcctactgtggtgccatcatctacttgttcttcattggctccaacaaacgacaggagccccaggtcctggagaatcccttggcgtttgaagaccacaaaatatctgtgttgcccaagaccacgtctcaccagcatggggtcgcttccctcacccctgtttccgtgtaccccgaagatagaccctcagccttgtaccacccttaagtgactccatctgcctagagcccctctaagactatttgtgaccctacatccaccccaataaagaaagccttgtcccacagtggcaaccccgcttcttgggggcctcctgtggtggggcttacctcctgggttctctcagaagctcccaggttatgctgtagcccaaggctgggcaggagctgtggggcctccaactcctgccggccctgggagctgggtggcccccaggggagcaagtggaagggccgatcagtggcctcattaagacaggactagattagaagagcccggacatggaagggtgggctgccaggcacagggcagggaggggatggcatgggggagggattccagctggcaggtgggggctcagccctggaggaaccttgagctgccttgggagaagactctggactctccttcaatcctggccagtccttggaccaggcaaggcaaggccccagtgtgtggatcagagtttctcaacatttttgttttccttagtctcccctcctcaggagccttttaggcacttttcccccttaattgcatgatattttaataccacagatataccgtatatctgtttgtgtactgtacatatatctttgctttatacaaaaaaagtaagattttaaaaaatctctccctttcaaggatccaattttttcacccatgggagcataatcaccccatccttgaaaatgcctgaagtagaatgagaggaggtaagaggcaggctctgccttcttcgtgggggcagagctctcccaccgtggggaattccagagttggaaaggaagaagcagagaggaaaatcctgtcaaaaggcagggggcaggaaatggtctcatgaggttttggggaacaggtgcagagccggggcagccagggtccaccgatcccaggctgggaggatcatcctgcttctgggactcagaaggcagcatcacccatggcttctgtccctgcttctgcttttagcctttcagcagccattgagcacctccggtgtacaggctctgcgggtaaggttgggtctctgtcttcaaggagcacagtccctactcaggacaggtgatcacaaggaaagagctgaggagtgaagaaggagctgtgtgtggggctcagagggggctcacagctcagtcttgagctgaggccacagatggcttcctagaggatgagacagagcaaagggtggtggggtggtcctttctggggctggatgccgaacttcatggtttgaggagacagggcagggtggggggtgggtagaggacgctggttttccttctggaggtggggacctggtgagatgaggtttagaaagcccacatgagctgcagtttgcaggatgctggaaaagggggagactggggcctgtaaggaagtgtggggcaaggctgttccaggtggggcagcaggacagctcacaggtcagtgctggagggtggacagaagagaaaggattcgcctgacaccaaggaggtaaaagcctcaggctaggttcagactggagggaggagtgggtgaggatgggccaggggccaaggctgccttccagttgctggcttgggcctggggtattggagatgtccccactgaacatgaagaatgggaaaggcactggtttgaagaggaagatgataatttatactgaagacacatcgactttgagttgcctatgggacttgggcaataggtgtgtagagtcctgggcaagttactctgacttgctgggcctcggttttctcatttgtaaagtatcaaaatacctaccttttgtgttgatgtgattagatgaaacaaaacatgtacaatgctgaggatggtgcttggcacagagtcagtgctcaataaatgattatgccttt

SEQ ID NO: 119 - DNA, 1906 bp, Bos taurus
  source 1..1906: mol_type = other DNA
cagattctctggggctggttcatcacctccgaatactcctgtgacaggaggcgtttacaaaacccgcctccagccccaagaagcaataaatagaaggcgcccagcccacgagaagcaaggagaagtttctaggcctgcgtccccgggtttattaagctctcggcttcactcagtggctctggctgggagcctggaccgcttgggaggacagtggcggctggctgatctccggcgaggctgtgtggagaggggtccttgagaattctgctggaccaaccggaggtcgccttcgtggctggagtgtctttgggggctcggaccccagccaagtgtgtgcctctgtccctggcagtggatggttctgaatctgaagaataagcttggaaggccagcgcacccacctcaccttgctgtgtgaccttgggcaggtggctccctctctctgagcctctgtttccccttcagctcagcagccatcatggctgacggtcagatgcccttcccctgccactacacgagccgccggcgccgggaccccttcagggactcgcccctgtcctcccgcctgctggacgatggctttggcatggaccccttccccgacgacttgactgcctcttggcctgactgggccctgcctcgactctcctccgcctggccggggaccctgaggtcgggcatggtgccccgggggcccactgccatgaccagatttggggtgcctgctgagggcaggagccccccaccattccccggggagccctggaaagtgtgtgtcaacgtgcacagctttaagccggaggagctgatggtaaagaccaaggacggatacgtggaggtgtctggtaaacacgaagaaaagcagcaagaggggggcatcgtctccaagaacttcacaaagaaaatccagcttcctgcagaggtggatcctgtgacagtgtttgcctccctttccccagaaggcctgctgatcatcgaagctccccaggttcccccttactcaccgtttggagagagcagtttcaacaatgagcttcctcaggacggccaggaagtcacttgtacctgagaccccagtcttggcccttccttgttcccgccccaacccctgggcttctctgattccaggatacattactatagctgaactcatagatttagtgtggctaaaatgttgggggtcggggtggattgaccagattccctggatagtgttagtagtaggtttttctgcaagatggcgcaattgacaggtcatgctccattgcattaggccaaaatgctgggagttttcccttctttaccttttctatcatgatgaaaatgttgcacattttacagttgcaaaacaaataaaaggggacataacatttcactttgtatcttatcttgcagctaatgcaagagttgcttttctctggggaccgggattcccattctgggctcccaatgtccaacctgccccttggtttggttgatggagccctcgtagctcaccccacagtgtttctgcagccctggtttacaaagggttatagacaggtcttatatccccatatgggatttatcctgcaaccacatgaaaacagacagtgccgtgtgccctccacccaggcatttatagcatgttcccaagctggcactccccaaggactttagaagtcctttagtttattctctggttaaagtcccccctttcttgtgtctcctatgtccaagtcgggatttttacagagggggctgtctccagacagctccaccaggaaccaagcaaaggccaaatagtctggcaggcaggctagtggtgttgtgtatatgggtgggacgtgcgtgtcattgttatttgaattgtgctgttgtttagggaaaataacagtaaacaataataaaaggaactgatgttatt

SEQ ID NO: 120 - DNA, 6447 bp, Bos taurus
  source 1..6447: mol_type = other DNA
ctctgcgggatttcccccctgctctttggctgcttgttgagaggtttgcttatgggttagctttgaaaagattcaggttttactttccaatgagttttacattttagcagaactaggagattacttgcctggttcgtttcatttctgtaacaatgtttgaaagttacttctttagtagtcagataaatttctgtggttctaaatagttcgacgaggtctgaaaaatactaacatgtattaatacttttttattatattacaattttttattatgtttagtagaagagacacctttgccatttaaactttaacttttctcagtccttcagtgaatctgcagacctattttctcaggagctcagcctgaccctacttcagtgataaaggaggaaaggctggctacagtaaatatcattcaagatgtccagcaaagggagcagcacagatggcagaacagacttagctaatggaagcctgtctagcagtccagaggagatgtctggtgctgaggaggggagggagacatcctcaggcattgaagtggaggcttcagacctgagcttgagcttgaccggggacgatggtggccccaaccgcaccagcacagagagtcgaggcacagatacagagagctcaggtgaagacaaggactctgacagcatggaggacaccggccattactcggtcaatgacgagagccaagtccatgaccgctcggaggaggaggaggaggaggagcatcctcgtcgtcgtgtgcagcgcaagcgggccaatcgtgaccaggactcatcagacgatgagcgggccctggaagactgggtgtcctcagagacatcagccctgccccgacctcgctggcaagcccttcctgcccttcgggagcgggagctgggctcaagtgcccgctttgtgtatgaggcttgtggggcaagagtctttgtgcagcgtttccgcctgcagcatgggcttgaaggccatactggctgtgtcaatactctgcactttaaccaacgcggcacctggctggccagtggcagcgatgacctgaaagtggtggtttgggactgggtgcggcggcagccagtattggactttgagagcggccacaagagcaatgtcttccaggccaagttccttcctaacagtggtgattccaccctggccatgtgtgcccgtgatggacaggttcgggtagcagagctgtctgctacacagtgctgcaagaacacaaagcgtgtggcccagcacaagggagcatcccacaagctagccctggaaccagattctccctgtacgttcctgtctgccggtgaagatgcagttgtcttcaccattgacctcagacaagaccgaccagcttcgaaactggtggtgacaaaagaaaaggagaagaaggtggggctgtatacaatctatgtgaatcctgccaacacccaccagtttgcagtgggtggacgagaccagtttgtaaggatttatgaccagaggaagattgatgagaatgaaaacaatggagtactcaagaaattctgccctcatcacctggtgaacagtgagtccaaagcaaacatcacctgtcttgtgtacagccacgacggcacagagctcctggccagttacaatgatgaagacatttacctcttcaactcctctcacagtgatggggcccagtatattaagagatataagggccacagaaataatgccacagtaaaaggcgtcaatttctatggccccaagagtgagtttgtggtgagcggtagtgactgtgggcacatcttcctctgggagaaatcatcctgccagatcattcagttcatggagggggacaagggaggcgtggtaaactgtcttgagccccaccctcacctgcctgtgctggcaaccagtggcctagaccatgatgtcaaaatctgggcgcccacagctgaaacttccactgaacttacagggttaaaggatgtgattaagaagaacaagcgggaacgggatgaagatagcttgcaccacactgacctgtttgatagccacatgctctggttcctcatgcaccacctgagacagaggcgccatcaccggcgctggcgagaacctggggttggggccacagacgccgactctgatgagtctcccagctcctcagacacatcggacgaggaggagggtcctgaccgggtgcagtgcatgccatcctgaggcctcatacccaggaggggtgggctggggctgccaacccaatcctgcctgggcagccctttcctgtgccaggccctaacattcagcagaaacgcactttggacttttttgctttagatttaaaaaaaaaaaaaaaagacatcccaggaggacaagccagaggggagtgaacctacagagagtgtctaggagtgggagttgagccctggaatggggctccatggagaggtgcataggactcagcagaaatggcttctccccaaaccctttttgggggggaagggagagcctattttattaactggtttggggtagggaatggggtttccttttctttggtctcccttgtttcttgggtggcgggtggggggataactggctgttcagtaccgaggggccagactgggggtggtagaaatgccacactctctttggtttaggtttttgactttattttcttttattttttaaaagtctatgacagttgcgttgtttttctccgcccgcctgccccccacccccgagcaaccccatcccaggatcctgtctttctgggaagtctttttaatacgagtccctaaccatcctacactcttcactttcctttccaccccattcattctctgaacttatcacagtgttttgcacttgattatgtatccttcactttcttctttatcccatcatcccctcagtaggtctggtgagggaggttggggggaggtgggaggaggggcagaagtggaagcataggaaggatgttacctcttctcttactttaaaaaaaaaagttgtttggtggcagcaatctccctgtccctatcactgttagaggcctaattttatatctataaatatattaaaaagcaactcaaaattggatgtatcccgttaacattattgtcaaagtttaaatacctatatattctctatgactgcaataaagggacttccaggagagagtgagtgaaagtaatggtatggaggtgtcacctggggtcagcctaccctctgtgcatggccattggagattggggcttaactctttaggtttcctggaggctcaggaatggaaggattctgcccttccatgctttcccaccatggtttcagggttgaggaaaactcctttcagctttttgccccagatatcctacctctccttaaatcttgtaagggttcttaggatggctcttctagccagagactggcttatctttaaaacttgactgaactatgacttcaggaagggtcctgccactgcagaccatttctcctagtaccgtgtgtgatagaagacacactcactctgacctttatctgcttgattctttaagctatccagcttcacaccagctcccttcctaatggaagagtatagggaaagacctcctgtgtttgactttctaccttgtccaccttcccatatcttgggattgaaggacaaggcattaatctaaggagtgattaaggtggctggggctcagacatctgttgtgtcagtggtcaataaataggaaagtcccagttgaattcttgcccttaaatgcttttgctgctatttgtttgttcccagctaggaattctgcatcctgggacagtcagattccactagaacaggccaggaaggaagggaagagtgaaaggatggaattcccagacactccttcctcctgtcccttttcctagcagctctcagaccagatgttagctgctacacttccttccagaggtagggaatgtgtggtggctgtgagtggtctgtgttcttattgctggtagggtgatggagtgggctgaaaccatgcactctggaatttgttgtgtattttctctcagtaaagcttttttttgtttttcctgactgctgctctatgtgtgatatgtgagccattcacttgtgatggtctatttttgctcctttcagtatggtgcctgaccttagttttacaattttaggaagtccagcttggtatatccatgtttccaacagttgggctccagctggagactgtgtaagtggaacaggcctcacagttcagtgatacttcgaagatgagggaagggagagatactacttagggatattttcctgacctctgttccaggctgcagttaaggatttgggggtaaattttgctctccctcccttcccagtcatccaaagtctccaaaatcttggtcactcacagaaattctaaaatcagttaggtacagtacaatcaatcacctttattccagggttagaacaaggccgtgcacactgcagacagaggagcagagaatggggcagtctcacagcagtatgggggtaggagggcaggttagtctttttagattattttgccttacagagaaatttactacactccgctgaaaatgaaccctgtctcctttctctcatctcatcctctcctctcttcaaaggaagcctcgctttgaagagcctctttattccccgcttctctctctctctgactccatctcacgtaacgtatcctggcttggaaggacaattcatgctcattttccacctcccaattctacaagtatctctctctccttgagggagaaagccagacagggctggggaaacttgggaaggtcctttaaggcaaaactgtggagtactcgaggtgtatgttttaaggcaggtgtccgcagcagtggccagaggggacgggctgggctgggctgggctggtagagggtgctctccagccagtctggcttcttccctctccaacagccagcccttccctcccactgaagttttgttcccaacatcccaaaagctccccacctcacttaaactctctaaatagtctcttgagattgcatgtgaaattaaagtttaaaaaagttttctttttcgactatatagcaatttgactctgccactcccttcactgtggcattctccttccacccccgcccgggccttgacaataagttaatgctagacaccacagaaaaagggtacacatggcagtgcgggtttaatatacatatatgtagaatatatatgtacacacagttagcacggtcagggtggggagggctcactcgggacaggcaggaggctggggtcactgtactctagtgatttggctaggaggcgttcacaagcccaaggtgccaggatattgagggagaggggttactaagccccctcctggactgaattggggatcagtgcaacggaaacactggagttagcttcttcccttctttctaccttgggggaggggcttctagaccacggagagagggaacgttgcctgcaataaccacgttgggactctgtaggtggaaacaggactggaattaaaacctaaggatttctattctaggaactcccaaggctagtatgtgcaacagtctgagtaggaataaaggagtagaaaagaggactcctctctgccttcatcccctcaggctcagtggtgggggtcggtctgttcccagcattgtctctccagaggtgggggatggggctggaatctagtgctgggttctcatggggtttgaacttgggggccgggaaaggatccagtgatctctctcttcaggtccctggctcacacccaccctaggcctatgaaaggggagcaaaaggggacctgctggtgcccaggccctctaacctgaggaacagtcggaaacaggactcctcagccagtccagcattggggtacaaacacaaactctggcttctctttgcccctgccaggtgggggttgggtggcttcttcatctttcctctcactgcttttcattagattcacccaactctcccatgccttactcttgccttgcctcctcaatgtaggaaaacacaggtcctttctgctcccacacttggaggtgggggaagagtggaggaaagggaagtaagagaatgtgtctatttctatttgcatagaaatagcccccaactccccctggagtggggaaactgagccccaccaccacccccagcccctgacccccatcccctcacaaggaggaggaactagatgagctagacatgtagacagacagccagagcgtgcgcacacgcacacacacgcgcacagtaaggggttaggaccaggttagtaagtgggtagatgggggagggctgcccttgcccctcccctgcaggatggggaaagagtagtctgatgaaggtccaaccttcctcctcttcctcccccttgctcaggctttcttcggtggaggagccaatttgatgatctcttcctcagag

SEQ ID NO: 121 - DNA, 2374 bp, Bos taurus
  source 1..2374: mol_type = other DNA
agaggcgcgcggcgaccgagcggcagcagggagctggcttatagctcaccacgggtggggagacaaggagcttcagtgggcagcgagtggctggatccagcaagccctcccttccccacgcctgcagcatagaggcctactctgtccttggaacagcctctgtcaggacgcagaatcagccctcctggccatgggaggggcagtgatcgacgagggcccgaccggcatcaaggccccggatgggggctggggctgggccatcctttggggctgtttcgtcatcacgggcttctcctacgccttccccaaggcggtcagcgtcttcttcaaggaactcatgcgagagtttgggataggctacagtgacacagcctggatctcctccatcctgctggccatgctgtatgggacaggcccactctgcagcgtgtgtgtgaatcgctttggctgccggccagtcatgcttgcgggcggcctcttggcgtccctgggcatggtgtctgcgtccttctgtggaagcatcatccagctctacctcaccacaggggtccttactggcttgggtttggcgctcaacttccagccctcactcatcatgctcaaccgctacttcaacaagcggcgccccatggccaacgggctggcggcagcaggcagcccggtgttcctgtgcgccctgtccccgctggggcaggtgctgcaggaccactatggatggcggggcggcttcctcatcctgggtggcctgctgctcaattgctgcgtgtgtgccgcgctcatgcggcccctggaggcgccccggcagagttcaggttcggggcctgcaccccagcggccgccccggcggctgttggacctgagcgtcttcagggaccgtggcttcgtcatctatgccctggccgcctccatcatggtgctggggctctttgtaccacctgtgtttgtggtgagctatgccaaggacctgggggtgcccgacactcaggcggccttcctgctcaccatcctgggcttcgtcgacatctttgctaggcccaccgctggcttcatcacggggctcaagaaggtgcggccctactctgtctacctcttcagctttgccatgttcttcaacggcttcgctgacctcacggggtccactgccagcaactacggtgggctggtggtcttctgcatcttcttcggcatctcctatggcatggtgggggccctgcagttcgaggtgctcatggccatcgtgggcacccagaagttctccagcgccattggcctcgtgctgctgctggaggccatagctgtgctcattgggccgccgtccggaggcaagctcctggatgcgacgcacgtctaccagtacgtgtttatcctggcgggggccgaggtgctggcctcctcccttgtgctggtgctgggtaacttcttctgcattaggaagaggcccgagacggccacggaggaggagcgccacaagcccccggaggatgtcagggtggactcccgggaggtggagcagttcctgaagacagagcctgagaagaacggggaggtggttcacaccccggaaacgagcgtctgagcccagggaggacggcaggggtgcagggaaccggacagagaccgtcaacgctcgtatttatttcacaaacaggaccagcttgggcggggccttcggctctgcgccggctgcccgggcagtggatcatcacccgatcagtgtttttcaggggaaggtggcggggtgggaacgtgtcattccgaggcggatctgtggtgaagccaagccgcgaggttatgagccgtctgcaccagggaacccacctgctgaccccagggagcccagtgcccaccgctgggctcaaggacccagagacccgacgctttggagacaacacgactttaatcagggggcggggccggggacagctacggagcgggcactgccacaacaggcctccccatgtgcccatgagtgccctgtcctaccgggtctcctgtgtgccccagcccaccccttttgagtgtcttagtggacaaagctccctccttcgcctctgaaaggtttgagataaacctgctcgctcttagggacagcggtctaaccgttgaccccaaatctctttctcaaaagacagcctgcttatttttacaaattggtttcagtcctcagcagccacgtcagccttctaagtactgctcgttctggaccctgtcctgctggacctcaaacggctcagcgtgttcacacagcaccctggtggtcggcagggaaggggccacactgcggggatgtggacacctaggctgggatgtacatgtggtttctgttcactggtgtgtgtttgaagaaaatgatctgcctgcttttgtttattca

SEQ ID NO: 122 - DNA, 1305 bp, Bos taurus
  source 1..1305: mol_type = other DNA
cgctgaggtccttggtcctctggctccgggcgtgggcgggatgacgcgcttcgctctgaccgtggtccggcatggagaaacaagacttaacaaggagaaaataattcaaggacaaggaatagatgagcctctttcagaaactggatttaaacaagcagctgctgctggtatatttcttaaagatgtgaagtttactcatgttttctccagtgaccttacaaggacaaagcagaccgtgcatgggattttggagaagagcaaattttgcaaagatatgacagtaaagtatgattcaagactccgagaaaggaaatatggggttgcagaaggcaggccgctgagcgagctgagggccatggccaaagcggcaggggaggagtgcccagcattcacaccgcctggaggagagaccttggaccagctgaaaaggcgtgggaaagacttttttgaatttctttgtcaactgatcctgaaagaagccggtcagaatgaacagttttcccaggaagccccgagcagctgtctggaaagttccctggcagagatatttcctttagggaagaactgtgcctccacgtttaactctgacagcggcaccccgggcctagcagccagtgtcttagtcgtgagtcatggcgcctacatacgaagcctgttggattactttctgactgacctcaagtgctcgttcccagcgactctgagcaggtccgagctcacgtcagtcagccctaacacagggatgactgtcttcatcctaaactttgagaaaggaggcaaagggagaccaactgcccagtgtgtgtgtgtgaacctccagggccacctggccggggtgaacaaaactccctaaatctgaatcatctgcatggaaatcagcacatgtgagcctctgagggagtgcctcttgactttacttccgtcctctgctaatgcagatttggaaacagttaaaaagctgtctgttacagcaggttataaaactcgagtggaatcagagccatgtaaaacactaatagaaaaccatcgaaaatggacttctttggtgcaagtacagttggaattttccagggtaattttaccaccctgctcagtgacctctctgtattgtaaatggatgagggaactcagtattgtgaattgaggcatgaataaccctgttactatggtttggggtttttttttggttttttttttcacctttttaatatcttttttttttttttaatgtttttcttttatgtctgaaaaatctaacttgttttcttggaaaaaaaaaaaaaaaaaaaaaaaaaaaaaaaaaaa

SEQ ID NO: 123 - DNA, 1068 bp, Bos taurus
  source 1..1068: mol_type = other DNA
atgcagtccaaacgcgaatgtgagctgtggtgtgagagggtgaatcccgaaaacaaggcggcgttggaggcgtgggtcagggagacgggcatccggctggtgcaggtgaacgggcagagaaagtatggcgggccacccccaggatgggtgggcagcccgccgccggccgggtcggaggtgtttatcgggcggctgccccaggacgtgtacgagcaccagctgattccactgttccagcgcgtgggccgcctctacgagtttcgtctgatgatgaccttcagcggcctgaaccgcggcttcgcttacgcccgctacagctcgcggcgcggcgcccaagccgccatcgccacgctgcacaaccacctgctgcggccgtcctgcccgctgctcgtgtgccgcagcaccgagaagtgcgagctgagcgtggacgggctgccgccgggcctgagccaccgcgcgctactccgcgcgctgcagccgctgggacccggcctgcaggaggcgctgctgctgcctagccccgggccgacgcccgcacagatagcactgctcaagttcagctcgcaccgggccgcagccatggccaaaaaggccctggtggaagggcagtcccacctctgtggagaacaggtggctgtggagtggcttaagccagacctgaagcagcgactccgccagcagctcatgggtacctcgctacagtgcctacagcctgagggcaaccgattagccctggccagggaccaggggctcgagttccaagggtctcaggttgccttgcagctgctgtgccaacggatgaagctgggcagcccagtgttcctcaccaagtgtttgggcacaagccctgctggctggcaccgcttctggtaccaggtggtgatccccgggcatccggtgcccttcagtggcctcatctgggttgtgctgaccccagatgggcaggatgggcatgaggtggctaaggatgcagtatctgcacggctgctggaagcaatgagtgagtctagggccagcctcctttggtctactggggctgaggcaggaggtaccatggttcaacagtga

SEQ ID NO: 124 - DNA, 20 bp, Sus scrofa
  source 1..20: mol_type = other DNA
tgggctgaaaaatagcattt

SEQ ID NO: 125 - DNA, 20 bp, Sus scrofa
  source 1..20: mol_type = other DNA
aaccagcctgggtttcctgt

SEQ ID NO: 126 - DNA, 26 bp, Sus scrofa
  source 1..26: mol_type = other DNA
ttttcaacccacaggaaacccaggct

SEQ ID NO: 127 - DNA, 20 bp, Sus scrofa
  source 1..20: mol_type = other DNA
caaccagcctgggtttcctg

SEQ ID NO: 128 - DNA, 26 bp, Sus scrofa
  source 1..26: mol_type = other DNA
tttcaacccacaggaaacccaggctg

SEQ ID NO: 129 - DNA, 20 bp, Sus scrofa
  source 1..20: mol_type = other DNA
aatgctatttttcaacccac

SEQ ID NO: 130 - DNA, 20 bp, Sus scrofa
  source 1..20: mol_type = other DNA
tttcaacccacaggaaaccc

SEQ ID NO: 131 - DNA, 20 bp, Sus scrofa
  source 1..20: mol_type = other DNA
ctgtgattctgacttctctc

SEQ ID NO: 132 - DNA, 20 bp, Sus scrofa
  source 1..20: mol_type = other DNA
aagtacaacatggagacacg

SEQ ID NO: 133 - DNA, 20 bp, Sus scrofa
  source 1..20: mol_type = other DNA
ggtcgtgttgaagtacaaca

SEQ ID NO: 134 - DNA, 20 bp, Sus scrofa
  source 1..20: mol_type = other DNA
agtacaacatggagacacgt

SEQ ID NO: 135 - DNA, 20 bp, Sus scrofa
  source 1..20: mol_type = other DNA
gtacaacatggagacacgtg

SEQ ID NO: 136 - DNA, 20 bp, Sus scrofa
  source 1..20: mol_type = other DNA
tgattctgacttctctctgg

SEQ ID NO: 137 - DNA, 20 bp, Sus scrofa
  source 1..20: mol_type = other DNA
ctttctcacttttcactctc

SEQ ID NO: 138 - DNA, 20 bp, Sus scrofa
  source 1..20: mol_type = other DNA
ctggcttactcctatcatga

SEQ ID NO: 139 - DNA, 20 bp, Sus scrofa
  source 1..20: mol_type = other DNA
cctatcatgaaggaaaatat

SEQ ID NO: 140 - DNA, 20 bp, Bos taurus
  source 1..20: mol_type = other DNA
agcataggtgggtcccgctg